(19) United States
(12) Patent Application Publication
(10) Pub. No.: US 2026/0123274 A1
(43) Pub. Date: Apr. 30, 2026

(54) COMPOUND FOR ORGANIC OPTOELECTRONIC DEVICE, COMPOSITION FOR ORGANIC OPTOELECTRONIC DEVICE, ORGANIC OPTOELECTRONIC DEVICE, AND DISPLAY DEVICE

(71) Applicant: SAMSUNG SDI CO., LTD., Yongin-si, Gyeonggi-do (KR)

(72) Inventors: Hanill LEE, Suwon-si, Gyeonggi-do (KR); Sunghyun JUNG, Suwon-si, Gyeonggi-do (KR); Hyung Sun KIM, Suwon-si, Gyeonggi-do (KR); Yoonman LEE, Suwon-si, Gyeonggi-do (KR); Byungku KIM, Suwon-si, Gyeonggi-do (KE); Wook KIM, Suwon-si, Gyeonggi-do (KR); Seungjae LEE, Suwon-si, Gyeonggi-do (KR); Seonyeong GWAK, Suwon-si, Gyeonggi-do (KR); Suheon IM, Suwon-si, Gyeonggi-do (KR); Hyungyu LEE, Suwon-si, Gyeonggi-do (KR); Dongkyu RYU, Suwon-si, Gyeonggi-do (KR); Chang ju SHIN, Suwon-si, Gyeonggi-do (KR)

(21) Appl. No.: 19/116,016

(22) PCT Filed: Oct. 17, 2023
(86) PCT No.: PCT/KR2023/016006

§ 371 Date: Mar. 27, 2025

(30) Foreign Application Priority Data
Oct. 18, 2022 (KR) 10-2022-0133894

(51) Int. Cl. H10K 85/60; C07D 209/86; C07D 405/14; C07D 409/14; C07D 487/04; C07F 7/08; C09K 11/02; H10K 50/12; H10K 85/40

(52) U.S. Cl. CPC H10K 85/622; C07D 209/86; C07D 405/14; C07D 409/14; C07D 487/04; C07F 7/0816; C09K 11/02; H10K 85/40; H10K 85/654; H10K 85/6572; H10K 85/6574; H10K 85/6576; H10K 50/12

(57) ABSTRACT

Disclosed are a compound for an organic optoelectronic device represented by Chemical Formula 1, an organic optoelectronic device including the same, and a display device. The details of Chemical Formula 1 are as defined in the specification.

### TECHNICAL FIELD

A compound for an organic optoelectronic device, a composition for an organic optoelectronic device, an organic optoelectronic device, and a display device are disclosed.

### BACKGROUND ART

An organic optoelectronic device (organic optoelectronic diode) is a device capable of converting electrical energy and optical energy to each other.

Organic optoelectronic devices may be largely divided into two types according to a principle of operation. One is a photoelectric device that generates electrical energy by separating excitons formed by light energy into electrons and holes, and transferring the electrons and holes to different electrodes, respectively and the other is light emitting device that generates light energy from electrical energy by supplying voltage or current to the electrodes.

Examples of the organic optoelectronic device include an organic photoelectric device, an organic light emitting diode, an organic solar cell, and an organic photoconductor drum.

Among them, organic light emitting diodes (OLEDs) are attracting much attention in recent years due to increasing demands for flat panel display devices. The organic light emitting diode is a device that converts electrical energy into light, and the performance of the organic light emitting diode is greatly influenced by an organic material between electrodes.

### DISCLOSURE

### Technical Problem

An embodiment provides a compound for an organic optoelectronic device capable of realizing high-efficiency and long life-span organic optoelectronic device.

Another embodiment provides a composition for an organic optoelectronic device including the compound for an organic optoelectronic device.

Another embodiment provides an organic optoelectronic device including the compound for an organic optoelectronic device.

Another embodiment provides a display device including the organic optoelectronic device.

### Technical Solution

According to an embodiment, a compound for an organic optoelectronic device represented by Chemical Formula 1 is provided.

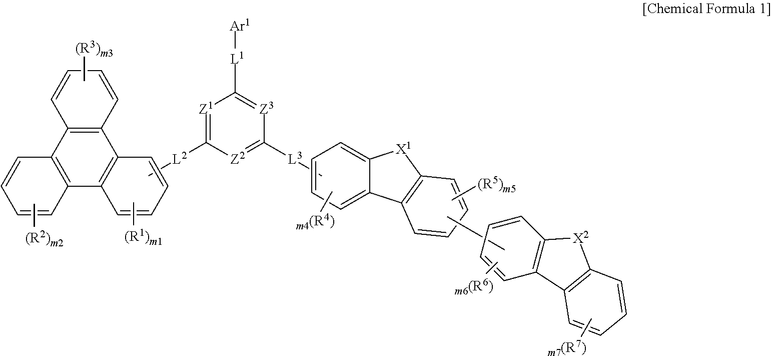

In Chemical Formula 1,
X¹ and X² are each independently O, S, or SiR^{a}R^{b},
Z¹ to Z³ are each independently N or CR^{c},
at least one of Z¹ to Z³ is N,
R^{a}, R^{b}, R^{c}, and R¹ to R⁷ are each independently hydrogen, deuterium, a cyano group, a substituted or unsubstituted C1 to C10 alkyl group, a substituted or unsubstituted C6 to C20 aryl group, or a substituted or unsubstituted C2 to C20 heterocyclic group,
R⁴ to R⁷ are each independently present or adjacent groups are linked to form a substituted or unsubstituted aromatic monocyclic ring or a substituted or unsubstituted aromatic polycyclic ring,
Ar¹ is a substituted or unsubstituted C6 to C20 aryl group or a substituted or unsubstituted C2 to C20 heterocyclic group,
L¹ to L³ are each independently a single bond or a substituted or unsubstituted C6 to C20 arylene group,
m1, m4, m5, and m6 are each independently one of integers of 1 to 3, and
m2, m3, and m7 are each independently one of integers of 1 to 4.

According to another embodiment, a composition for an organic optoelectronic device includes a first compound and a second compound.

The first compound is the aforementioned compound for the organic optoelectronic device, and the second compound may be represented by Chemical Formula 2; or a combination of Chemical Formula 3 and Chemical Formula 4.

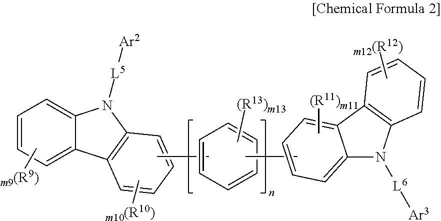

In Chemical Formula 2,
R⁹ to R¹³ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group,
Ar² and Ar³ are each independently a substituted or unsubstituted C6 to C20 aryl group or a substituted or unsubstituted C2 to C30 heterocyclic group,
L⁵ and L⁶ are each independently a single bond or a substituted or unsubstituted C6 to C20 arylene group,
m9, m12, and m13 are each independently one of integers of 1 to 4,
m10 and m11 are each independently one of integers of 1 to 3, and
n is one of integers of 0 to 2;

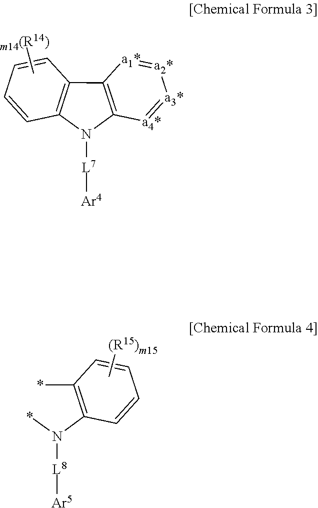

wherein, in Chemical Formula 3 and Chemical Formula 4,
a₁* to a₄* in Chemical Formula 3 are each independently a linking carbon (C) or C-L^{a}-R^{d},
among a₁* to a₄* in Chemical Formula 3, two adjacent ones are each linked to *s in Chemical Formula 4,
L^{a}, L⁷, and L⁸ are each independently a single bond or a substituted or unsubstituted C6 to C20 arylene group,
R^{d}, R¹⁴, and R¹⁵ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group,
Ar⁴ and Ar⁵ are each independently a substituted or unsubstituted C6 to C20 aryl group or a substituted or unsubstituted C2 to C30 heterocyclic group, and
m14 and m15 are each independently one of integers of 1 to 4.

According to another embodiment, an organic optoelectronic device includes an anode and a cathode facing each other, and at least one organic layer between the anode and the cathode, wherein the organic layer includes the aforementioned compound for an organic optoelectronic device.

According to another embodiment, a display device including the organic optoelectronic device is provided.

### Advantageous Effects

High-efficiency and long life-span organic optoelectronic devices may be realized.

### DESCRIPTION OF THE DRAWINGS

FIG. 1 is a cross-sectional view illustrating an organic light emitting diode according to an embodiment.

### DESCRIPTION OF SYMBOLS

100: organic light emitting diode
105: organic layer
110: cathode
120: anode
130: light emitting layer
140: hole transport region
150: electron transport region

### BEST MODE

Hereinafter, embodiments of the present invention are described in detail. However, these embodiments are exemplary, and this disclosure is not limited thereto.

As used herein, when a definition is not otherwise provided, “substituted” refers to replacement of at least one hydrogen of a substituent or a compound by deuterium, a halogen, a hydroxyl group, an amino group, a substituted or unsubstituted C1 to C30 amine group, a nitro group, a substituted or unsubstituted C1 to C40 silyl group, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, a C1 to C20 alkoxy group, a C1 to C10 trifluoroalkyl group, a cyano group, or a combination thereof.

In one example of the present invention, the “substituted” refers to replacement of at least one hydrogen of a substituent or a compound by deuterium, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, or a cyano group. In specific example of the present invention, the “substituted” refers to replacement of at least one hydrogen of a substituent or a compound by deuterium, a C1 to C20 alkyl group, a C6 to C30 aryl group, or a cyano group. In specific example of the present invention, the “substituted” refers to replacement of at least one hydrogen of a substituent or a compound by deuterium, a C1 to C5 alkyl group, a C6 to C18 aryl group, or a cyano group. In specific example of the present invention, the “substituted” refers to replacement of at least one hydrogen of a substituent or a compound by deuterium, a cyano group, a methyl group, an ethyl group, a propyl group, a butyl group, a phenyl group, a biphenyl group, a terphenyl group, or a naphthyl group.

In the present specification, “unsubstituted” refers to non-replacement of a hydrogen atom by another substituent and remaining of the hydrogen atom.

In the present specification, “hydrogen substitution (—H)” may include “deuterium substitution (-D)” or “tritium substitution (-T).”

In the present specification, when a definition is not otherwise provided, “hetero” refers to one including one to three heteroatoms selected from N, O, S, P, and Si, and remaining carbons in one functional group.

In the present specification, “aryl group” refers to a group including at least one hydrocarbon aromatic moiety, and all elements of the hydrocarbon aromatic moiety have p-orbitals which form conjugation, for example a phenyl group, a naphthyl group, and the like, two or more hydrocarbon aromatic moieties may be linked by a sigma bond and may be, for example a biphenyl group, a terphenyl group, a quaterphenyl group, and the like, and two or more hydrocarbon aromatic moieties are fused directly or indirectly to provide a non-aromatic fused ring, for example a fluorenyl group.

The aryl group may include a monocyclic, polycyclic, or fused ring polycyclic (i.e., rings sharing adjacent pairs of carbon atoms) functional group.

As used herein, “heterocyclic group” is a generic concept of a heteroaryl group, and may include at least one heteroatom selected from N, O, S, P, and Si instead of carbon (C) in a cyclic compound such as aryl group, a cycloalkyl group, a fused ring thereof, or a combination thereof. When the heterocyclic group is a fused ring, the entire ring or each ring of the heterocyclic group may include one or more heteroatoms.

For example, “heteroaryl group” may refer to aryl group including at least one heteroatom selected from N, O, S, P, and Si. Two or more heteroaryl groups are linked by a sigma bond directly, or when the heteroaryl group includes two or more rings, the two or more rings may be fused. When the heteroaryl group is a fused ring, each ring may include one to three heteroatoms.

More specifically, the substituted or unsubstituted C6 to C30 aryl group may be a substituted or unsubstituted phenyl group, a substituted or unsubstituted naphthyl group, a substituted or unsubstituted anthracenyl group, a substituted or unsubstituted phenanthrenyl group, a substituted or unsubstituted naphthacenyl group, a substituted or unsubstituted pyrenyl group, a substituted or unsubstituted biphenyl group, a substituted or unsubstituted p-terphenyl group, a substituted or unsubstituted m-terphenyl group, a substituted or unsubstituted o-terphenyl group, a substituted or unsubstituted chrysenyl group, a substituted or unsubstituted benzophenanthrenyl group, a substituted or unsubstituted triphenylene group, a substituted or unsubstituted perylenyl group, a substituted or unsubstituted fluorenyl group, a substituted or unsubstituted indenyl group, or a combination thereof, but is not limited thereto.

More specifically, the substituted or unsubstituted C2 to C30 heterocyclic group may be a substituted or unsubstituted furanyl group, a substituted or unsubstituted thiophenyl group, a substituted or unsubstituted pyrrolyl group, a substituted or unsubstituted pyrazolyl group, a substituted or unsubstituted imidazolyl group, a substituted or unsubstituted triazolyl group, a substituted or unsubstituted oxazolyl group, a substituted or unsubstituted thiazolyl group, a substituted or unsubstituted oxadiazolyl group, a substituted or unsubstituted thiadiazolyl group, a substituted or unsubstituted pyridyl group, a substituted or unsubstituted pyrimidinyl group, a substituted or unsubstituted pyrazinyl group, a substituted or unsubstituted triazinyl group, a substituted or unsubstituted benzofuranyl group, a substituted or unsubstituted benzothiophenyl group, a substituted or unsubstituted benzimidazolyl group, a substituted or unsubstituted indolyl group, a substituted or unsubstituted quinolinyl group, a substituted or unsubstituted isoquinolinyl group, a substituted or unsubstituted quinazolinyl group, a substituted or unsubstituted quinoxalinyl group, a substituted or unsubstituted naphthyridinyl group, a substituted or unsubstituted benzoxazinyl group, a substituted or unsubstituted benzthiazinyl group, a substituted or unsubstituted acridinyl group, a substituted or unsubstituted phenazinyl group, a substituted or unsubstituted phenothiazinyl group, a substituted or unsubstituted phenoxazinyl group, a substituted or unsubstituted carbazolyl group, a substituted or unsubstituted dibenzofuranyl group, or a substituted or unsubstituted dibenzothiophenyl group, a substituted or unsubstituted benzonaphthofuranyl group, a substituted or unsubstituted benzonaphthothiophenyl group, a substituted or unsubstituted benzofuranofluorenyl group, a substituted or unsubstituted benzothiophenefluorenyl group, or a combination thereof, but is not limited thereto.

As used herein, hole characteristics refer to an ability to donate an electron to form a hole when an electric field is applied and that a hole formed in the anode may be easily injected into the light emitting layer and transported in the light emitting layer due to conductive characteristics according to a highest occupied molecular orbital (HOMO) level.

In addition, electron characteristics refer to an ability to accept an electron when an electric field is applied and that electron formed in the cathode may be easily injected into the light emitting layer and transported in the light emitting layer due to conductive characteristics according to a lowest unoccupied molecular orbital (LUMO) level.

Hereinafter, a compound for an organic optoelectronic device according to an embodiment is described.

A compound for an organic optoelectronic device according to an embodiment is represented by Chemical Formula 1.

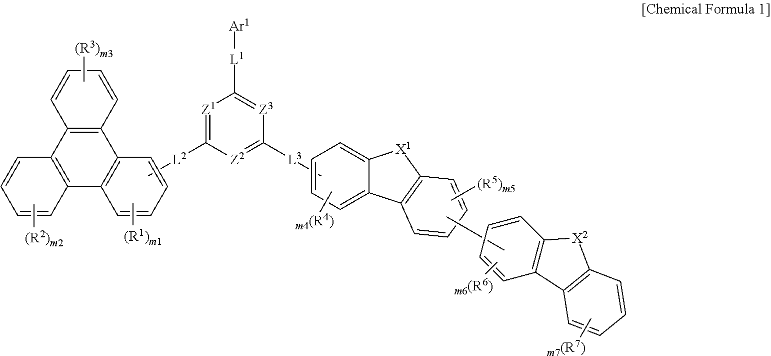

In Chemical Formula 1,
X¹ and X² are each independently O, S, or SiR^{a}R^{b},
Z¹ to Z³ are each independently N or CR^{c},
at least one of Z¹ to Z³ is N,
R^{a}, R^{b}, R^{c} and R¹ to R⁷ are each independently hydrogen, deuterium, a cyano group, a substituted or unsubstituted C1 to C10 alkyl group, a substituted or unsubstituted C6 to C20 aryl group or a substituted or unsubstituted C2 to C20 heterocyclic group,
R⁴ to R⁷ are each independently present or adjacent groups are linked to form a substituted or unsubstituted aromatic monocyclic ring or a substituted or unsubstituted aromatic polycyclic ring,
Ar¹ is a substituted or unsubstituted C6 to C20 aryl group or a substituted or unsubstituted C2 to C20 heterocyclic group,
L¹ to L³ are each independently a single bond or a substituted or unsubstituted C6 to C20 arylene group,
m1, m4, m5, and m6 are each independently one of integers of 1 to 3, and
m2 and m3 are each independently one of integers of 1 to 4.

The compound represented by Chemical Formula 1 has a structure including triphenylene, and two consecutive dibenzofuran derivatives (substituents selected from dibenzofuran, dibenzothiophene, and dibenzosilole) in the center of a 6-membered nitrogen-containing ring including at least one nitrogen atom.

By including the two consecutive dibenzofuran derivatives, electron delocalization may be achieved, thereby increasing charge mobility. Accordingly, the hole transport characteristics may be further improved, so that low-driving and high-efficiency performances of organic optoelectronic devices including the same may be realized.

In addition, by introducing the triphenylene, the LUMO electron cloud of the nitrogen-containing six-membered ring may be expanded, thereby preventing deterioration by the anion.

In addition, by designing the three substituents differently in the center of the 6-membered nitrogen-containing ring, steric hindrance is achieved, resulting in a low deposition temperature, and thus life-span characteristics of the organic light emitting diode to which it is applied may be significantly improved.

In Chemical Formula 1, when two or more R¹s are substituted, each R¹ may be the same or different from each other.

In Chemical Formula 1, when two or more R²s are substituted, each R² may be the same or different from each other.

In Chemical Formula 1, when two or more R³s are substituted, each R³ may be the same or different from each other.

In Chemical Formula 1, when two or more R⁴s are substituted, each R⁴ may be the same or different from each other.

In Chemical Formula 1, when two or more R⁵s are substituted, each R⁵ may be the same or different from each other.

In Chemical Formula 1, when two or more R⁶s are substituted, each R⁶ may be the same or different from each other.

In the above chemical formula 1, when two or more R⁷s are substituted, each R⁷ may be the same or different from each other.

In the above chemical formula 1, when two or more R^{c}s are substituted, each R^{c} may be the same or different from each other.

For example, Chemical Formula 1 may be represented by any one of Chemical Formula 1-1 to Chemical Formula 1-4.

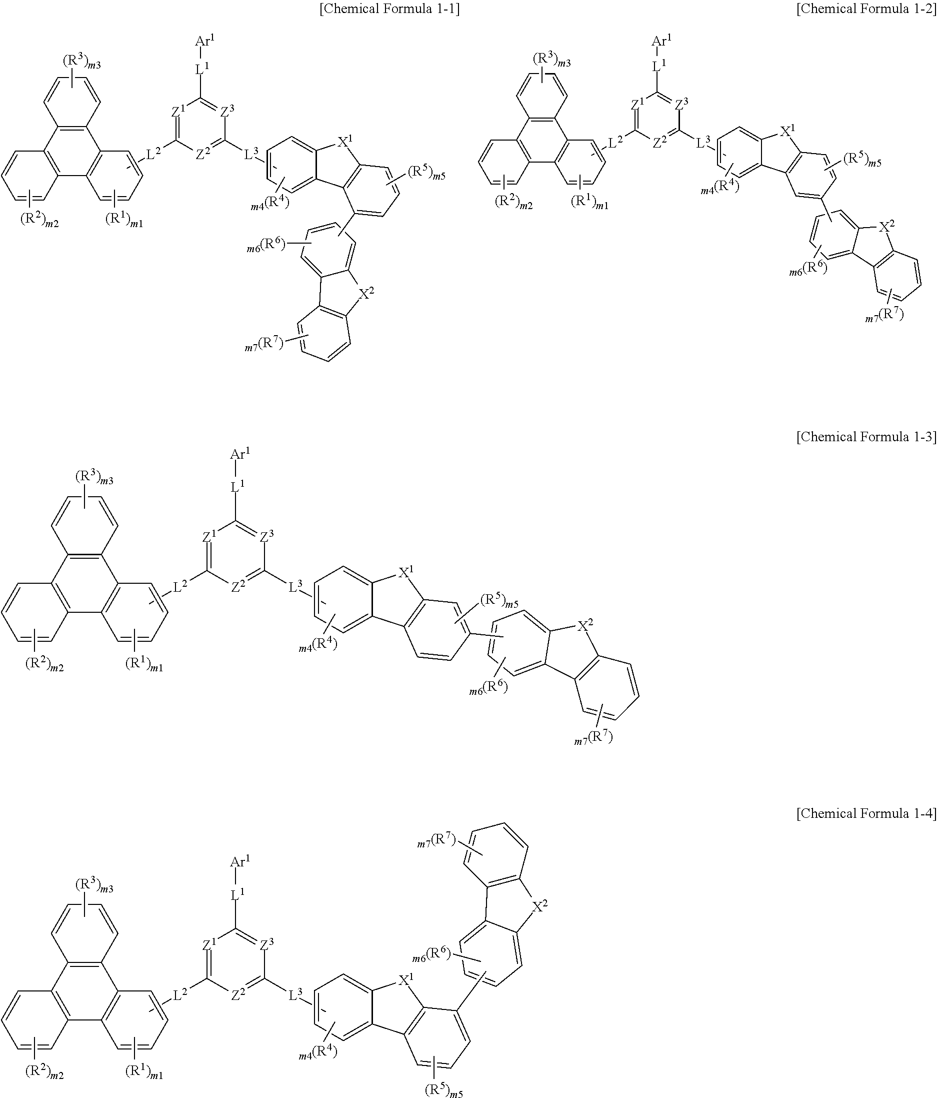

In Chemical Formula 1-1 to Chemical Formula 1-4,
the definitions of X¹, X², Z¹ to Z³, R¹ to R⁷, Ar¹, L¹ to L³, and m1 to m7 are the same as described above.

For example, Chemical Formula 1 may be represented by Chemical Formula 1-1 or Chemical Formula 1-4.

Chemical Formula 1-1 may be represented by any one of Chemical Formula 1-1-(i), Chemical Formula 1-1-(ii), Chemical Formula 1-1-(iii), and Chemical Formula 1-1-(iv).

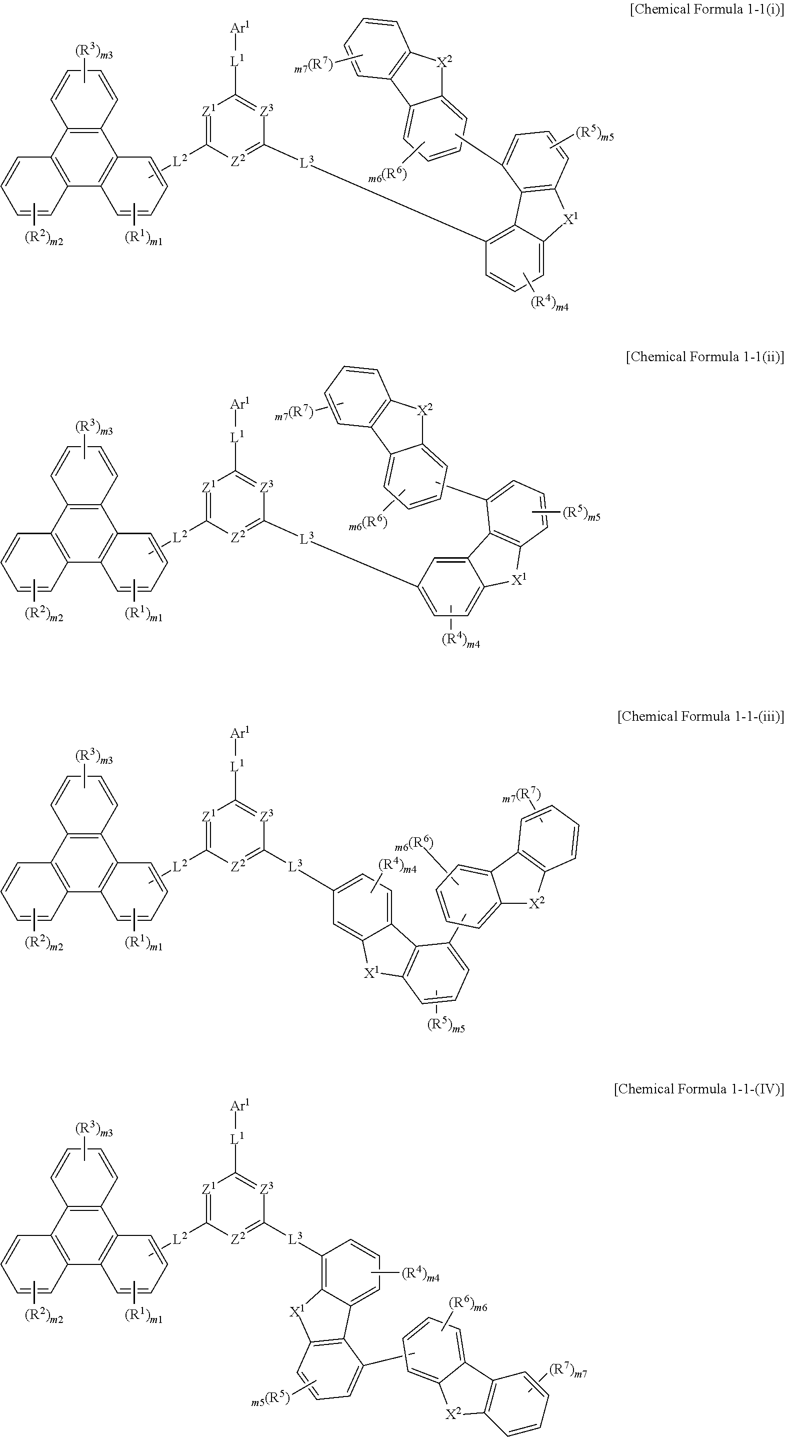

In Chemical Formula 1-1-(i), Chemical Formula 1-1-(ii), Chemical Formula 1-1-(iii), and Chemical Formula 1-1-(iv),
the definitions of X¹, X², Z¹ to Z³, R¹ to R⁷, Ar¹, L¹ to L³, and m1 to m7 are the same as described above.

Chemical Formula 1-4 may be represented by any one of Chemical Formula 1-4-(i), Chemical Formula 1-4-(ii), Chemical Formula 1-4-(iii), and Chemical Formula 1-4-(iv).

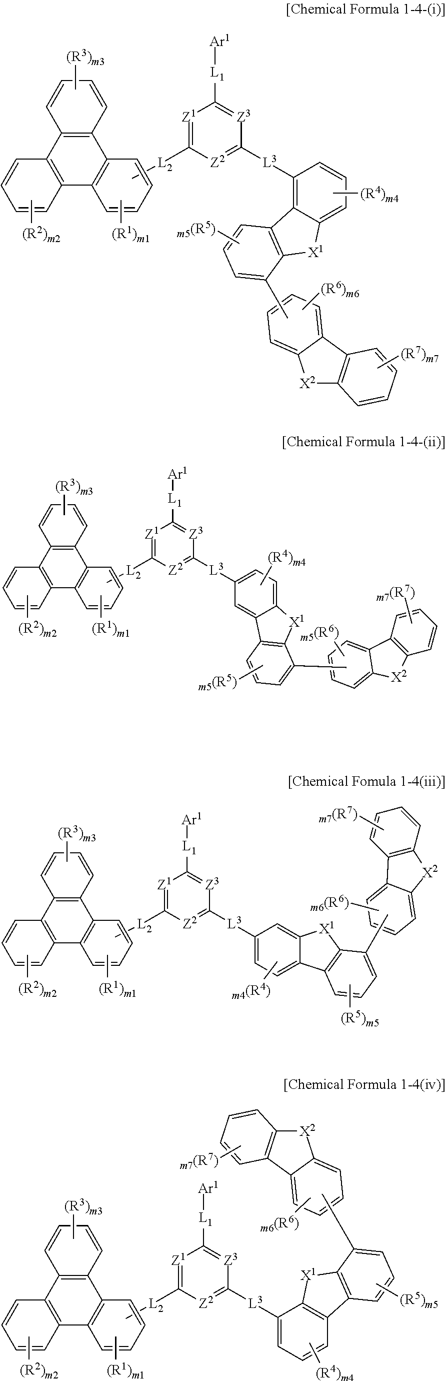

In Chemical Formula 1-4-(i), Chemical Formula 1-4-(ii), Chemical Formula 1-4-(iii), and Chemical Formula 1-4-(iv),
the definitions of X¹, X², Z¹ to Z³, R¹ to R⁷, Ar¹, L¹ to L³, and m1 to m7 are the same as described above.

For example, Chemical Formula 1 may be represented by any one of Chemical Formula 1-1-(i), Chemical Formula 1-1-(ii), Chemical Formula 1-1-(iii), Chemical Formula 1-1-(iv), and Chemical Formula 1-4-(iv).

In particular, when a dibenzofuran derivative adjacent to a nitrogen-containing 6-membered ring is linked at 1st or 2nd position in the direction of the nitrogen-containing 6-membered ring, as in Chemical Formula 1-1-(i) or Chemical Formula 1-1-(ii), structural torsion may be maximized as steric hindrance with respect to 9th position increases further, and this structural torsion significantly lowers the energy barrier that the unshared electron pairs of O, S, and Si in the dibenzofuran derivative can hope for, thereby facilitating charge transfer between molecules. In addition, the molecular stability of the dibenzofuran derivative itself may be increased by Ar¹, which can contribute to improving the life-span of the device.

For example, Ar¹ may be a substituted or unsubstituted phenyl group, a substituted or unsubstituted biphenyl group, a substituted or unsubstituted fluorenyl group, a substituted or unsubstituted dibenzofuranyl group, a substituted or unsubstituted dibenzothiophenyl group, a substituted or unsubstituted dibenzosilolyl group, or a substituted or unsubstituted carbazolyl group.

As Ar¹, which may further increase the molecular stability of the dibenzofuran derivative itself, examples thereof may include a substituent other than triphenylene and two consecutive dibenzofuran derivatives, which are substituted or unsubstituted within the above-mentioned range.

For example, in Chemical Formula 1, Li may be a single bond or a substituted or unsubstituted phenylene group.

For example, in Chemical Formula 1, L² and L³ may each independently be a single bond or a substituted or unsubstituted C6 to C12 arylene group.

For example, in Chemical Formula 1, L² and L³ may each independently be a single bond, or a substituted or unsubstituted phenylene group.

For example, at least two of Z¹ to Z³ may be N.

For example, Z¹ to Z³ may each be N.

For example, R^{a} and R^{b} may each independently be a substituted or unsubstituted C1 to C10 alkyl group, or a substituted or unsubstituted C6 to C12 aryl group.

For example, R¹ to R⁷ may each independently be hydrogen, deuterium, a cyano group, a substituted or unsubstituted C1 to C10 alkyl group, a substituted or unsubstituted phenyl group, a substituted or unsubstituted biphenyl group, a substituted or unsubstituted fluorenyl group, a substituted or unsubstituted carbazolyl group, a substituted or unsubstituted dibenzofuranyl group, a substituted or unsubstituted dibenzothiophenyl group, or a substituted or unsubstituted dibenzosilolyl group.

In an embodiment, when R⁴ to R⁷ are each independently present, they may include a form in which two consecutive dibenzofuran derivatives are linked.

In another embodiment, R⁴ to R⁷ may be linked to adjacent groups to form a substituted or unsubstituted aromatic monocyclic ring, and
for example, they may be represented by any one of Chemical Formula 1A to Chemical Formula 1D D.

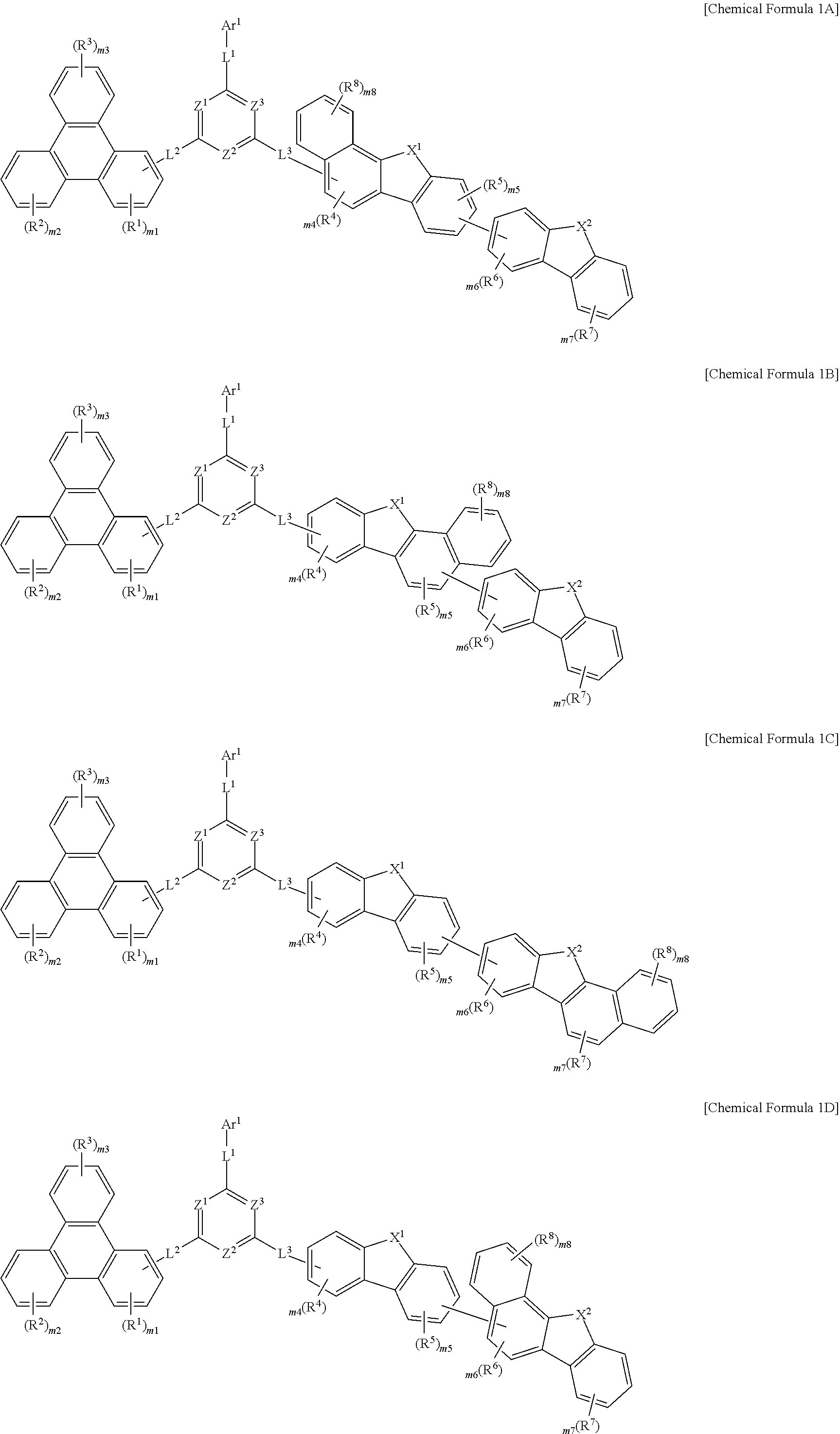

In Chemical Formula 1A to Chemical Formula 1D, the definitions of X¹, X², Z¹ to Z³, Ar¹, L¹ to L³, and m1 to m7 are the same as described above,
R¹ to R⁸ are each independently hydrogen, deuterium, a cyano group, a substituted or unsubstituted C1 to C10 alkyl group, a substituted or unsubstituted C6 to C20 aryl group or a substituted or unsubstituted C2 to C20 heterocyclic group, and
m8 is one of integers of 1 to 4.

In Chemical Formula 1A to Chemical Formula 1D, when two or more R⁸s are substituted, each R⁸ may be the same or different from each other.

As a most specific example, Chemical Formula 1 may be represented by any one of the aforementioned Chemical Formula 1-1-(i), Chemical Formula 1-1-(ii), Chemical Formula 1-1-(iii), Chemical Formula 1-1-(iv), and Chemical Formula 1-4-(iv).

In the most specific embodiment, the compound represented by Chemical Formula 1 may be one selected from the compounds listed in Group 1, but is not limited thereto.

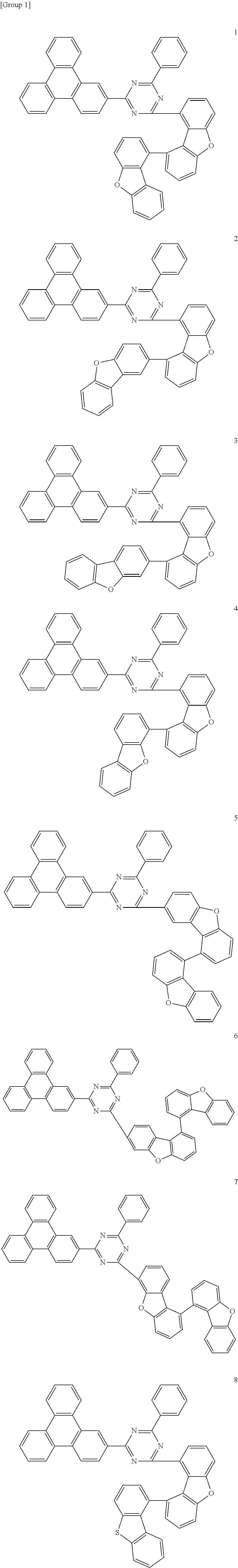

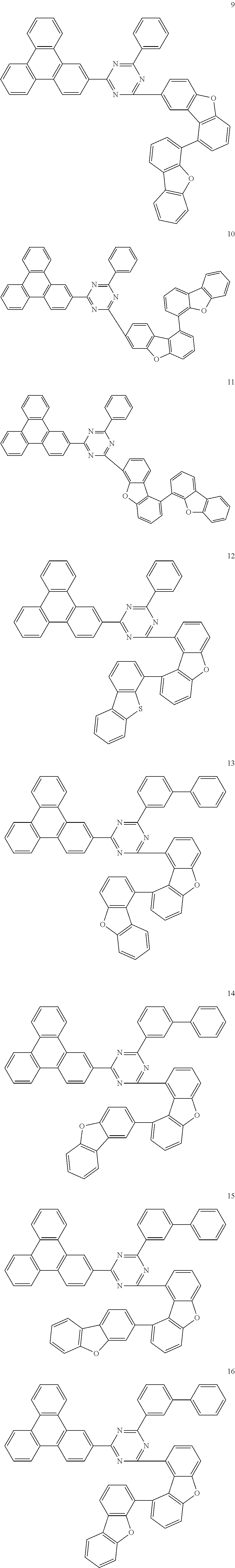

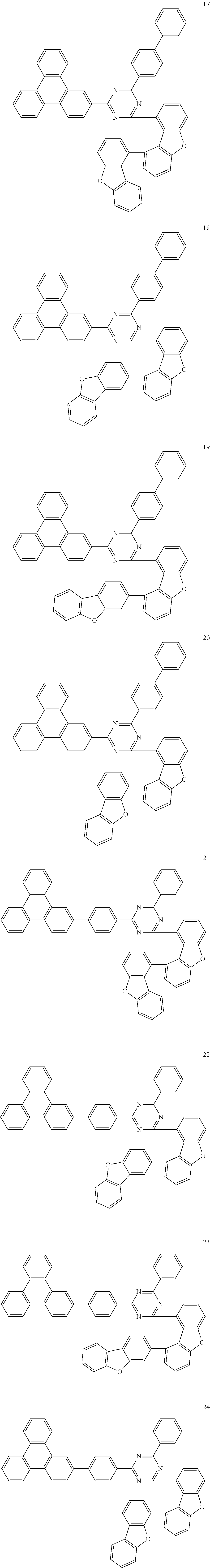

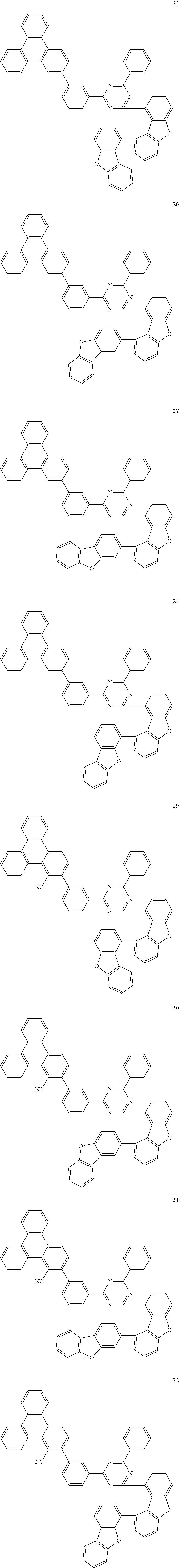

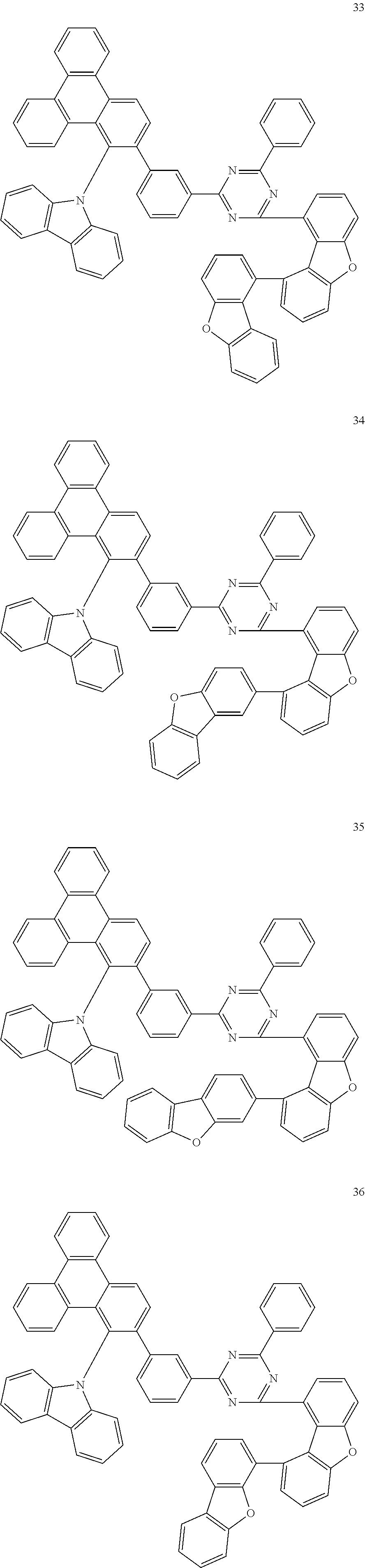

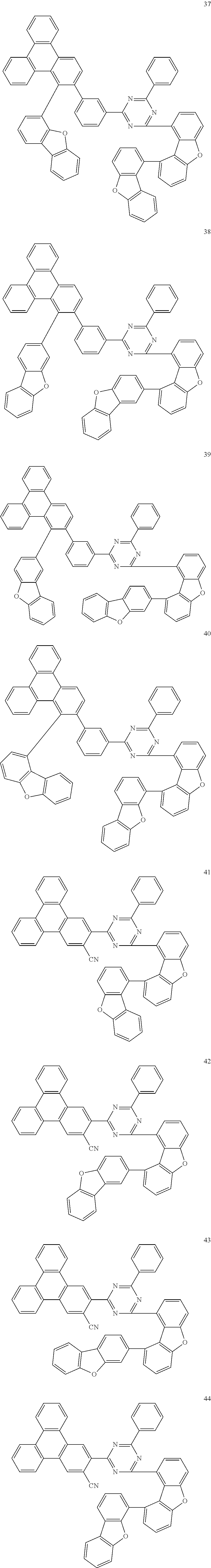

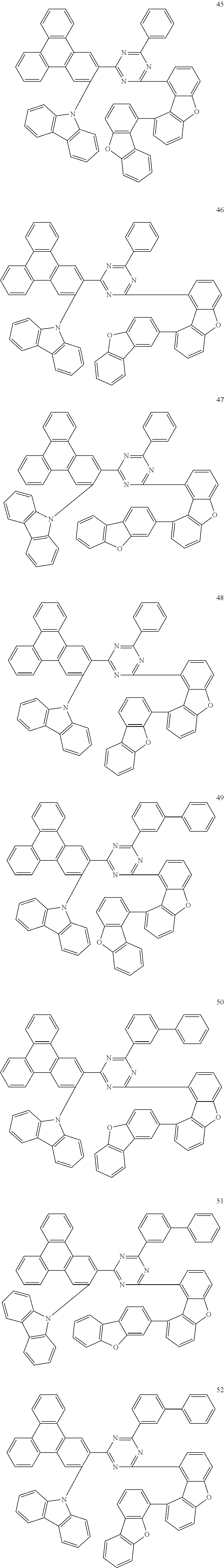

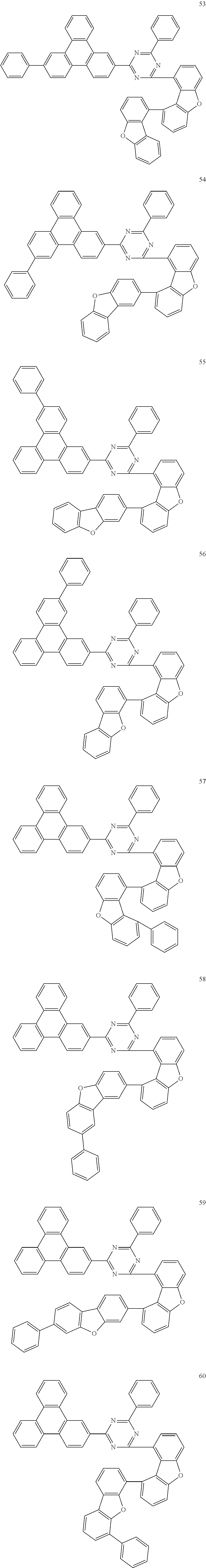

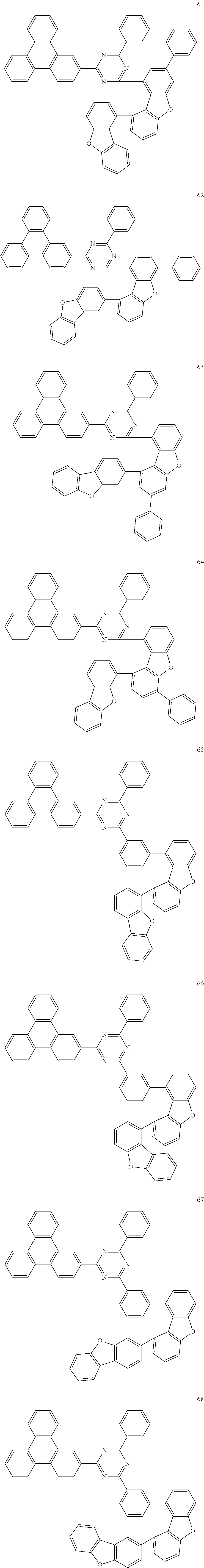

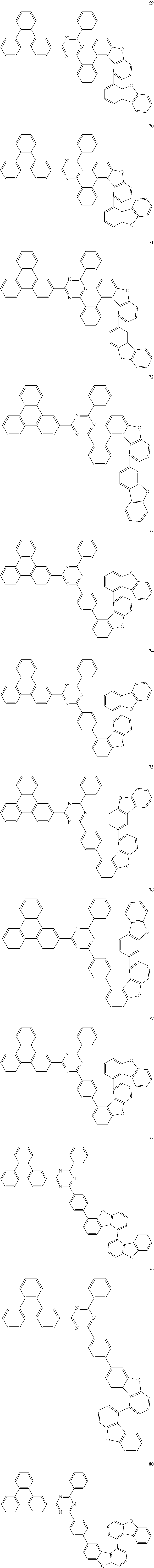

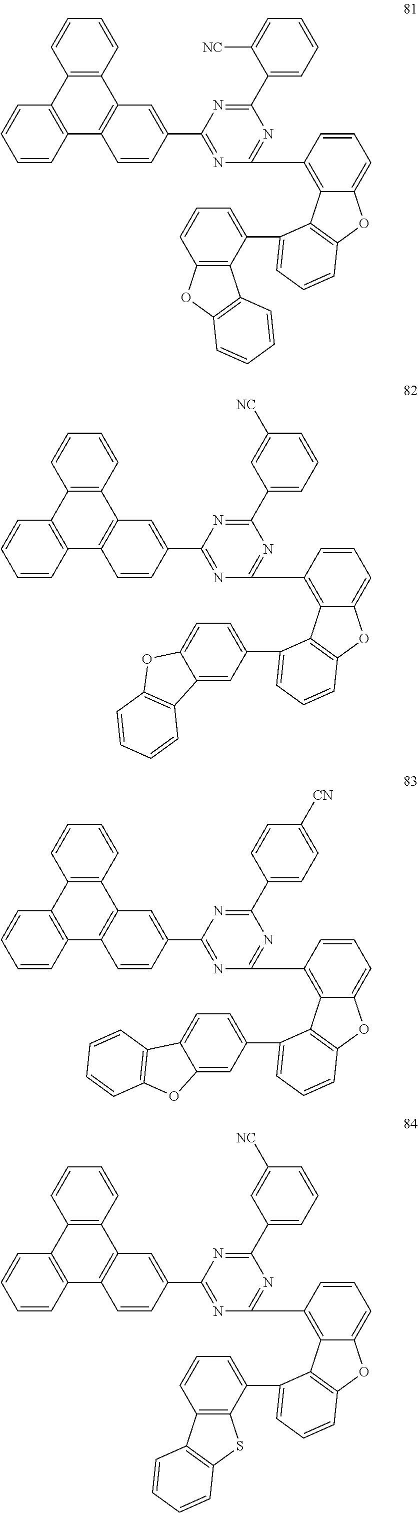

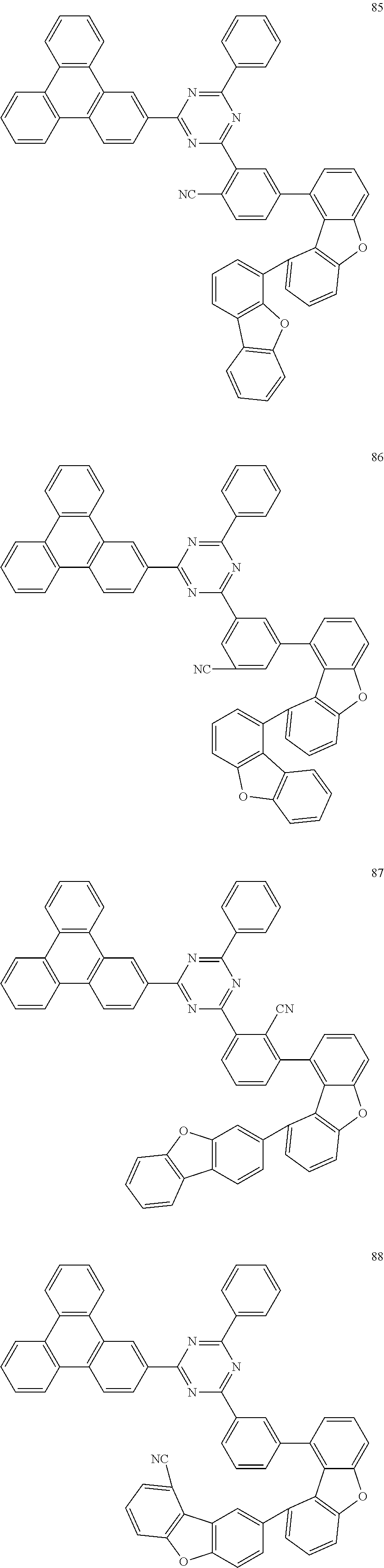 [SMILES: N#Cc1ccc(-c2cccc3oc4cccc(-c5cccc6c5oc5ccccc56)c4c23)cc1-c1nc(-c2ccccc2)nc(-c2ccc3c4ccccc4c4ccccc4c3c2)n1 ; N#Cc1cc(-c2nc(-c3ccccc3)nc(-c3ccc4c5ccccc5c5ccccc5c4c3)n2)cc(-c2cccc3oc4cccc(-c5cccc6oc7ccccc7c56)c4c23)c1 ; N#Cc1c(-c2nc(-c3ccccc3)nc(-c3ccc4c5ccccc5c5ccccc5c4c3)n2)cccc1-c1cccc2oc3cccc(-c4ccc5c(c4)oc4ccccc45)c3c12 ; N#Cc1cccc2oc3ccc(-c4cccc5oc6cccc(-c7cccc(-c8nc(-c9ccccc9)nc(-c9ccc%10c%11ccccc%11c%11ccccc%11c%10c9)n8)c7)c6c45)cc3c12]

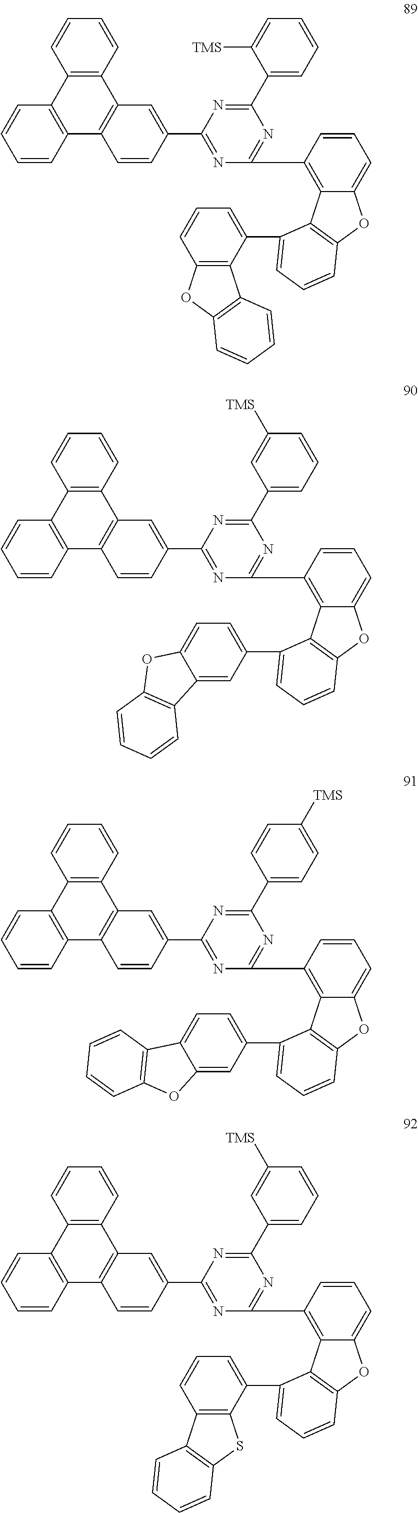

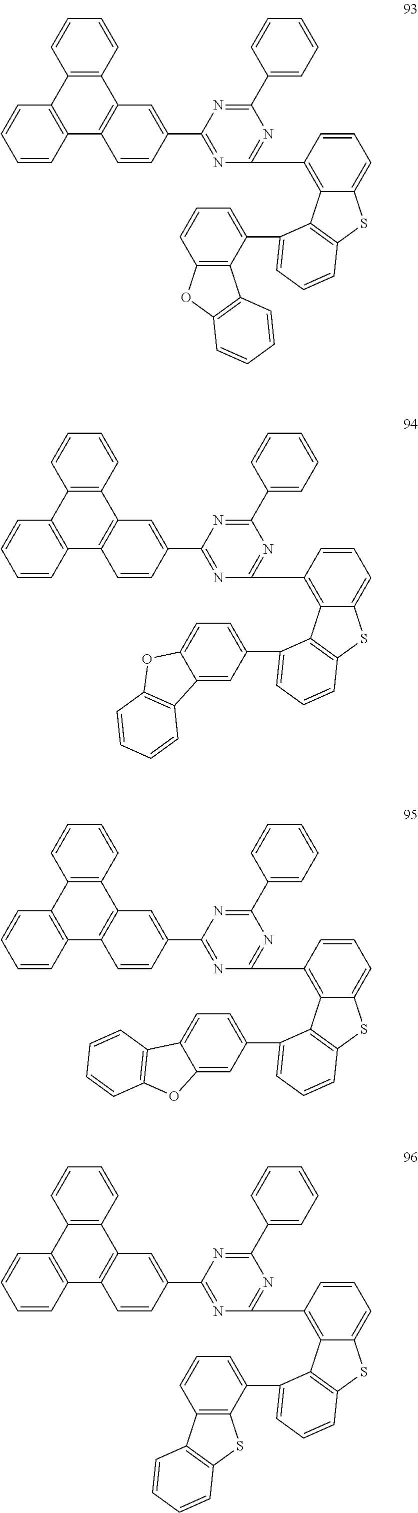 [SMILES: c1ccc(-c2nc(-c3ccc4c5ccccc5c5ccccc5c4c3)nc(-c3cccc4sc5cccc(-c6cccc7oc8ccccc8c67)c5c34)n2)cc1 ; c1ccc(-c2nc(-c3ccc4c5ccccc5c5ccccc5c4c3)nc(-c3cccc4sc5cccc(-c6ccc7oc8ccccc8c7c6)c5c34)n2)cc1 ; c1ccc(-c2nc(-c3ccc4c5ccccc5c5ccccc5c4c3)nc(-c3cccc4sc5cccc(-c6ccc7c(c6)oc6ccccc67)c5c34)n2)cc1 ; c1ccc(-c2nc(-c3ccc4c5ccccc5c5ccccc5c4c3)nc(-c3cccc4sc5cccc(-c6cccc7c6sc6ccccc67)c5c34)n2)cc1]

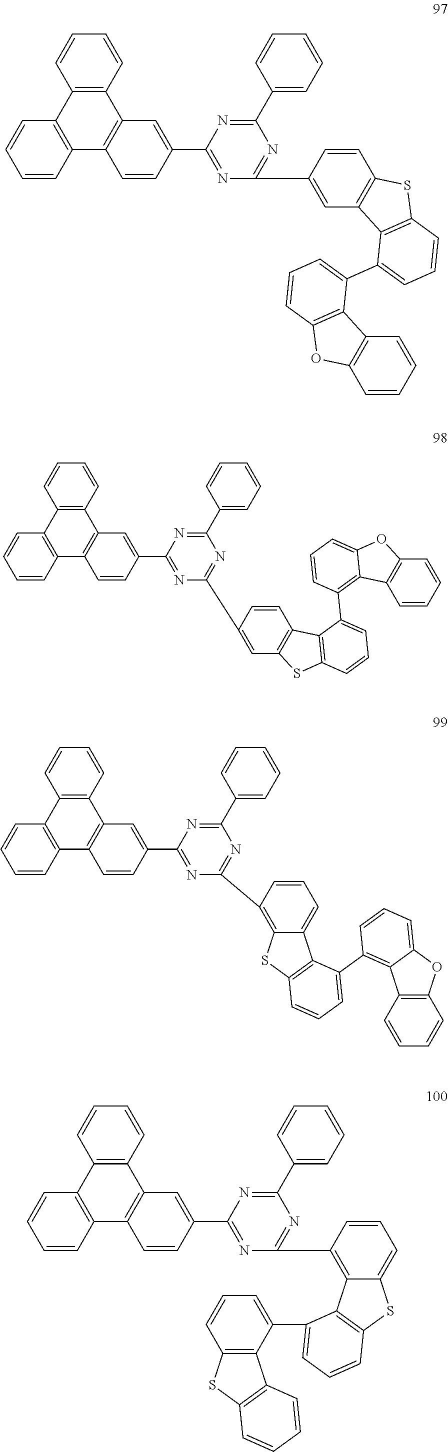 [SMILES: c1ccc(-c2nc(-c3ccc4c5ccccc5c5ccccc5c4c3)nc(-c3ccc4sc5cccc(-c6cccc7oc8ccccc8c67)c5c4c3)n2)cc1 ; c1ccc(-c2nc(-c3ccc4c(c3)sc3cccc(-c5cccc6oc7ccccc7c56)c34)nc(-c3ccc4c5ccccc5c5ccccc5c4c3)n2)cc1 ; c1ccc(-c2nc(-c3ccc4c5ccccc5c5ccccc5c4c3)nc(-c3cccc4c3sc3cccc(-c5cccc6oc7ccccc7c56)c34)n2)cc1 ; c1ccc(-c2nc(-c3ccc4c5ccccc5c5ccccc5c4c3)nc(-c3cccc4sc5cccc(-c6cccc7sc8ccccc8c67)c5c34)n2)cc1]

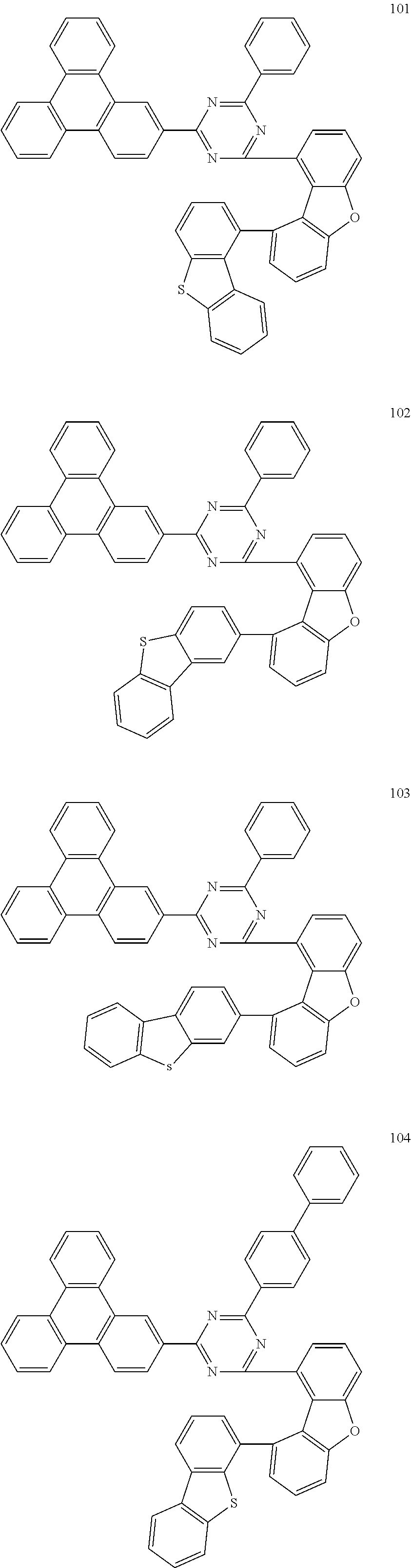

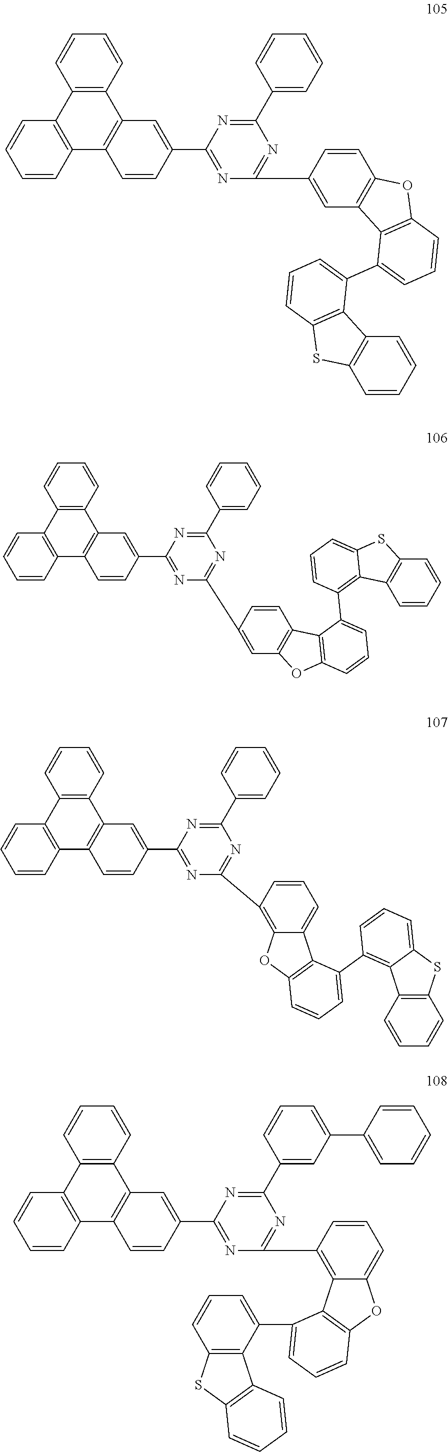 [SMILES: c1ccc(-c2nc(-c3ccc4c5ccccc5c5ccccc5c4c3)nc(-c3ccc4oc5cccc(-c6cccc7sc8ccccc8c67)c5c4c3)n2)cc1 ; c1ccc(-c2nc(-c3ccc4c(c3)oc3cccc(-c5cccc6sc7ccccc7c56)c34)nc(-c3ccc4c5ccccc5c5ccccc5c4c3)n2)cc1 ; c1ccc(-c2nc(-c3ccc4c5ccccc5c5ccccc5c4c3)nc(-c3cccc4c3oc3cccc(-c5cccc6sc7ccccc7c56)c34)n2)cc1 ; c1ccc(-c2cccc(-c3nc(-c4ccc5c6ccccc6c6ccccc6c5c4)nc(-c4cccc5oc6cccc(-c7cccc8sc9ccccc9c78)c6c45)n3)c2)cc1]

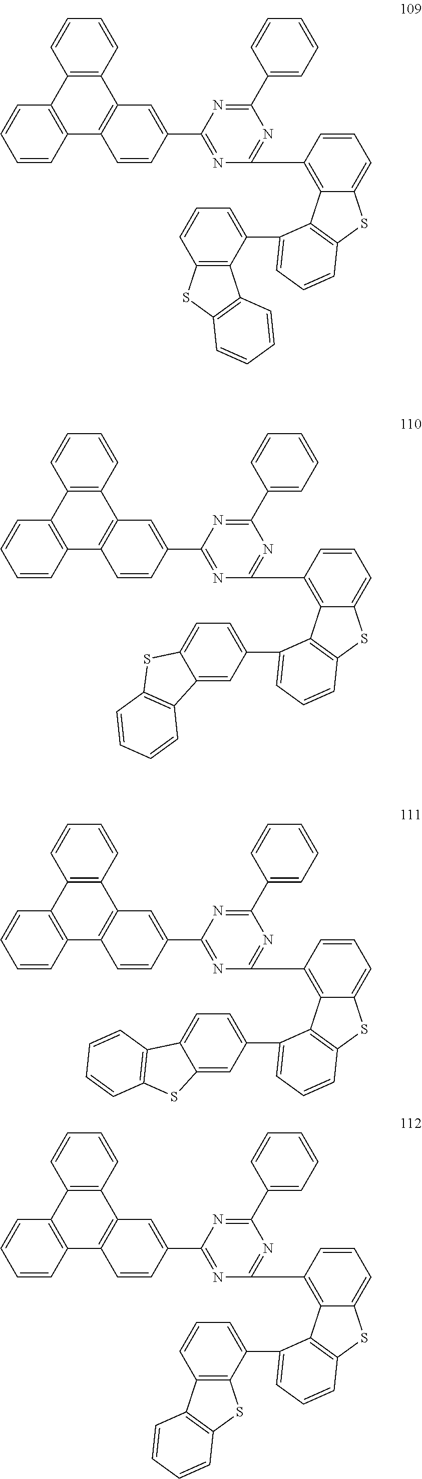

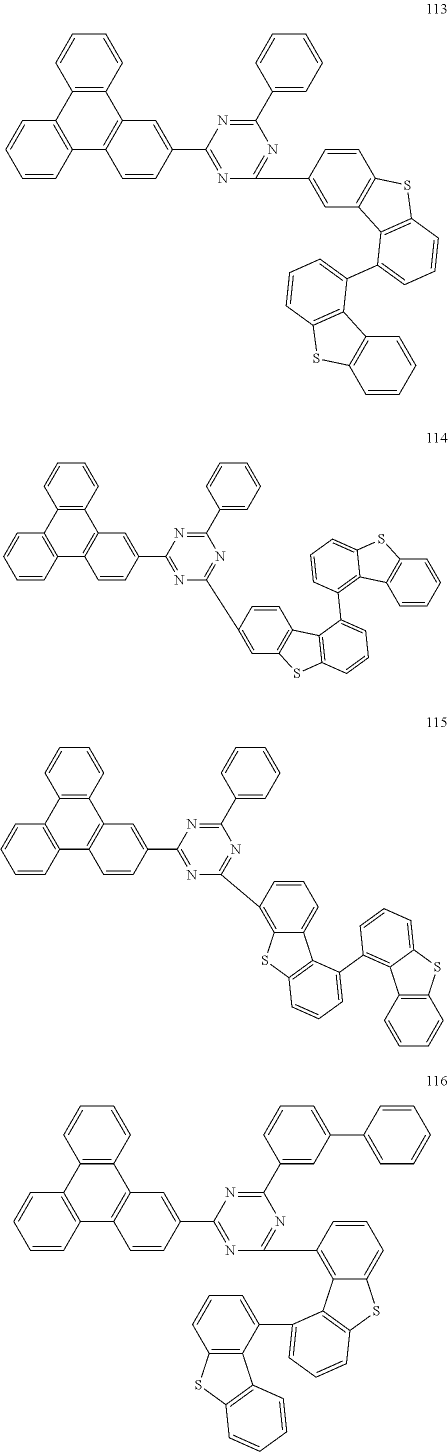 [SMILES: c1ccc(-c2nc(-c3ccc4c5ccccc5c5ccccc5c4c3)nc(-c3ccc4sc5cccc(-c6cccc7sc8ccccc8c67)c5c4c3)n2)cc1 ; c1ccc(-c2nc(-c3ccc4c(c3)sc3cccc(-c5cccc6sc7ccccc7c56)c34)nc(-c3ccc4c5ccccc5c5ccccc5c4c3)n2)cc1 ; c1ccc(-c2nc(-c3ccc4c5ccccc5c5ccccc5c4c3)nc(-c3cccc4c3sc3cccc(-c5cccc6sc7ccccc7c56)c34)n2)cc1 ; c1ccc(-c2cccc(-c3nc(-c4ccc5c6ccccc6c6ccccc6c5c4)nc(-c4cccc5sc6cccc(-c7cccc8sc9ccccc9c78)c6c45)n3)c2)cc1]

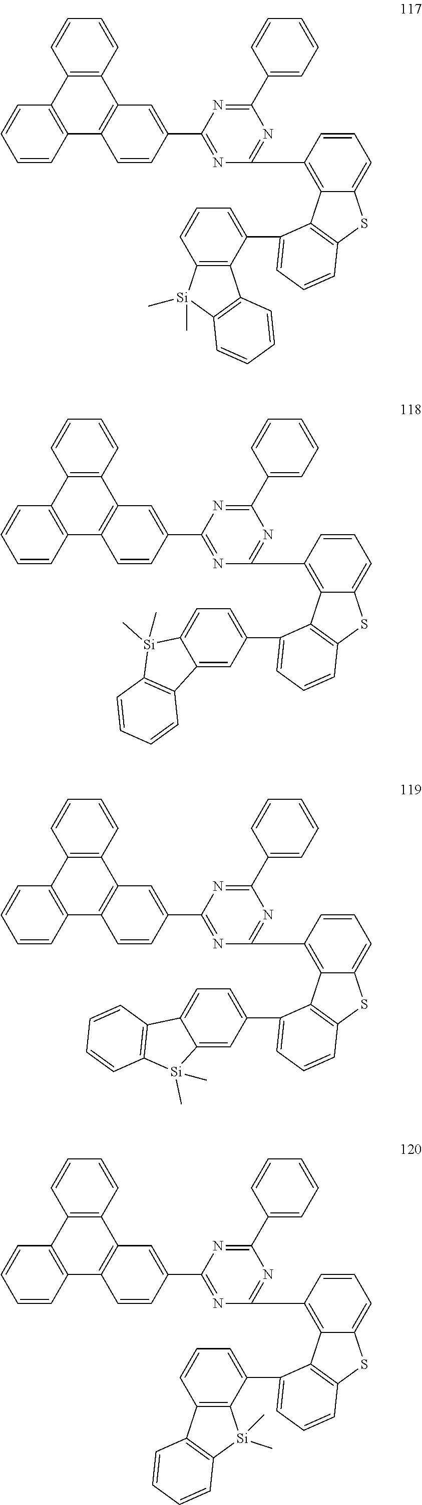 [SMILES: C[Si]1(C)c2ccccc2-c2c(-c3cccc4sc5cccc(-c6nc(-c7ccccc7)nc(-c7ccc8c9ccccc9c9ccccc9c8c7)n6)c5c34)cccc21 ; C[Si]1(C)c2ccccc2-c2cc(-c3cccc4sc5cccc(-c6nc(-c7ccccc7)nc(-c7ccc8c9ccccc9c9ccccc9c8c7)n6)c5c34)ccc21 ; C[Si]1(C)c2ccccc2-c2ccc(-c3cccc4sc5cccc(-c6nc(-c7ccccc7)nc(-c7ccc8c9ccccc9c9ccccc9c8c7)n6)c5c34)cc21 ; C[Si]1(C)c2ccccc2-c2cccc(-c3cccc4sc5cccc(-c6nc(-c7ccccc7)nc(-c7ccc8c9ccccc9c9ccccc9c8c7)n6)c5c34)c21]

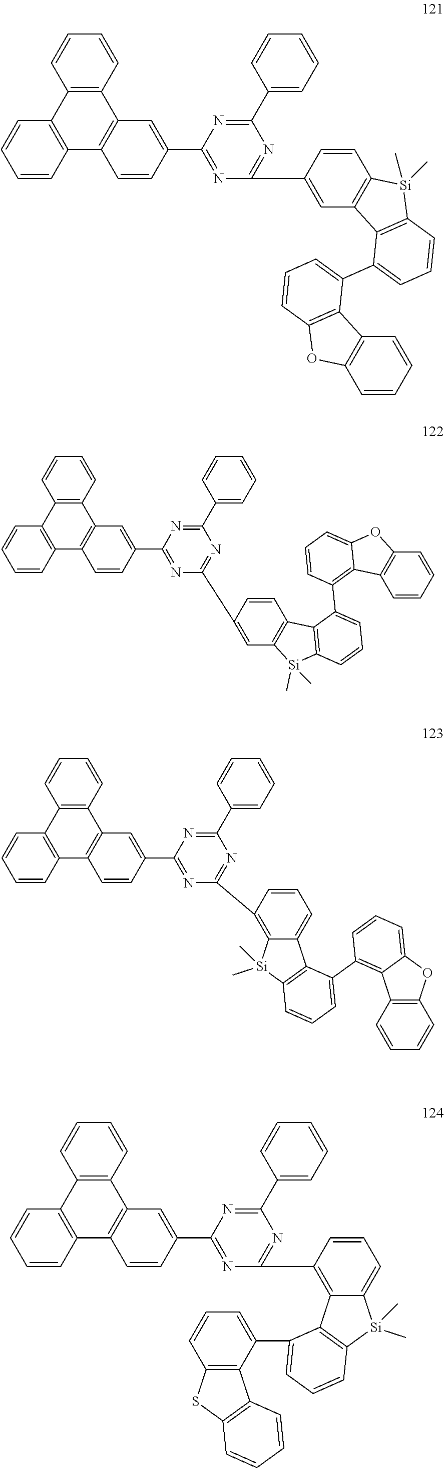 [SMILES: C[Si]1(C)c2ccc(-c3nc(-c4ccccc4)nc(-c4ccc5c6ccccc6c6ccccc6c5c4)n3)cc2-c2c(-c3cccc4oc5ccccc5c34)cccc21 ; C[Si]1(C)c2cc(-c3nc(-c4ccccc4)nc(-c4ccc5c6ccccc6c6ccccc6c5c4)n3)ccc2-c2c(-c3cccc4oc5ccccc5c34)cccc21 ; C[Si]1(C)c2cccc(-c3cccc4oc5ccccc5c34)c2-c2cccc(-c3nc(-c4ccccc4)nc(-c4ccc5c6ccccc6c6ccccc6c5c4)n3)c21 ; C[Si]1(C)c2cccc(-c3nc(-c4ccccc4)nc(-c4ccc5c6ccccc6c6ccccc6c5c4)n3)c2-c2c(-c3cccc4sc5ccccc5c34)cccc21]

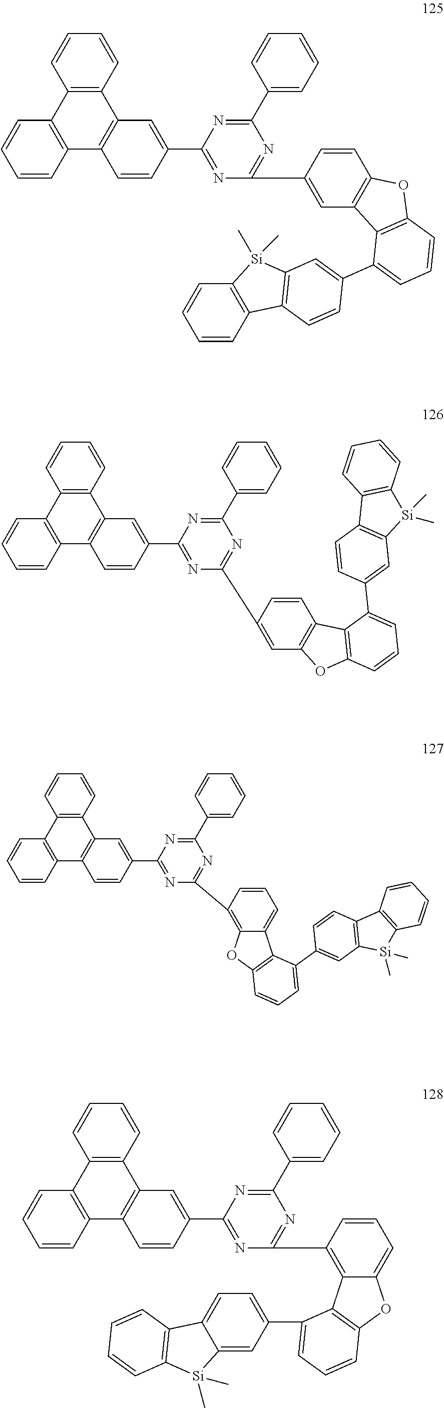 [SMILES: C[Si]1(C)c2ccccc2-c2ccc(-c3cccc4oc5ccc(-c6nc(-c7ccccc7)nc(-c7ccc8c9ccccc9c9ccccc9c8c7)n6)cc5c34)cc21 ; C[Si]1(C)c2ccccc2-c2ccc(-c3cccc4oc5cc(-c6nc(-c7ccccc7)nc(-c7ccc8c9ccccc9c9ccccc9c8c7)n6)ccc5c34)cc21 ; C[Si]1(C)c2ccccc2-c2ccc(-c3cccc4oc5c(-c6nc(-c7ccccc7)nc(-c7ccc8c9ccccc9c9ccccc9c8c7)n6)cccc5c34)cc21 ; C[Si]1(C)c2ccccc2-c2ccc(-c3cccc4oc5cccc(-c6nc(-c7ccccc7)nc(-c7ccc8c9ccccc9c9ccccc9c8c7)n6)c5c34)cc21]

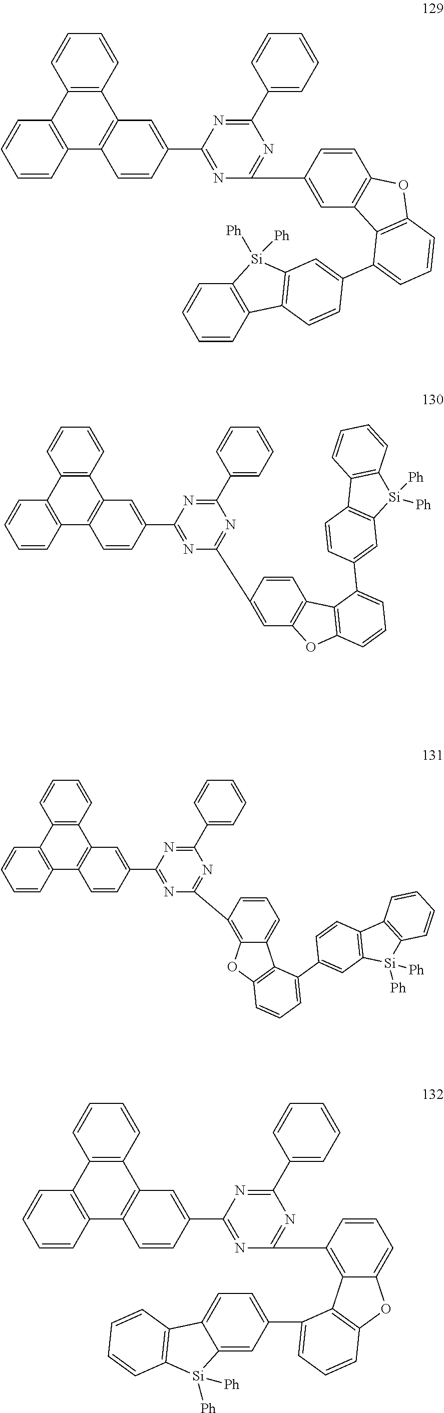 [SMILES: c1ccc(-c2nc(-c3ccc4c5ccccc5c5ccccc5c4c3)nc(-c3ccc4oc5cccc(-c6ccc7c(c6)[Si](c6ccccc6)(c6ccccc6)c6ccccc6-7)c5c4c3)n2)cc1 ; c1ccc(-c2nc(-c3ccc4c(c3)oc3cccc(-c5ccc6c(c5)[Si](c5ccccc5)(c5ccccc5)c5ccccc5-6)c34)nc(-c3ccc4c5ccccc5c5ccccc5c4c3)n2)cc1 ; c1ccc(-c2nc(-c3ccc4c5ccccc5c5ccccc5c4c3)nc(-c3cccc4c3oc3cccc(-c5ccc6c(c5)[Si](c5ccccc5)(c5ccccc5)c5ccccc5-6)c34)n2)cc1 ; c1ccc(-c2nc(-c3ccc4c5ccccc5c5ccccc5c4c3)nc(-c3cccc4oc5cccc(-c6ccc7c(c6)[Si](c6ccccc6)(c6ccccc6)c6ccccc6-7)c5c34)n2)cc1]

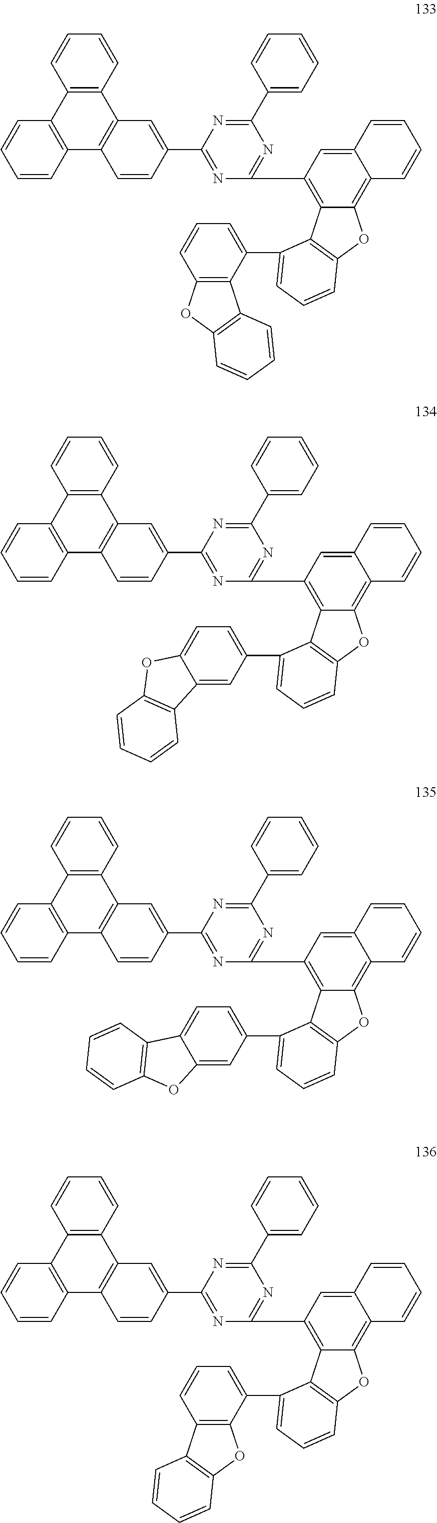 [SMILES: c1ccc(-c2nc(-c3ccc4c5ccccc5c5ccccc5c4c3)nc(-c3cc4ccccc4c4oc5cccc(-c6cccc7oc8ccccc8c67)c5c34)n2)cc1 ; c1ccc(-c2nc(-c3ccc4c5ccccc5c5ccccc5c4c3)nc(-c3cc4ccccc4c4oc5cccc(-c6ccc7oc8ccccc8c7c6)c5c34)n2)cc1 ; c1ccc(-c2nc(-c3ccc4c5ccccc5c5ccccc5c4c3)nc(-c3cc4ccccc4c4oc5cccc(-c6ccc7c(c6)oc6ccccc67)c5c34)n2)cc1 ; c1ccc(-c2nc(-c3ccc4c5ccccc5c5ccccc5c4c3)nc(-c3cc4ccccc4c4oc5cccc(-c6cccc7c6oc6ccccc67)c5c34)n2)cc1]

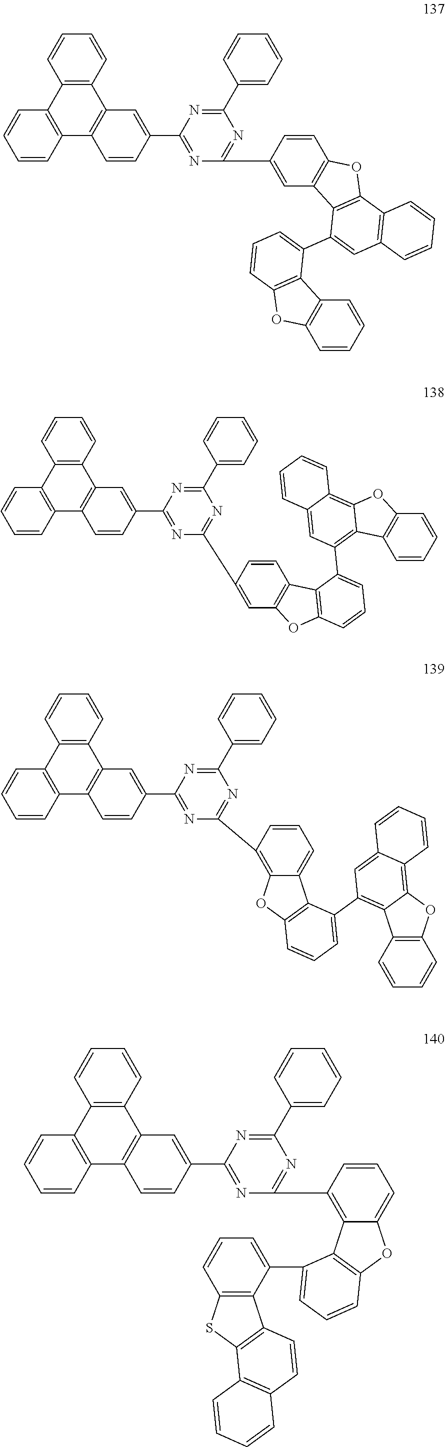 [SMILES: c1ccc(-c2nc(-c3ccc4c5ccccc5c5ccccc5c4c3)nc(-c3ccc4oc5c6ccccc6cc(-c6cccc7oc8ccccc8c67)c5c4c3)n2)cc1 ; c1ccc(-c2nc(-c3ccc4c(c3)oc3cccc(-c5cc6ccccc6c6oc7ccccc7c56)c34)nc(-c3ccc4c5ccccc5c5ccccc5c4c3)n2)cc1 ; c1ccc(-c2nc(-c3ccc4c5ccccc5c5ccccc5c4c3)nc(-c3cccc4c3oc3cccc(-c5cc6ccccc6c6oc7ccccc7c56)c34)n2)cc1 ; c1ccc(-c2nc(-c3ccc4c5ccccc5c5ccccc5c4c3)nc(-c3cccc4oc5cccc(-c6cccc7sc8c9ccccc9ccc8c67)c5c34)n2)cc1]

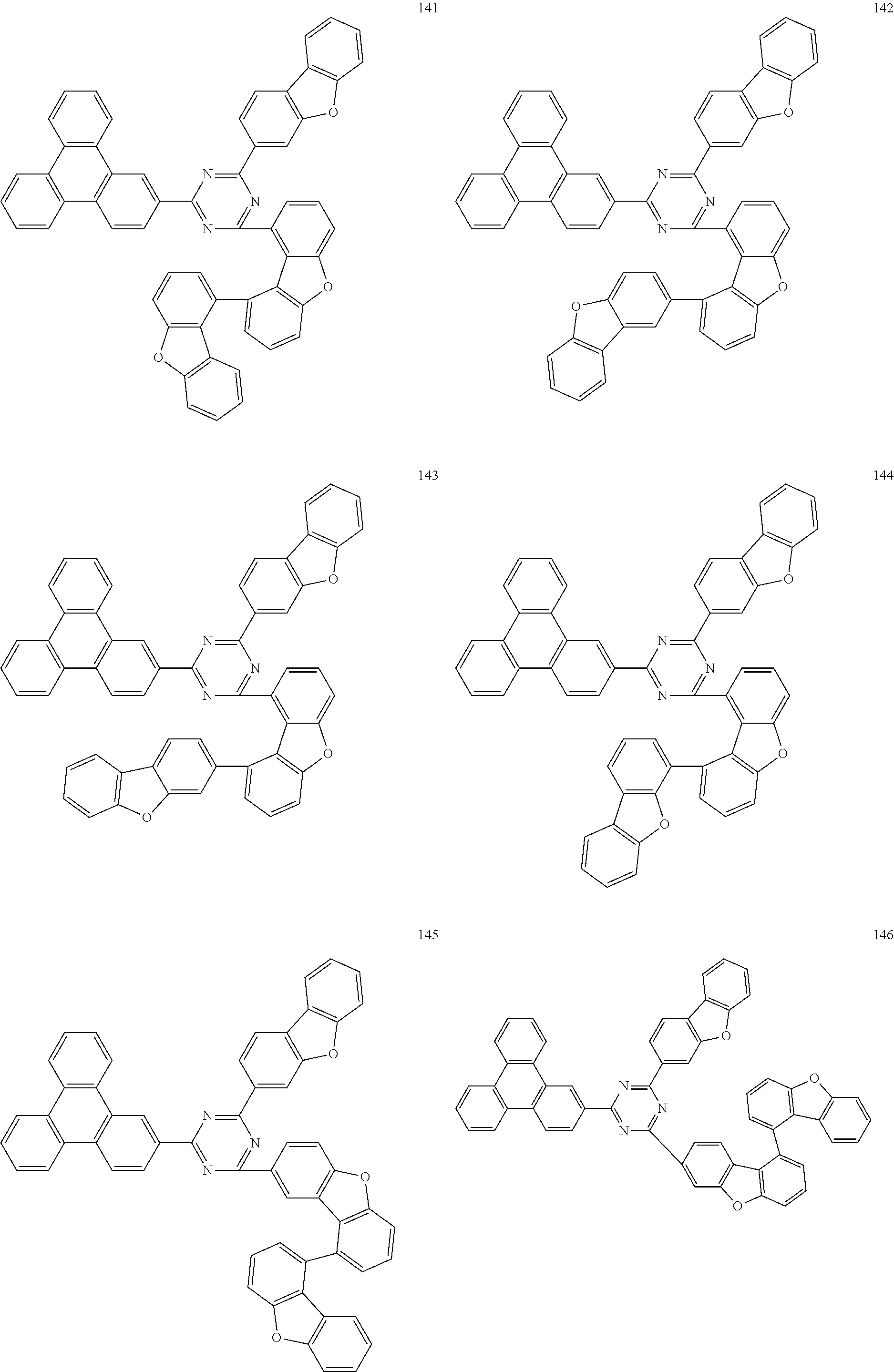

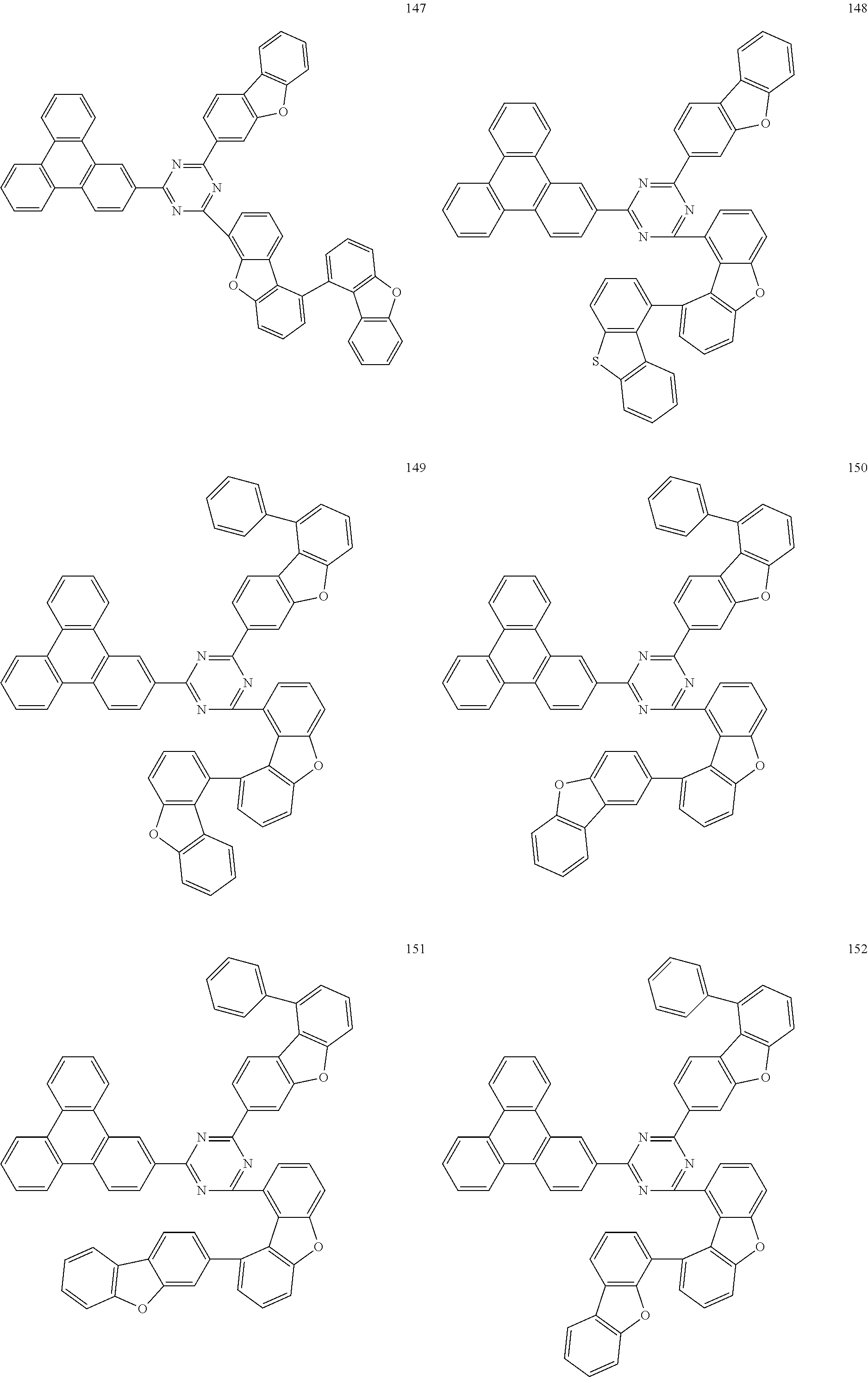

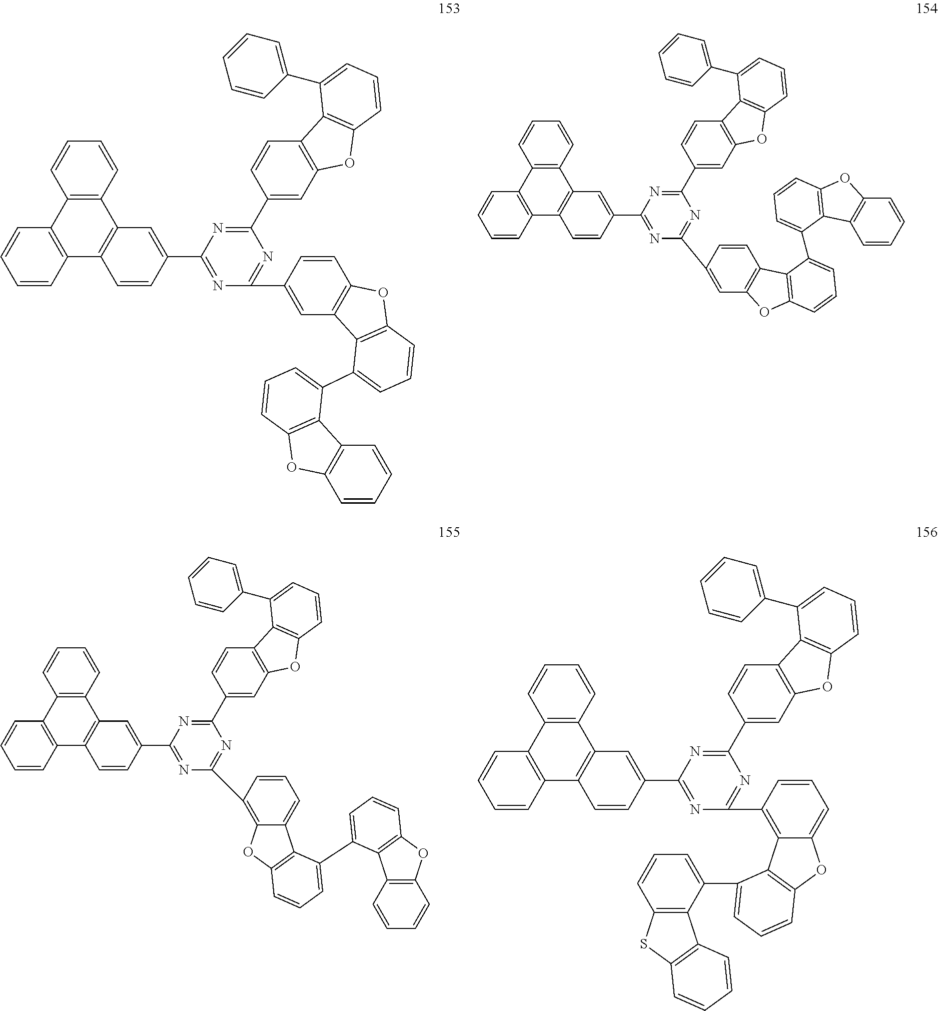 [SMILES: c1ccc(-c2cccc3oc4cc(-c5nc(-c6ccc7c8ccccc8c8ccccc8c7c6)nc(-c6ccc7oc8cccc(-c9cccc%10oc%11ccccc%11c9%10)c8c7c6)n5)ccc4c23)cc1 ; c1ccc(-c2cccc3oc4cc(-c5nc(-c6ccc7c(c6)oc6cccc(-c8cccc9oc%10ccccc%10c89)c67)nc(-c6ccc7c8ccccc8c8ccccc8c7c6)n5)ccc4c23)cc1 ; c1ccc(-c2cccc3oc4cc(-c5nc(-c6ccc7c8ccccc8c8ccccc8c7c6)nc(-c6cccc7c6oc6cccc(-c8cccc9oc%10ccccc%10c89)c67)n5)ccc4c23)cc1 ; c1ccc(-c2cccc3oc4cc(-c5nc(-c6ccc7c8ccccc8c8ccccc8c7c6)nc(-c6cccc7oc8cccc(-c9cccc%10sc%11ccccc%11c9%10)c8c67)n5)ccc4c23)cc1]

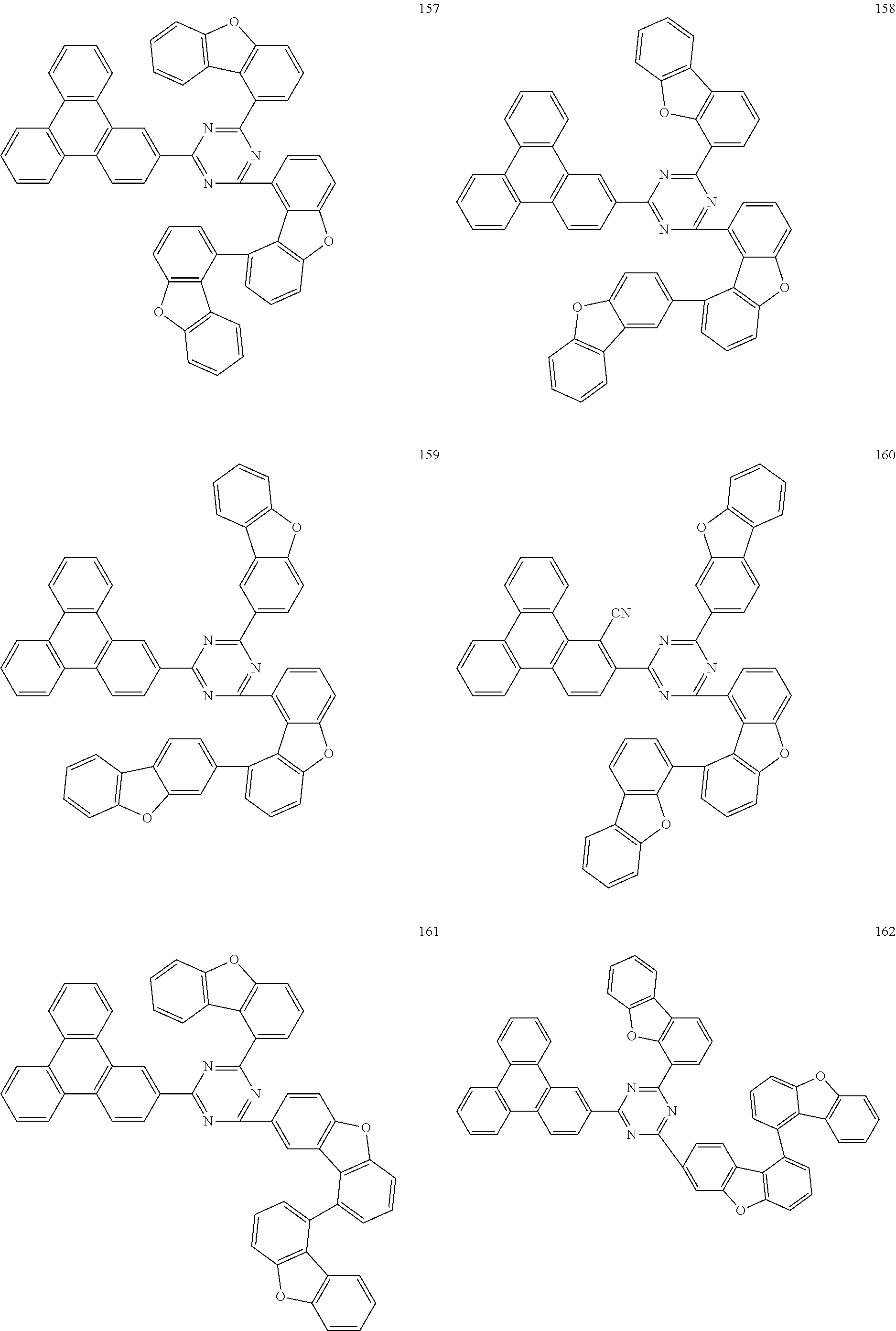

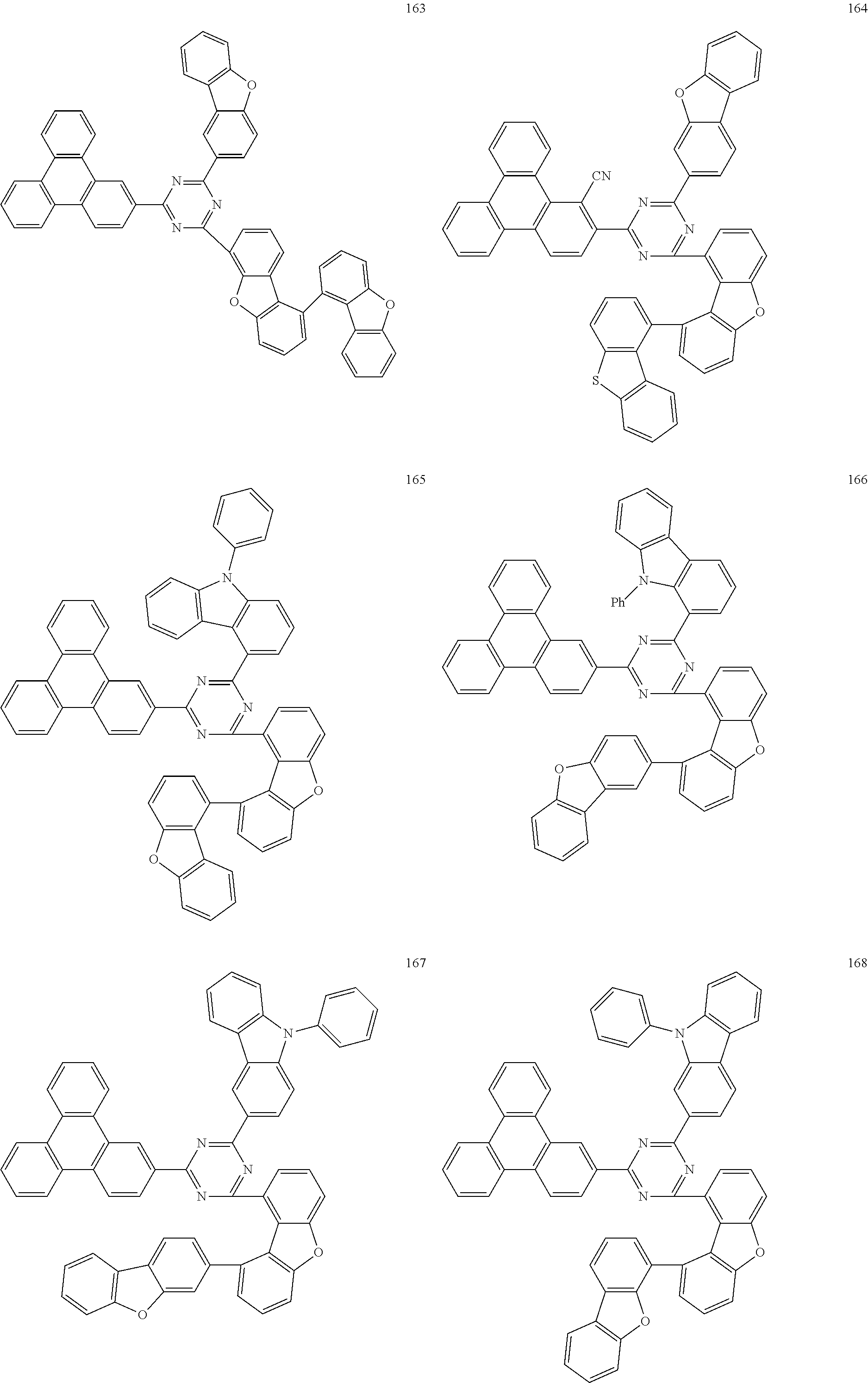

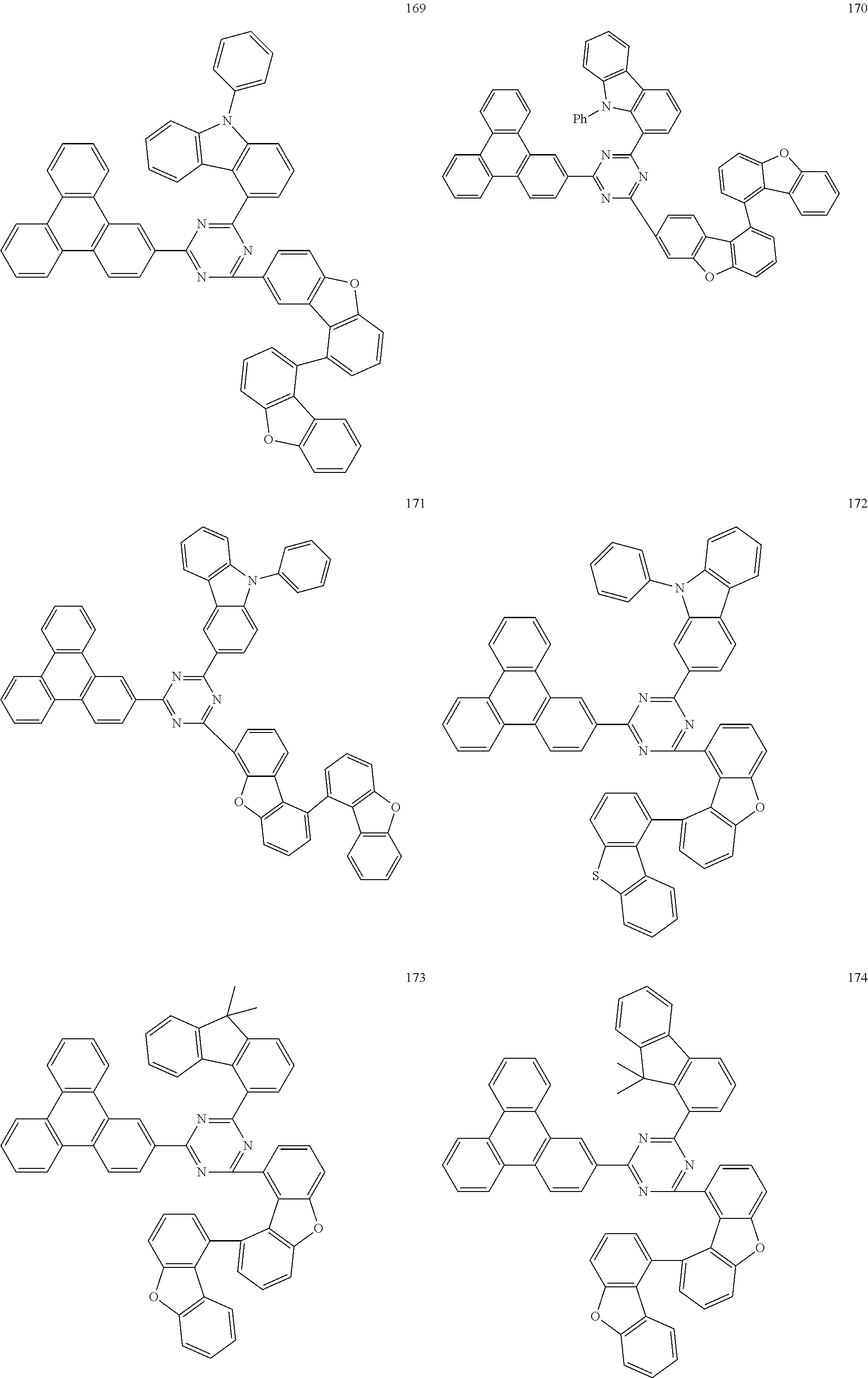

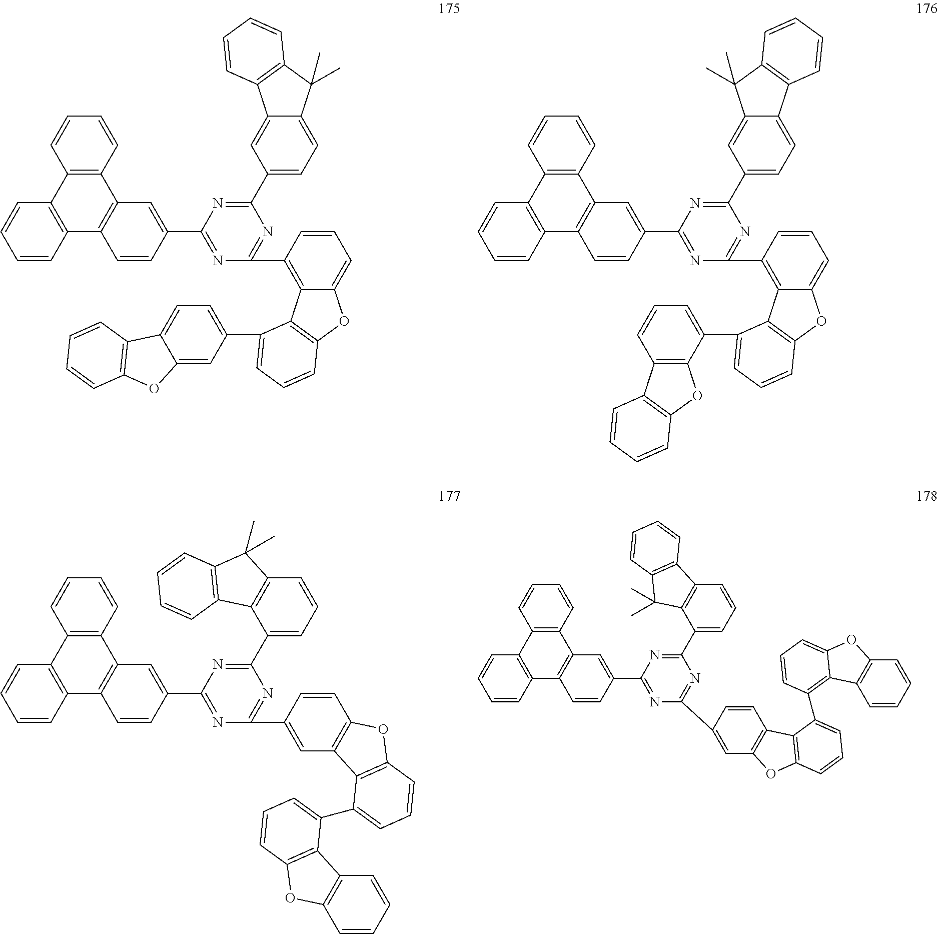 [SMILES: CC1(C)c2ccccc2-c2cc(-c3nc(-c4ccc5c6ccccc6c6ccccc6c5c4)nc(-c4cccc5oc6cccc(-c7ccc8c(c7)oc7ccccc78)c6c45)n3)ccc21 ; CC1(C)c2ccccc2-c2ccc(-c3nc(-c4ccc5c6ccccc6c6ccccc6c5c4)nc(-c4cccc5oc6cccc(-c7cccc8c7oc7ccccc78)c6c45)n3)cc21 ; CC1(C)c2ccccc2-c2c(-c3nc(-c4ccc5c6ccccc6c6ccccc6c5c4)nc(-c4ccc5oc6cccc(-c7cccc8oc9ccccc9c78)c6c5c4)n3)cccc21 ; CC1(C)c2ccccc2-c2cccc(-c3nc(-c4ccc5c(c4)oc4cccc(-c6cccc7oc8ccccc8c67)c45)nc(-c4ccc5c6ccccc6c6ccccc6c5c4)n3)c21]

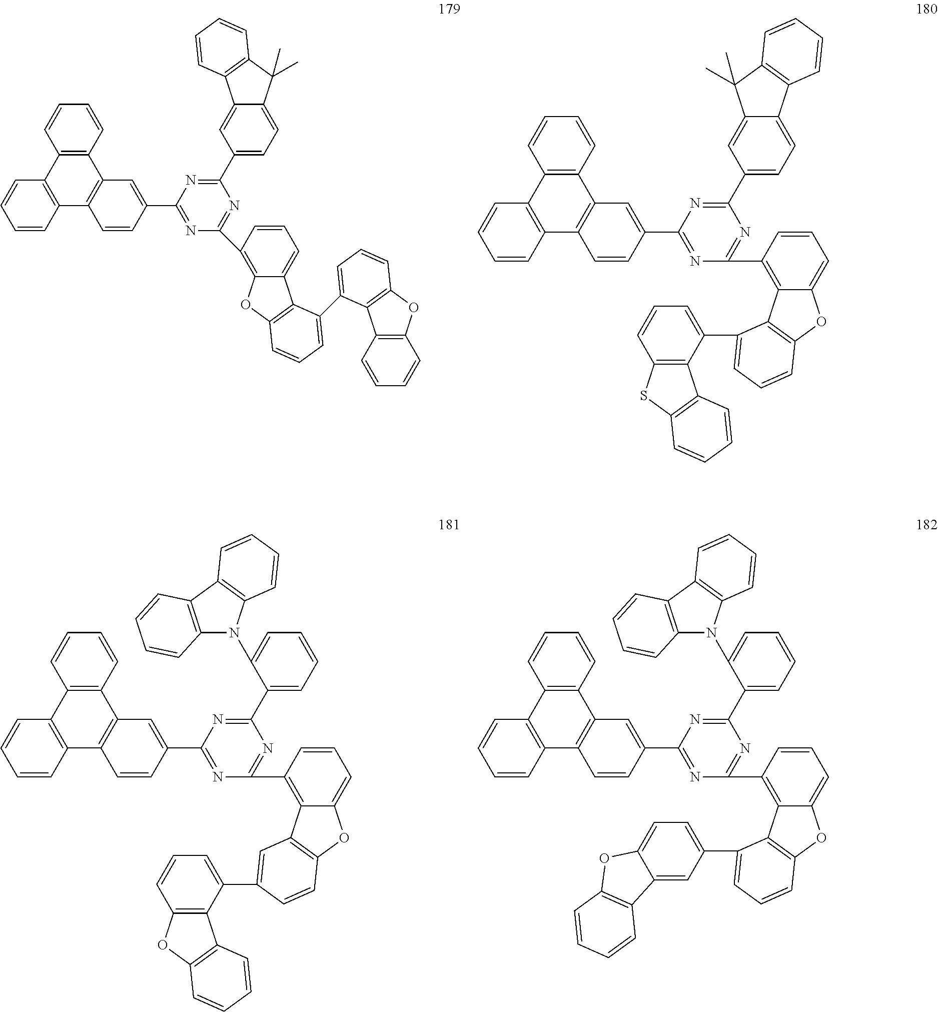 [SMILES: CC1(C)c2ccccc2-c2cc(-c3nc(-c4ccc5c6ccccc6c6ccccc6c5c4)nc(-c4cccc5c4oc4cccc(-c6cccc7oc8ccccc8c67)c45)n3)ccc21 ; CC1(C)c2ccccc2-c2ccc(-c3nc(-c4ccc5c6ccccc6c6ccccc6c5c4)nc(-c4cccc5oc6cccc(-c7cccc8sc9ccccc9c78)c6c45)n3)cc21 ; c1ccc(-n2c3ccccc3c3ccccc32)c(-c2nc(-c3ccc4c5ccccc5c5ccccc5c4c3)nc(-c3cccc4oc5ccc(-c6cccc7oc8ccccc8c67)cc5c34)n2)c1 ; c1ccc(-n2c3ccccc3c3ccccc32)c(-c2nc(-c3ccc4c5ccccc5c5ccccc5c4c3)nc(-c3cccc4oc5cccc(-c6ccc7oc8ccccc8c7c6)c5c34)n2)c1]

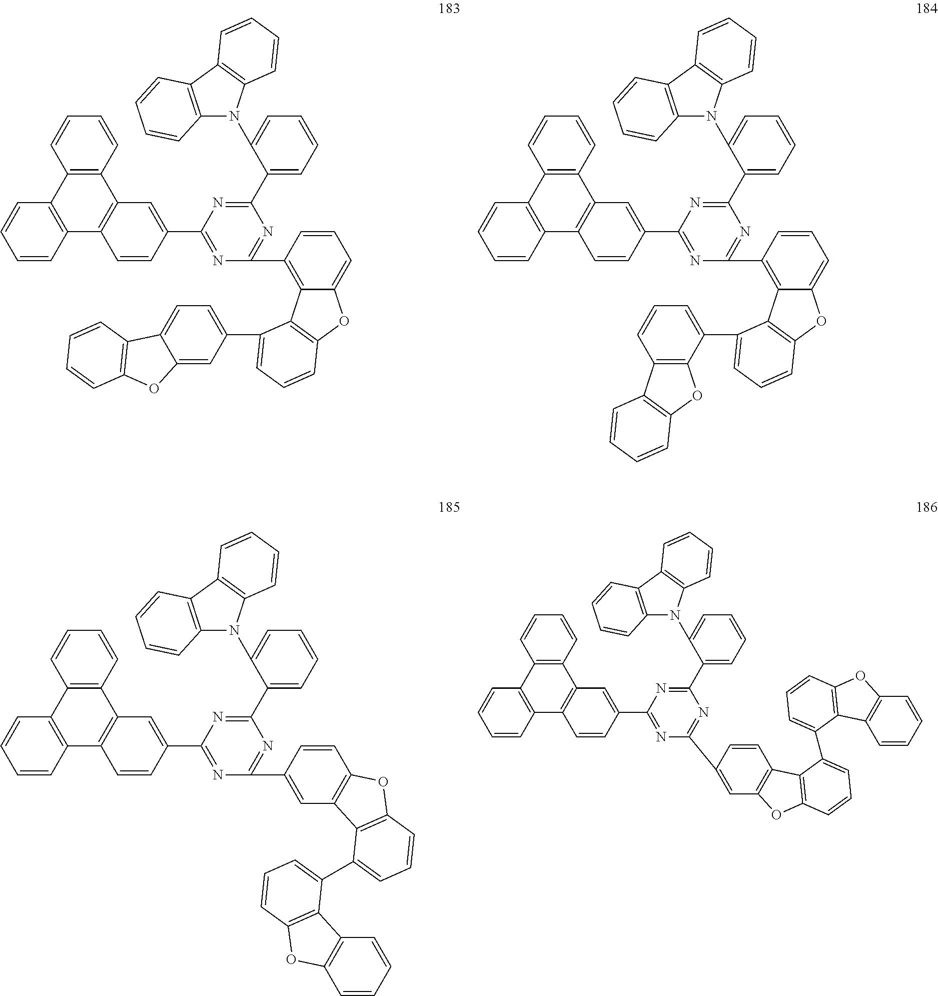 [SMILES: c1ccc(-n2c3ccccc3c3ccccc32)c(-c2nc(-c3ccc4c5ccccc5c5ccccc5c4c3)nc(-c3cccc4oc5cccc(-c6ccc7c(c6)oc6ccccc67)c5c34)n2)c1 ; c1ccc(-n2c3ccccc3c3ccccc32)c(-c2nc(-c3ccc4c5ccccc5c5ccccc5c4c3)nc(-c3cccc4oc5cccc(-c6cccc7c6oc6ccccc67)c5c34)n2)c1 ; c1ccc(-n2c3ccccc3c3ccccc32)c(-c2nc(-c3ccc4c5ccccc5c5ccccc5c4c3)nc(-c3ccc4oc5cccc(-c6cccc7oc8ccccc8c67)c5c4c3)n2)c1 ; c1ccc(-n2c3ccccc3c3ccccc32)c(-c2nc(-c3ccc4c(c3)oc3cccc(-c5cccc6oc7ccccc7c56)c34)nc(-c3ccc4c5ccccc5c5ccccc5c4c3)n2)c1]

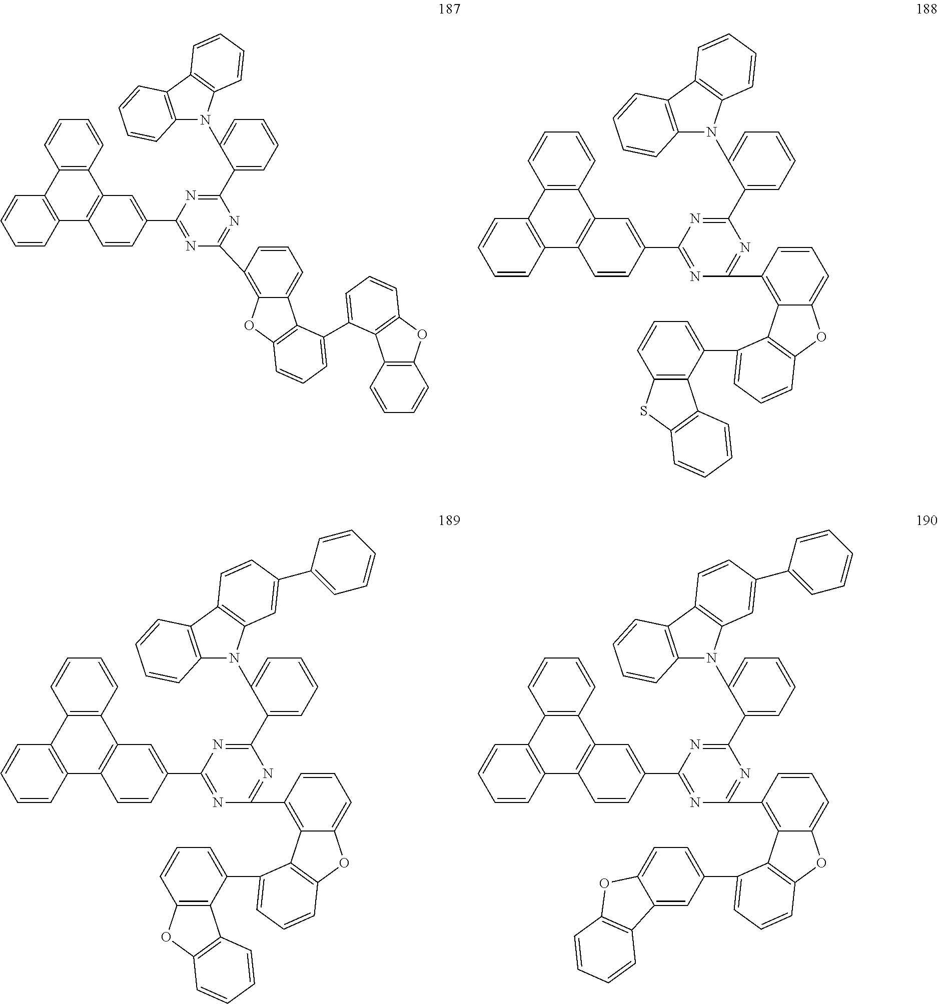 [SMILES: c1ccc(-n2c3ccccc3c3ccccc32)c(-c2nc(-c3ccc4c5ccccc5c5ccccc5c4c3)nc(-c3cccc4c3oc3cccc(-c5cccc6oc7ccccc7c56)c34)n2)c1 ; c1ccc(-n2c3ccccc3c3ccccc32)c(-c2nc(-c3ccc4c5ccccc5c5ccccc5c4c3)nc(-c3cccc4oc5cccc(-c6cccc7sc8ccccc8c67)c5c34)n2)c1 ; c1ccc(-c2ccc3c4ccccc4n(-c4ccccc4-c4nc(-c5ccc6c7ccccc7c7ccccc7c6c5)nc(-c5cccc6oc7cccc(-c8cccc9oc%10ccccc%10c89)c7c56)n4)c3c2)cc1 ; c1ccc(-c2ccc3c4ccccc4n(-c4ccccc4-c4nc(-c5ccc6c7ccccc7c7ccccc7c6c5)nc(-c5cccc6oc7cccc(-c8ccc9oc%10ccccc%10c9c8)c7c56)n4)c3c2)cc1]

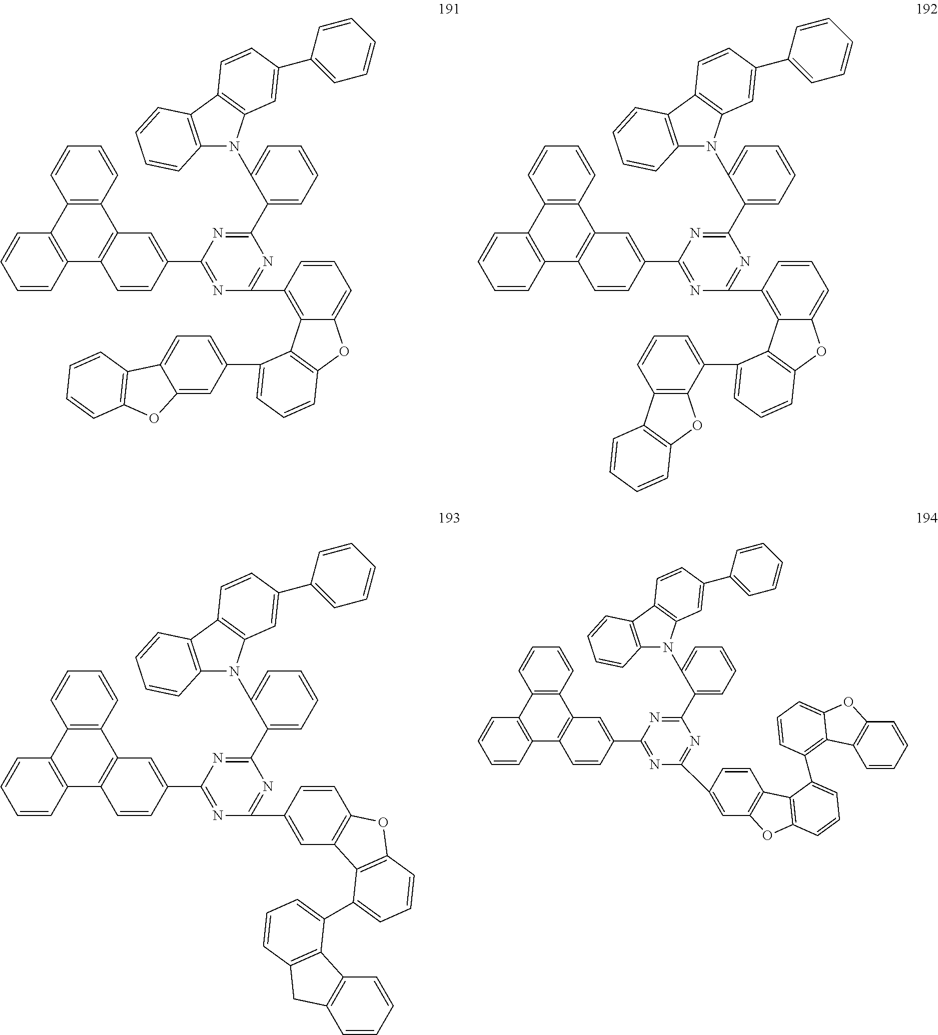

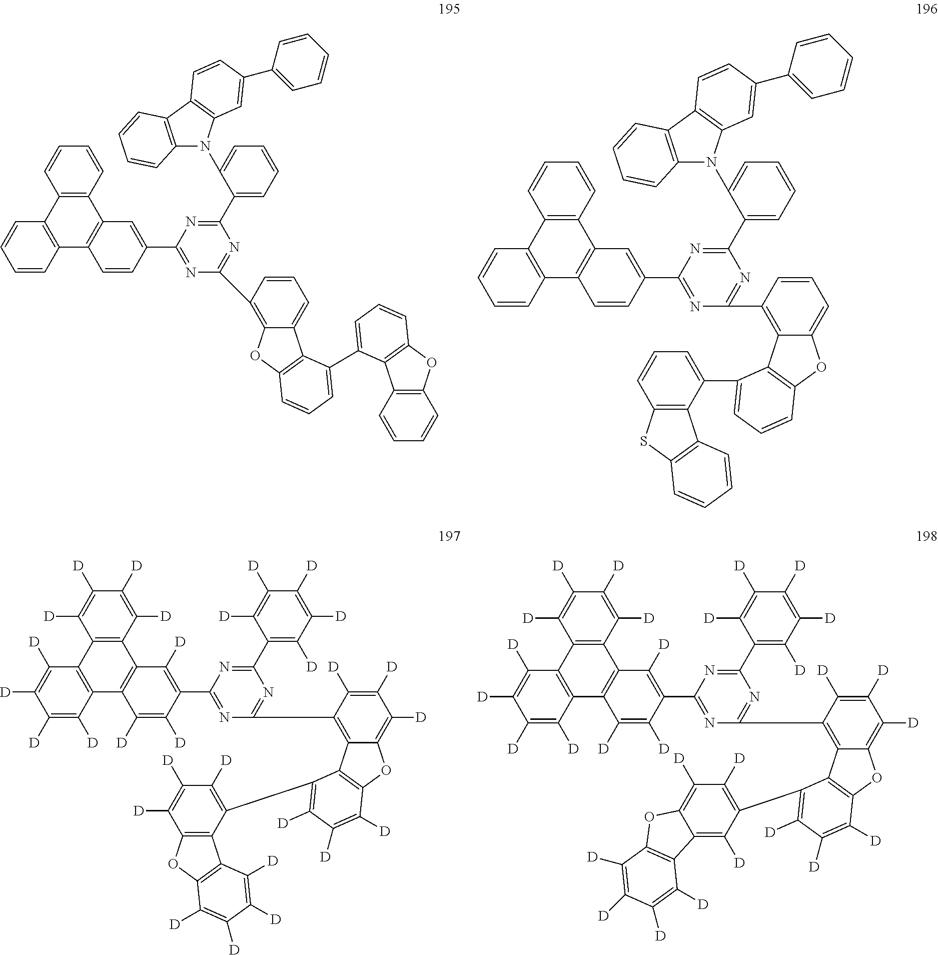

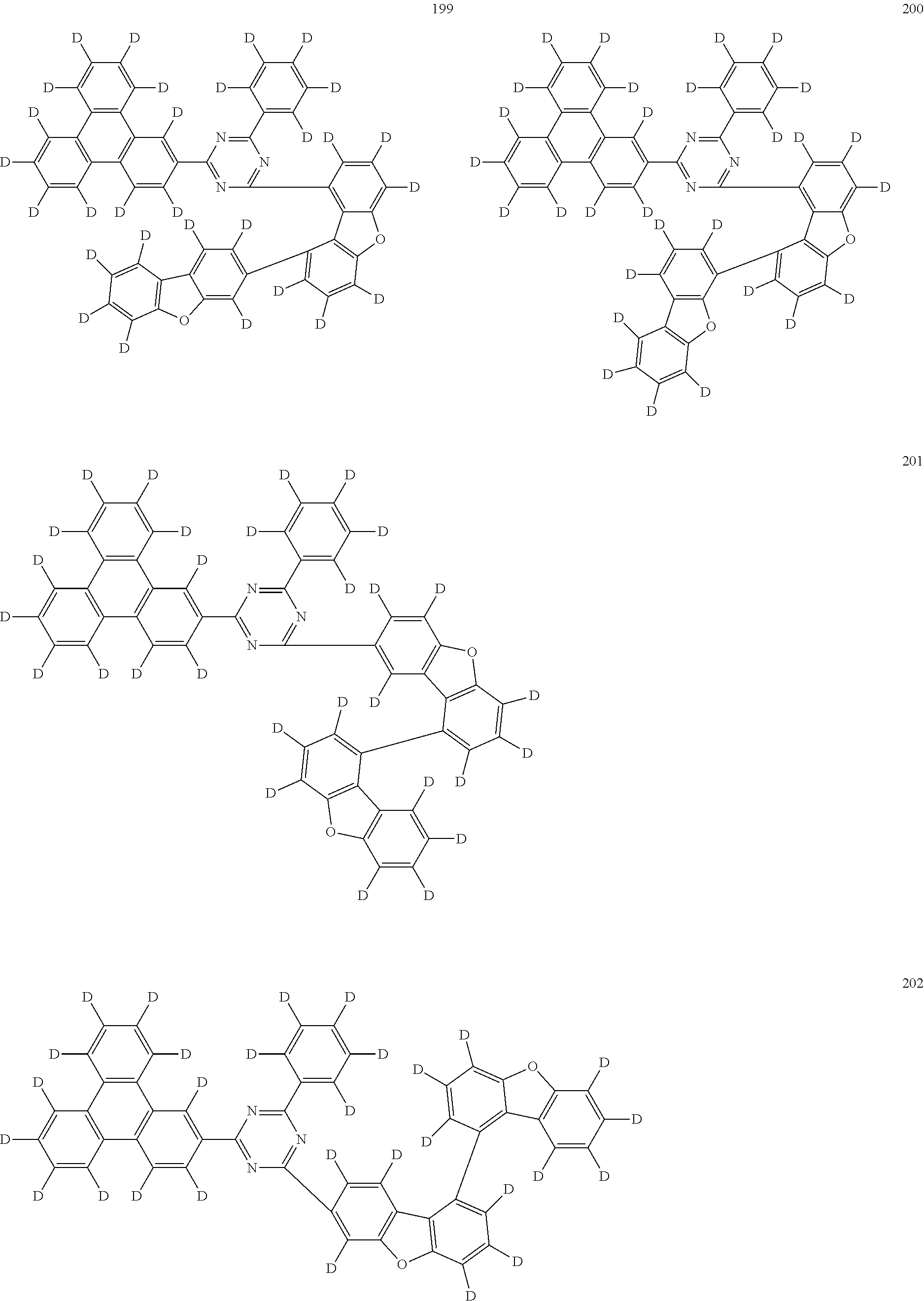

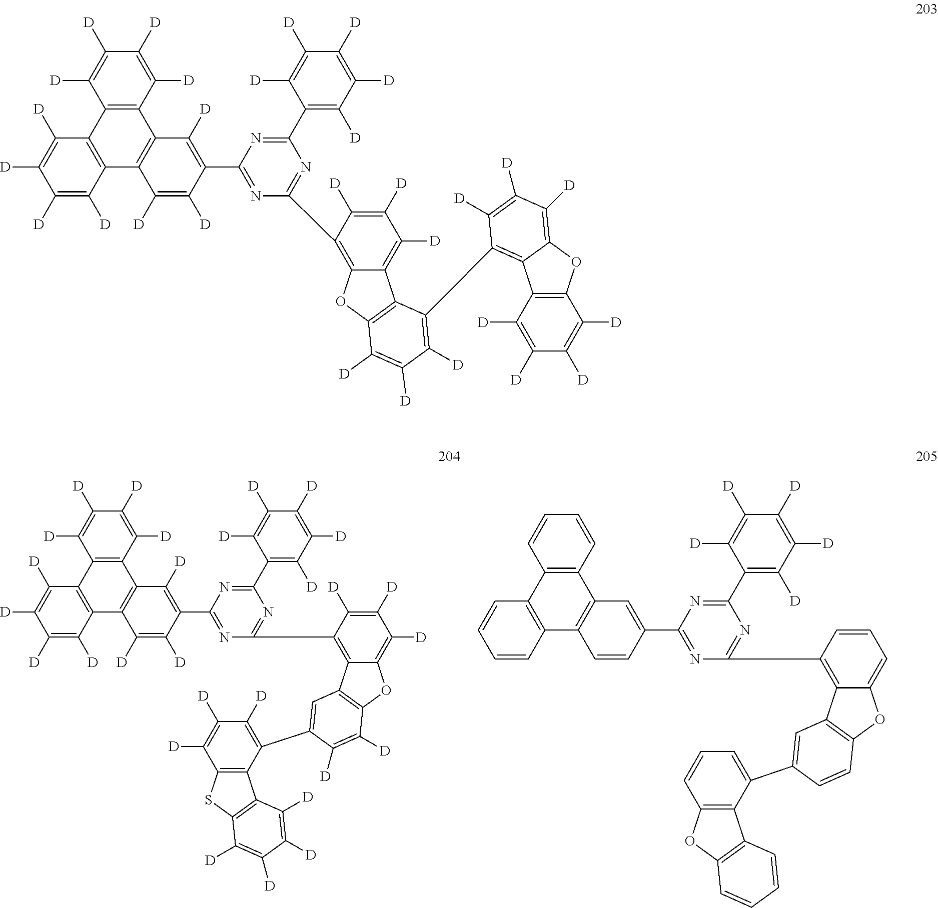

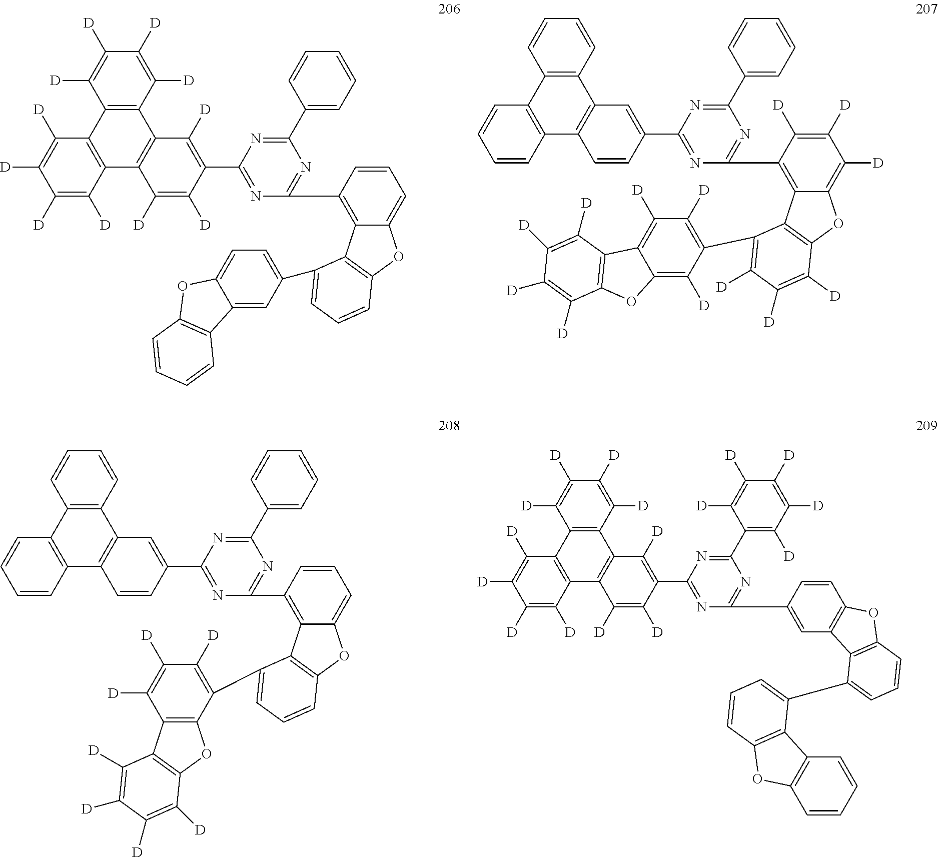

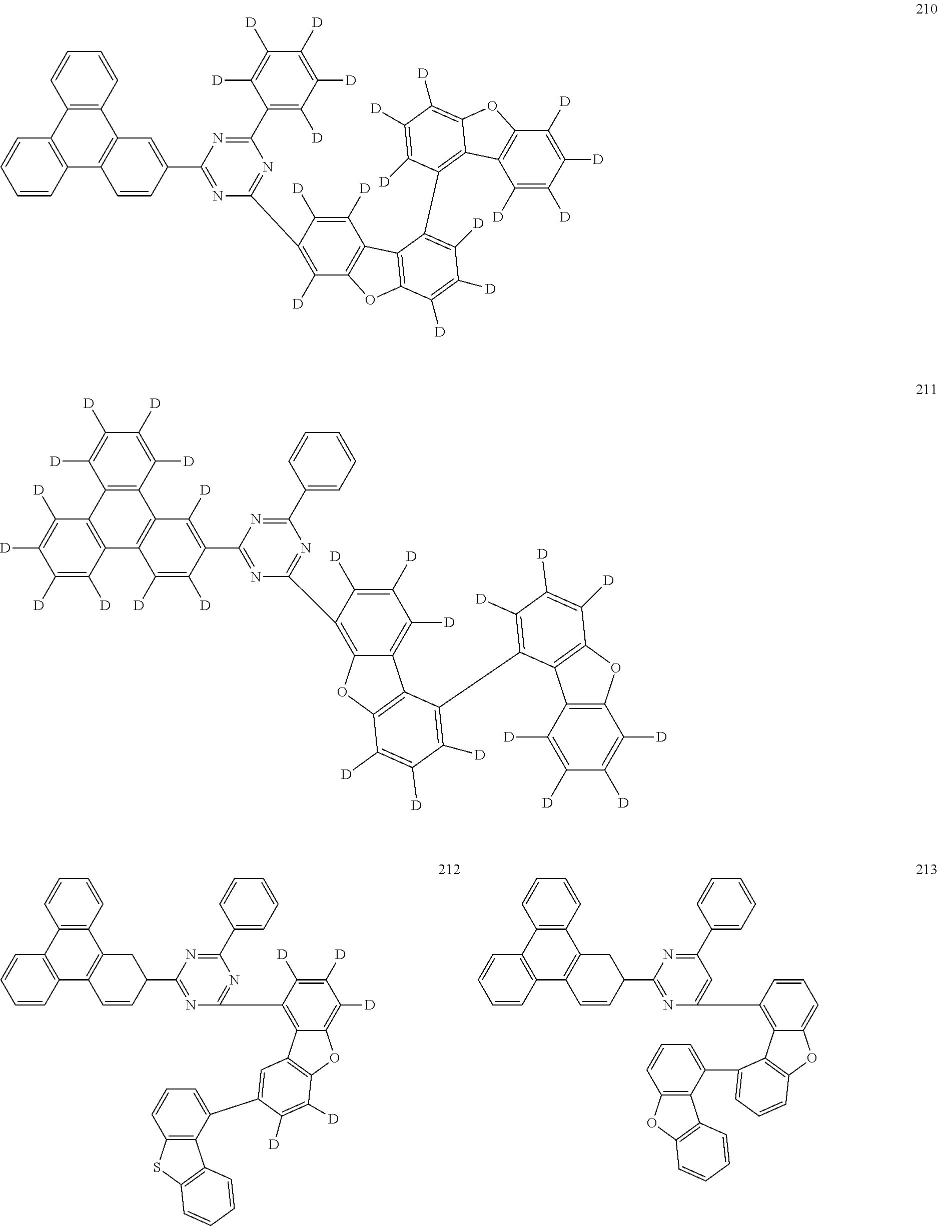

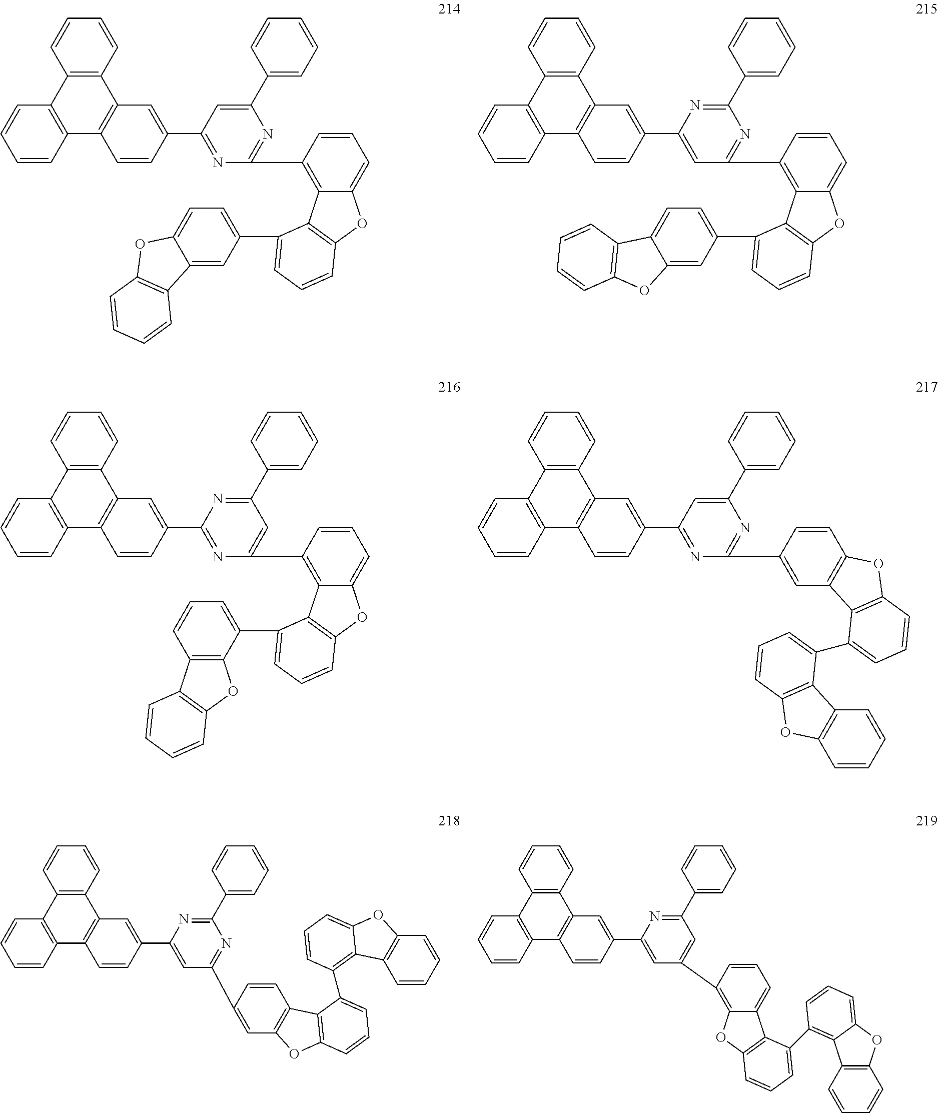

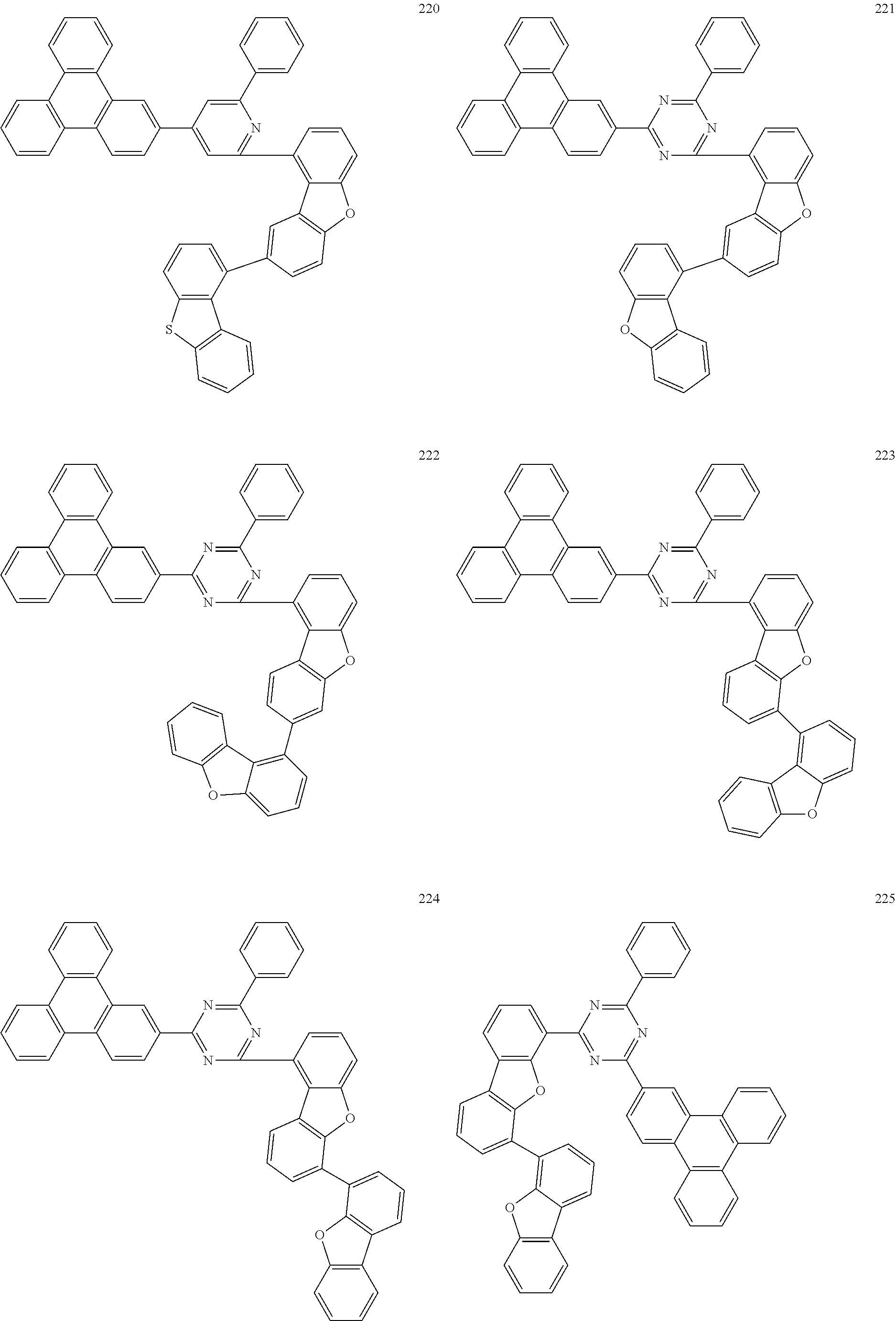

A composition for an organic optoelectronic device according to an embodiment includes a first compound and a second compound, wherein the first compound may be the aforementioned compound for an organic optoelectronic device and the second compound may be represented by Chemical Formula 2; or a combination of Chemical Formula 3 and Chemical Formula 4.

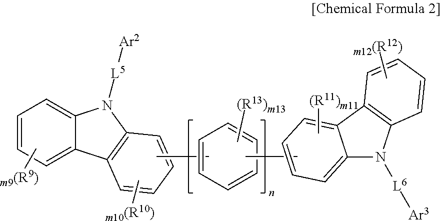

In Chemical Formula 2,
R⁹ to R¹³ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group,
Ar² and Ar³ are each independently a substituted or unsubstituted C6 to C20 aryl group or a substituted or unsubstituted C2 to C30 heterocyclic group,
L⁵ and L⁶ are each independently a single bond or a substituted or unsubstituted C6 to C20 arylene group,
m9, m12, and m13 are each independently one of integers of 1 to 4,
m10 and m11 are each independently one of integers of 1 to 3, and
n is one of integers of 0 to 2;

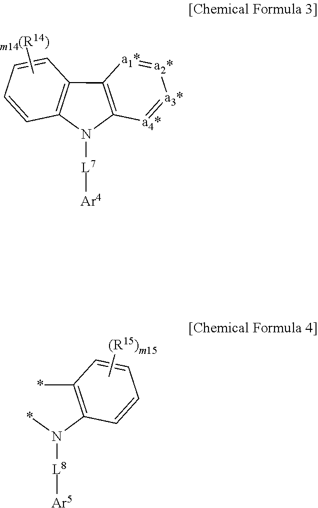

wherein, in Chemical Formula 3 and Chemical Formula 4,
a₁* to a₄* in Chemical Formula 3 are each independently a linking carbon (C) or C-L^{a}-R^{d},
among a₁* to a₄* in Chemical Formula 3, two adjacent ones are each linked to *s in Chemical Formula 4,
L^{a}, L⁷, and L⁸ are each independently a single bond or a substituted or unsubstituted C6 to C20 arylene group,
R^{d}, R¹⁴, and R¹⁵ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group,
Ar⁴ and Ar⁵ are each independently a substituted or unsubstituted C6 to C20 aryl group or a substituted or unsubstituted C2 to C30 heterocyclic group, and
m14 and m15 are each independently one of integers of 1 to 4.

The second compound may be used in the light emitting layer together with the first compound to improve luminous efficiency and life-span characteristics by increasing charge mobility and stability.

In Chemical Formula 2, when two or more R⁹s are substituted, each R⁹ may be the same or different from each other.

In Chemical Formula 2, when two or more R¹⁰s are substituted, each R¹⁰ may be the same or different from each other.

In Chemical Formula 2, when two or more R¹¹s are substituted, each R¹¹ may be the same or different from each other.

In Chemical Formula 2, when two or more R¹²s are substituted, each R¹² may be the same or different from each other.

In Chemical Formula 2, when two or more R¹³s are substituted, each R¹³ may be the same or different from each other.

In Chemical Formula 3 and Chemical Formula 4, when two or more R¹⁴s are substituted, each R¹⁴ may be the same or different from each other.

In Chemical Formula 3 and Chemical Formula 4, when two or more R¹⁵s are substituted, each R¹⁵ may be the same or different from each other.

In Chemical Formula 3 and Chemical Formula 4, when two or more R^{d}s are substituted, each R^{d} may be the same or different from each other.

For example, in Chemical Formula 2, Ar² and Ar³ may each independently be a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted triphenylenyl group, a substituted or unsubstituted carbazolyl group, a substituted or unsubstituted dibenzothiophenyl group, a substituted or unsubstituted dibenzofuranyl group, or a substituted or unsubstituted fluorenyl group,
in Chemical Formula 2, L⁵ and L⁶ may each independently be a single bond, a substituted or unsubstituted phenylene group, or a substituted or unsubstituted biphenylene group,
in Chemical Formula 2, R⁹ to R¹³ may each independently be hydrogen, deuterium, or a substituted or unsubstituted C6 to C12 aryl group, and
n may be 0 or 1.

As an example, in Chemical Formula 2, “substituted” refers to replacement of at least one hydrogen by deuterium, a C1 to C4 alkyl group, a C6 to C18 aryl group, or a C2 to C30 heteroaryl group.

For example, in Chemical Formula 2, Ar² and Ar³ may each independently be a substituted or unsubstituted carbazolyl group, a substituted or unsubstituted dibenzothiophenyl group, a substituted or unsubstituted dibenzofuranyl group, or a substituted or unsubstituted fluorenyl group.

In a specific embodiment of the present invention, Chemical Formula 2 may be represented by one of Chemical Formula 2-1 to Chemical Formula 2-15.

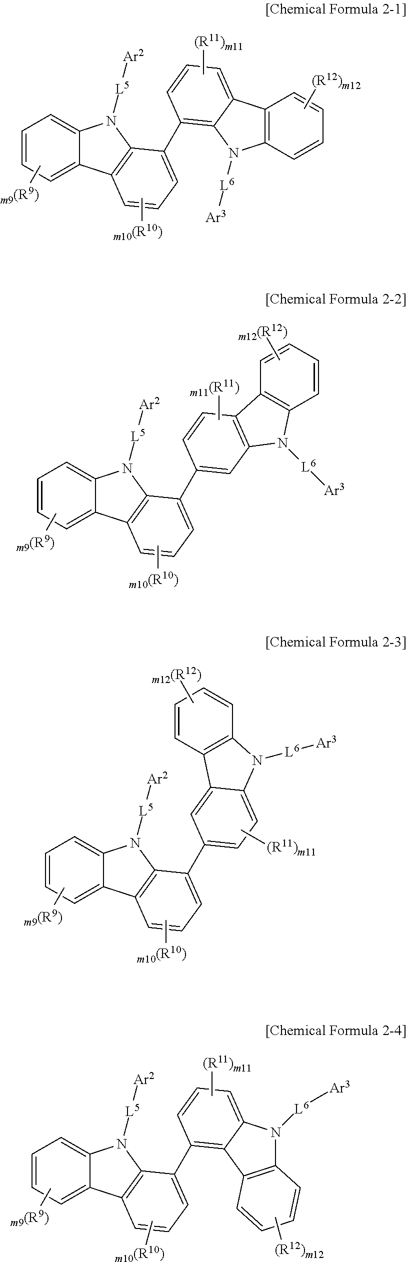

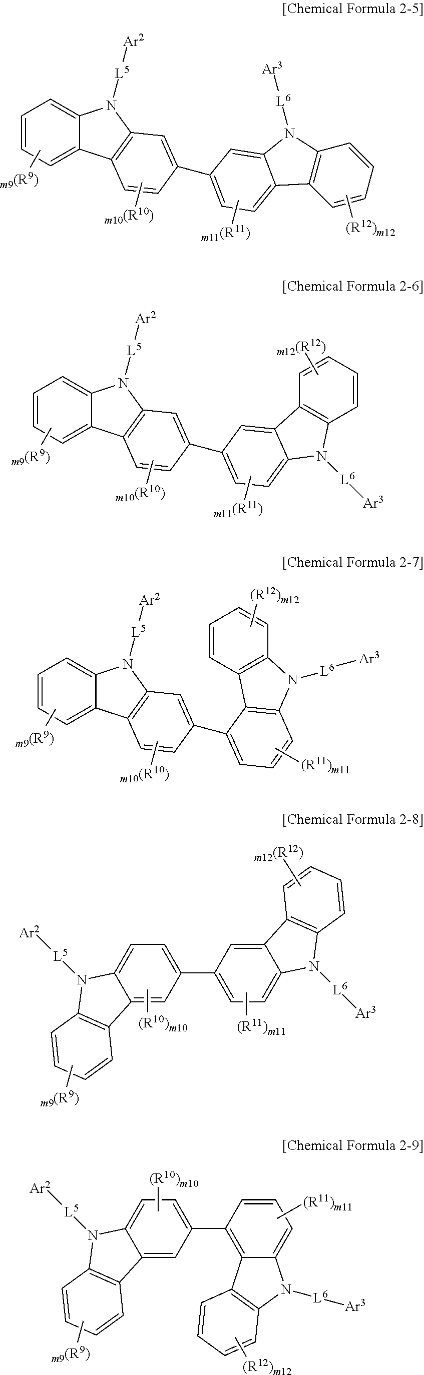

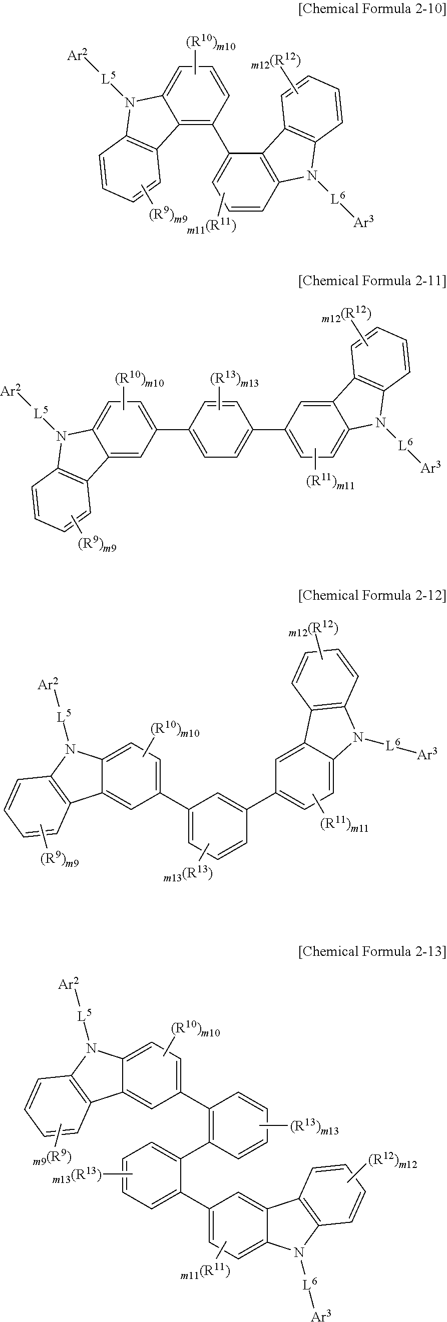

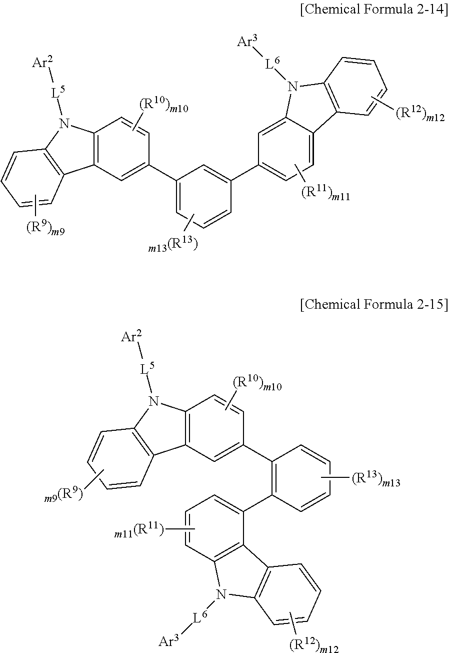

In Chemical Formula 2-1 to Chemical Formula 2-15, R⁹ to R¹³ are each independently hydrogen, deuterium, or a substituted or unsubstituted C6 to C12 aryl group, *-L²-Ar⁵ and *-L³-Ar⁶ may each independently be one of the substituents listed in Group I.

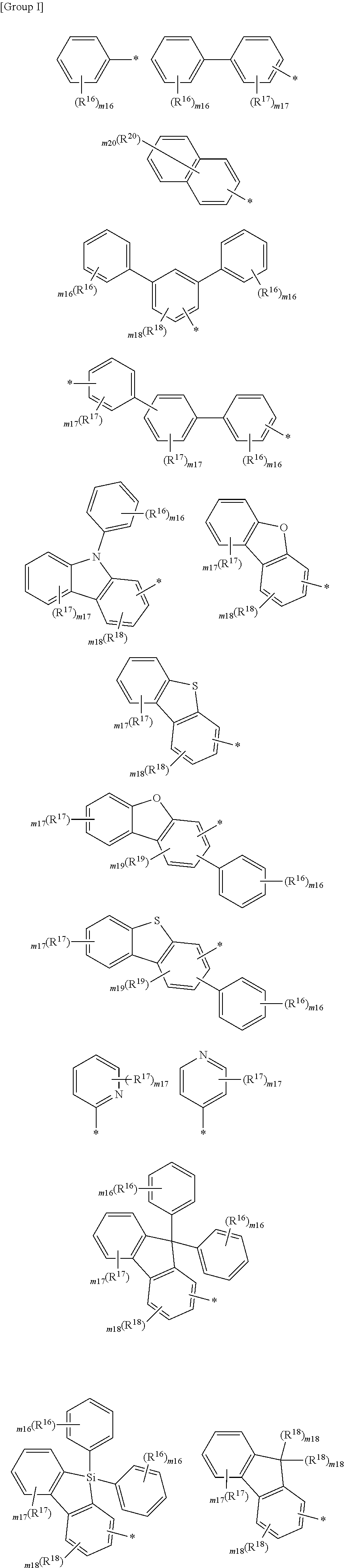

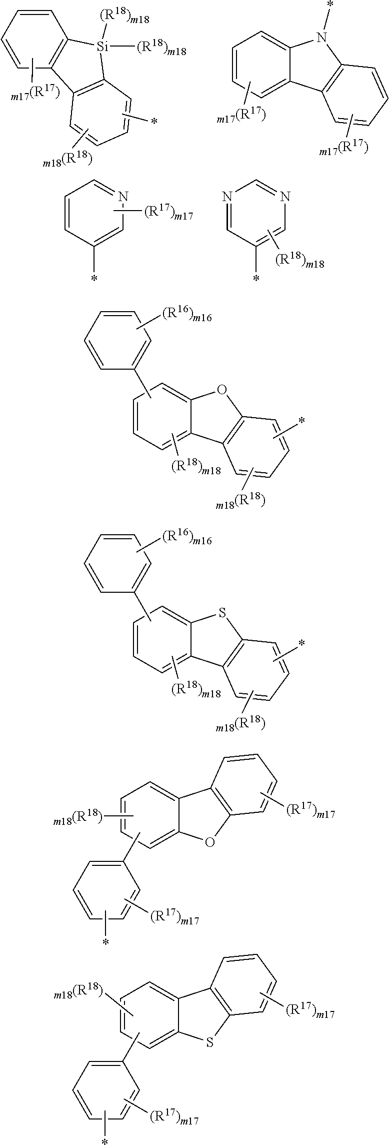

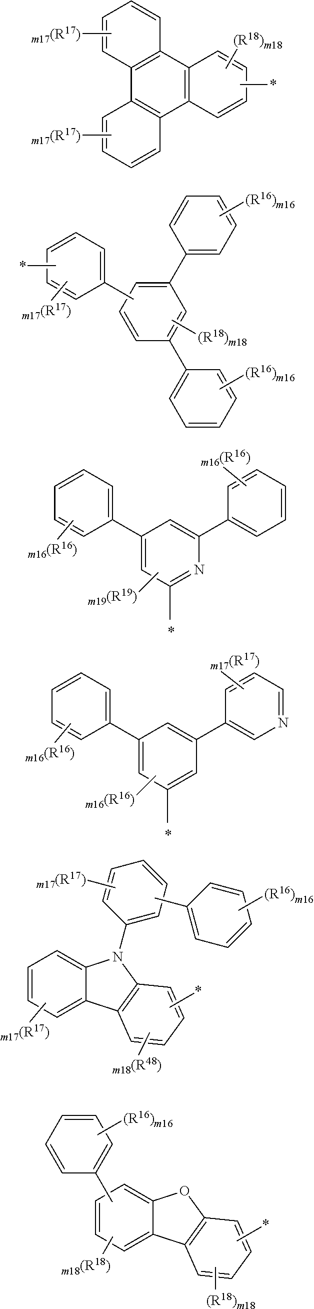

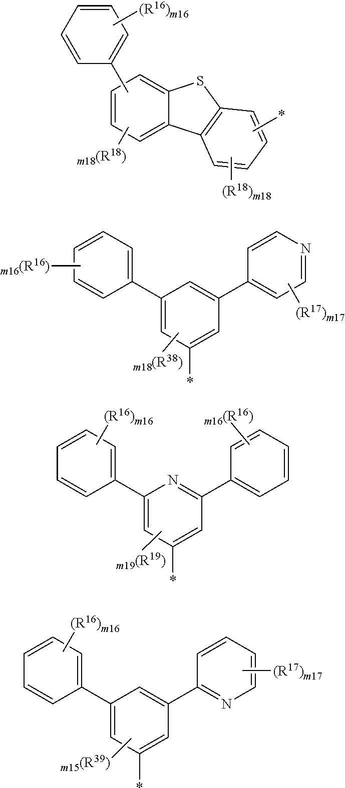

In Group I,
R¹⁶to R²⁰ are each independently hydrogen, deuterium, a cyano group, a C1 to C10 alkyl group, or a C6 to C12 aryl group,
m16 is one of integers of 1 to 5,
m17 is one of integers of 1 to 4,
m18 is one of integers of 1 to 3,
m19 is an integer of 1 or 2,
m20 is one of integers of 1 to 7, and
* is a linking point.

In Group I, when two or more R¹⁶s are substituted, each R¹⁶ may be the same or different from each other.

In Group I, when two or more R¹⁷s are substituted, each R¹⁷ may be the same or different from each other.

In Group I, when two or more R¹⁸s are substituted, each R¹⁸ may be the same or different from each other.

In Group I, when two or more R¹⁹s are substituted, each R¹⁹ may be the same or different from each other.

In Group I, when two or more R²⁰s are substituted, each R²⁰ may be the same or different from each other.

The second compound may be, for example, represented by any one of Chemical Formula 3A, Chemical Formula 3B, Chemical Formula 3C, Chemical Formula 3D, and Chemical Formula 3E.

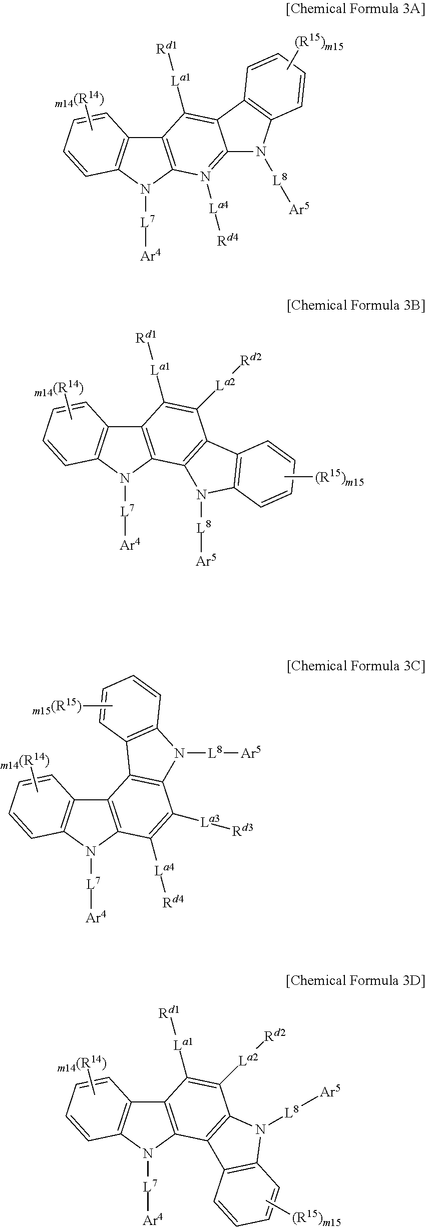

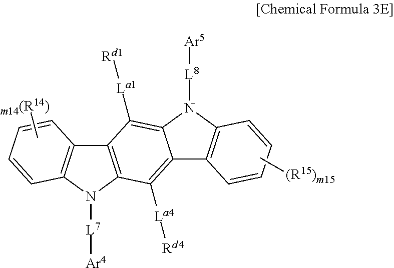

In Chemical Formula 3A to Chemical Formula 3E, L⁷, L⁸, Ar⁴, Ar⁵, R¹⁴, and R¹⁵ are the same as described above,
L^{a1} to L^{a4} are defined as the aforementioned L⁷ and L⁸, and
R^{d1} to R^{d4} are defined as the aforementioned R¹⁴ and R¹⁵.

For example, in Chemical Formulas 3 and 4, Ar⁴ and Ar⁵ may each independently be a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted triphenylenyl group, a substituted or unsubstituted carbazolyl group, a substituted or unsubstituted dibenzothiophenyl group, a substituted or unsubstituted dibenzofuranyl group, or a substituted or unsubstituted fluorenyl group, and
R^{d1} to R^{d4}, R¹⁴, and R¹⁵ may each independently be hydrogen, deuterium, a cyano group, a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group.

In a specific embodiment of the present invention, in Chemical Formulas 3 and 4, *-L⁷-Ar⁴ and *-L⁸-Ar⁵ may each independently be selected from the substituents listed in Group I.

In an embodiment, R^{d1} to R^{d4}, R¹⁴, and R¹⁵ may each independently be hydrogen, deuterium, a cyano group, a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group.

For example, R^{d1} to R^{d4}, R¹⁴, and R¹⁵ may each independently be hydrogen, deuterium, a cyano group, or a substituted or unsubstituted phenyl group, and
in a specific embodiment, R^{d1} to R^{d4}, R¹⁴, and R¹⁵ may each independently be hydrogen, deuterium, or a substituted or unsubstituted phenyl group.

In a specific embodiment of the present invention, the second compound may be represented by Chemical Formula 2-8, wherein in Chemical Formula 2-8, Ar² and Ar³ may each independently be a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group, L⁵ and L⁶ may each independently be a single bond, or a substituted or unsubstituted C6 to C20 arylene group, and R⁹ to R¹² may each independently be hydrogen, deuterium, a cyano group, a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group.

For example, in Chemical Formula 2-8, R⁹ to R¹² may each independently be hydrogen, deuterium, or a substituted or unsubstituted C6 to C12 aryl group, and *-L⁵-Ar² and *-L⁶-Ar³ may each independently be one of the substituents listed in Group I.

In another specific embodiment of the present invention, the second compound may be represented by Chemical Formula 3C, wherein in Chemical Formula 3C, L^{a3} and L^{a4} may be a single bond, L⁷ and L⁸ may each independently be a single bond or a substituted or unsubstituted C6 to C12 arylene group, R¹⁴, R¹, R^{a3}, and R^{d4} are each hydrogen, deuterium or phenyl group, and Ar⁴ and Ar⁵ may each independently be a substituted or unsubstituted phenyl group, a substituted or unsubstituted biphenyl group, a substituted or unsubstituted pyridinyl group, a substituted or unsubstituted carbazolyl group, a substituted or unsubstituted dibenzofuranyl group, or a substituted or unsubstituted dibenzothiophenyl group.

For example, in Chemical Formula 3C, L^{c3} and L^{c4} may be a single bond, R¹⁴, R¹⁵, R^{d3}, and R^{d4} may each independently be hydrogen, deuterium or a C6 to C12 aryl group, and *-L⁷-Ar⁴ and *-L⁸-Ar⁵ may each independently be one of the substituents listed in Group I.

For example, the compound for the second organic optoelectronic device may be one selected from the compounds listed in Group 2, but is not limited thereto.

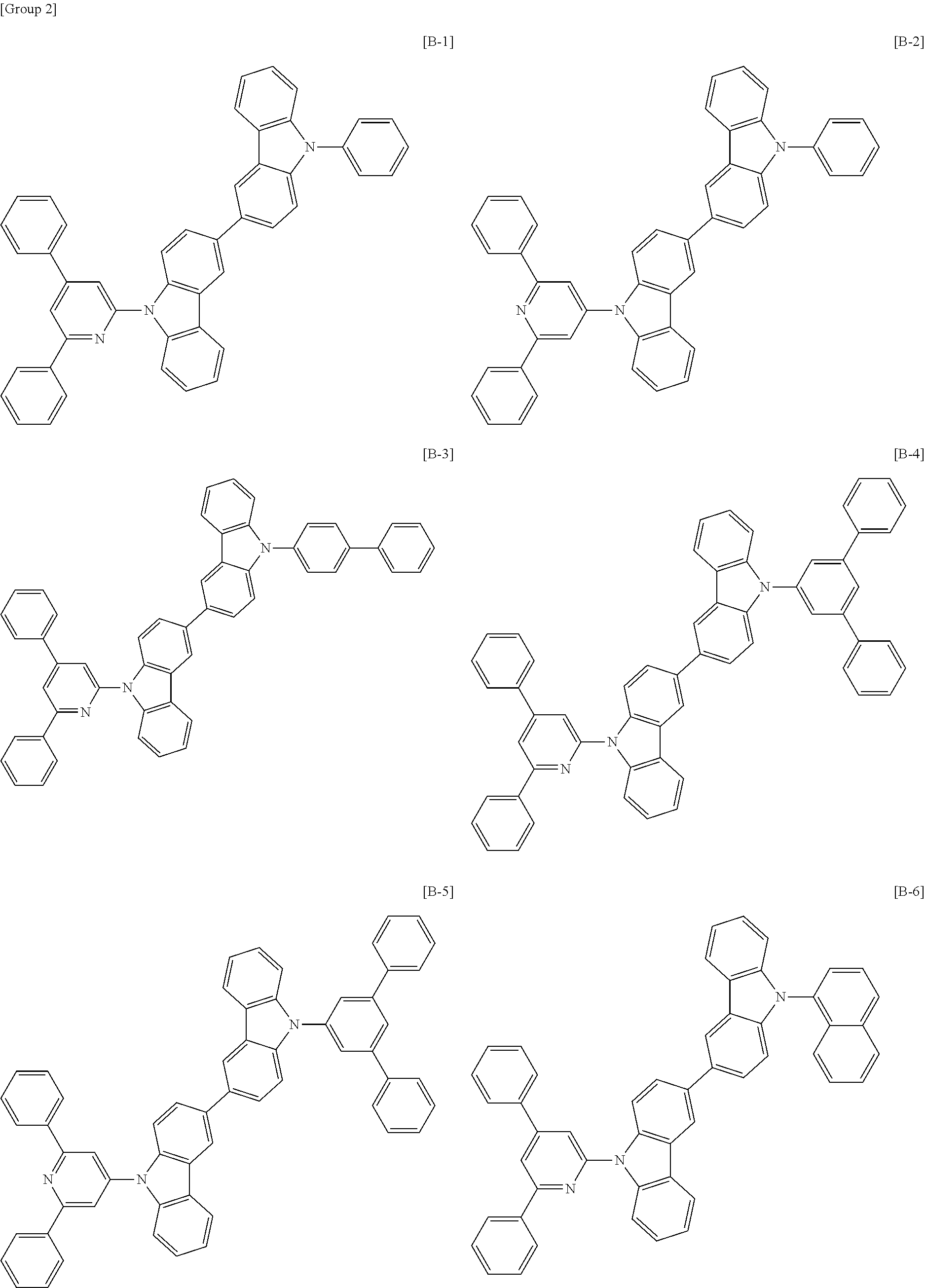

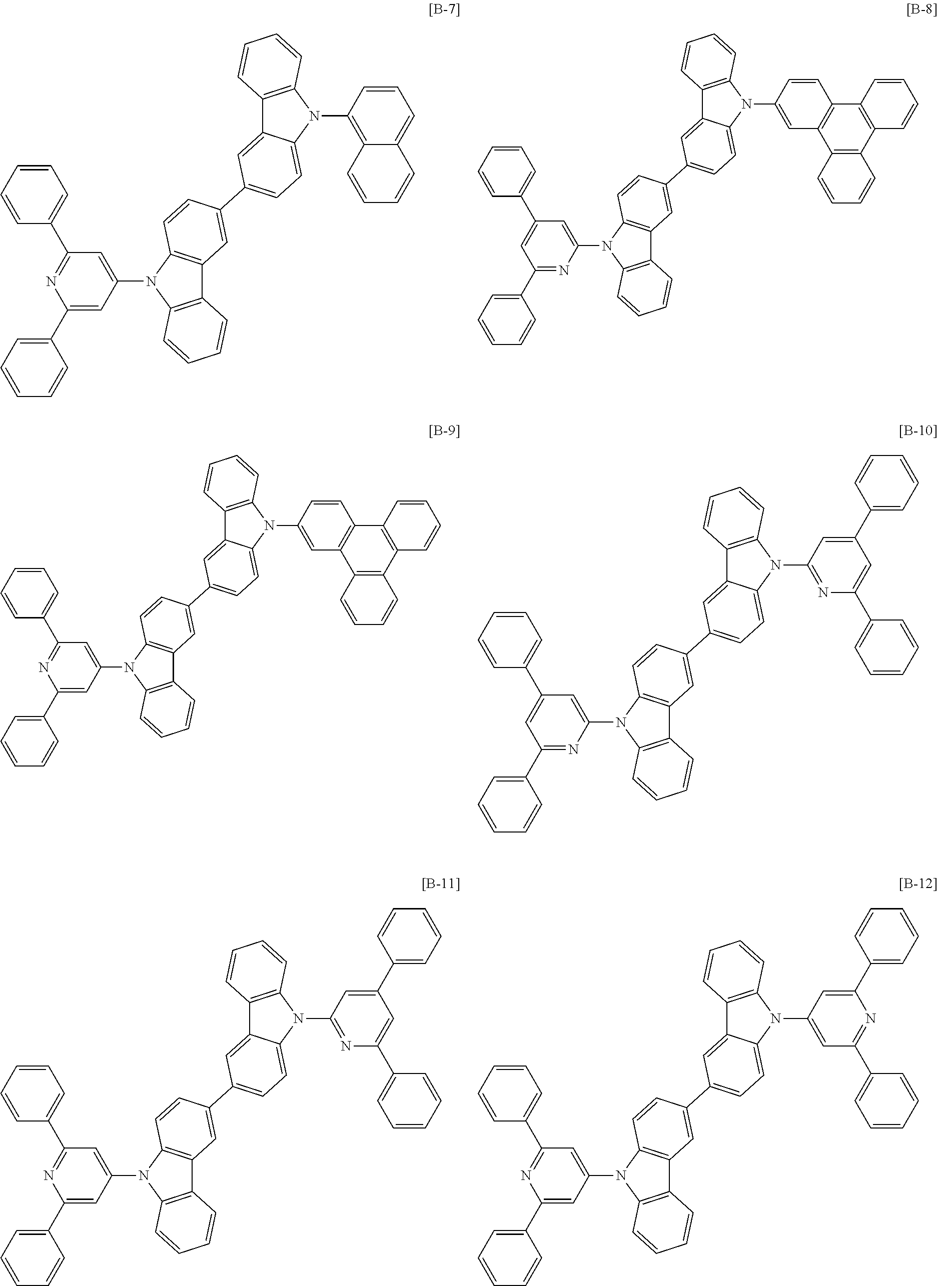

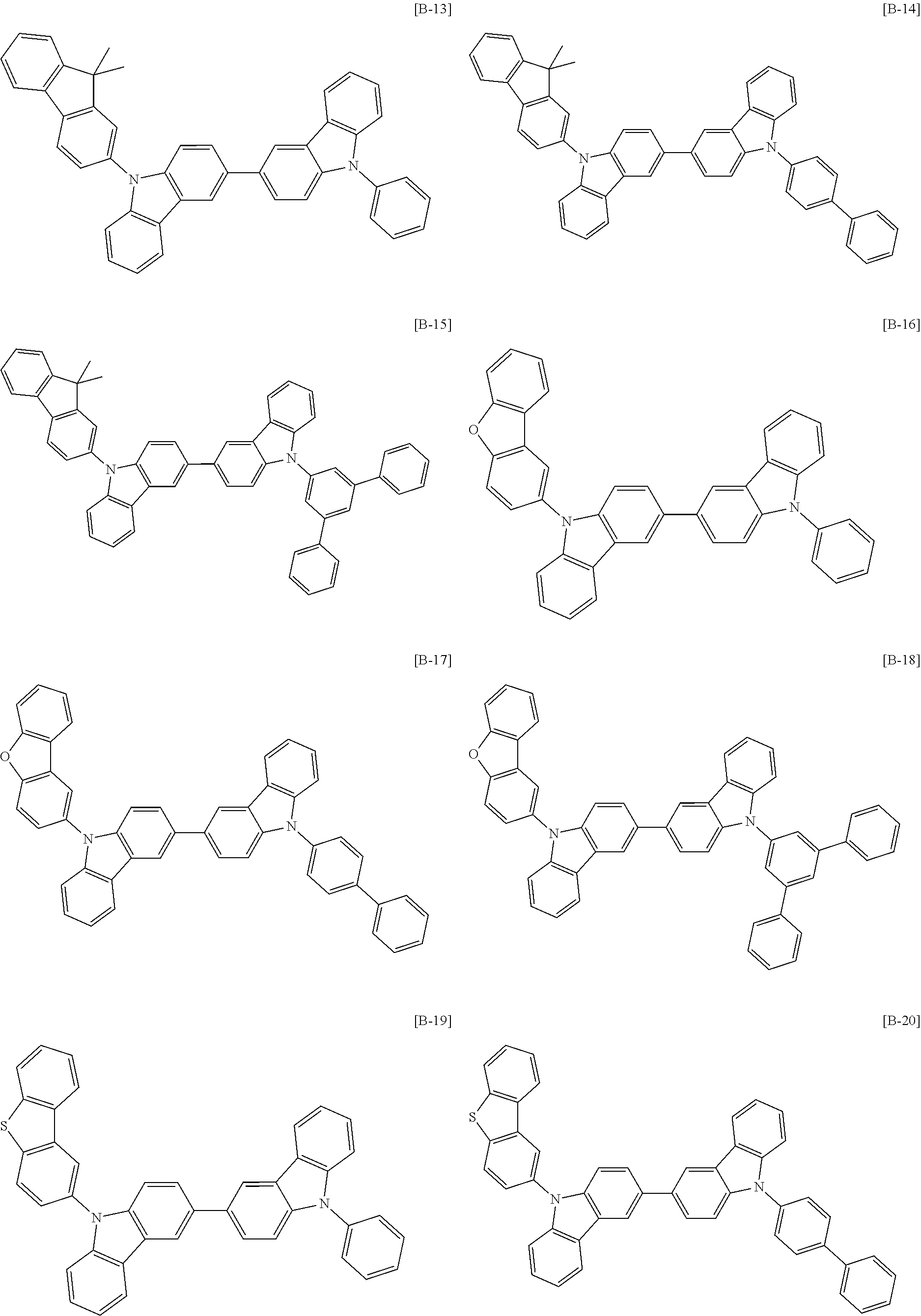

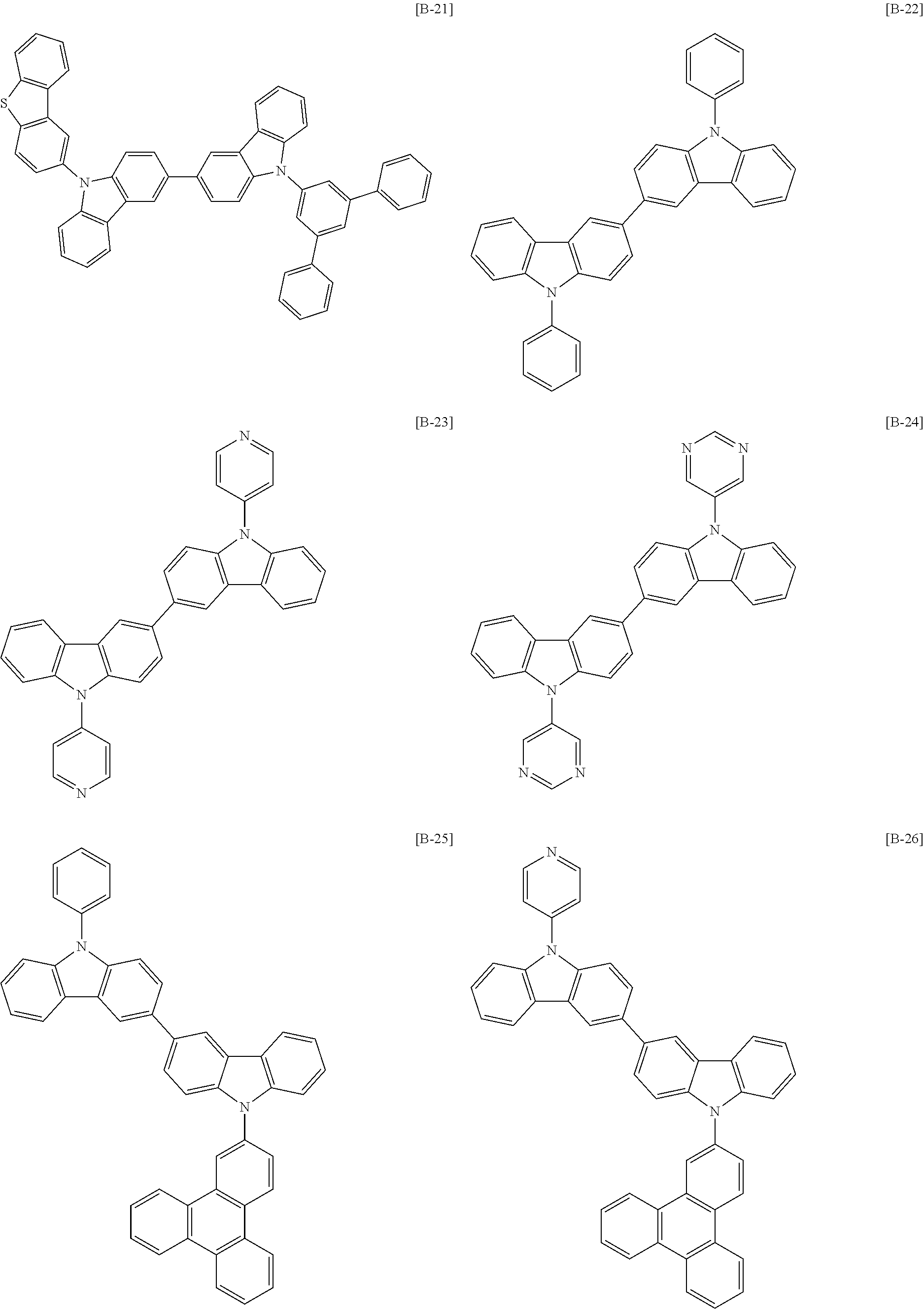 [SMILES: c1ccc(-c2cc(-c3ccccc3)cc(-n3c4ccccc4c4cc(-c5ccc6c(c5)c5ccccc5n6-c5ccc6sc7ccccc7c6c5)ccc43)c2)cc1 ; c1ccc(-n2c3ccccc3c3cc(-c4ccc5c(c4)c4ccccc4n5-c4ccccc4)ccc32)cc1 ; c1ccc2c(c1)c1cc(-c3ccc4c(c3)c3ccccc3n4-c3ccncc3)ccc1n2-c1ccncc1 ; c1ccc2c(c1)c1cc(-c3ccc4c(c3)c3ccccc3n4-c3cncnc3)ccc1n2-c1cncnc1 ; c1ccc(-n2c3ccccc3c3cc(-c4ccc5c(c4)c4ccccc4n5-c4ccc5c6ccccc6c6ccccc6c5c4)ccc32)cc1 ; c1ccc2c(c1)c1ccccc1c1cc(-n3c4ccccc4c4cc(-c5ccc6c(c5)c5ccccc5n6-c5ccncc5)ccc43)ccc21]

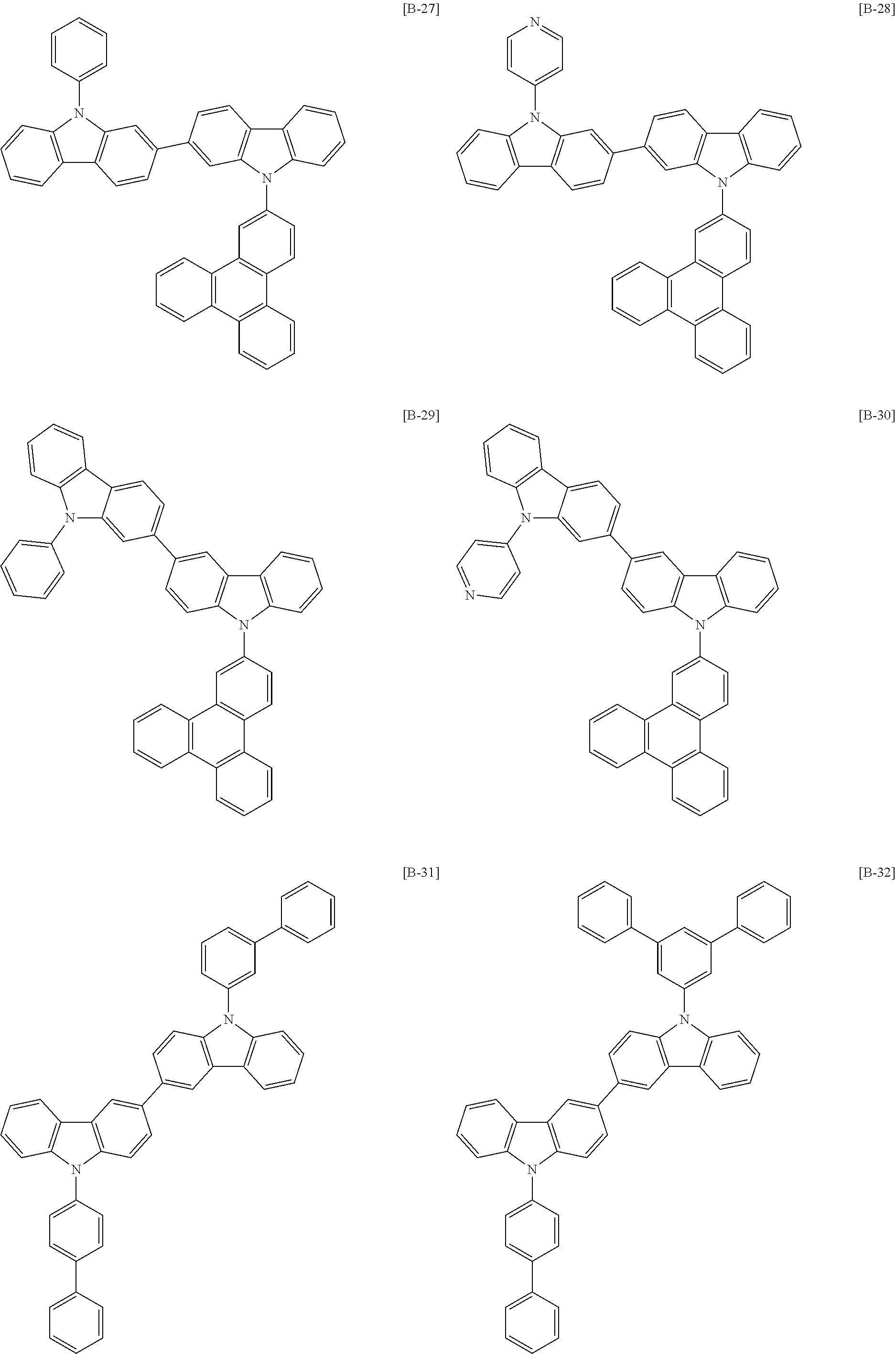

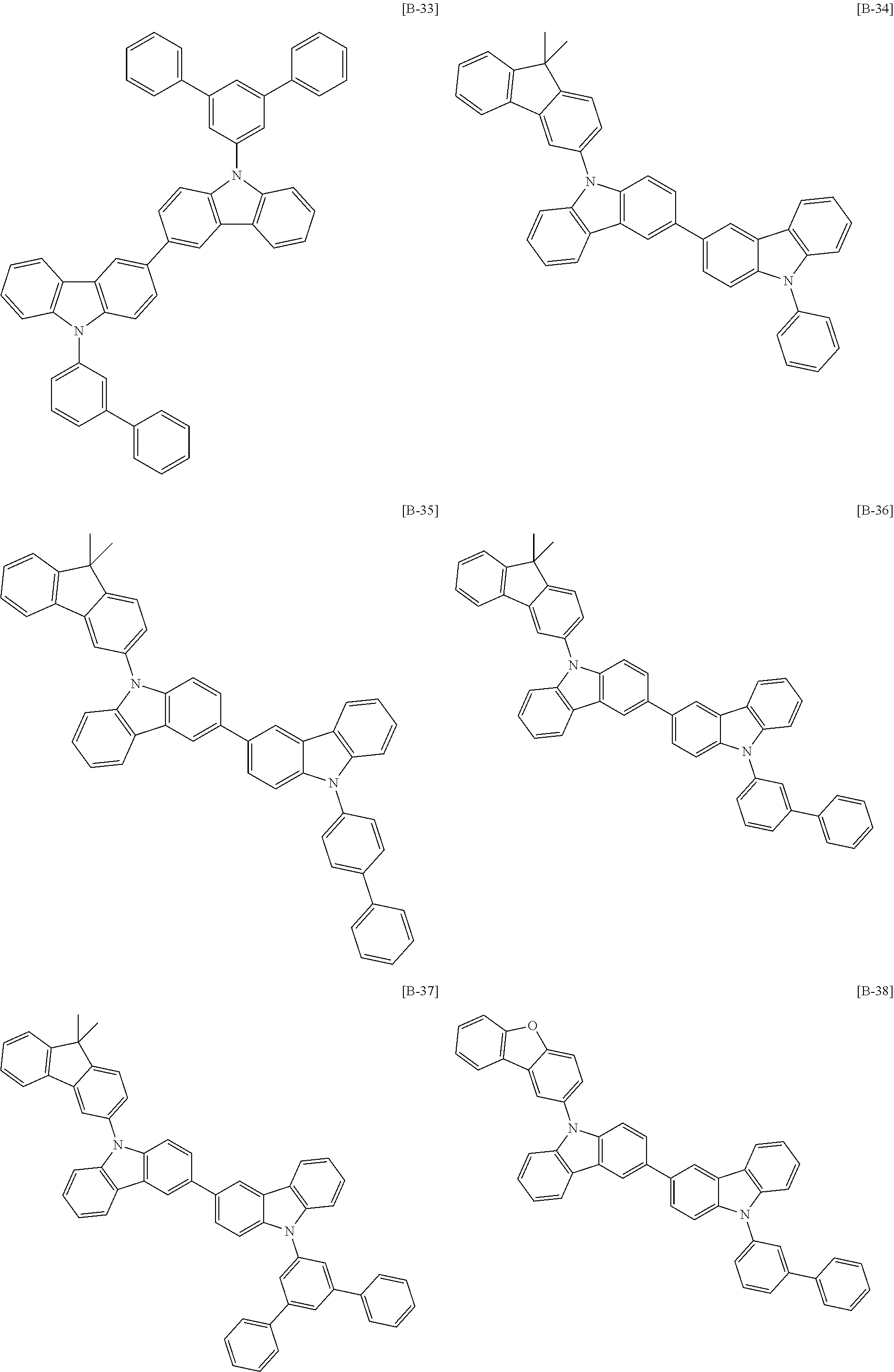

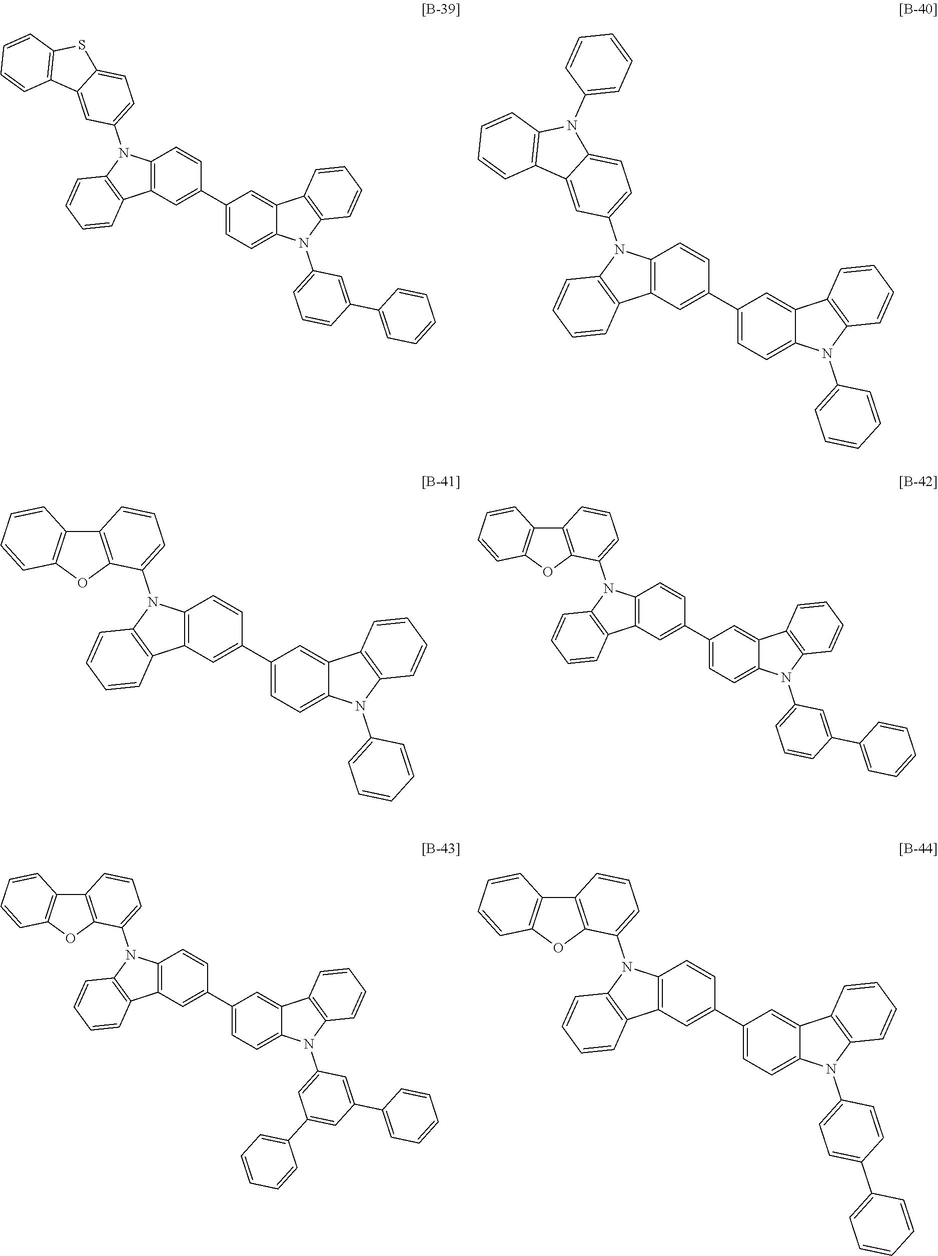

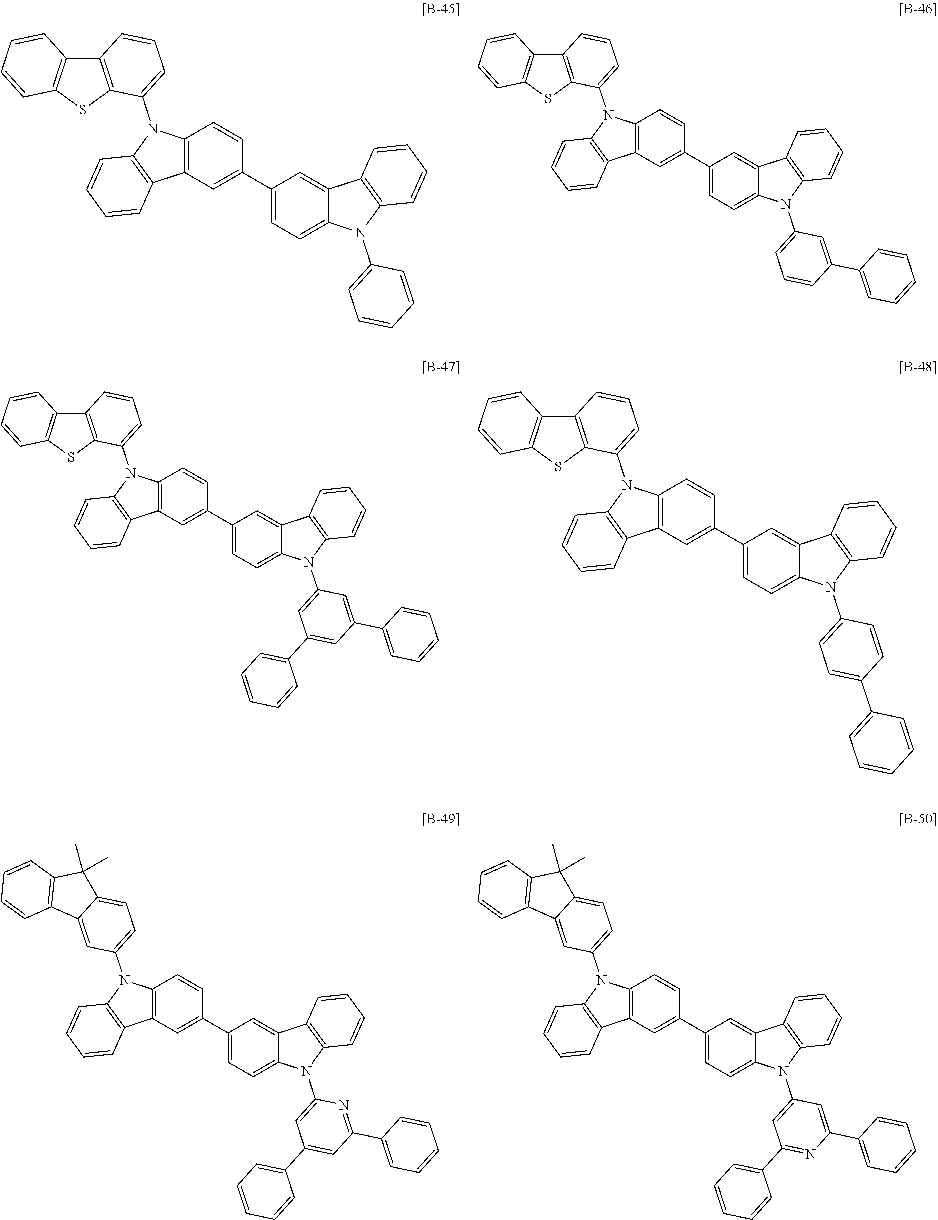

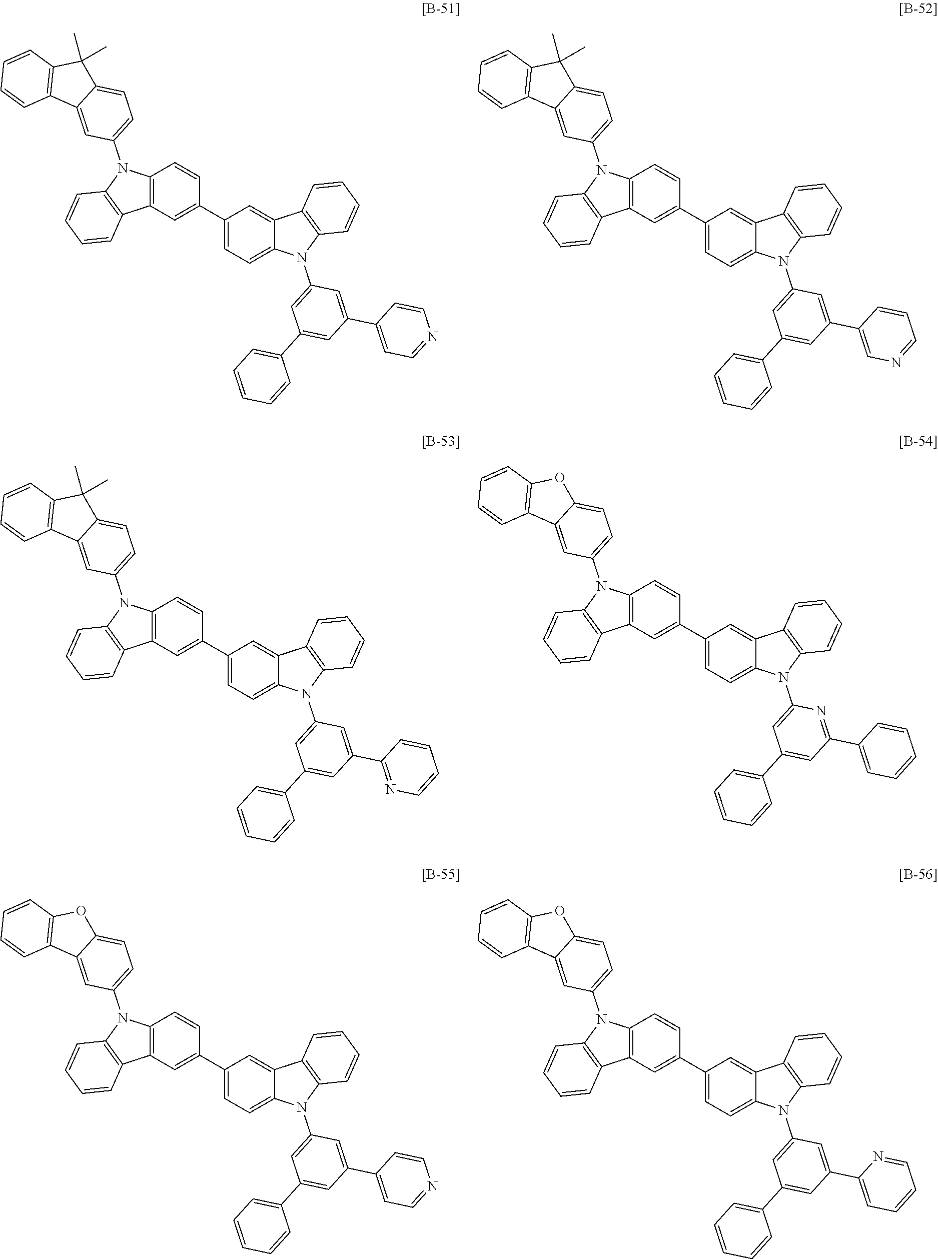

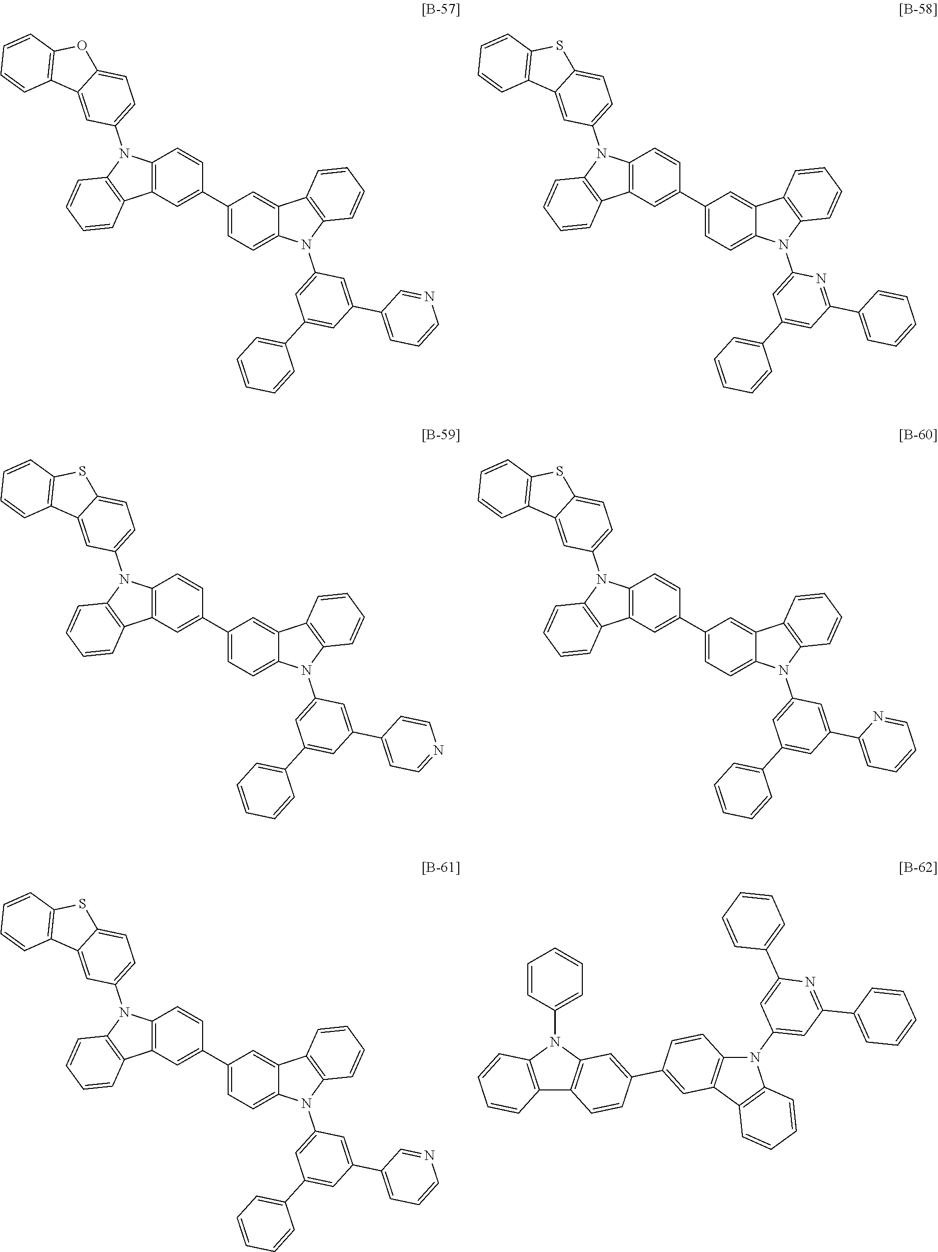

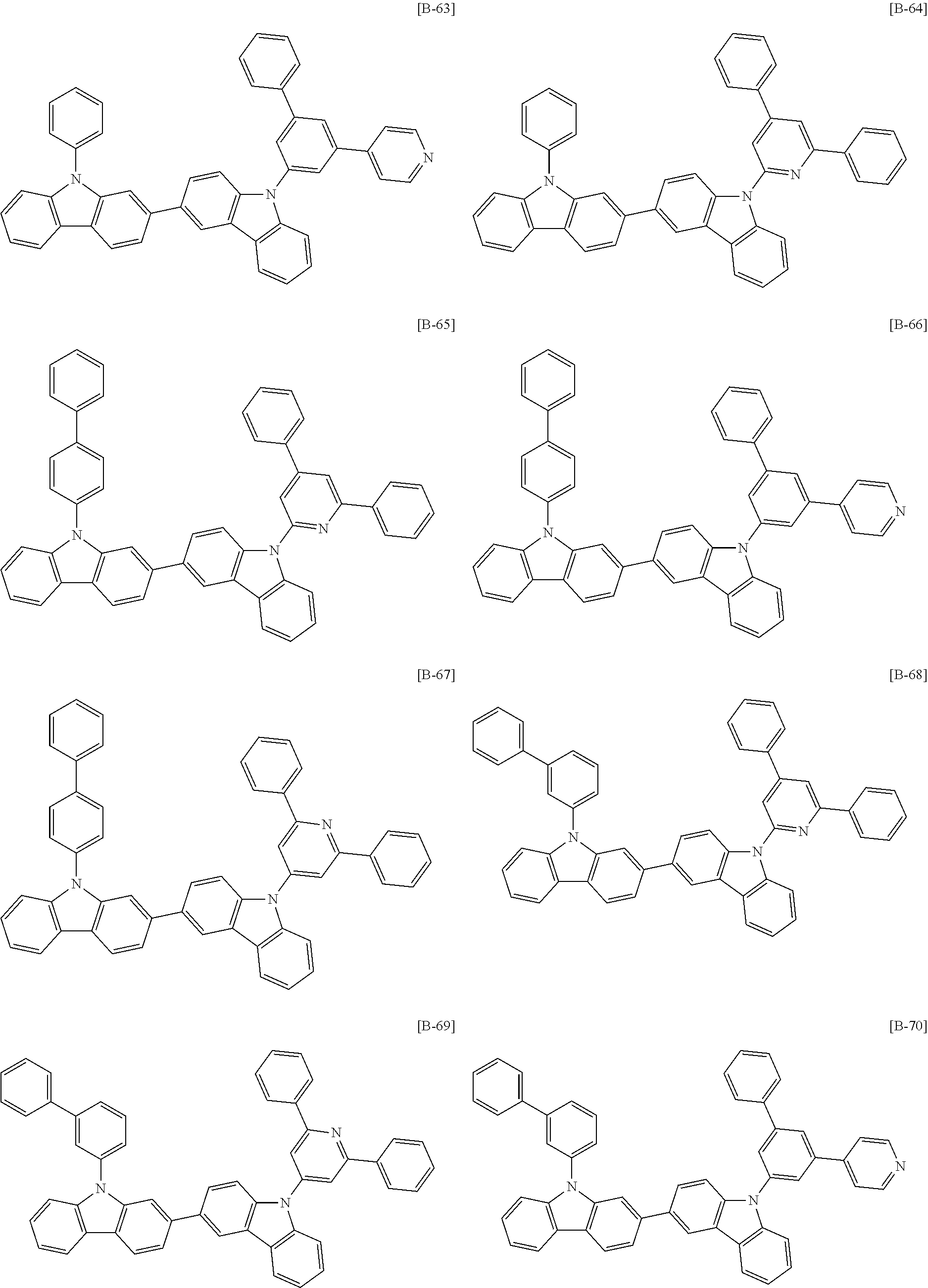

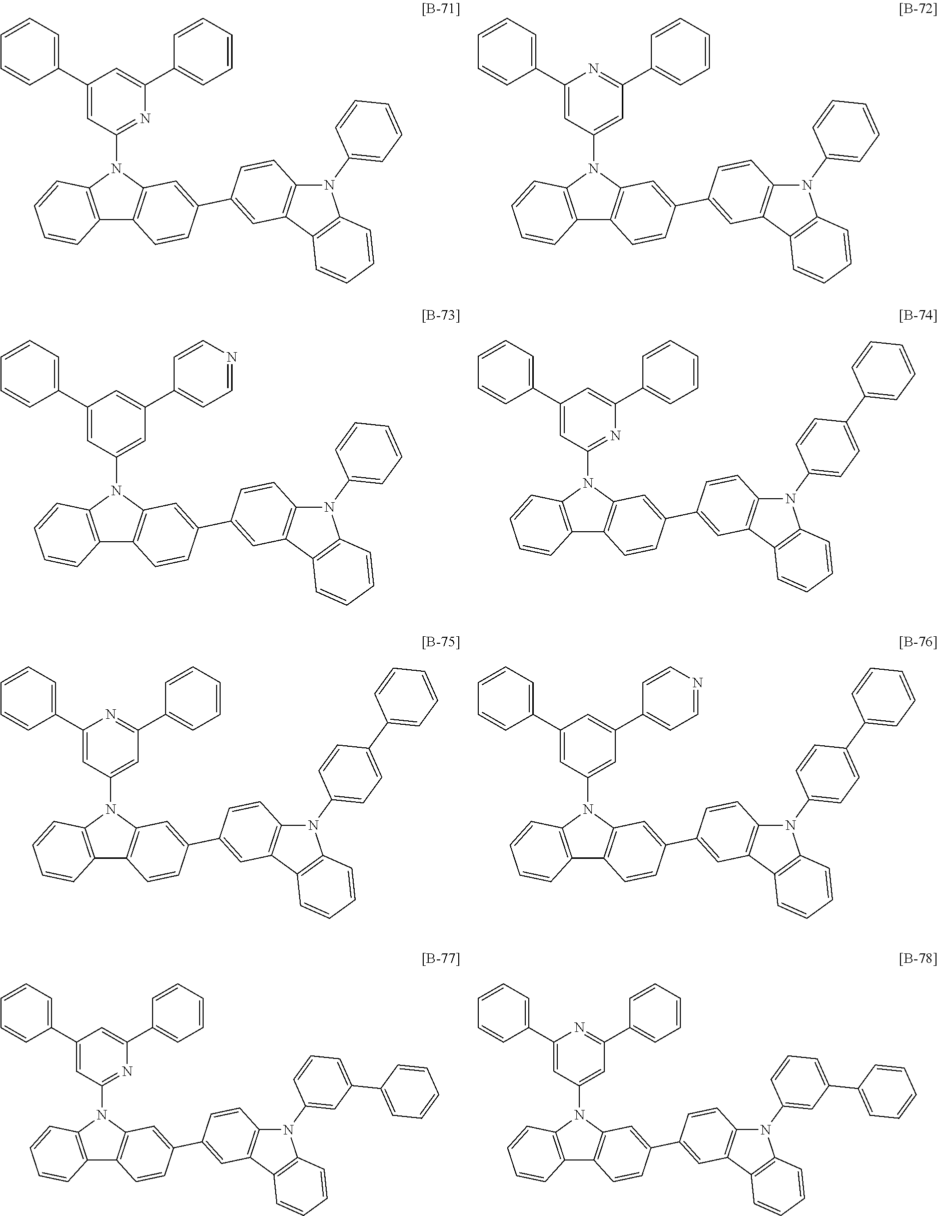

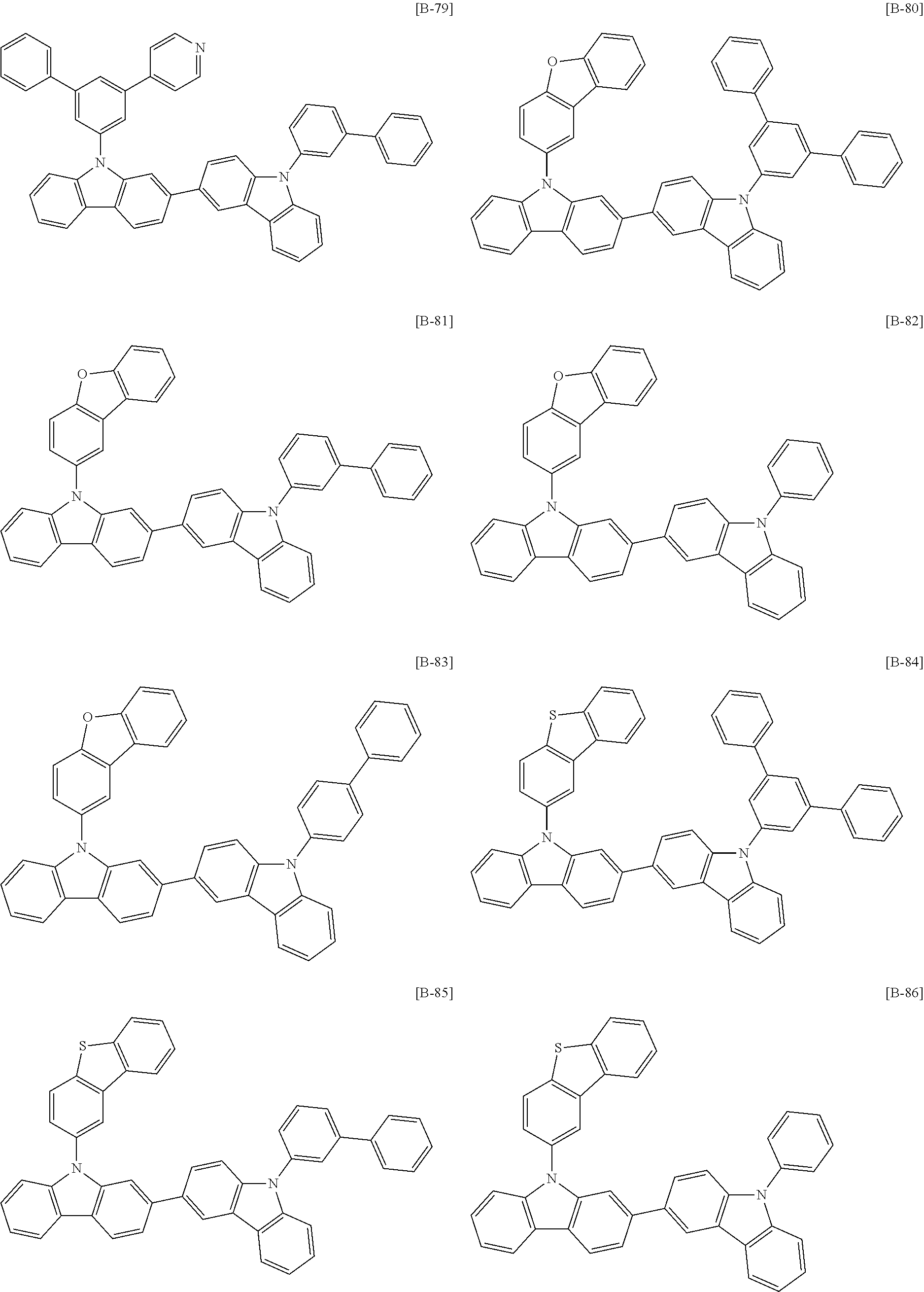

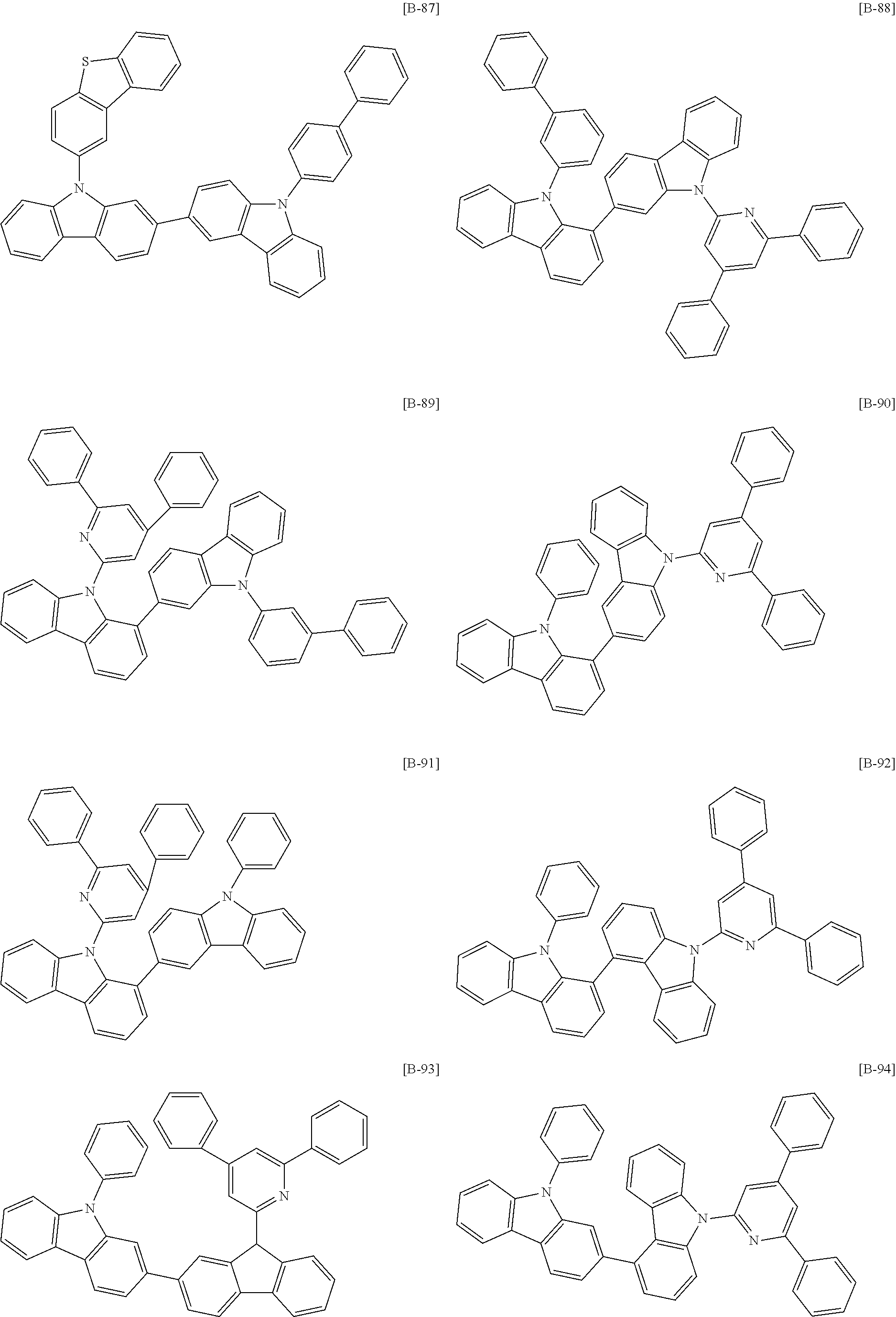

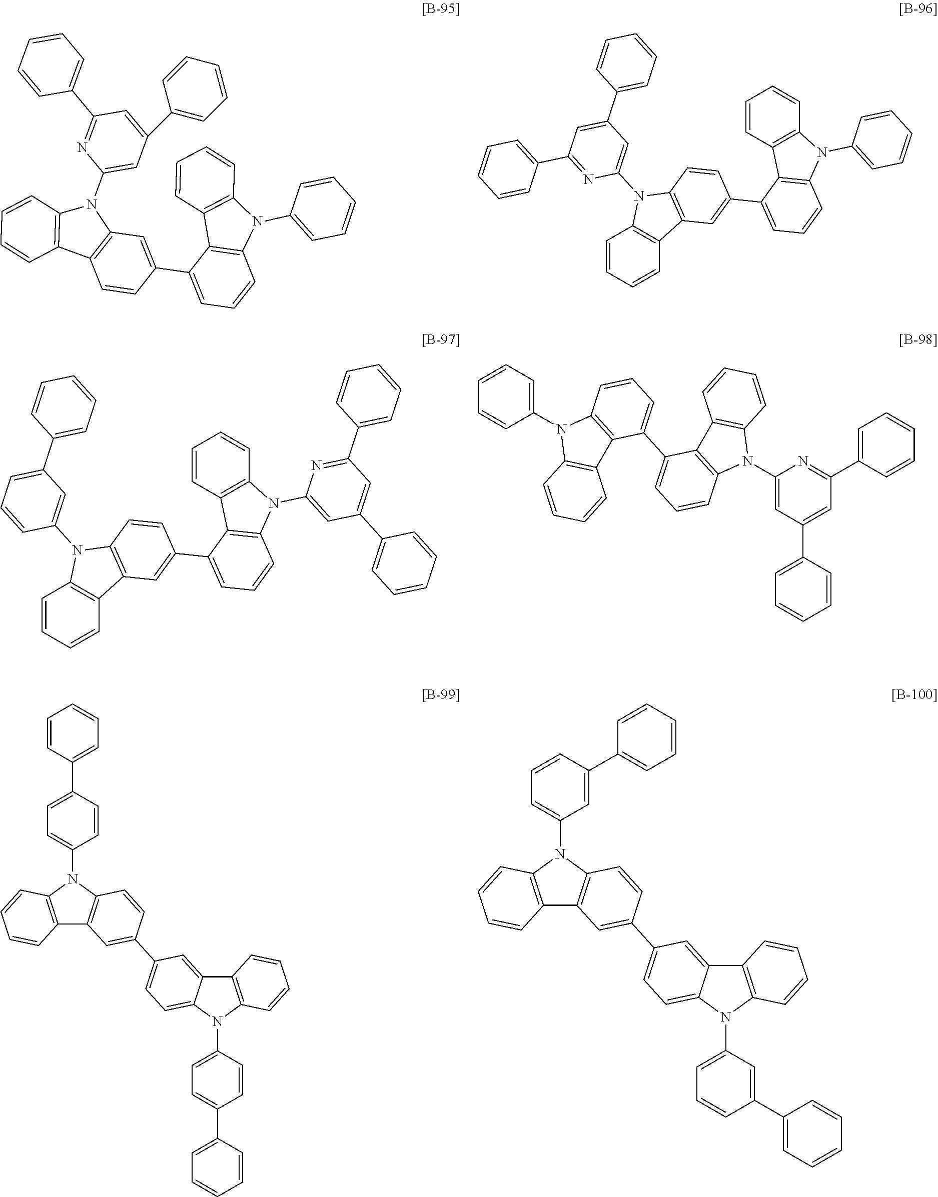

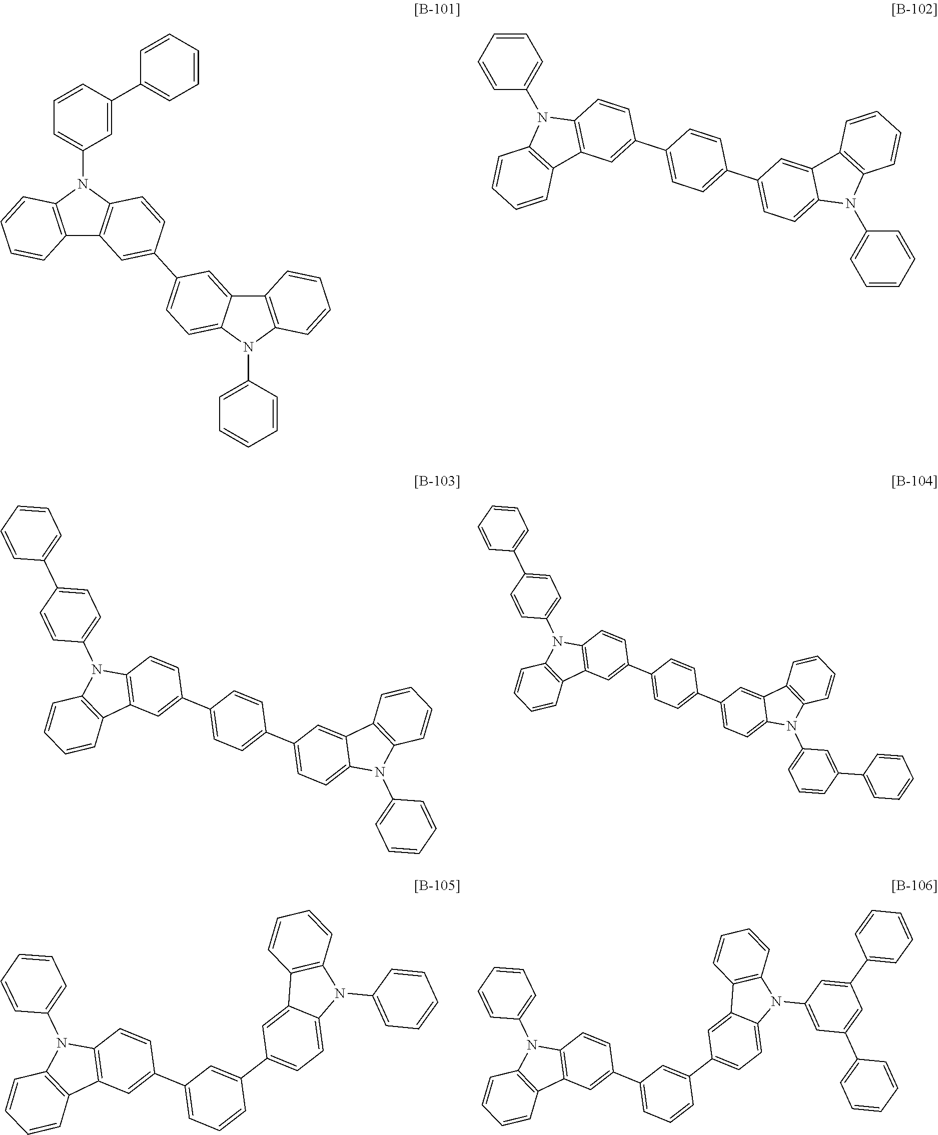

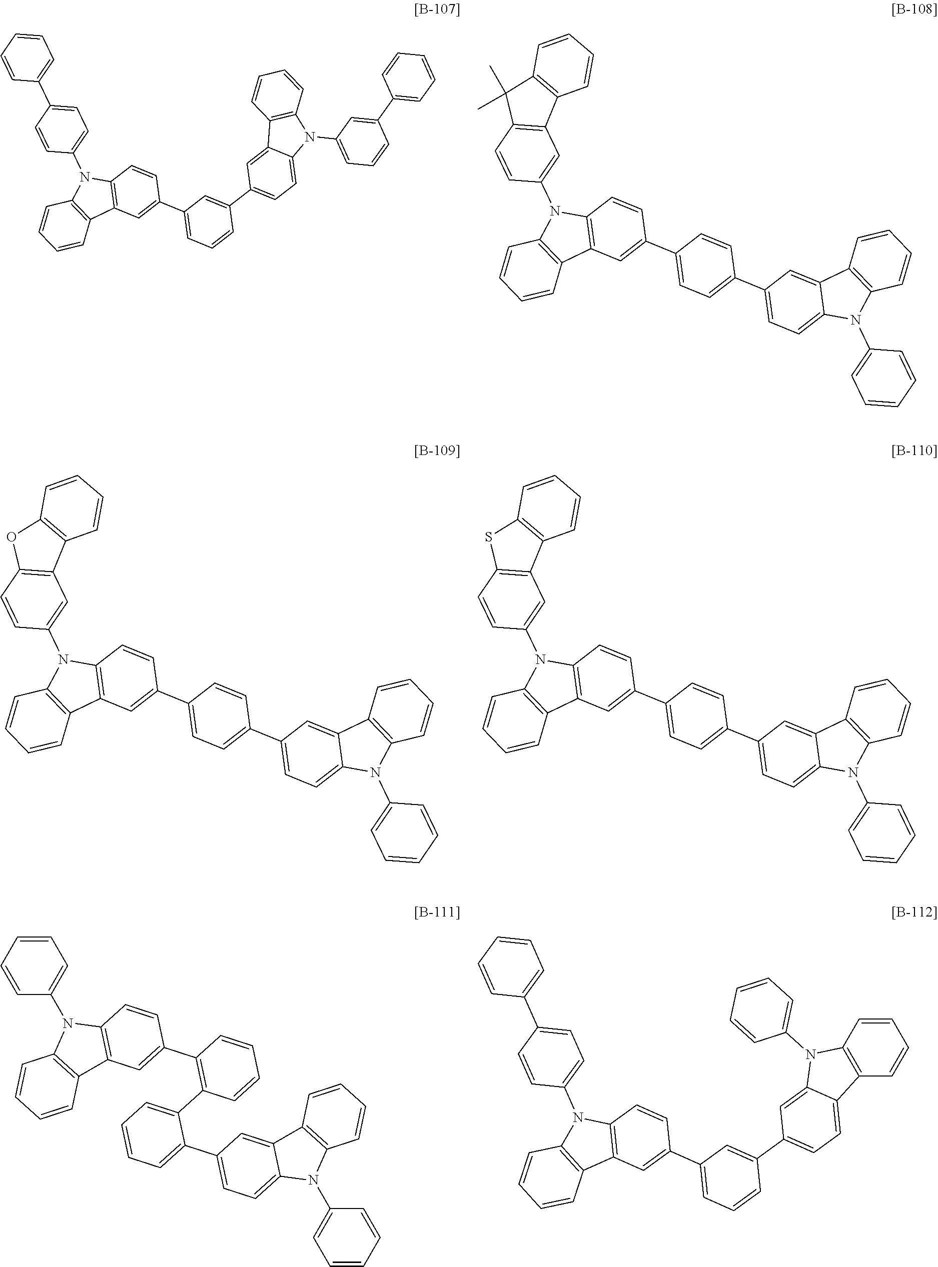

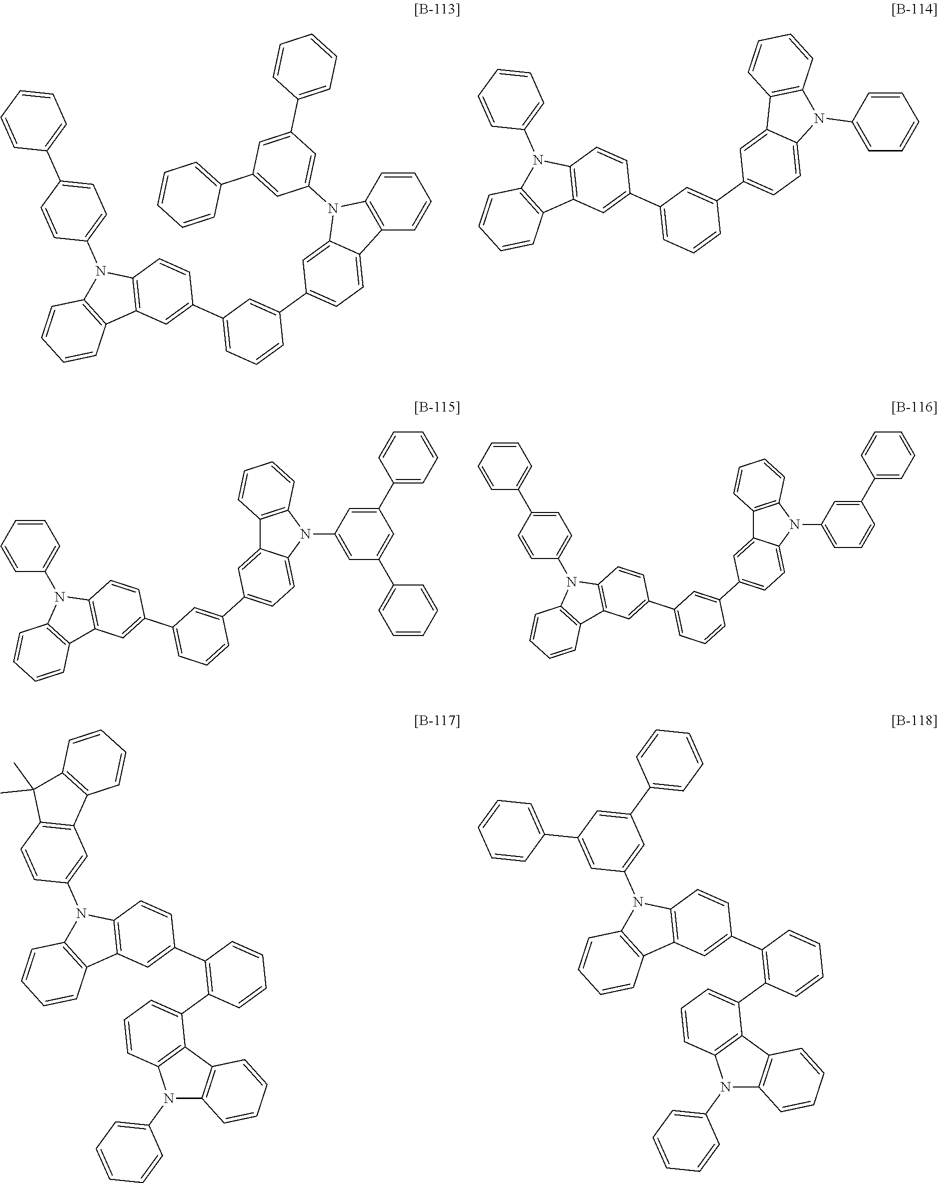

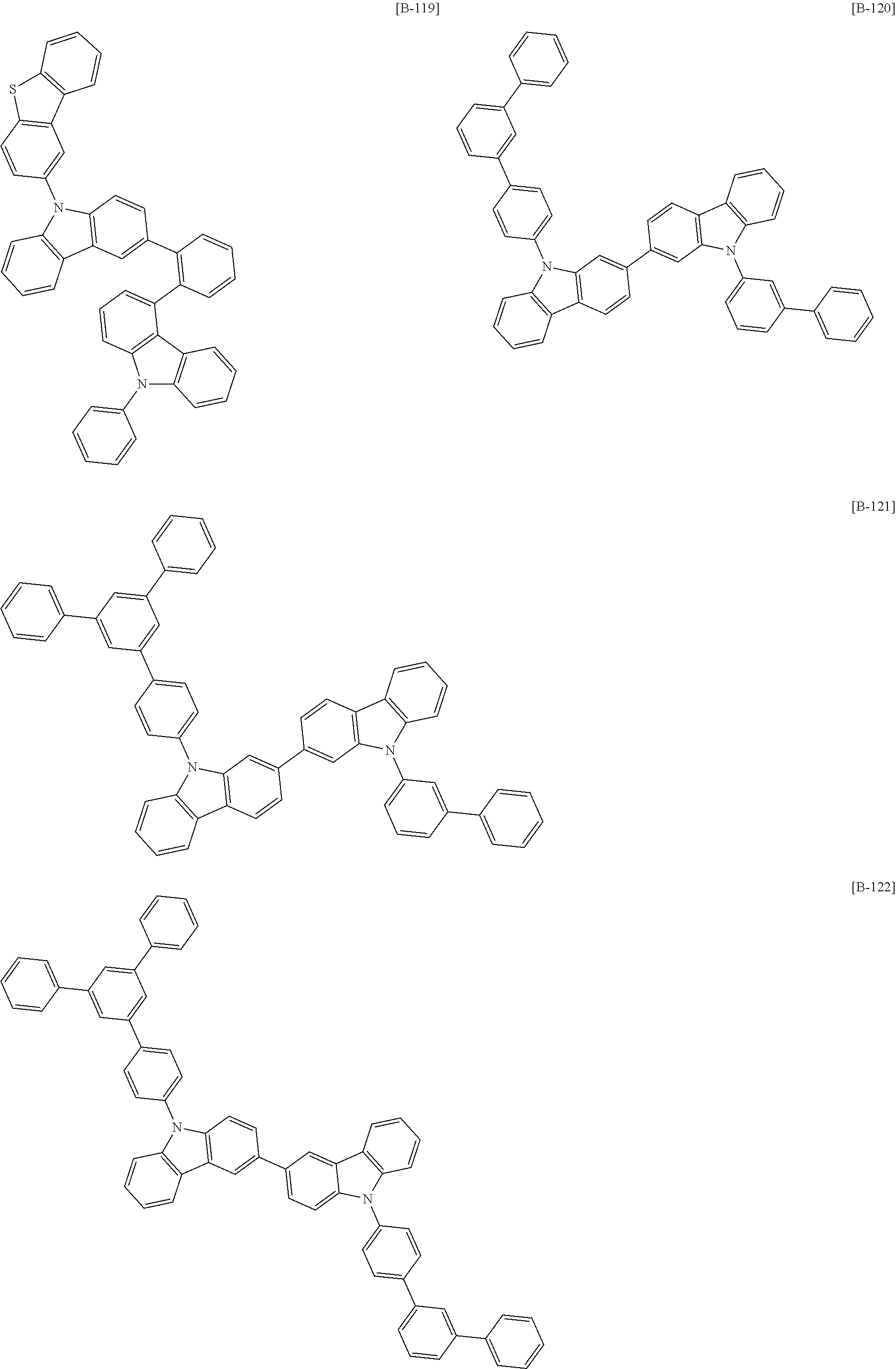

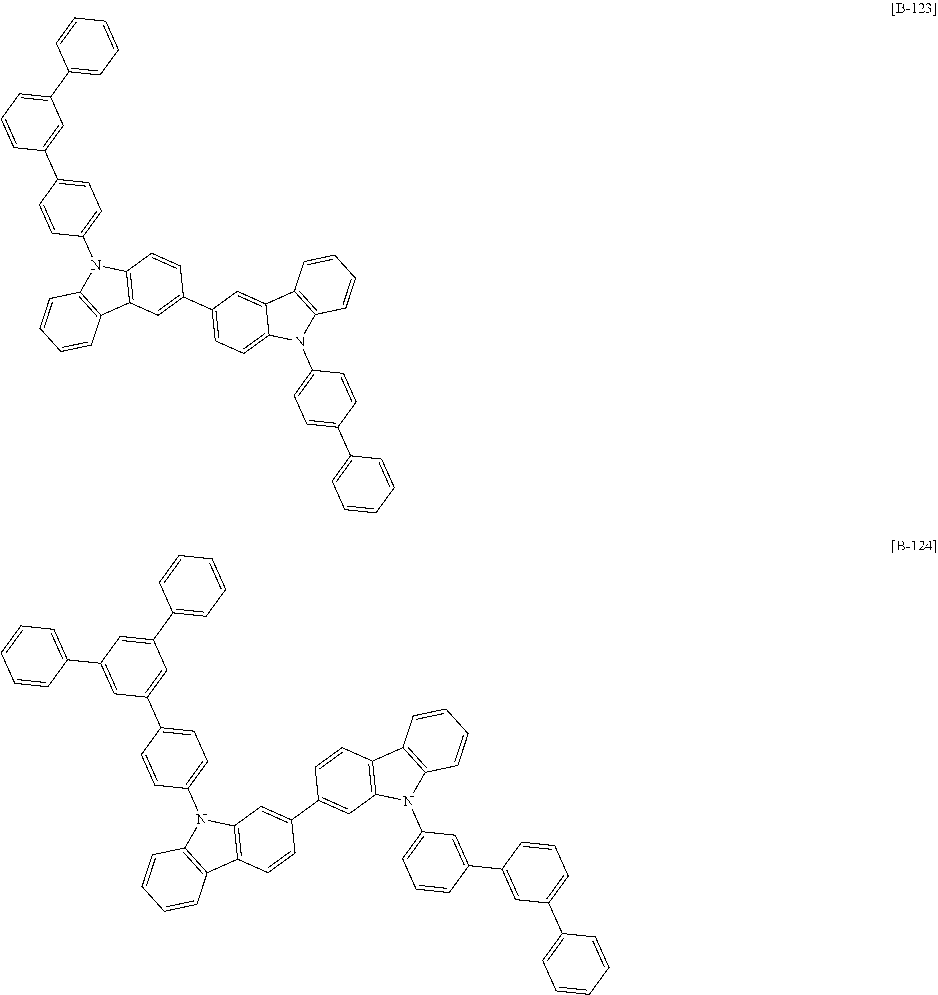 [SMILES: c1ccc(-c2ccc(-n3c4ccccc4c4cc(-c5ccc6c(c5)c5ccccc5n6-c5ccc(-c6cccc(-c7ccccc7)c6)cc5)ccc43)cc2)cc1 ; c1ccc(-c2cccc(-c3cccc(-n4c5ccccc5c5ccc(-c6ccc7c8ccccc8n(-c8ccc(-c9cc(-c%10ccccc%10)cc(-c%10ccccc%10)c9)cc8)c7c6)cc54)c3)c2)cc1]

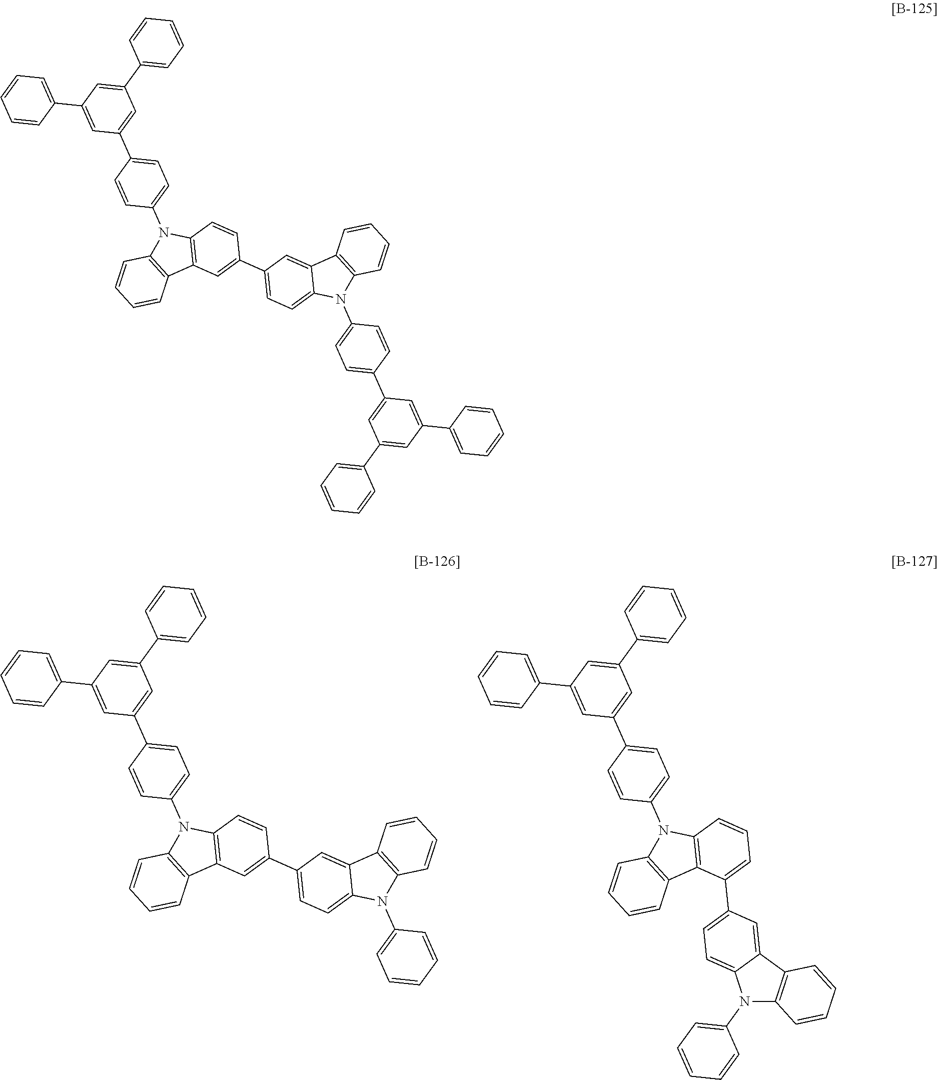 [SMILES: c1ccc(-c2cc(-c3ccccc3)cc(-c3ccc(-n4c5ccccc5c5cc(-c6ccc7c(c6)c6ccccc6n7-c6ccc(-c7cc(-c8ccccc8)cc(-c8ccccc8)c7)cc6)ccc54)cc3)c2)cc1 ; c1ccc(-c2cc(-c3ccccc3)cc(-c3ccc(-n4c5ccccc5c5cc(-c6ccc7c(c6)c6ccccc6n7-c6ccccc6)ccc54)cc3)c2)cc1 ; c1ccc(-c2cc(-c3ccccc3)cc(-c3ccc(-n4c5ccccc5c5c(-c6ccc7c(c6)c6ccccc6n7-c6ccccc6)cccc54)cc3)c2)cc1]

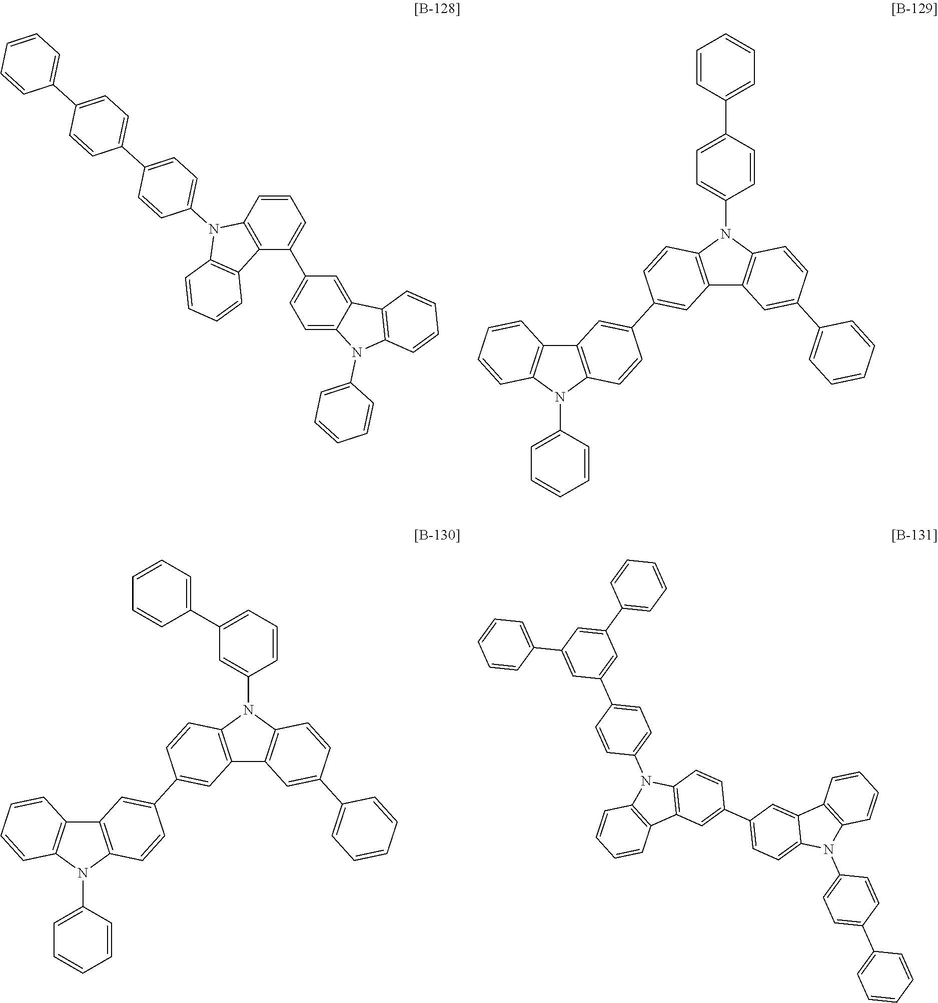 [SMILES: c1ccc(-c2ccc(-c3ccc(-n4c5ccccc5c5c(-c6ccc7c(c6)c6ccccc6n7-c6ccccc6)cccc54)cc3)cc2)cc1 ; c1ccc(-c2ccc(-n3c4ccc(-c5ccccc5)cc4c4cc(-c5ccc6c(c5)c5ccccc5n6-c5ccccc5)ccc43)cc2)cc1 ; c1ccc(-c2cccc(-n3c4ccc(-c5ccccc5)cc4c4cc(-c5ccc6c(c5)c5ccccc5n6-c5ccccc5)ccc43)c2)cc1 ; c1ccc(-c2ccc(-n3c4ccccc4c4cc(-c5ccc6c(c5)c5ccccc5n6-c5ccc(-c6cc(-c7ccccc7)cc(-c7ccccc7)c6)cc5)ccc43)cc2)cc1]

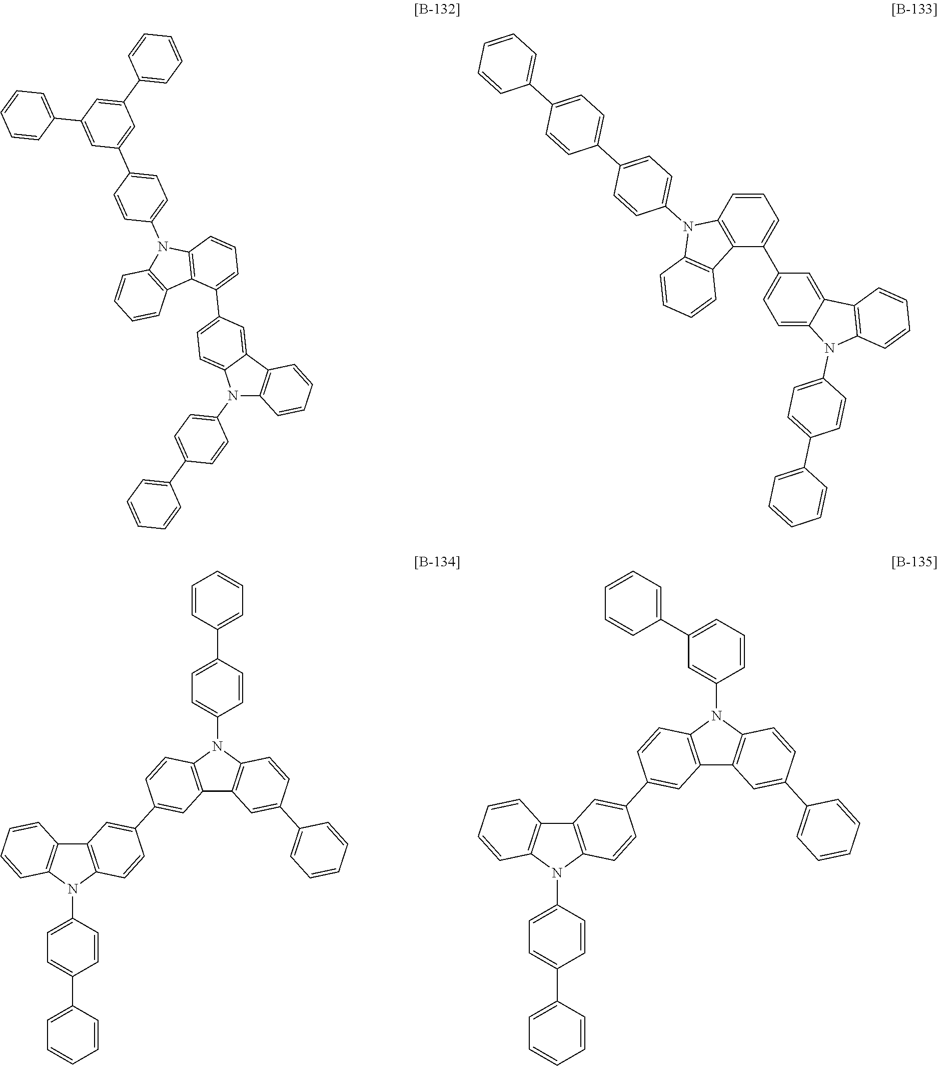 [SMILES: c1ccc(-c2ccc(-n3c4ccccc4c4cc(-c5cccc6c5c5ccccc5n6-c5ccc(-c6cc(-c7ccccc7)cc(-c7ccccc7)c6)cc5)ccc43)cc2)cc1 ; c1ccc(-c2ccc(-c3ccc(-n4c5ccccc5c5c(-c6ccc7c(c6)c6ccccc6n7-c6ccc(-c7ccccc7)cc6)cccc54)cc3)cc2)cc1 ; c1ccc(-c2ccc(-n3c4ccccc4c4cc(-c5ccc6c(c5)c5cc(-c7ccccc7)ccc5n6-c5ccc(-c6ccccc6)cc5)ccc43)cc2)cc1 ; c1ccc(-c2ccc(-n3c4ccccc4c4cc(-c5ccc6c(c5)c5cc(-c7ccccc7)ccc5n6-c5cccc(-c6ccccc6)c5)ccc43)cc2)cc1]

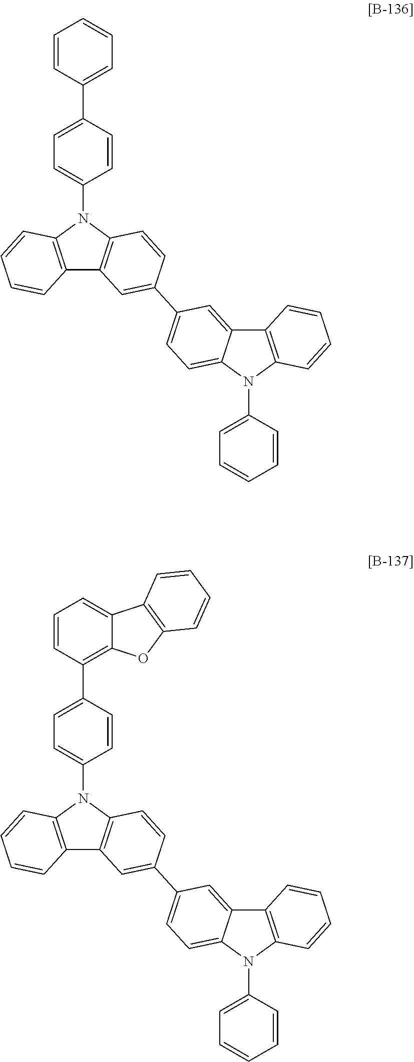 [SMILES: c1ccc(-c2ccc(-n3c4ccccc4c4cc(-c5ccc6c(c5)c5ccccc5n6-c5ccccc5)ccc43)cc2)cc1 ; c1ccc(-n2c3ccccc3c3cc(-c4ccc5c(c4)c4ccccc4n5-c4ccc(-c5cccc6c5oc5ccccc56)cc4)ccc32)cc1]

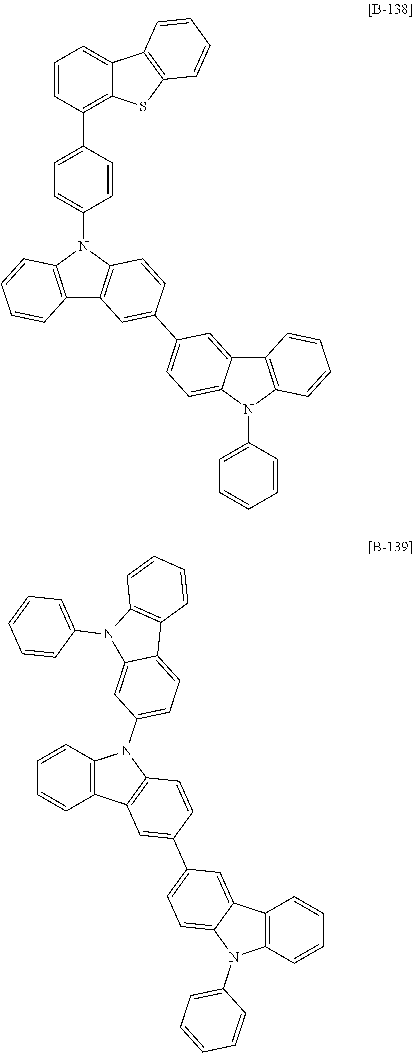 [SMILES: c1ccc(-n2c3ccccc3c3cc(-c4ccc5c(c4)c4ccccc4n5-c4ccc(-c5cccc6c5sc5ccccc56)cc4)ccc32)cc1 ; c1ccc(-n2c3ccccc3c3cc(-c4ccc5c(c4)c4ccccc4n5-c4ccc5c6ccccc6n(-c6ccccc6)c5c4)ccc32)cc1]

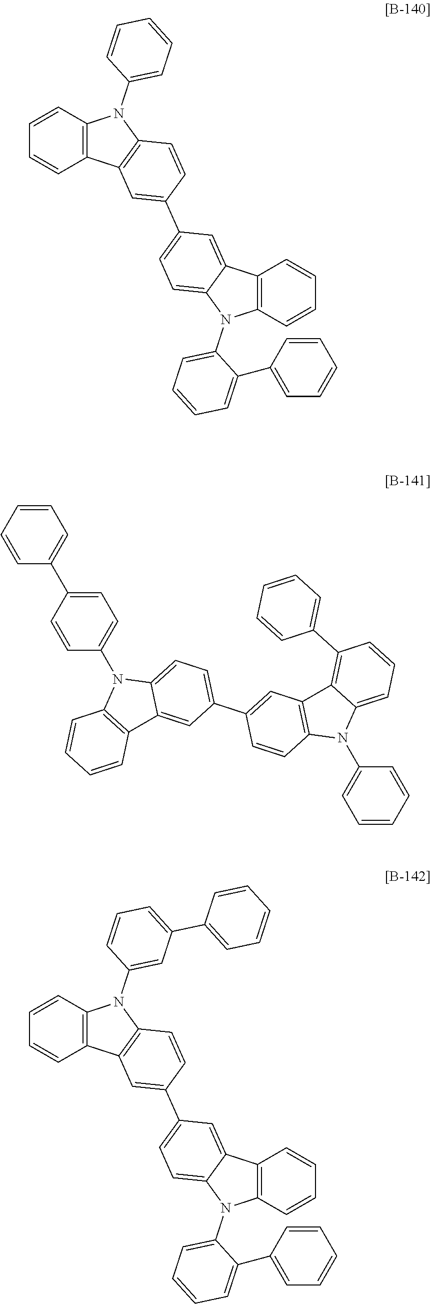 [SMILES: c1ccc(-c2ccccc2-n2c3ccccc3c3cc(-c4ccc5c(c4)c4ccccc4n5-c4ccccc4)ccc32)cc1 ; c1ccc(-c2ccc(-n3c4ccccc4c4cc(-c5ccc6c(c5)c5c(-c7ccccc7)cccc5n6-c5ccccc5)ccc43)cc2)cc1 ; c1ccc(-c2cccc(-n3c4ccccc4c4cc(-c5ccc6c(c5)c5ccccc5n6-c5ccccc5-c5ccccc5)ccc43)c2)cc1]

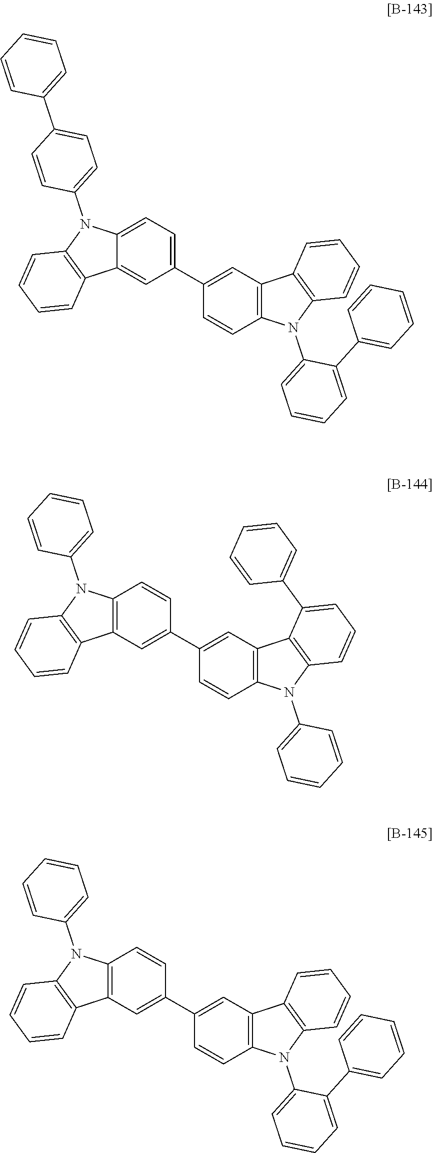 [SMILES: c1ccc(-c2ccc(-n3c4ccccc4c4cc(-c5ccc6c(c5)c5ccccc5n6-c5ccccc5-c5ccccc5)ccc43)cc2)cc1 ; c1ccc(-c2cccc3c2c2cc(-c4ccc5c(c4)c4ccccc4n5-c4ccccc4)ccc2n3-c2ccccc2)cc1 ; c1ccc(-c2ccccc2-n2c3ccccc3c3cc(-c4ccc5c(c4)c4ccccc4n5-c4ccccc4)ccc32)cc1]

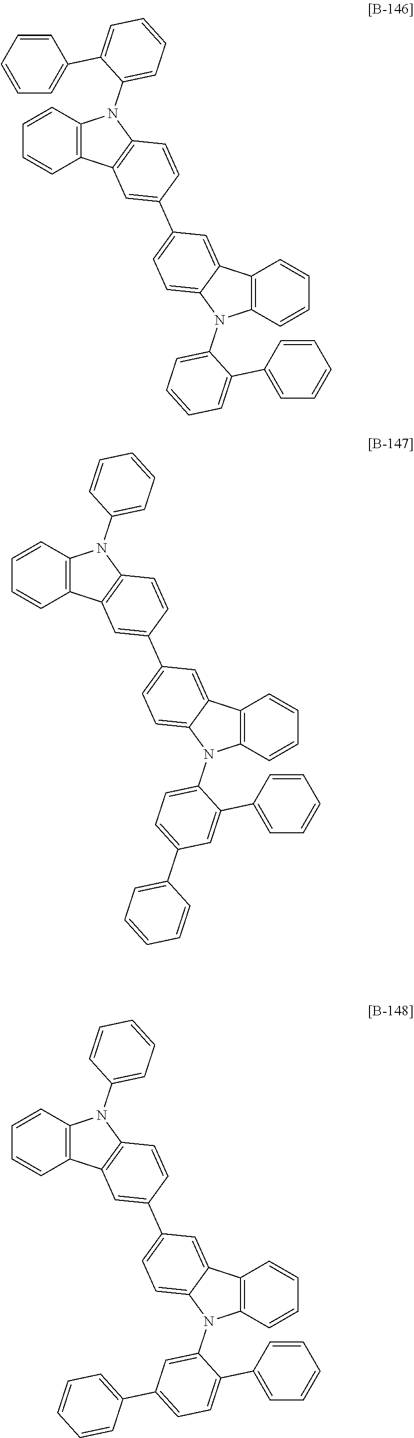 [SMILES: c1ccc(-c2ccccc2-n2c3ccccc3c3cc(-c4ccc5c(c4)c4ccccc4n5-c4ccccc4-c4ccccc4)ccc32)cc1 ; c1ccc(-c2ccc(-n3c4ccccc4c4cc(-c5ccc6c(c5)c5ccccc5n6-c5ccccc5)ccc43)c(-c3ccccc3)c2)cc1 ; c1ccc(-c2ccc(-c3ccccc3)c(-n3c4ccccc4c4cc(-c5ccc6c(c5)c5ccccc5n6-c5ccccc5)ccc43)c2)cc1]

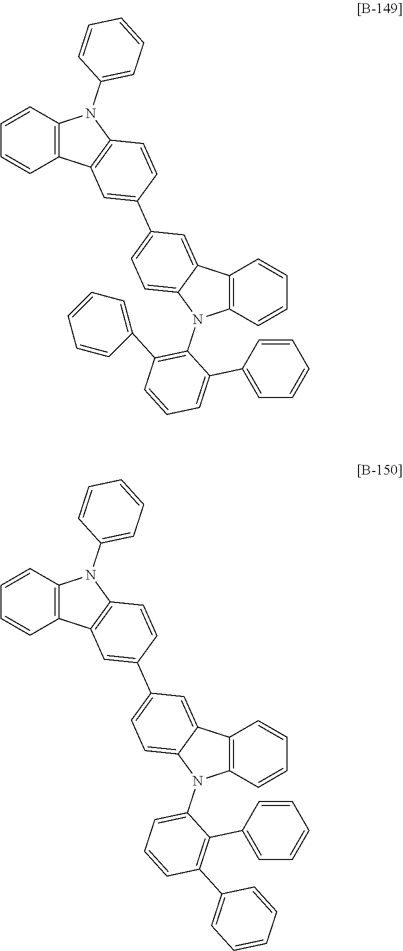 [SMILES: c1ccc(-c2cccc(-c3ccccc3)c2-n2c3ccccc3c3cc(-c4ccc5c(c4)c4ccccc4n5-c4ccccc4)ccc32)cc1 ; c1ccc(-c2cccc(-n3c4ccccc4c4cc(-c5ccc6c(c5)c5ccccc5n6-c5ccccc5)ccc43)c2-c2ccccc2)cc1]

Additionally, examples of Compound B-1 to Compound B-150 listed in Group 2 in which at least one hydrogen is replaced with deuterium are given below, but are not limited thereto.

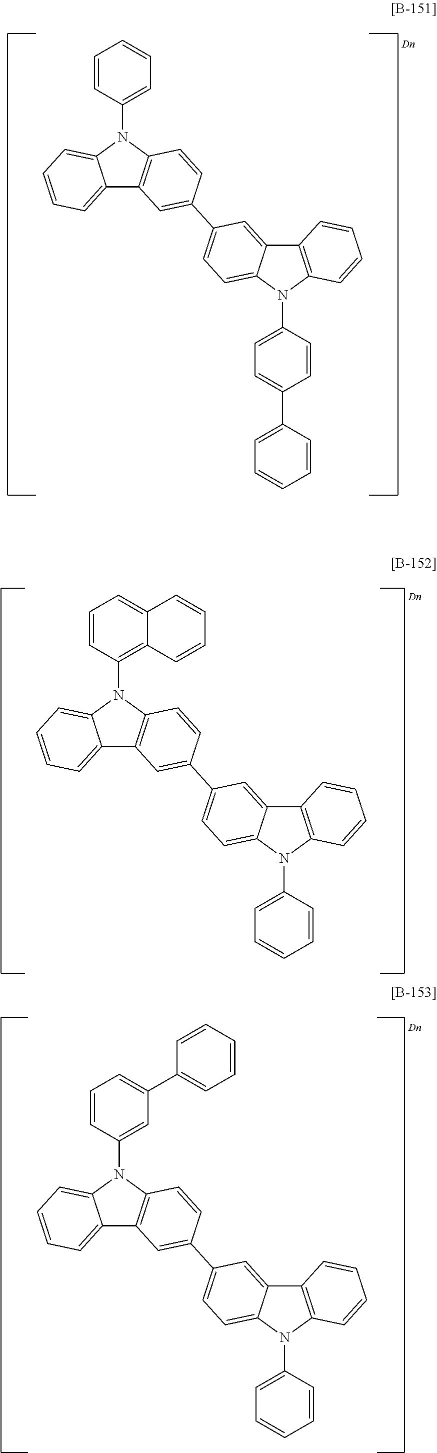

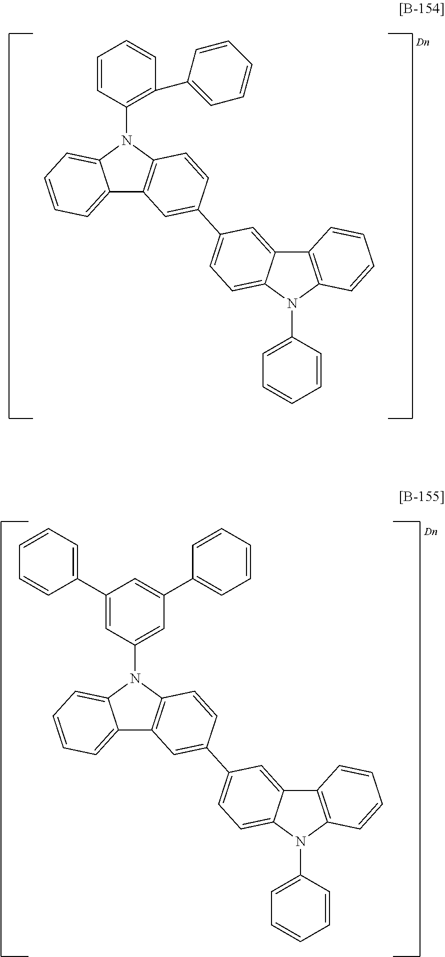

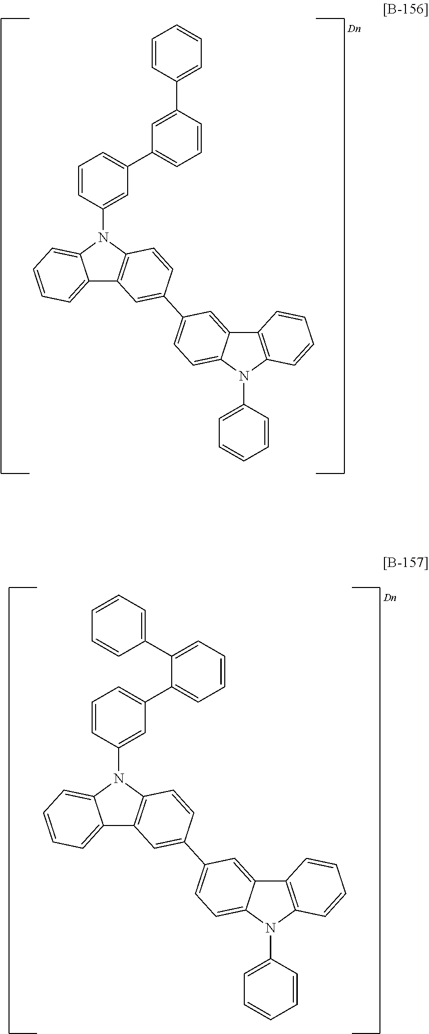

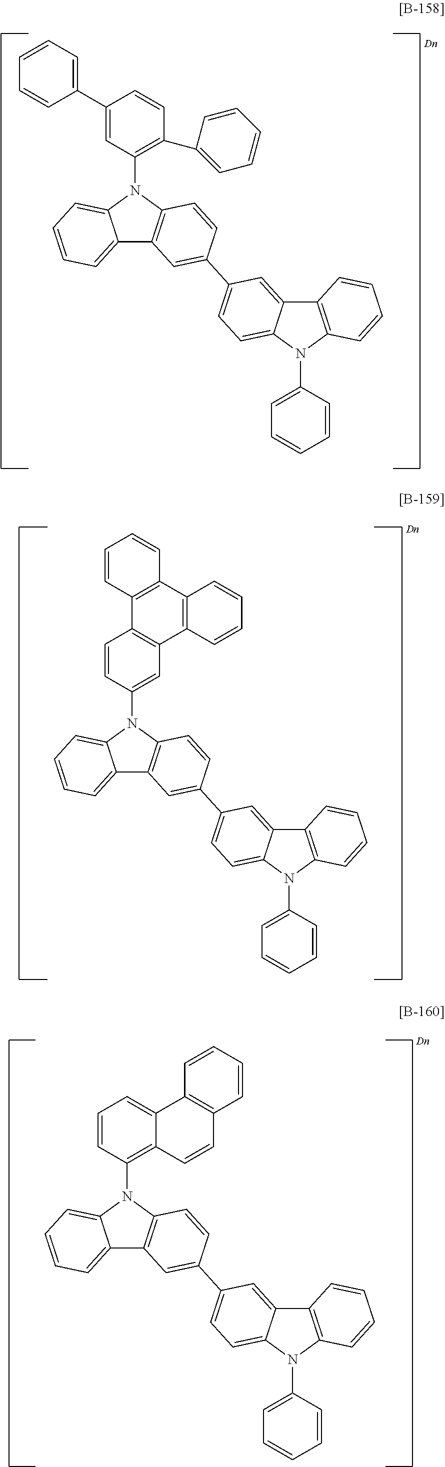

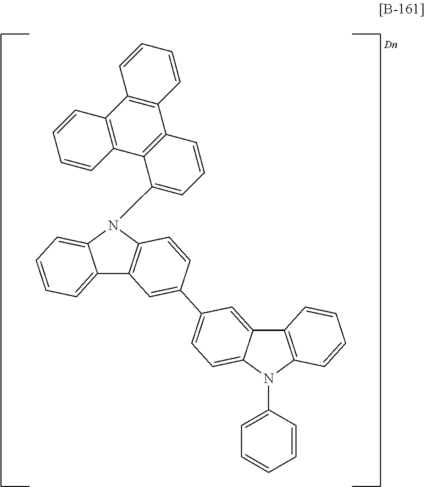

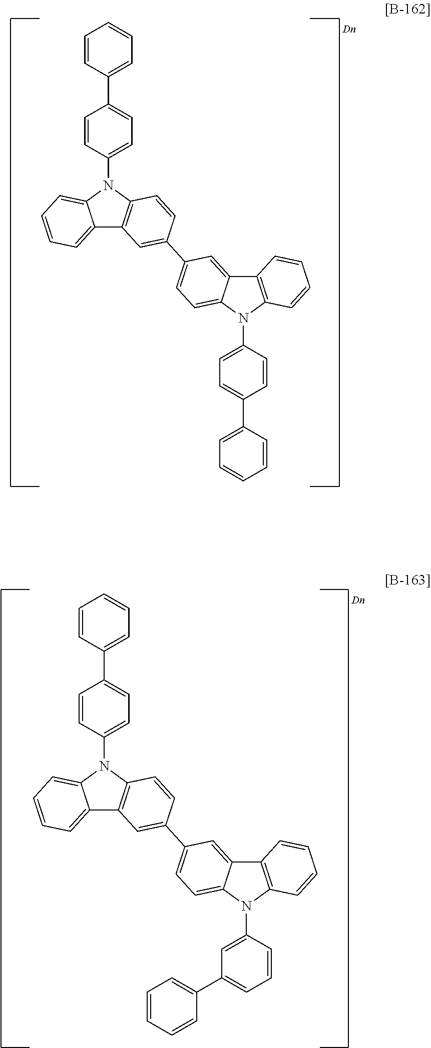

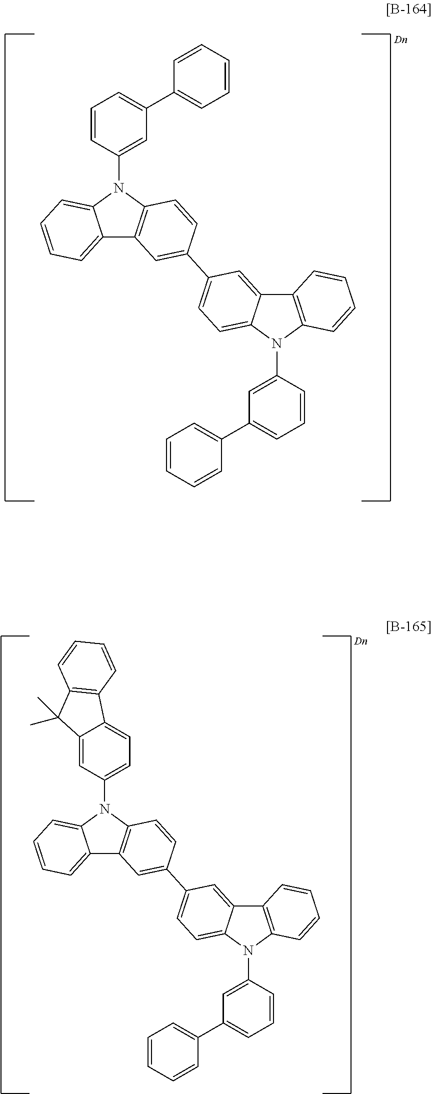

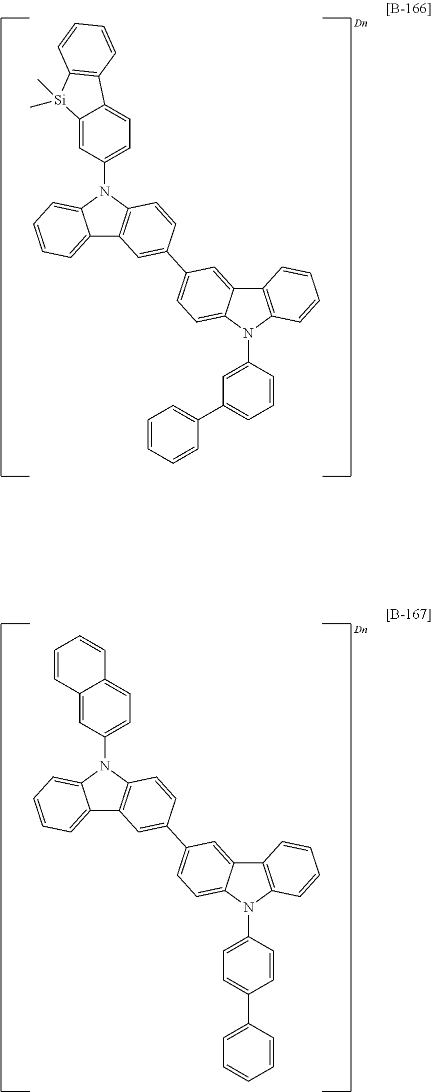

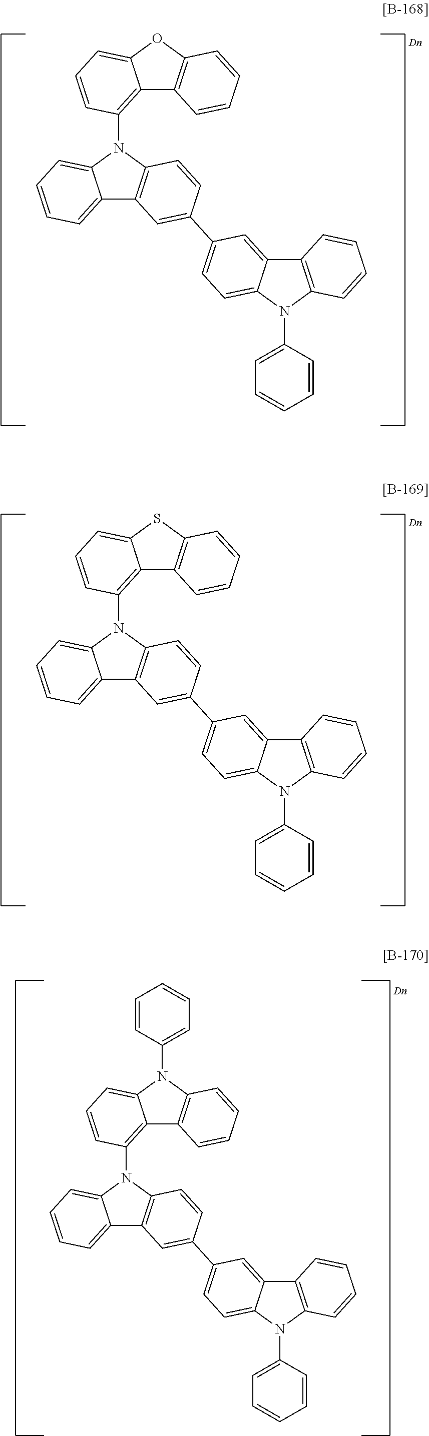

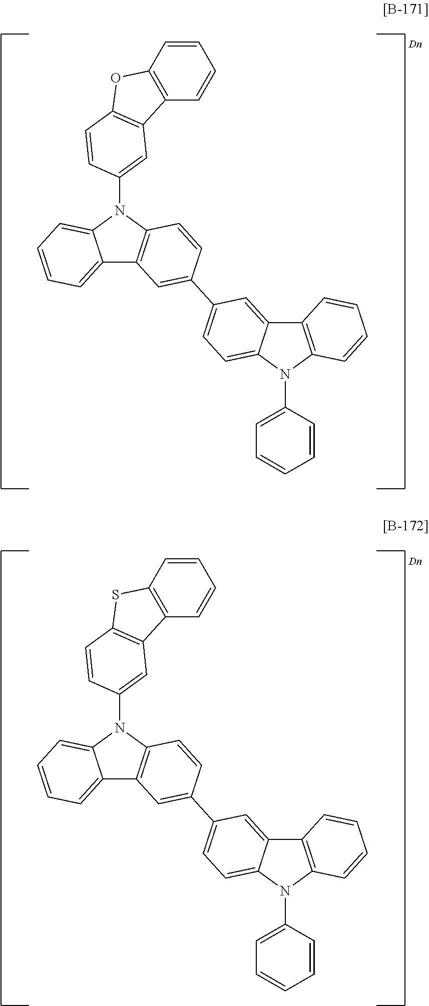

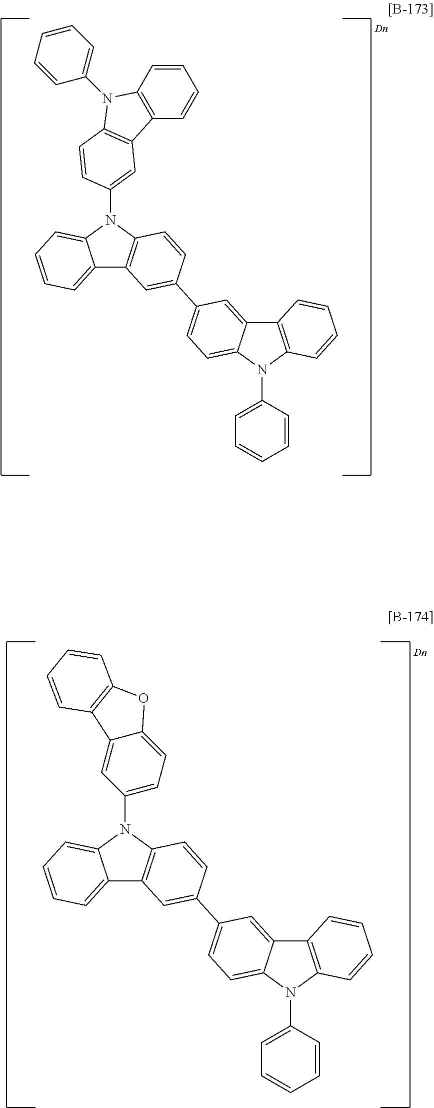

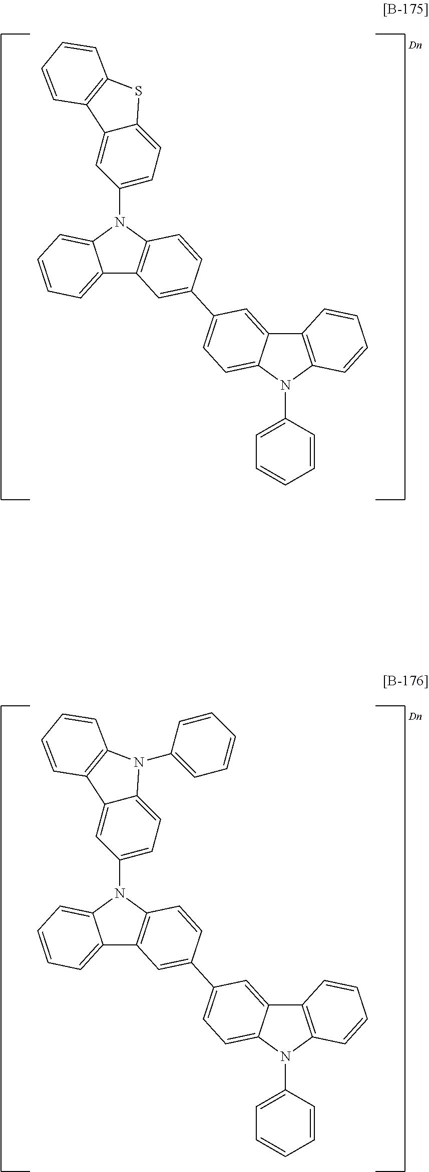

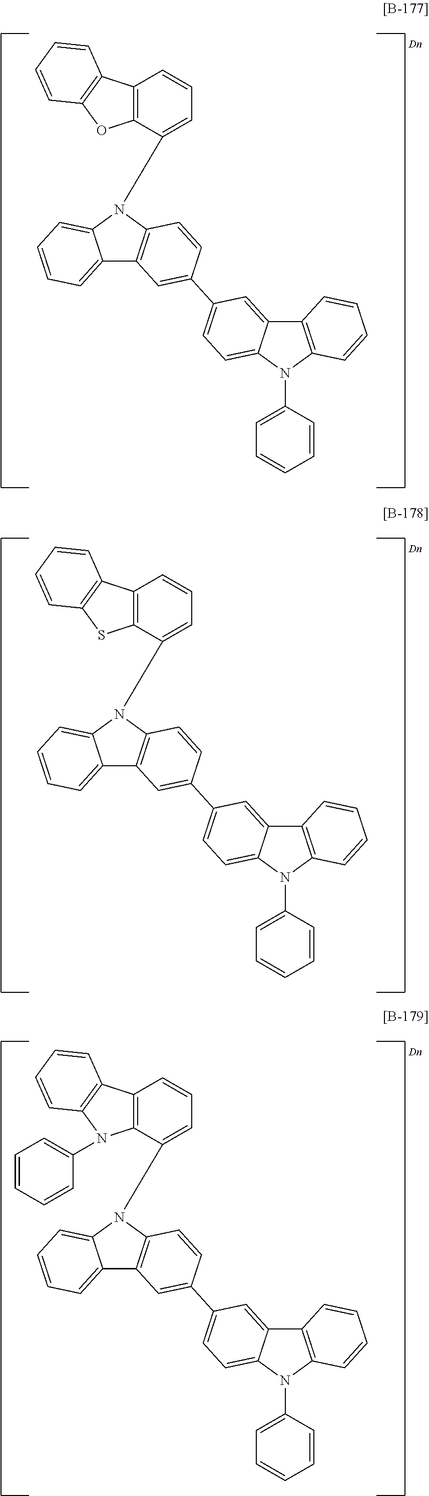

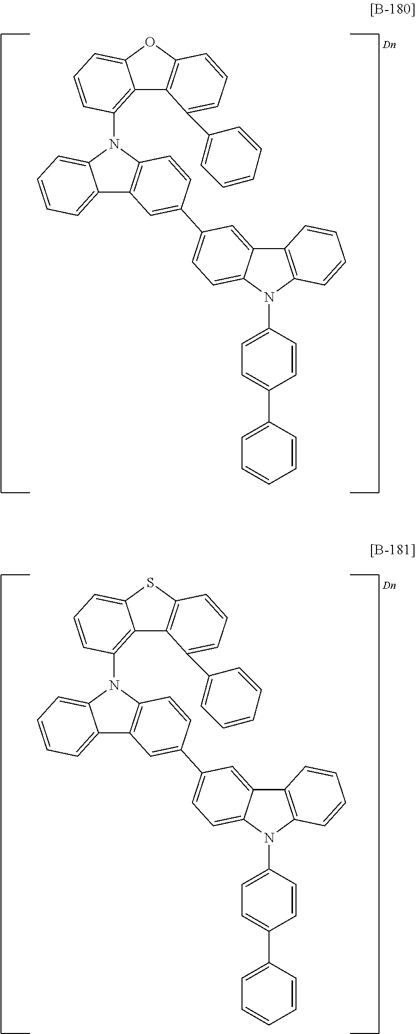

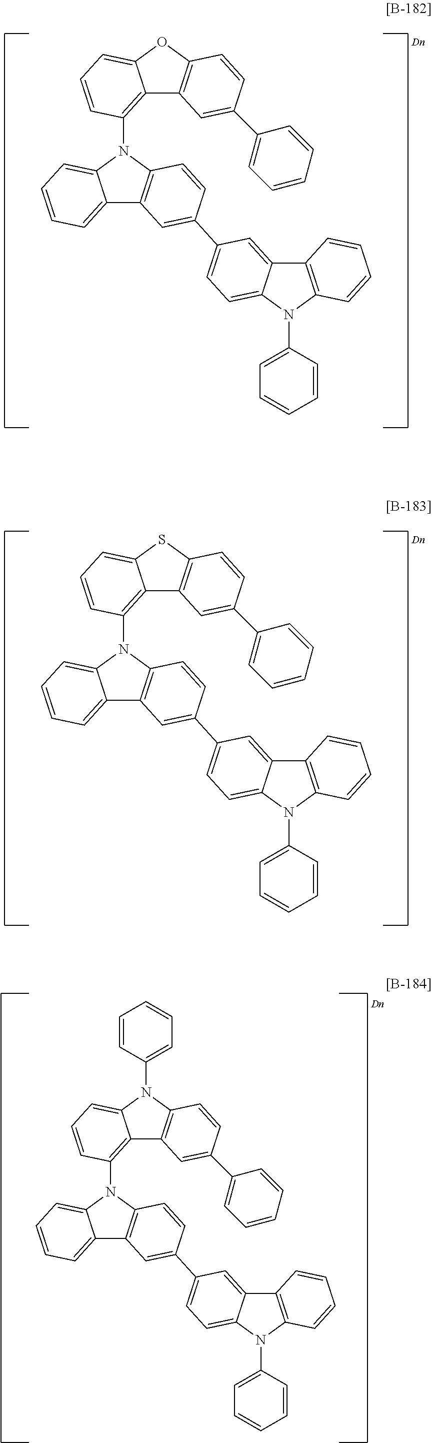

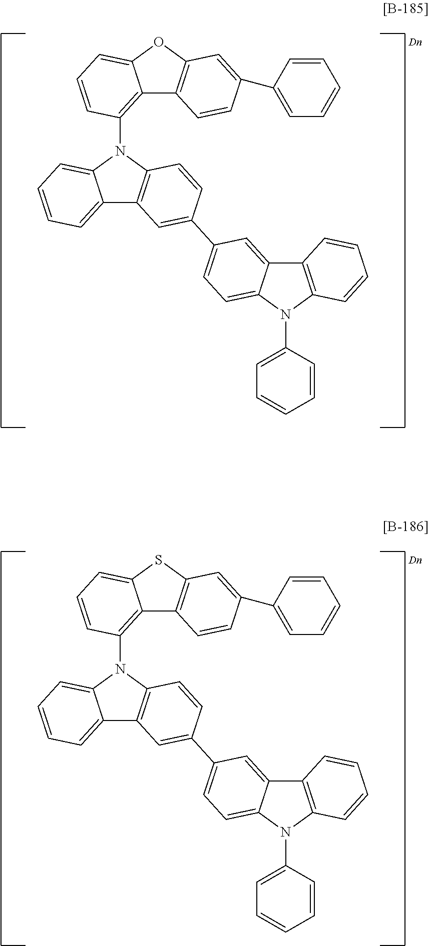

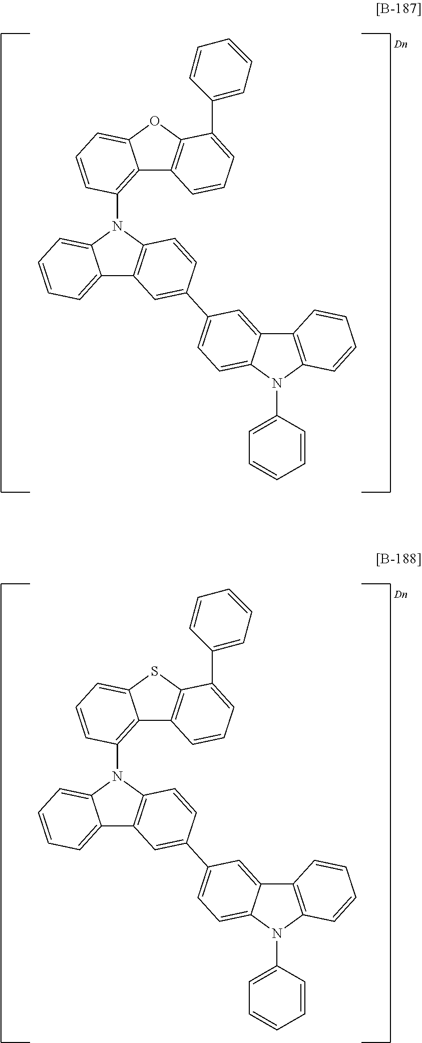

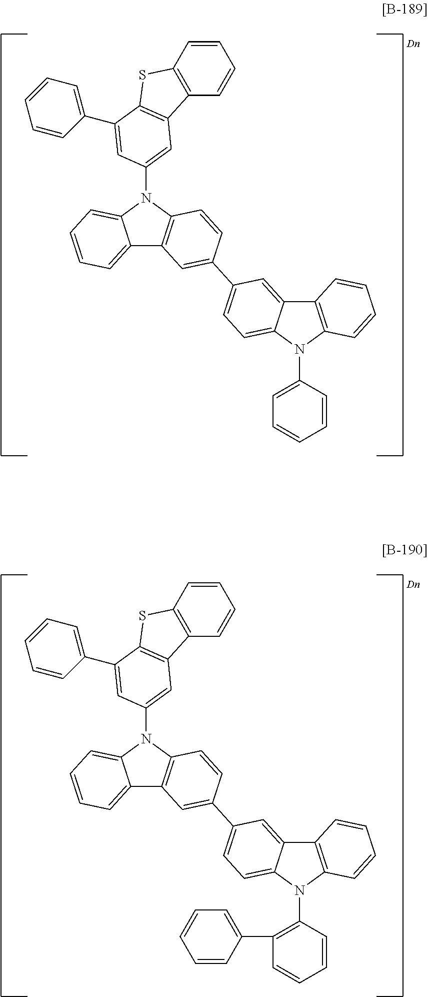

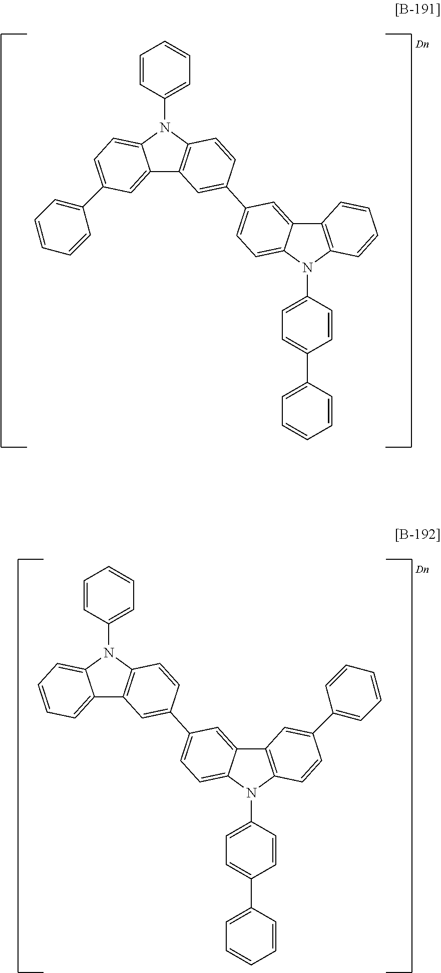

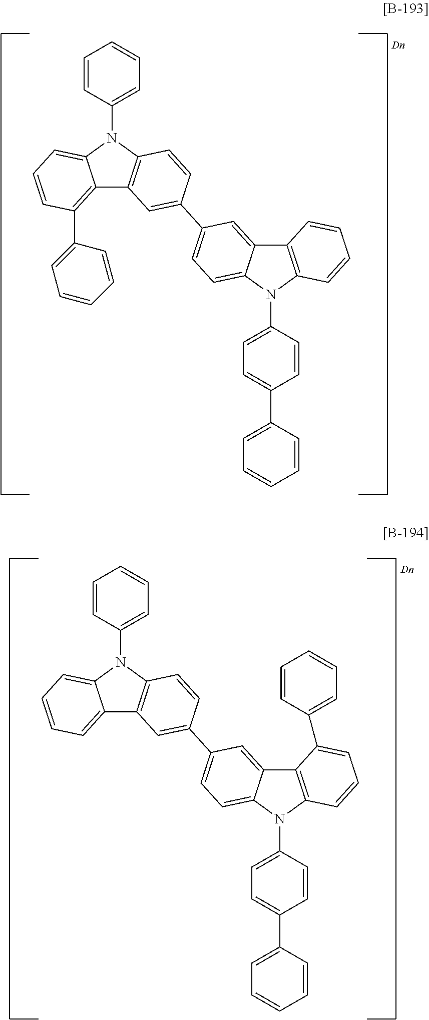

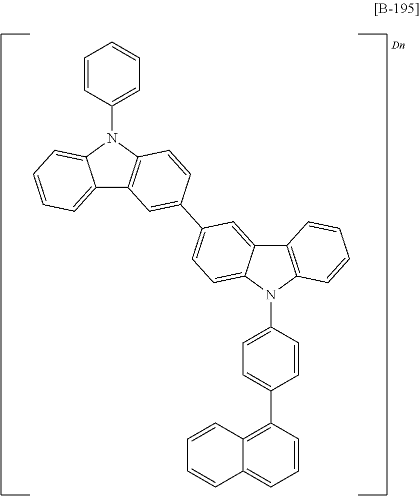

(Dn refers to the number of deuterium substitutions and indicates a structure substituted with one or more deuteriums)

The most specific structures for Compound B-151 to Compound B-195 of Group 2 are presented below as examples according to the position and substitution rate of deuterium substitution, and are not the intention to limit the scope of rights to compounds not listed below.

The scope of the present disclosure is determined by the claims, and when deuterium is substituted, it is not limited to the compounds exemplified below, and the deuterium substitution position, deuterium substitution rate, etc. may include all changeable ranges within the range of Compound B-1 to Compound B-195.

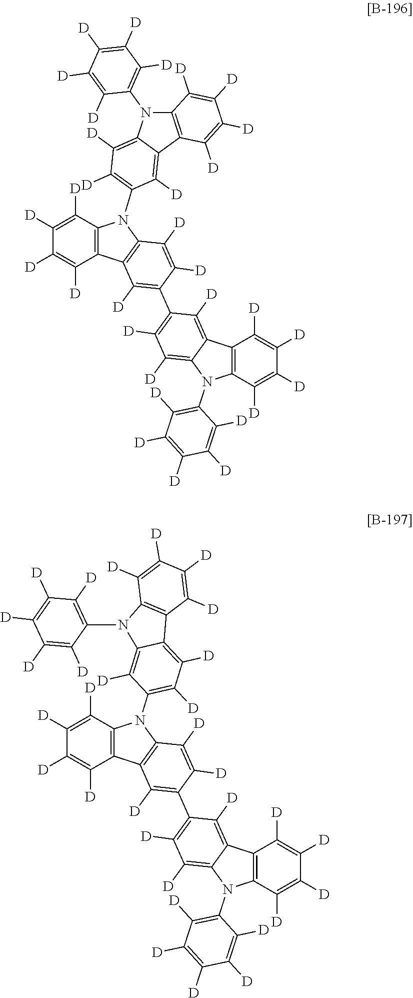

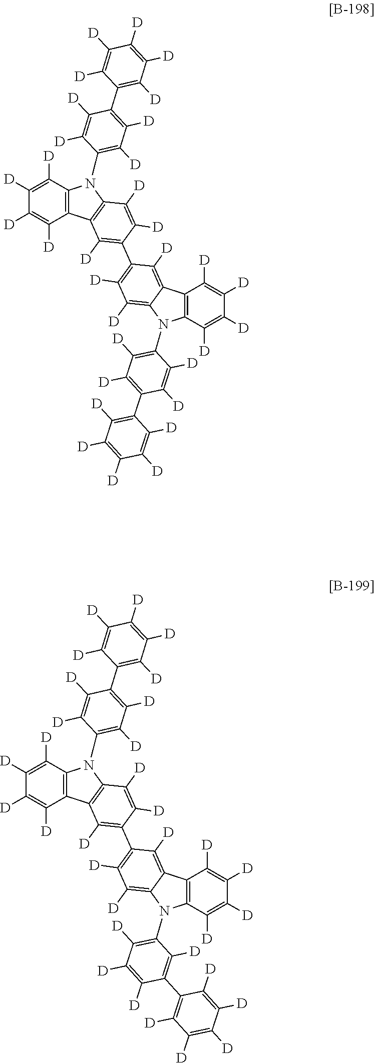

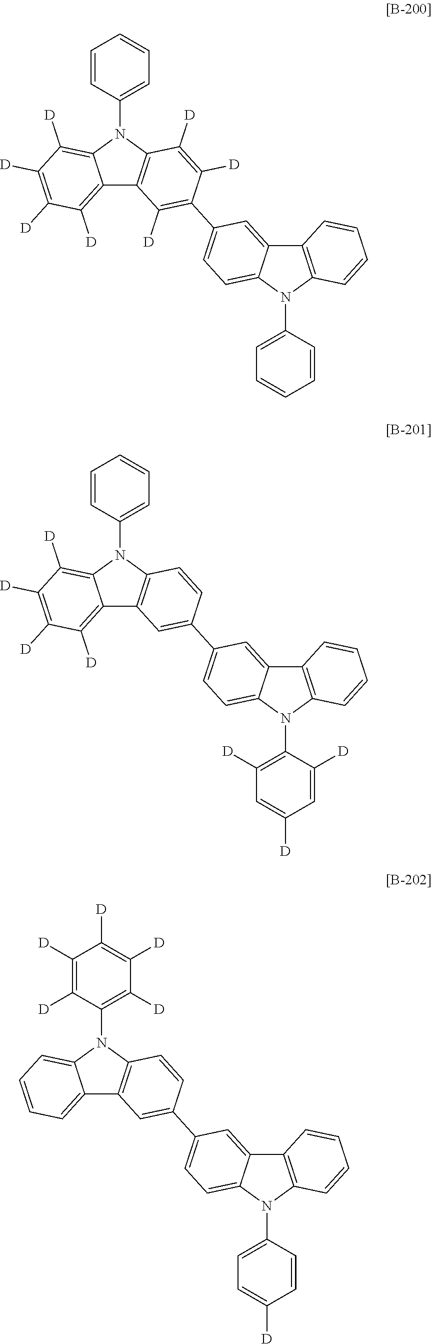 [SMILES: [2H]c1c([2H])c([2H])c2c(c1[2H])c1c([2H])c(-c3ccc4c(c3)c3ccccc3n4-c3ccccc3)c([2H])c([2H])c1n2-c1ccccc1 ; [2H]c1cc([2H])c(-n2c3ccccc3c3cc(-c4ccc5c(c4)c4c([2H])c([2H])c([2H])c([2H])c4n5-c4ccccc4)ccc32)c([2H])c1 ; [2H]c1ccc(-n2c3ccccc3c3cc(-c4ccc5c(c4)c4ccccc4n5-c4c([2H])c([2H])c([2H])c([2H])c4[2H])ccc32)cc1]

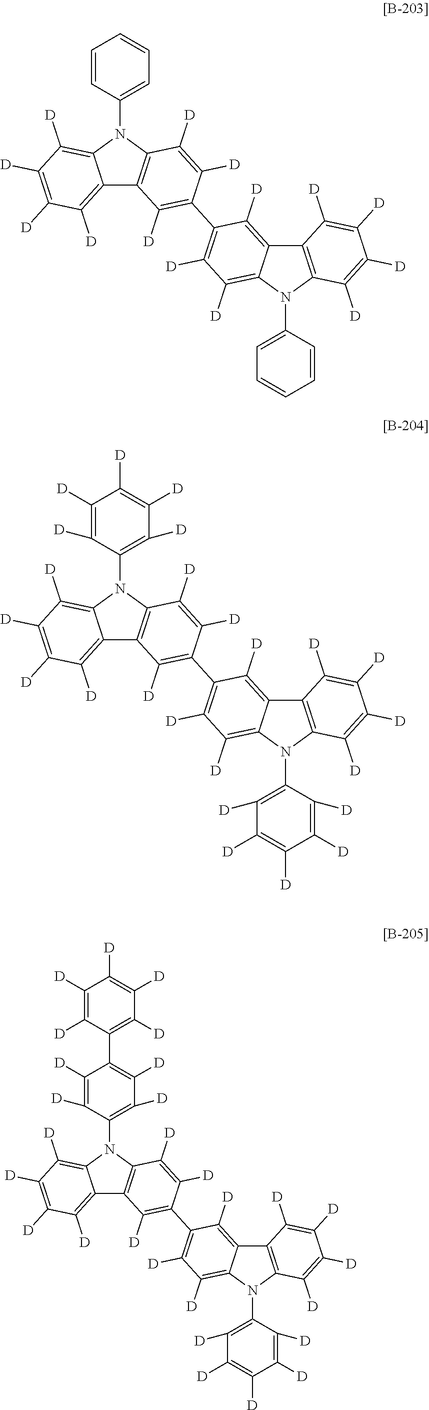

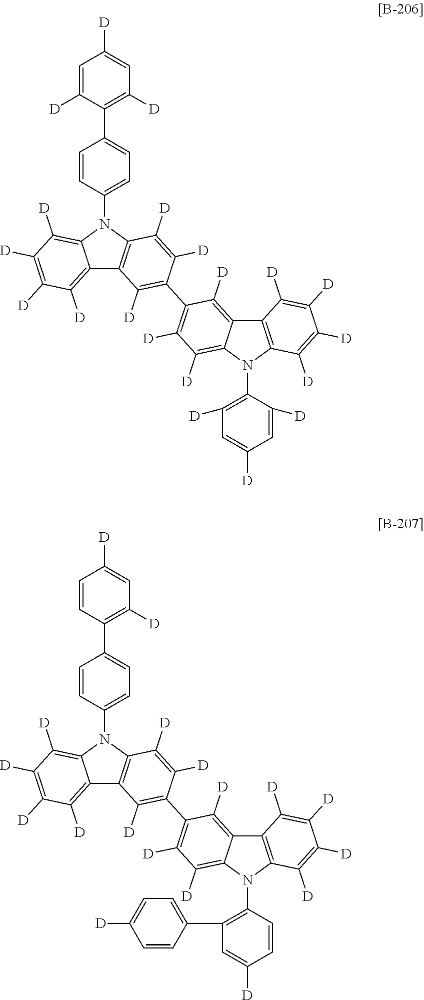 [SMILES: [2H]c1cc([2H])c(-c2ccc(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])cc([2H])cc5[2H])c([2H])c([2H])c43)cc2)c([2H])c1 ; [2H]c1ccc(-c2cc([2H])ccc2-n2c3c([2H])c([2H])c([2H])c([2H])c3c3c([2H])c(-c4c([2H])c([2H])c5c(c4[2H])c4c([2H])c([2H])c([2H])c([2H])c4n5-c4ccc(-c5ccc([2H])cc5[2H])cc4)c([2H])c([2H])c32)cc1]

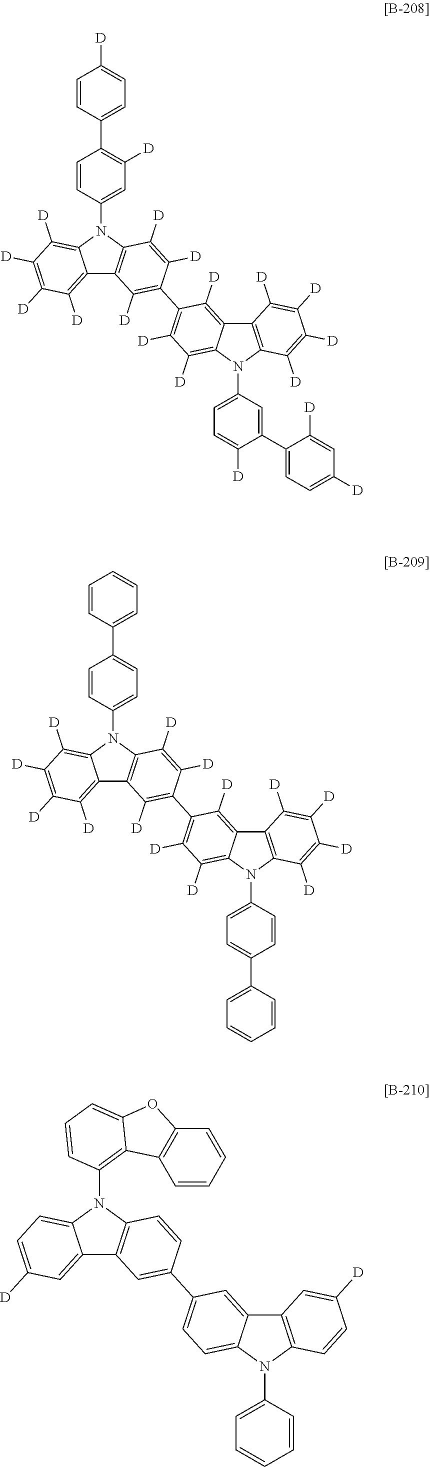 [SMILES: [2H]c1ccc(-c2ccc(-n3c4c([2H])c([2H])c([2H])c([2H])c4c4c([2H])c(-c5c([2H])c([2H])c6c(c5[2H])c5c([2H])c([2H])c([2H])c([2H])c5n6-c5ccc([2H])c(-c6ccc([2H])cc6[2H])c5)c([2H])c([2H])c43)cc2[2H])cc1 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3ccc(-c4ccccc4)cc3)c([2H])c([2H])c1n2-c1ccc(-c2ccccc2)cc1 ; [2H]c1ccc2c(c1)c1cc(-c3ccc4c(c3)c3cc([2H])ccc3n4-c3cccc4oc5ccccc5c34)ccc1n2-c1ccccc1]

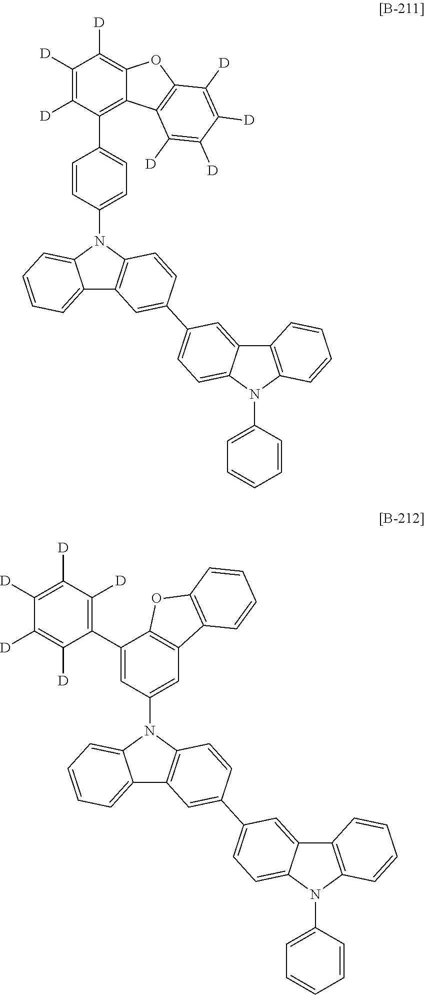 [SMILES: [2H]c1c([2H])c([2H])c2c(oc3c([2H])c([2H])c([2H])c(-c4ccc(-n5c6ccccc6c6cc(-c7ccc8c(c7)c7ccccc7n8-c7ccccc7)ccc65)cc4)c32)c1[2H] ; [2H]c1c([2H])c([2H])c(-c2cc(-n3c4ccccc4c4cc(-c5ccc6c(c5)c5ccccc5n6-c5ccccc5)ccc43)cc3c2oc2ccccc23)c([2H])c1[2H]]

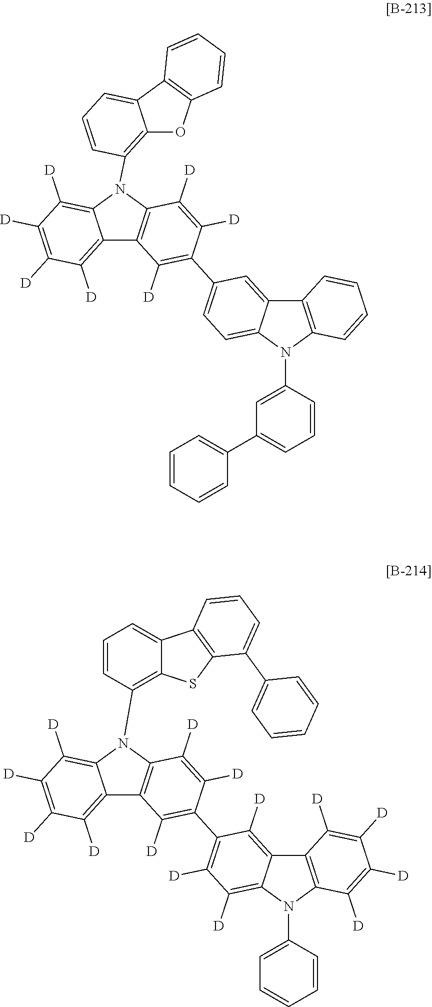 [SMILES: [2H]c1c([2H])c([2H])c2c(c1[2H])c1c([2H])c(-c3ccc4c(c3)c3ccccc3n4-c3cccc(-c4ccccc4)c3)c([2H])c([2H])c1n2-c1cccc2c1oc1ccccc12 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc4c3sc3c(-c5ccccc5)cccc34)c([2H])c([2H])c1n2-c1ccccc1]

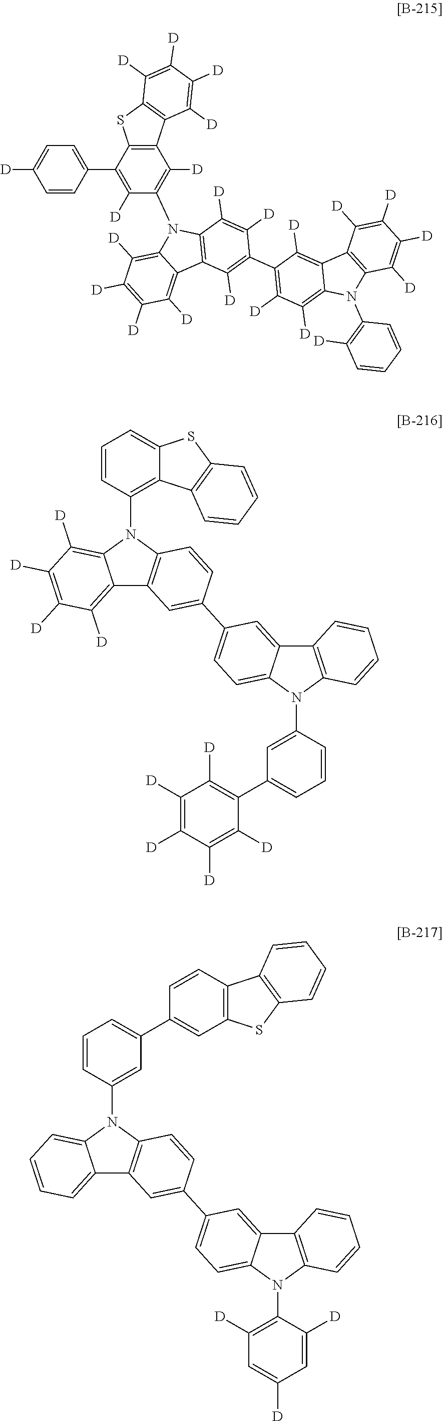 [SMILES: [2H]c1ccc(-c2c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5ccccc5[2H])c([2H])c([2H])c43)c([2H])c3c2sc2c([2H])c([2H])c([2H])c([2H])c23)cc1 ; [2H]c1c([2H])c([2H])c(-c2cccc(-n3c4ccccc4c4cc(-c5ccc6c(c5)c5c([2H])c([2H])c([2H])c([2H])c5n6-c5cccc6sc7ccccc7c56)ccc43)c2)c([2H])c1[2H] ; [2H]c1cc([2H])c(-n2c3ccccc3c3cc(-c4ccc5c(c4)c4ccccc4n5-c4cccc(-c5ccc6c(c5)sc5ccccc56)c4)ccc32)c([2H])c1]

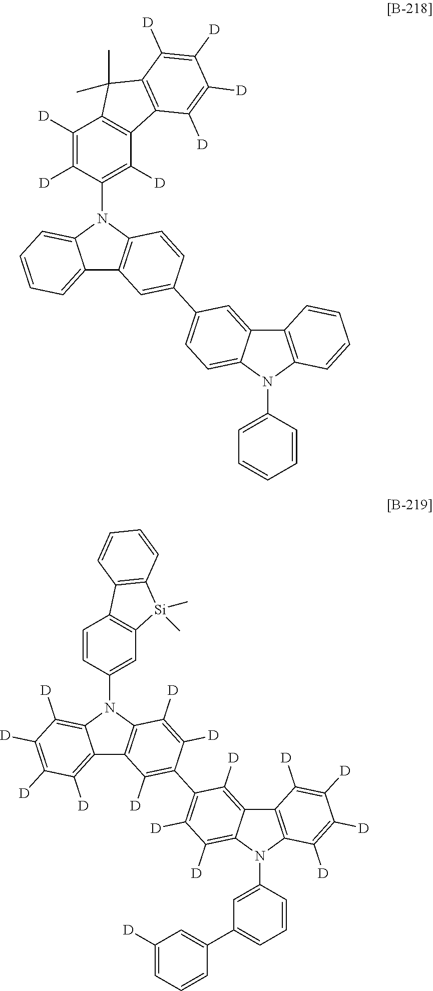 [SMILES: [2H]c1c([2H])c([2H])c2c(c1[2H])-c1c([2H])c(-n3c4ccccc4c4cc(-c5ccc6c(c5)c5ccccc5n6-c5ccccc5)ccc43)c([2H])c([2H])c1C2(C)C ; [2H]c1cccc(-c2cccc(-n3c4c([2H])c([2H])c([2H])c([2H])c4c4c([2H])c(-c5c([2H])c([2H])c6c(c5[2H])c5c([2H])c([2H])c([2H])c([2H])c5n6-c5ccc6c(c5)[Si](C)(C)c5ccccc5-6)c([2H])c([2H])c43)c2)c1]

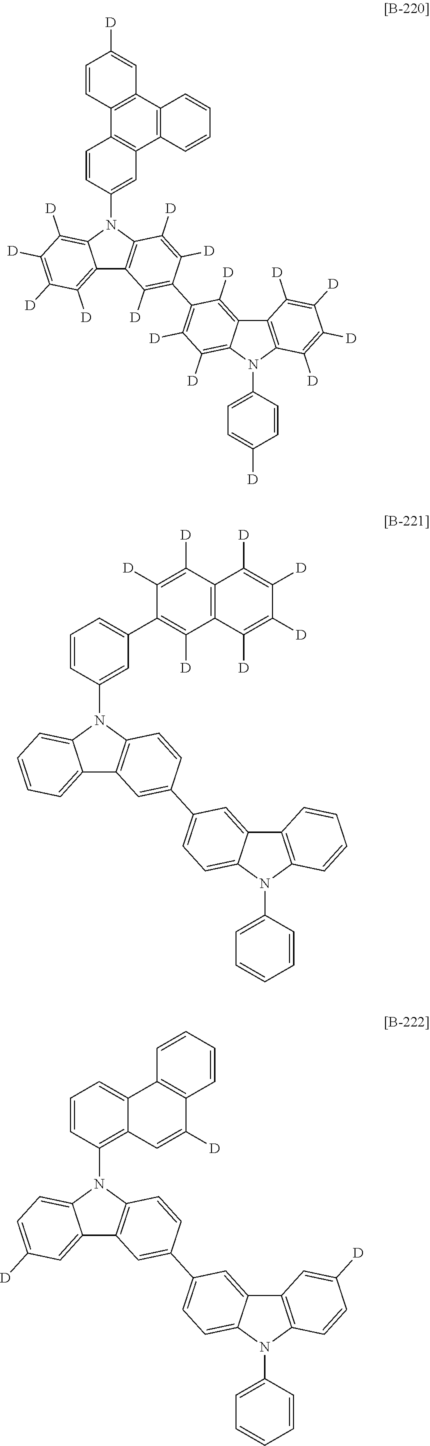 [SMILES: [2H]c1ccc(-n2c3c([2H])c([2H])c([2H])c([2H])c3c3c([2H])c(-c4c([2H])c([2H])c5c(c4[2H])c4c([2H])c([2H])c([2H])c([2H])c4n5-c4ccc5c6ccc([2H])cc6c6ccccc6c5c4)c([2H])c([2H])c32)cc1 ; [2H]c1c([2H])c([2H])c2c([2H])c(-c3cccc(-n4c5ccccc5c5cc(-c6ccc7c(c6)c6ccccc6n7-c6ccccc6)ccc54)c3)c([2H])c([2H])c2c1[2H] ; [2H]c1ccc2c(c1)c1cc(-c3ccc4c(c3)c3cc([2H])ccc3n4-c3cccc4c3cc([2H])c3ccccc34)ccc1n2-c1ccccc1]

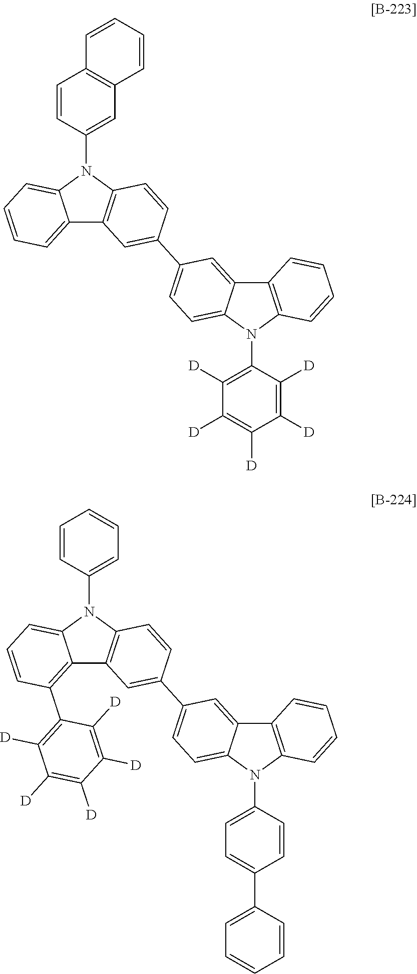 [SMILES: [2H]c1c([2H])c([2H])c(-n2c3ccccc3c3cc(-c4ccc5c(c4)c4ccccc4n5-c4ccc5ccccc5c4)ccc32)c([2H])c1[2H] ; [2H]c1c([2H])c([2H])c(-c2cccc3c2c2cc(-c4ccc5c(c4)c4ccccc4n5-c4ccc(-c5ccccc5)cc4)ccc2n3-c2ccccc2)c([2H])c1[2H]]

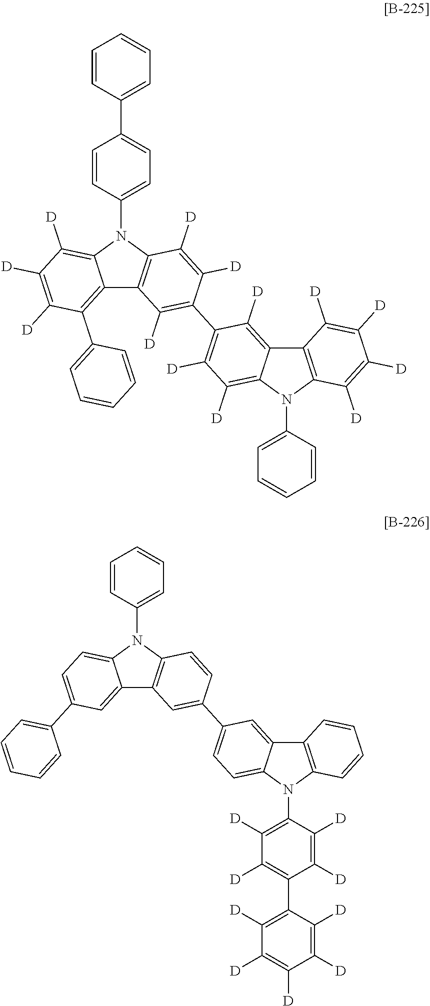 [SMILES: [2H]c1c([2H])c([2H])c2c(c1[2H])c1c([2H])c(-c3c([2H])c([2H])c4c(c3[2H])c3c(-c5ccccc5)c([2H])c([2H])c([2H])c3n4-c3ccc(-c4ccccc4)cc3)c([2H])c([2H])c1n2-c1ccccc1 ; [2H]c1c([2H])c([2H])c(-c2c([2H])c([2H])c(-n3c4ccccc4c4cc(-c5ccc6c(c5)c5cc(-c7ccccc7)ccc5n6-c5ccccc5)ccc43)c([2H])c2[2H])c([2H])c1[2H]]

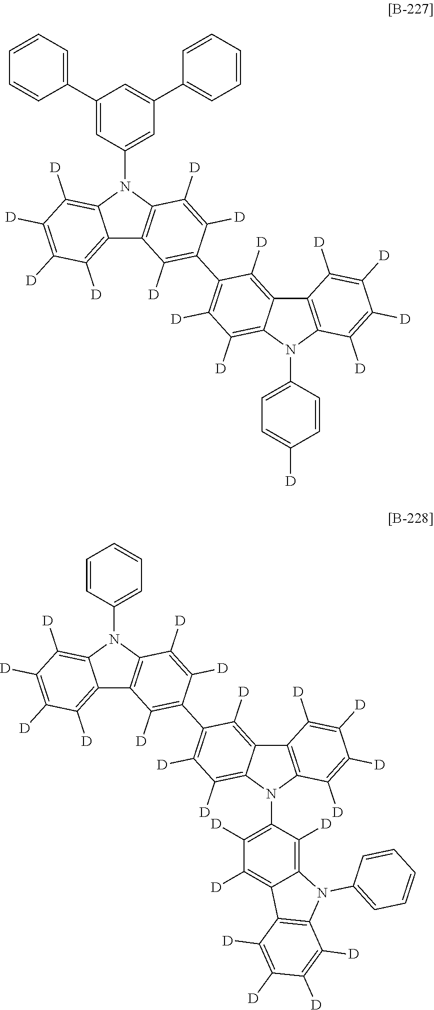 [SMILES: [2H]c1ccc(-n2c3c([2H])c([2H])c([2H])c([2H])c3c3c([2H])c(-c4c([2H])c([2H])c5c(c4[2H])c4c([2H])c([2H])c([2H])c([2H])c4n5-c4cc(-c5ccccc5)cc(-c5ccccc5)c4)c([2H])c([2H])c32)cc1 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3c([2H])c([2H])c4c5c([2H])c([2H])c([2H])c([2H])c5n(-c5ccccc5)c4c3[2H])c([2H])c([2H])c1n2-c1ccccc1]

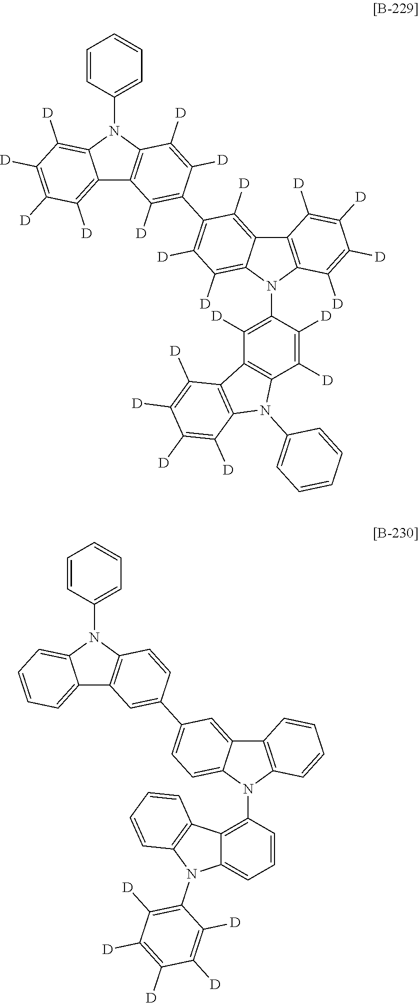 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3c([2H])c([2H])c4c(c3[2H])c3c([2H])c([2H])c([2H])c([2H])c3n4-c3ccccc3)c([2H])c([2H])c1n2-c1ccccc1 ; [2H]c1c([2H])c([2H])c(-n2c3ccccc3c3c(-n4c5ccccc5c5cc(-c6ccc7c(c6)c6ccccc6n7-c6ccccc6)ccc54)cccc32)c([2H])c1[2H]]

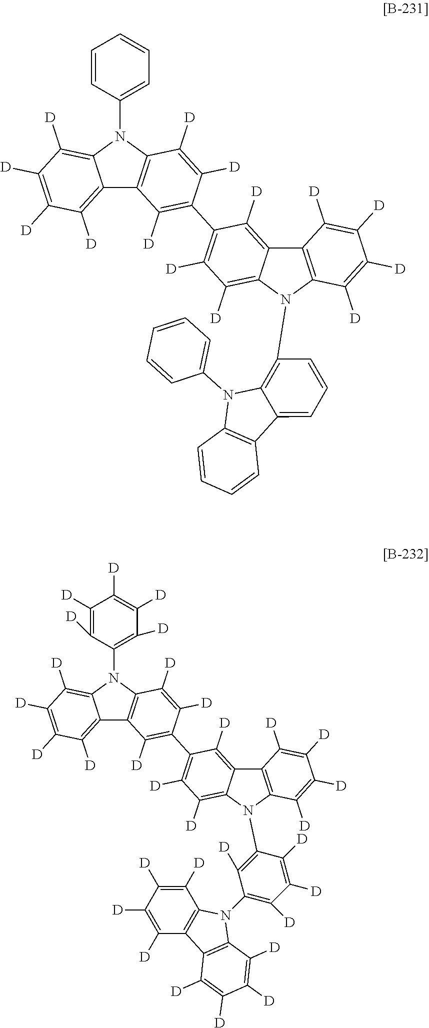 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc4c5ccccc5n(-c5ccccc5)c34)c([2H])c([2H])c1n2-c1ccccc1 ; [2H]c1c([2H])c([2H])c(-n2c3c([2H])c([2H])c([2H])c([2H])c3c3c([2H])c(-c4c([2H])c([2H])c5c(c4[2H])c4c([2H])c([2H])c([2H])c([2H])c4n5-c4c([2H])c([2H])c([2H])c(-n5c6c([2H])c([2H])c([2H])c([2H])c6c6c([2H])c([2H])c([2H])c([2H])c65)c4[2H])c([2H])c([2H])c32)c([2H])c1[2H]]

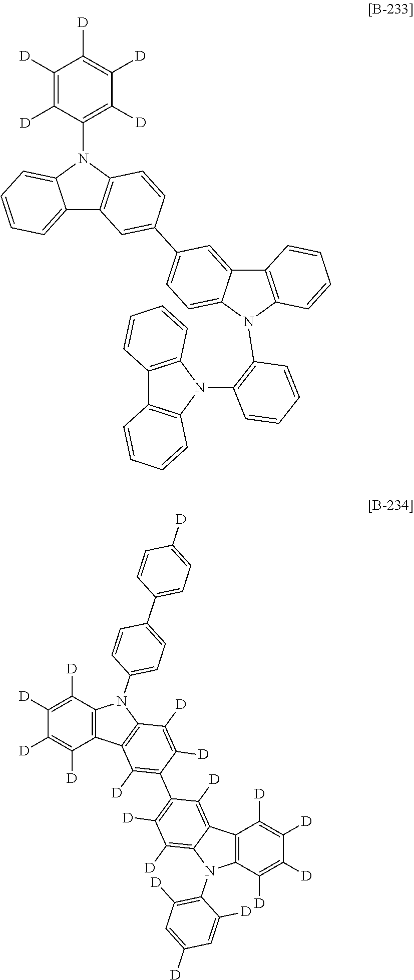 [SMILES: [2H]c1c([2H])c([2H])c(-n2c3ccccc3c3cc(-c4ccc5c(c4)c4ccccc4n5-c4ccccc4-n4c5ccccc5c5ccccc54)ccc32)c([2H])c1[2H] ; [2H]c1ccc(-c2ccc(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])cc([2H])cc5[2H])c([2H])c([2H])c43)cc2)cc1]

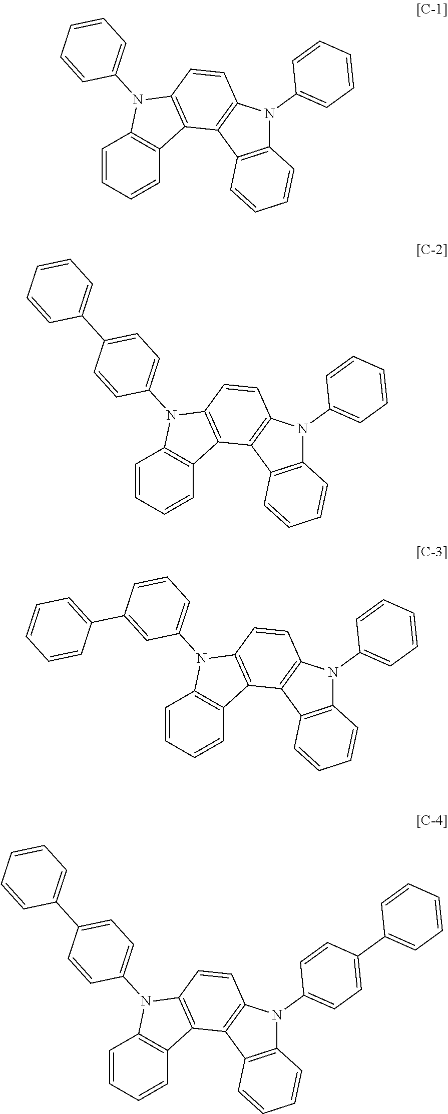 [SMILES: c1ccc(-n2c3ccccc3c3c4c5ccccc5n(-c5ccccc5)c4ccc32)cc1 ; c1ccc(-c2ccc(-n3c4ccccc4c4c5c6ccccc6n(-c6ccccc6)c5ccc43)cc2)cc1 ; c1ccc(-c2cccc(-n3c4ccccc4c4c5c6ccccc6n(-c6ccccc6)c5ccc43)c2)cc1 ; c1ccc(-c2ccc(-n3c4ccccc4c4c5c6ccccc6n(-c6ccc(-c7ccccc7)cc6)c5ccc43)cc2)cc1]

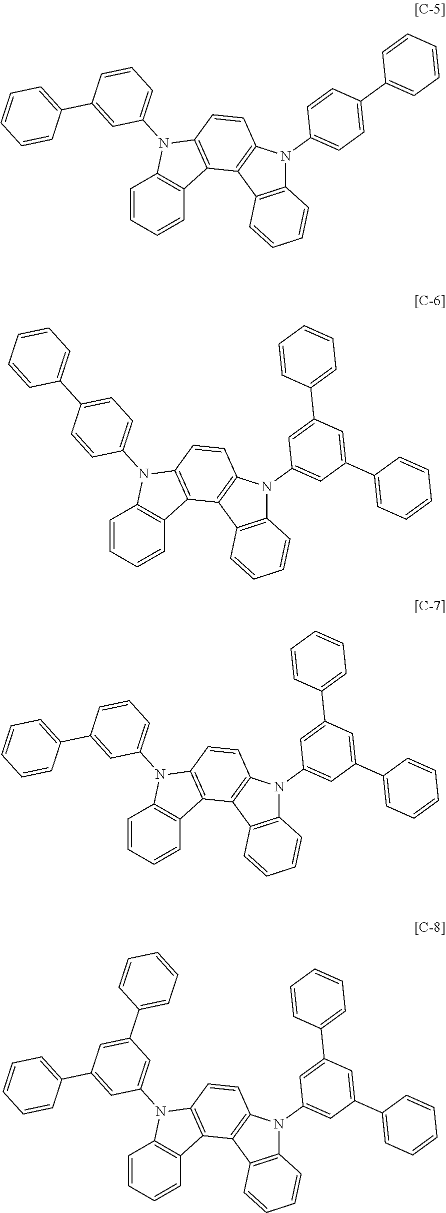 [SMILES: c1ccc(-c2ccc(-n3c4ccccc4c4c5c6ccccc6n(-c6cccc(-c7ccccc7)c6)c5ccc43)cc2)cc1 ; c1ccc(-c2ccc(-n3c4ccccc4c4c5c6ccccc6n(-c6cc(-c7ccccc7)cc(-c7ccccc7)c6)c5ccc43)cc2)cc1 ; c1ccc(-c2cccc(-n3c4ccccc4c4c5c6ccccc6n(-c6cc(-c7ccccc7)cc(-c7ccccc7)c6)c5ccc43)c2)cc1 ; c1ccc(-c2cc(-c3ccccc3)cc(-n3c4ccccc4c4c5c6ccccc6n(-c6cc(-c7ccccc7)cc(-c7ccccc7)c6)c5ccc43)c2)cc1]

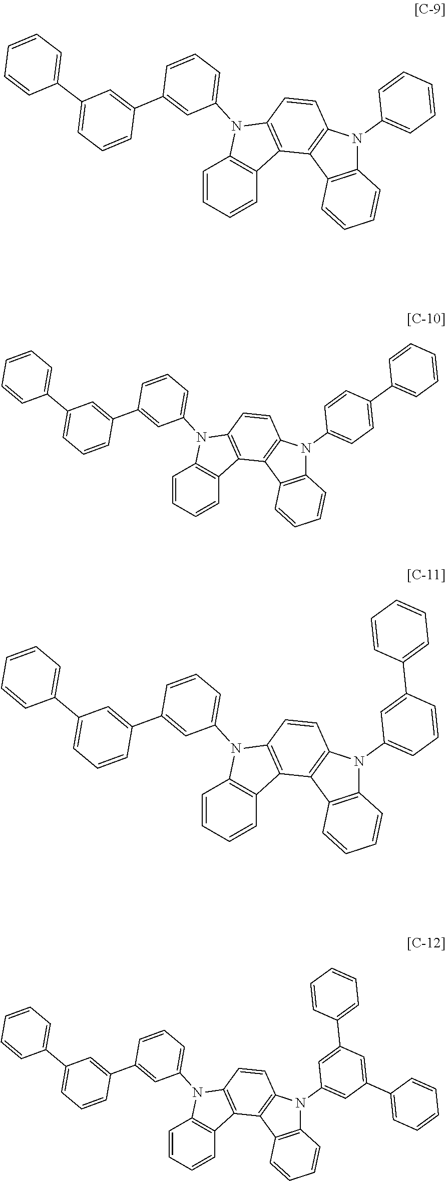 [SMILES: c1ccc(-c2cccc(-c3cccc(-n4c5ccccc5c5c6c7ccccc7n(-c7ccccc7)c6ccc54)c3)c2)cc1 ; c1ccc(-c2ccc(-n3c4ccccc4c4c5c6ccccc6n(-c6cccc(-c7cccc(-c8ccccc8)c7)c6)c5ccc43)cc2)cc1 ; c1ccc(-c2cccc(-c3cccc(-n4c5ccccc5c5c6c7ccccc7n(-c7cccc(-c8ccccc8)c7)c6ccc54)c3)c2)cc1 ; c1ccc(-c2cccc(-c3cccc(-n4c5ccccc5c5c6c7ccccc7n(-c7cc(-c8ccccc8)cc(-c8ccccc8)c7)c6ccc54)c3)c2)cc1]

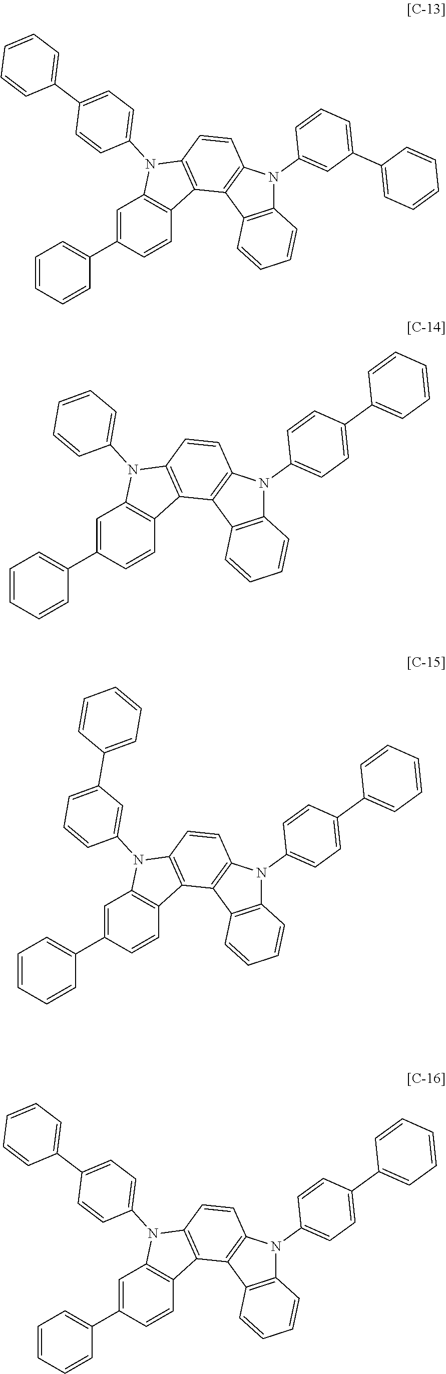 [SMILES: c1ccc(-c2ccc(-n3c4cc(-c5ccccc5)ccc4c4c5c6ccccc6n(-c6cccc(-c7ccccc7)c6)c5ccc43)cc2)cc1 ; c1ccc(-c2ccc(-n3c4ccccc4c4c5c6ccc(-c7ccccc7)cc6n(-c6ccccc6)c5ccc43)cc2)cc1 ; c1ccc(-c2ccc(-n3c4ccccc4c4c5c6ccc(-c7ccccc7)cc6n(-c6cccc(-c7ccccc7)c6)c5ccc43)cc2)cc1 ; c1ccc(-c2ccc(-n3c4ccccc4c4c5c6ccc(-c7ccccc7)cc6n(-c6ccc(-c7ccccc7)cc6)c5ccc43)cc2)cc1]

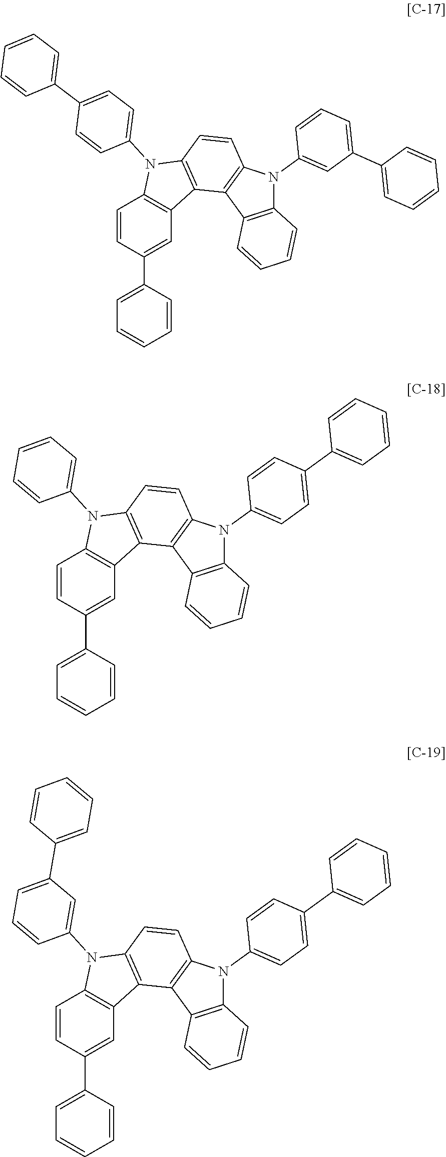 [SMILES: c1ccc(-c2ccc(-n3c4ccc(-c5ccccc5)cc4c4c5c6ccccc6n(-c6cccc(-c7ccccc7)c6)c5ccc43)cc2)cc1 ; c1ccc(-c2ccc(-n3c4ccccc4c4c5c6cc(-c7ccccc7)ccc6n(-c6ccccc6)c5ccc43)cc2)cc1 ; c1ccc(-c2ccc(-n3c4ccccc4c4c5c6cc(-c7ccccc7)ccc6n(-c6cccc(-c7ccccc7)c6)c5ccc43)cc2)cc1]

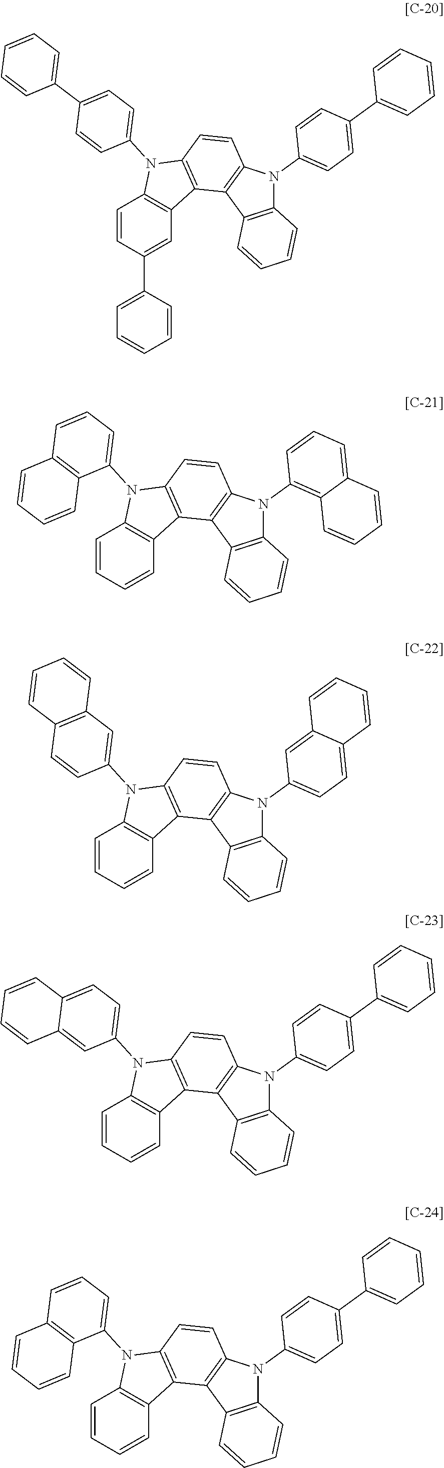 [SMILES: c1ccc(-c2ccc(-n3c4ccccc4c4c5c6cc(-c7ccccc7)ccc6n(-c6ccc(-c7ccccc7)cc6)c5ccc43)cc2)cc1 ; c1ccc2c(-n3c4ccccc4c4c5c6ccccc6n(-c6cccc7ccccc67)c5ccc43)cccc2c1 ; c1ccc2cc(-n3c4ccccc4c4c5c6ccccc6n(-c6ccc7ccccc7c6)c5ccc43)ccc2c1 ; c1ccc(-c2ccc(-n3c4ccccc4c4c5c6ccccc6n(-c6ccc7ccccc7c6)c5ccc43)cc2)cc1 ; c1ccc(-c2ccc(-n3c4ccccc4c4c5c6ccccc6n(-c6cccc7ccccc67)c5ccc43)cc2)cc1]

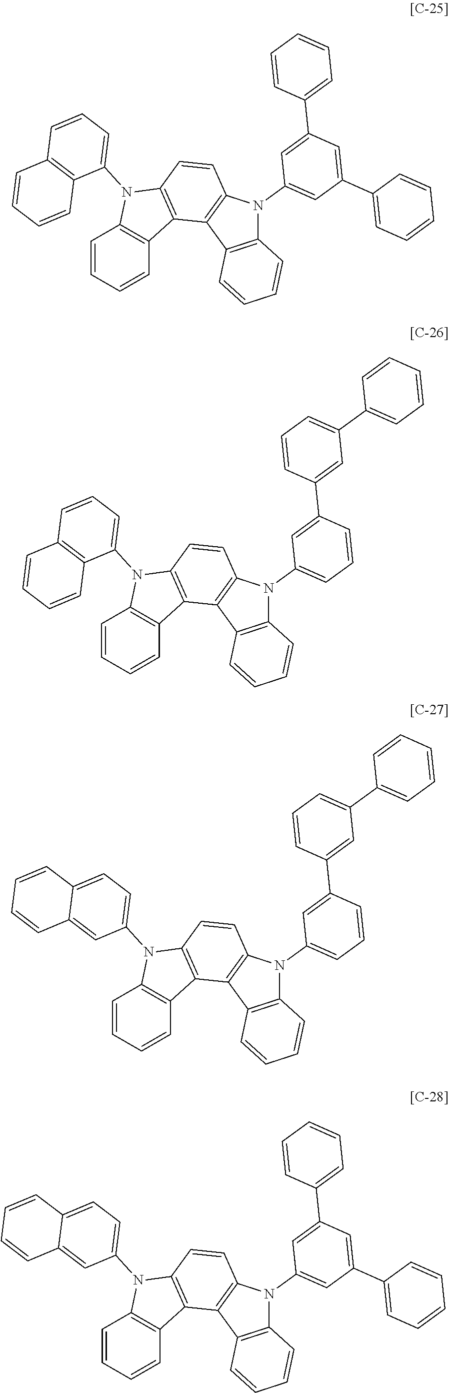 [SMILES: c1ccc(-c2cc(-c3ccccc3)cc(-n3c4ccccc4c4c5c6ccccc6n(-c6cccc7ccccc67)c5ccc43)c2)cc1 ; c1ccc(-c2cccc(-c3cccc(-n4c5ccccc5c5c6c7ccccc7n(-c7cccc8ccccc78)c6ccc54)c3)c2)cc1 ; c1ccc(-c2cccc(-c3cccc(-n4c5ccccc5c5c6c7ccccc7n(-c7ccc8ccccc8c7)c6ccc54)c3)c2)cc1 ; c1ccc(-c2cc(-c3ccccc3)cc(-n3c4ccccc4c4c5c6ccccc6n(-c6ccc7ccccc7c6)c5ccc43)c2)cc1]

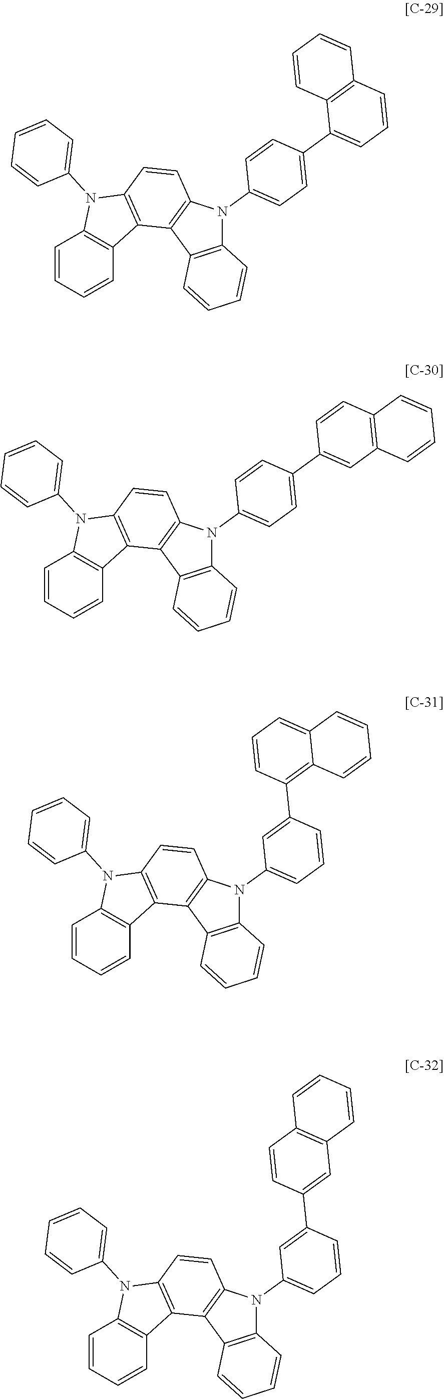 [SMILES: c1ccc(-n2c3ccccc3c3c4c5ccccc5n(-c5ccc(-c6cccc7ccccc67)cc5)c4ccc32)cc1 ; c1ccc(-n2c3ccccc3c3c4c5ccccc5n(-c5ccc(-c6ccc7ccccc7c6)cc5)c4ccc32)cc1 ; c1ccc(-n2c3ccccc3c3c4c5ccccc5n(-c5cccc(-c6cccc7ccccc67)c5)c4ccc32)cc1 ; c1ccc(-n2c3ccccc3c3c4c5ccccc5n(-c5cccc(-c6ccc7ccccc7c6)c5)c4ccc32)cc1]

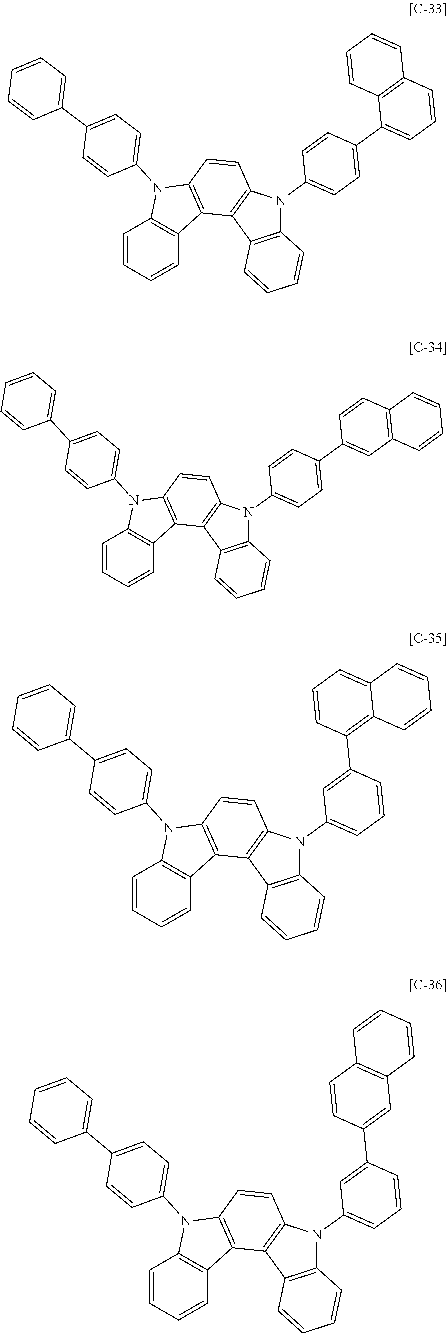 [SMILES: c1ccc(-c2ccc(-n3c4ccccc4c4c5c6ccccc6n(-c6ccc(-c7cccc8ccccc78)cc6)c5ccc43)cc2)cc1 ; c1ccc(-c2ccc(-n3c4ccccc4c4c5c6ccccc6n(-c6ccc(-c7ccc8ccccc8c7)cc6)c5ccc43)cc2)cc1 ; c1ccc(-c2ccc(-n3c4ccccc4c4c5c6ccccc6n(-c6cccc(-c7cccc8ccccc78)c6)c5ccc43)cc2)cc1 ; c1ccc(-c2ccc(-n3c4ccccc4c4c5c6ccccc6n(-c6cccc(-c7ccc8ccccc8c7)c6)c5ccc43)cc2)cc1]

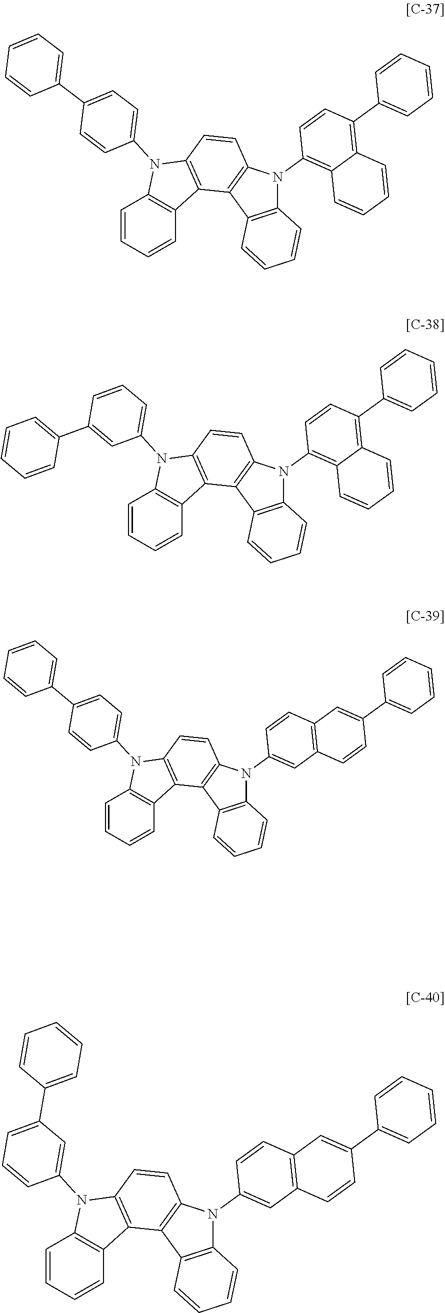 [SMILES: c1ccc(-c2ccc(-n3c4ccccc4c4c5c6ccccc6n(-c6ccc(-c7ccccc7)c7ccccc67)c5ccc43)cc2)cc1 ; c1ccc(-c2cccc(-n3c4ccccc4c4c5c6ccccc6n(-c6ccc(-c7ccccc7)c7ccccc67)c5ccc43)c2)cc1 ; c1ccc(-c2ccc(-n3c4ccccc4c4c5c6ccccc6n(-c6ccc7cc(-c8ccccc8)ccc7c6)c5ccc43)cc2)cc1 ; c1ccc(-c2cccc(-n3c4ccccc4c4c5c6ccccc6n(-c6ccc7cc(-c8ccccc8)ccc7c6)c5ccc43)c2)cc1]

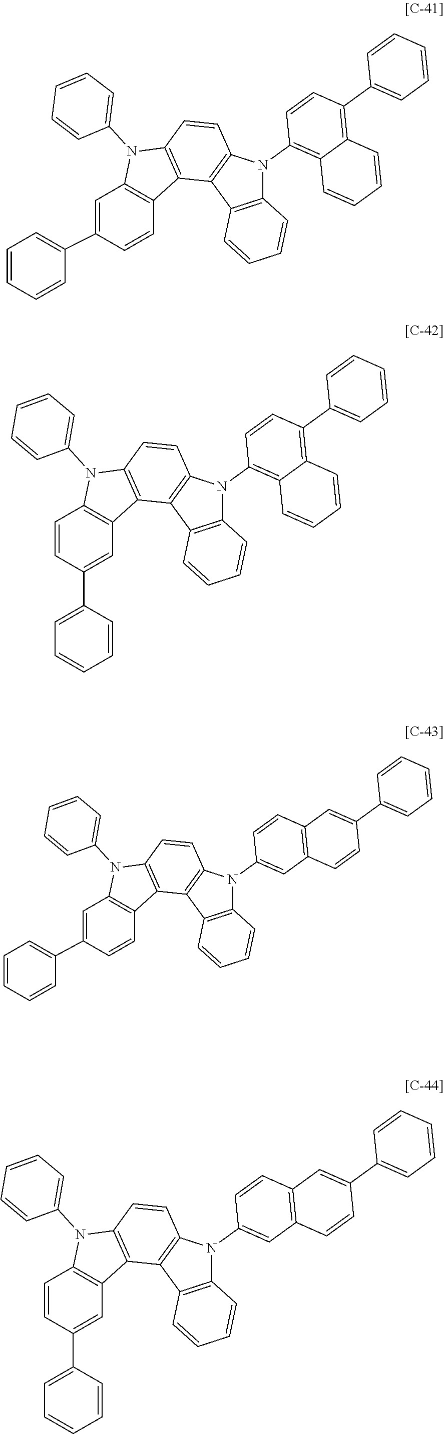 [SMILES: c1ccc(-c2ccc3c4c5c6ccccc6n(-c6ccc(-c7ccccc7)c7ccccc67)c5ccc4n(-c4ccccc4)c3c2)cc1 ; c1ccc(-c2ccc3c(c2)c2c4c5ccccc5n(-c5ccc(-c6ccccc6)c6ccccc56)c4ccc2n3-c2ccccc2)cc1 ; c1ccc(-c2ccc3cc(-n4c5ccccc5c5c6c7ccc(-c8ccccc8)cc7n(-c7ccccc7)c6ccc54)ccc3c2)cc1 ; c1ccc(-c2ccc3cc(-n4c5ccccc5c5c6c7cc(-c8ccccc8)ccc7n(-c7ccccc7)c6ccc54)ccc3c2)cc1]

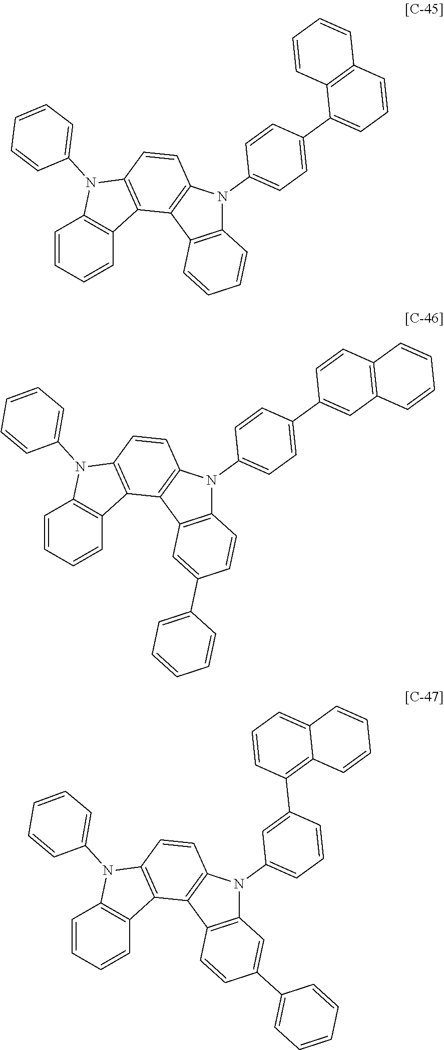 [SMILES: c1ccc(-n2c3ccccc3c3c4c5ccccc5n(-c5ccc(-c6cccc7ccccc67)cc5)c4ccc32)cc1 ; c1ccc(-c2ccc3c(c2)c2c4c5ccccc5n(-c5ccccc5)c4ccc2n3-c2ccc(-c3ccc4ccccc4c3)cc2)cc1 ; c1ccc(-c2ccc3c4c5c6ccccc6n(-c6ccccc6)c5ccc4n(-c4cccc(-c5cccc6ccccc56)c4)c3c2)cc1]

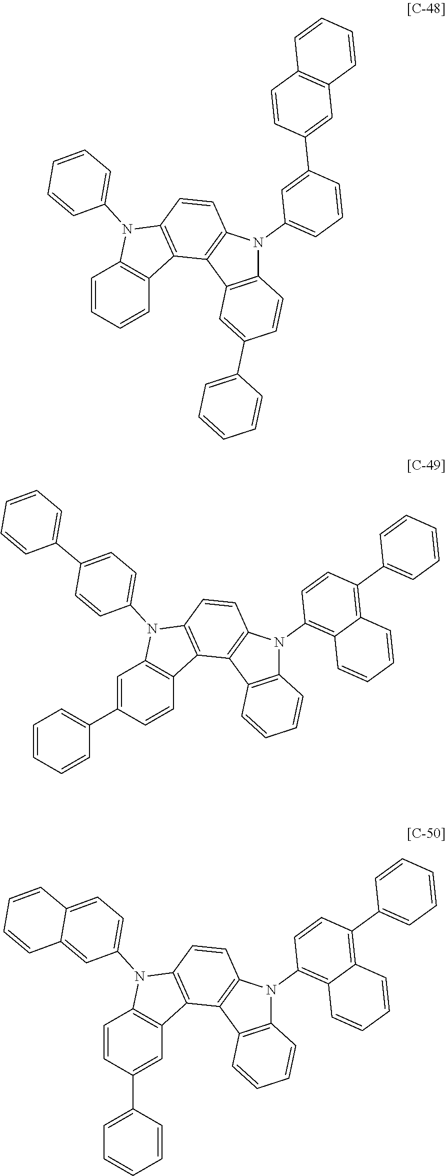 [SMILES: c1ccc(-c2ccc3c(c2)c2c4c5ccccc5n(-c5ccccc5)c4ccc2n3-c2cccc(-c3ccc4ccccc4c3)c2)cc1 ; c1ccc(-c2ccc(-n3c4cc(-c5ccccc5)ccc4c4c5c6ccccc6n(-c6ccc(-c7ccccc7)c7ccccc67)c5ccc43)cc2)cc1 ; c1ccc(-c2ccc3c(c2)c2c4c5ccccc5n(-c5ccc(-c6ccccc6)c6ccccc56)c4ccc2n3-c2ccc3ccccc3c2)cc1]

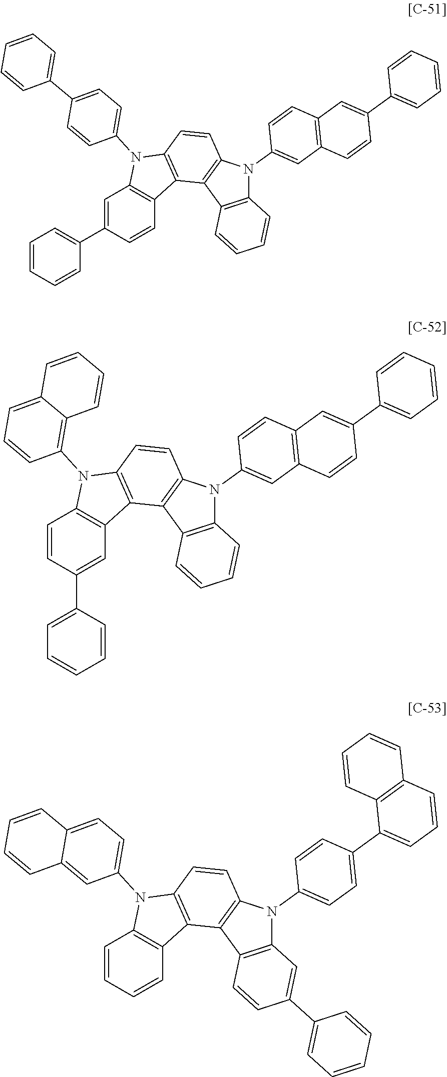 [SMILES: c1ccc(-c2ccc(-n3c4cc(-c5ccccc5)ccc4c4c5c6ccccc6n(-c6ccc7cc(-c8ccccc8)ccc7c6)c5ccc43)cc2)cc1 ; c1ccc(-c2ccc3cc(-n4c5ccccc5c5c6c7cc(-c8ccccc8)ccc7n(-c7cccc8ccccc78)c6ccc54)ccc3c2)cc1 ; c1ccc(-c2ccc3c4c5c6ccccc6n(-c6ccc7ccccc7c6)c5ccc4n(-c4ccc(-c5cccc6ccccc56)cc4)c3c2)cc1]

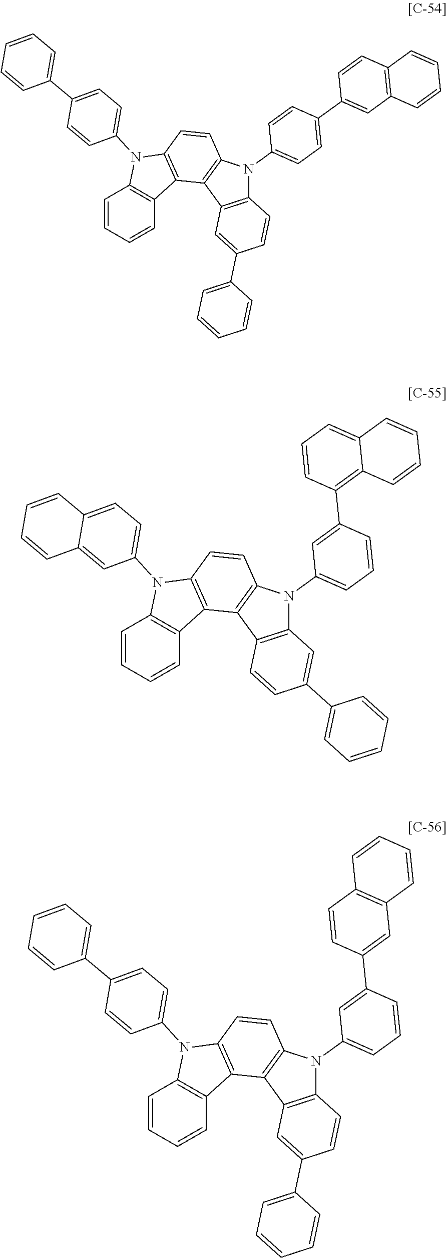 [SMILES: c1ccc(-c2ccc(-n3c4ccccc4c4c5c6cc(-c7ccccc7)ccc6n(-c6ccc(-c7ccc8ccccc8c7)cc6)c5ccc43)cc2)cc1 ; c1ccc(-c2ccc3c4c5c6ccccc6n(-c6ccc7ccccc7c6)c5ccc4n(-c4cccc(-c5cccc6ccccc56)c4)c3c2)cc1 ; c1ccc(-c2ccc(-n3c4ccccc4c4c5c6cc(-c7ccccc7)ccc6n(-c6cccc(-c7ccc8ccccc8c7)c6)c5ccc43)cc2)cc1]

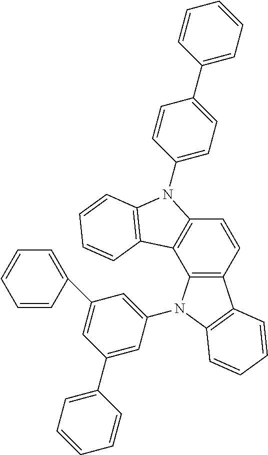 [SMILES: c1ccc(-c2ccc(-n3c4ccccc4c4c3ccc3c5ccccc5n(-c5cc(-c6ccccc6)cc(-c6ccccc6)c5)c34)cc2)cc1]

Additionally, examples of Compound C-1 to Compound C-57 listed in Group 2 in which at least one hydrogen is replaced with deuterium are given below, but are not limited thereto.

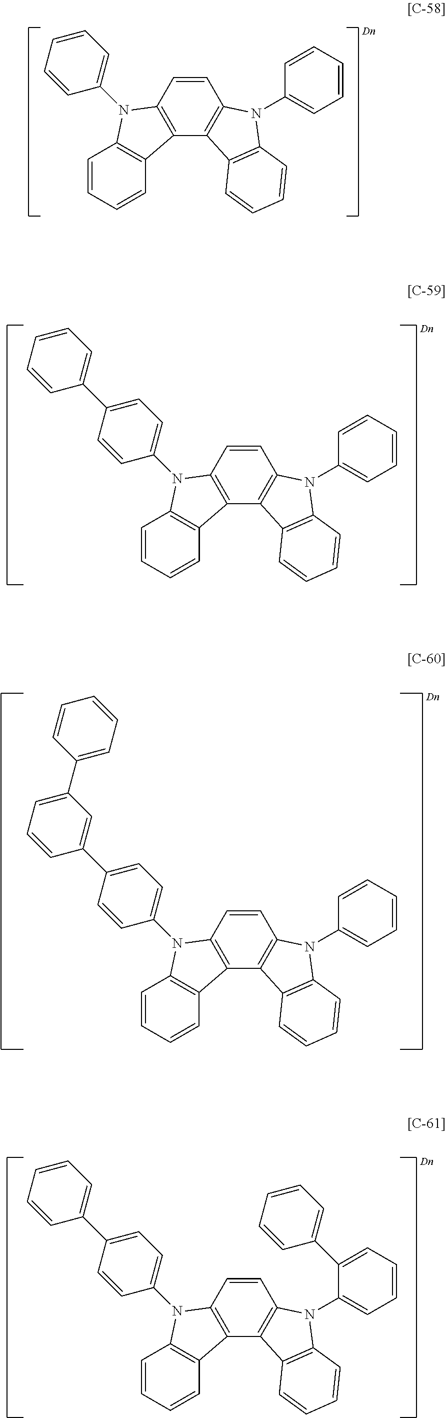

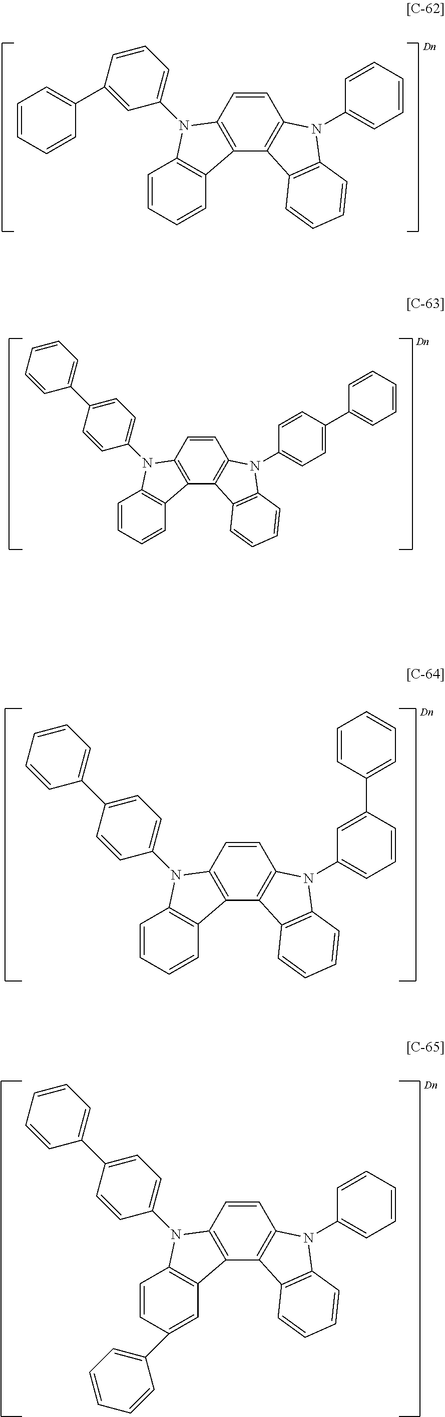

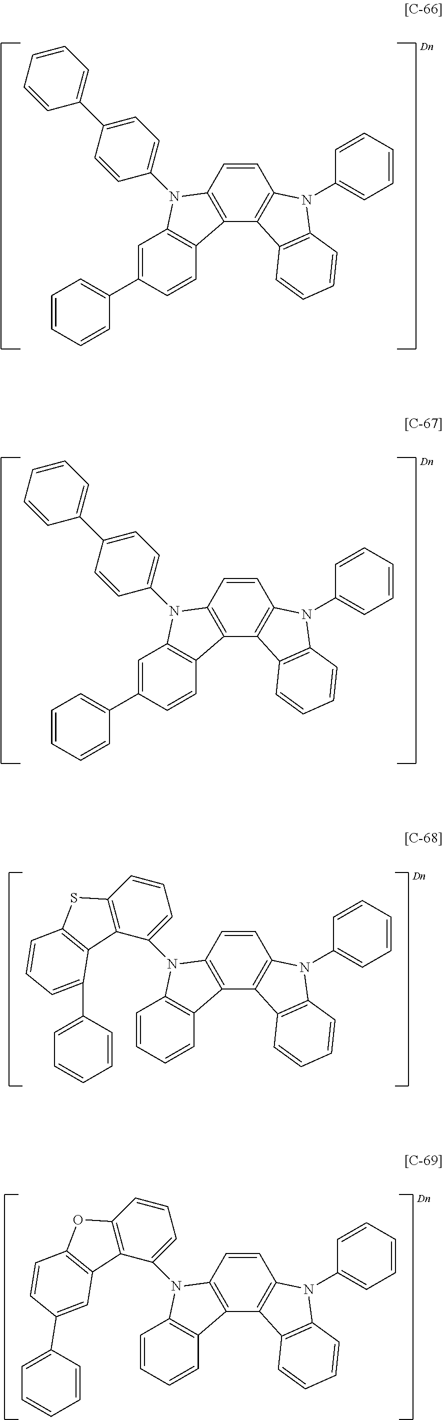

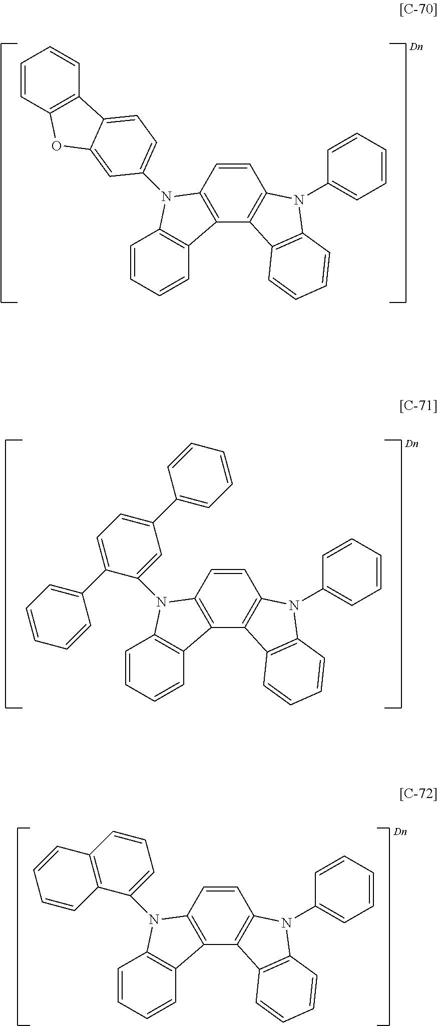

(Dn refers to the number of deuterium substitutions and indicates a structure substituted with one or more deuteriums)

The most specific structures for Compound C-58 to Compound C-72 of Group 2 are presented below as examples according to the position and substitution rate of deuterium substitution, and are not the intention to limit the scope of rights to compounds not listed below.

The scope of the present disclosure is determined by the claims, and when deuterium is substituted, it is not limited to the compounds exemplified below, and the deuterium substitution position, deuterium substitution rate, etc. may include all changeable ranges within the range of Compound C-58 to Compound C-72.

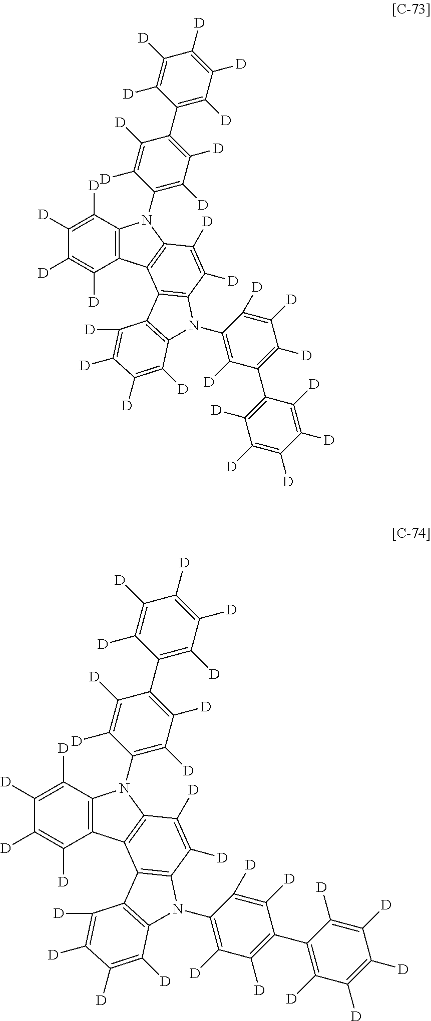 [SMILES: [2H]c1c([2H])c([2H])c(-c2c([2H])c([2H])c(-n3c4c([2H])c([2H])c([2H])c([2H])c4c4c5c6c([2H])c([2H])c([2H])c([2H])c6n(-c6c([2H])c([2H])c([2H])c(-c7c([2H])c([2H])c([2H])c([2H])c7[2H])c6[2H])c5c([2H])c([2H])c43)c([2H])c2[2H])c([2H])c1[2H] ; [2H]c1c([2H])c([2H])c(-c2c([2H])c([2H])c(-n3c4c([2H])c([2H])c([2H])c([2H])c4c4c5c6c([2H])c([2H])c([2H])c([2H])c6n(-c6c([2H])c([2H])c(-c7c([2H])c([2H])c([2H])c([2H])c7[2H])c([2H])c6[2H])c5c([2H])c([2H])c43)c([2H])c2[2H])c([2H])c1[2H]]

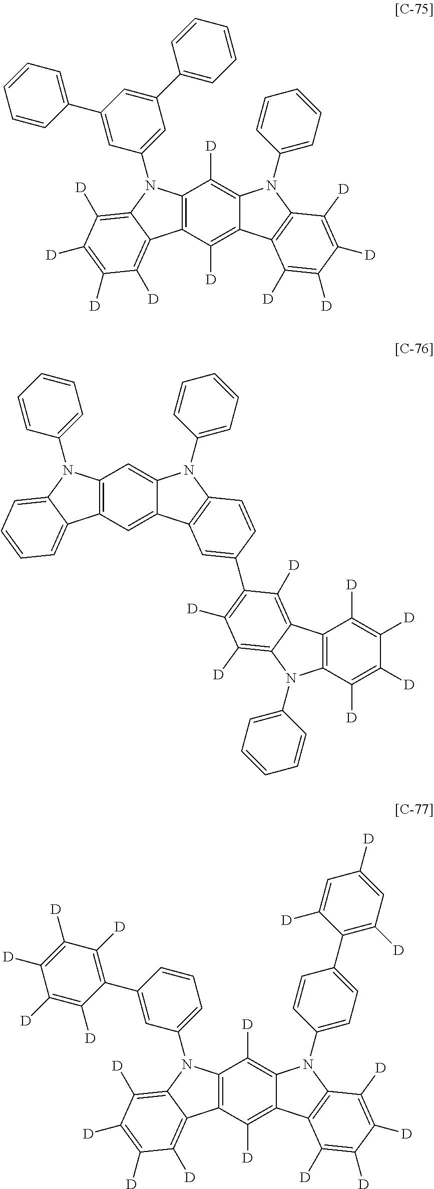 [SMILES: [2H]c1c([2H])c([2H])c2c(c1[2H])c1c([2H])c3c4c([2H])c([2H])c([2H])c([2H])c4n(-c4cc(-c5ccccc5)cc(-c5ccccc5)c4)c3c([2H])c1n2-c1ccccc1 ; [2H]c1c([2H])c([2H])c2c(c1[2H])c1c([2H])c(-c3ccc4c(c3)c3cc5c6ccccc6n(-c6ccccc6)c5cc3n4-c3ccccc3)c([2H])c([2H])c1n2-c1ccccc1 ; [2H]c1cc([2H])c(-c2ccc(-n3c4c([2H])c([2H])c([2H])c([2H])c4c4c([2H])c5c6c([2H])c([2H])c([2H])c([2H])c6n(-c6cccc(-c7c([2H])c([2H])c([2H])c([2H])c7[2H])c6)c5c([2H])c43)cc2)c([2H])c1]

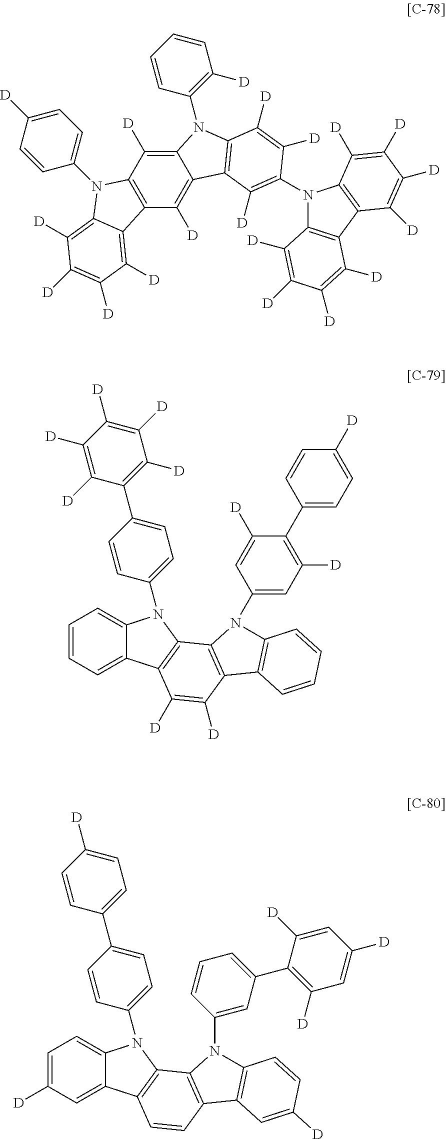 [SMILES: [2H]c1ccc(-n2c3c([2H])c([2H])c([2H])c([2H])c3c3c([2H])c4c5c([2H])c(-n6c7c([2H])c([2H])c([2H])c([2H])c7c7c([2H])c([2H])c([2H])c([2H])c76)c([2H])c([2H])c5n(-c5ccccc5[2H])c4c([2H])c32)cc1 ; [2H]c1ccc(-c2c([2H])cc(-n3c4ccccc4c4c([2H])c([2H])c5c6ccccc6n(-c6ccc(-c7c([2H])c([2H])c([2H])c([2H])c7[2H])cc6)c5c43)cc2[2H])cc1 ; [2H]c1ccc(-c2ccc(-n3c4ccc([2H])cc4c4ccc5c6cc([2H])ccc6n(-c6cccc(-c7c([2H])cc([2H])cc7[2H])c6)c5c43)cc2)cc1]

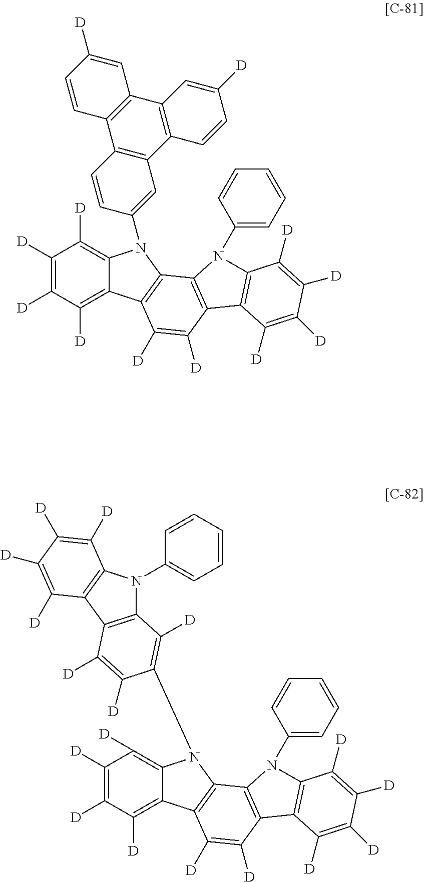 [SMILES: [2H]c1ccc2c3ccc(-n4c5c([2H])c([2H])c([2H])c([2H])c5c5c([2H])c([2H])c6c7c([2H])c([2H])c([2H])c([2H])c7n(-c7ccccc7)c6c54)cc3c3ccc([2H])cc3c2c1 ; [2H]c1c([2H])c([2H])c2c(c1[2H])c1c([2H])c([2H])c(-n3c4c([2H])c([2H])c([2H])c([2H])c4c4c([2H])c([2H])c5c6c([2H])c([2H])c([2H])c([2H])c6n(-c6ccccc6)c5c43)c([2H])c1n2-c1ccccc1]

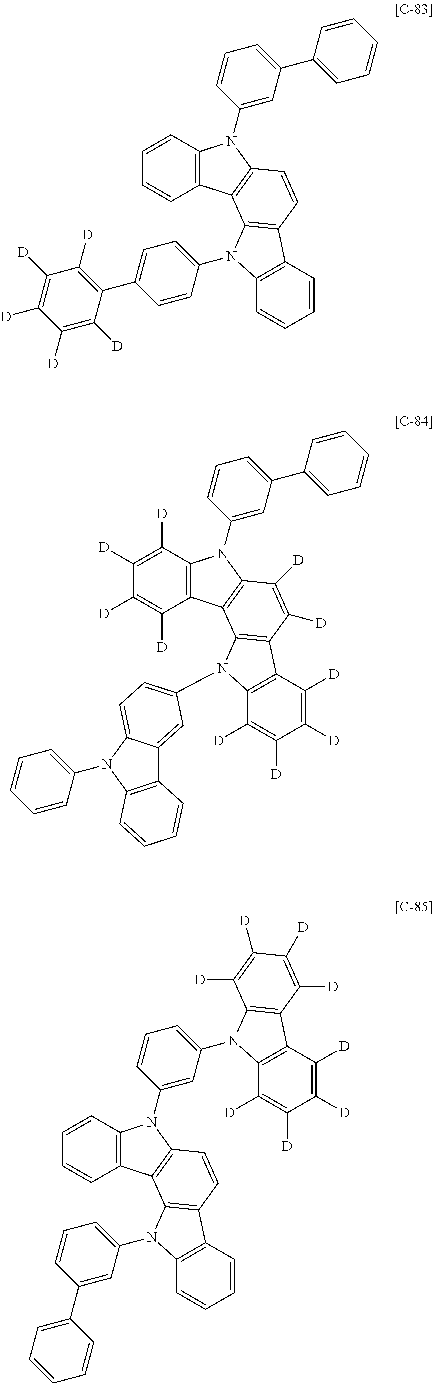 [SMILES: [2H]c1c([2H])c([2H])c(-c2ccc(-n3c4ccccc4c4ccc5c(c6ccccc6n5-c5cccc(-c6ccccc6)c5)c43)cc2)c([2H])c1[2H] ; [2H]c1c([2H])c([2H])c2c(c1[2H])c1c(c([2H])c([2H])c3c4c([2H])c([2H])c([2H])c([2H])c4n(-c4ccc5c(c4)c4ccccc4n5-c4ccccc4)c31)n2-c1cccc(-c2ccccc2)c1 ; [2H]c1c([2H])c([2H])c2c(c1[2H])c1c([2H])c([2H])c([2H])c([2H])c1n2-c1cccc(-n2c3ccccc3c3c2ccc2c4ccccc4n(-c4cccc(-c5ccccc5)c4)c23)c1]

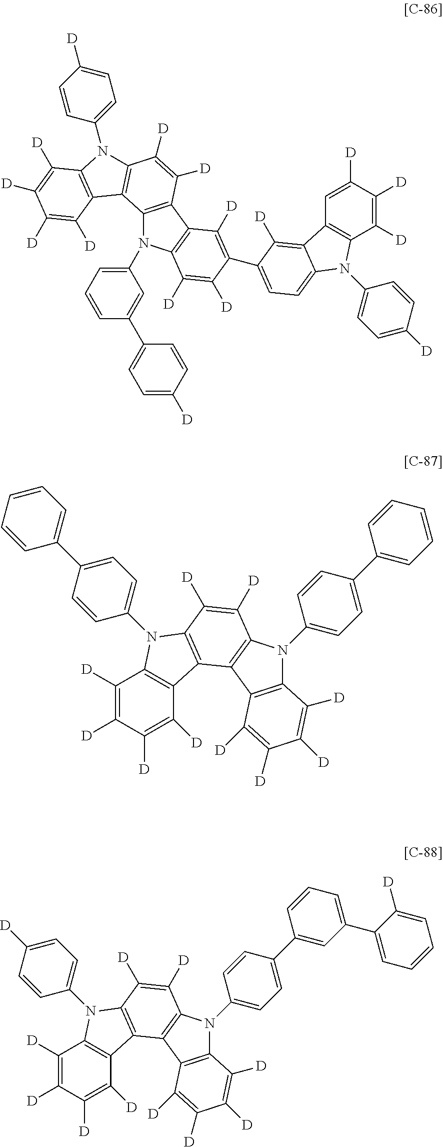 [SMILES: [2H]c1ccc(-c2cccc(-n3c4c([2H])c([2H])c(-c5ccc6c(c5[2H])c5cc([2H])c([2H])c([2H])c5n6-c5ccc([2H])cc5)c([2H])c4c4c([2H])c([2H])c5c(c6c([2H])c([2H])c([2H])c([2H])c6n5-c5ccc([2H])cc5)c43)c2)cc1 ; [2H]c1c([2H])c([2H])c2c(c1[2H])c1c3c4c([2H])c([2H])c([2H])c([2H])c4n(-c4ccc(-c5ccccc5)cc4)c3c([2H])c([2H])c1n2-c1ccc(-c2ccccc2)cc1 ; [2H]c1ccc(-n2c3c([2H])c([2H])c([2H])c([2H])c3c3c4c5c([2H])c([2H])c([2H])c([2H])c5n(-c5ccc(-c6cccc(-c7ccccc7[2H])c6)cc5)c4c([2H])c([2H])c32)cc1]

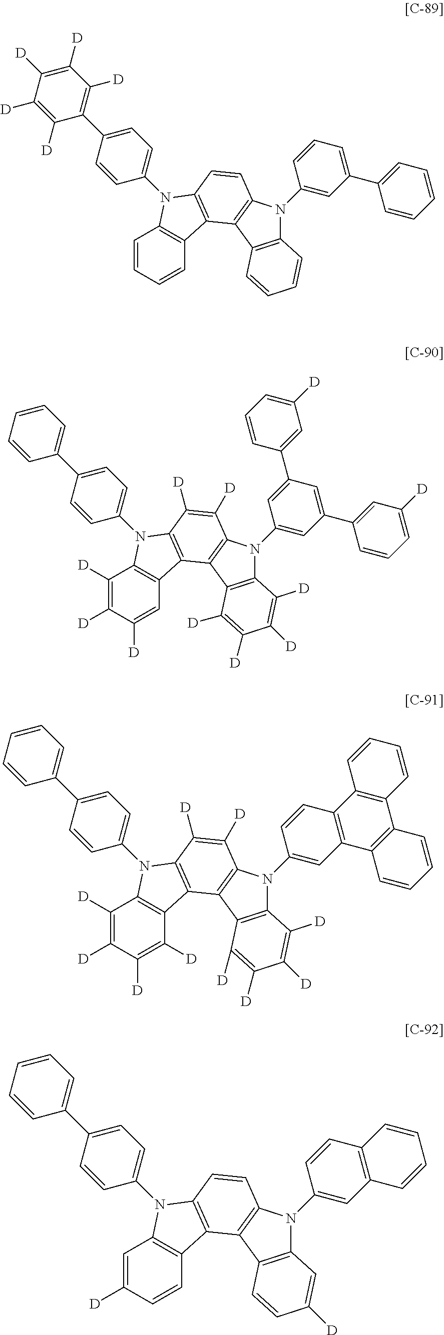 [SMILES: [2H]c1c([2H])c([2H])c(-c2ccc(-n3c4ccccc4c4c5c6ccccc6n(-c6cccc(-c7ccccc7)c6)c5ccc43)cc2)c([2H])c1[2H] ; [2H]c1cccc(-c2cc(-c3cccc([2H])c3)cc(-n3c4c([2H])c([2H])c([2H])c([2H])c4c4c5c6cc([2H])c([2H])c([2H])c6n(-c6ccc(-c7ccccc7)cc6)c5c([2H])c([2H])c43)c2)c1 ; [2H]c1c([2H])c([2H])c2c(c1[2H])c1c3c4c([2H])c([2H])c([2H])c([2H])c4n(-c4ccc5c6ccccc6c6ccccc6c5c4)c3c([2H])c([2H])c1n2-c1ccc(-c2ccccc2)cc1 ; [2H]c1ccc2c3c4c5ccc([2H])cc5n(-c5ccc6ccccc6c5)c4ccc3n(-c3ccc(-c4ccccc4)cc3)c2c1]

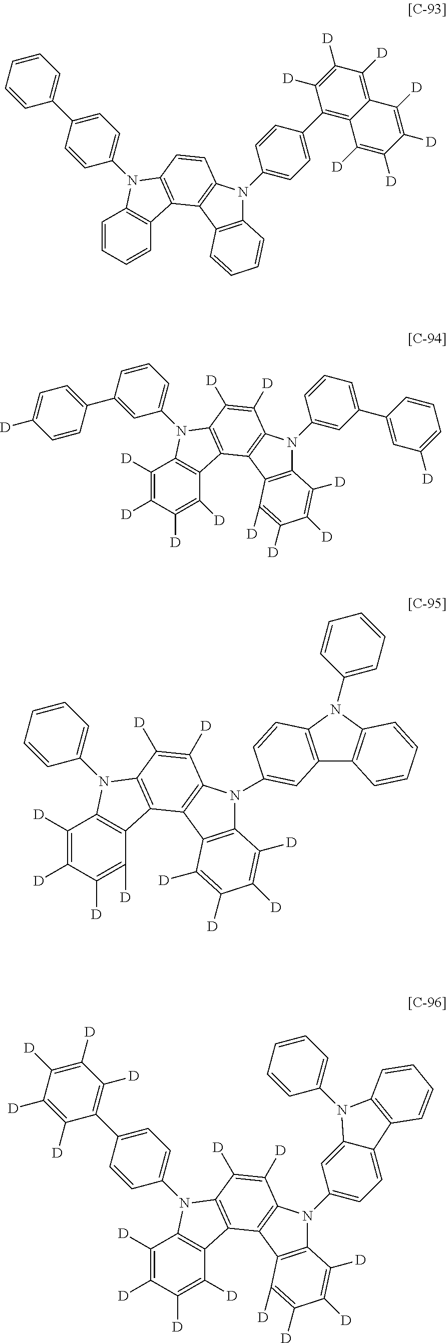

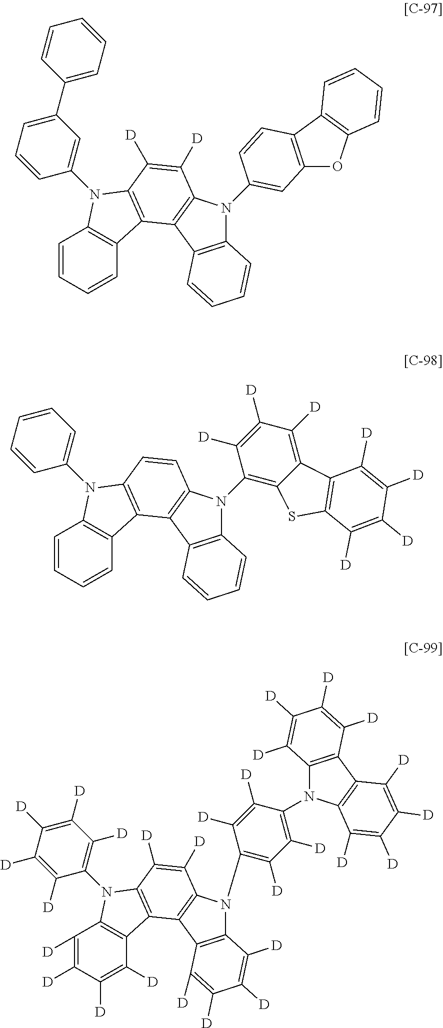 [SMILES: [2H]c1c([2H])c2c(c3ccccc3n2-c2ccc3c(c2)oc2ccccc23)c2c3ccccc3n(-c3cccc(-c4ccccc4)c3)c12 ; [2H]c1c([2H])c([2H])c2c(sc3c(-n4c5ccccc5c5c6c7ccccc7n(-c7ccccc7)c6ccc54)c([2H])c([2H])c([2H])c32)c1[2H] ; [2H]c1c([2H])c([2H])c(-n2c3c([2H])c([2H])c([2H])c([2H])c3c3c4c5c([2H])c([2H])c([2H])c([2H])c5n(-c5c([2H])c([2H])c(-n6c7c([2H])c([2H])c([2H])c([2H])c7c7c([2H])c([2H])c([2H])c([2H])c76)c([2H])c5[2H])c4c([2H])c([2H])c32)c([2H])c1[2H]]

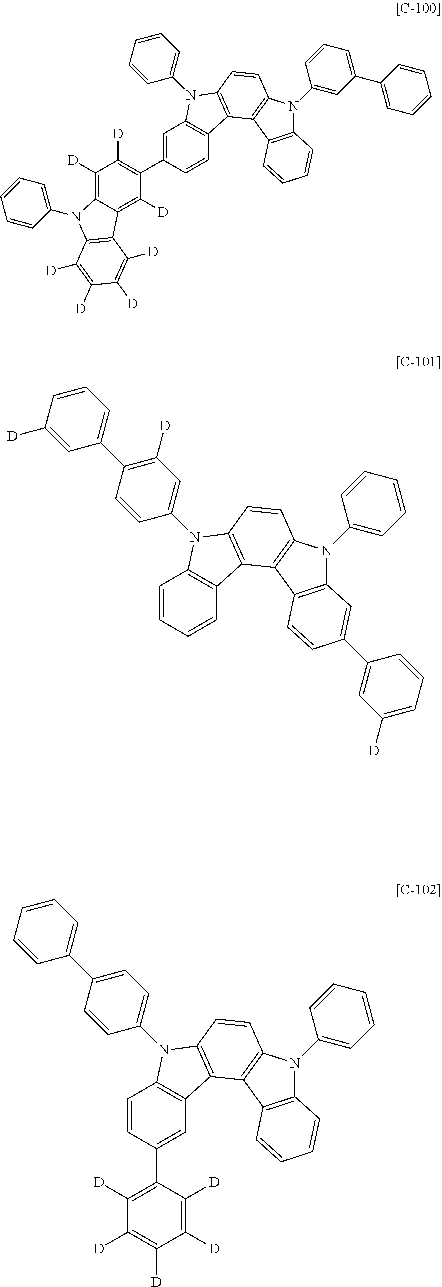 [SMILES: [2H]c1c([2H])c([2H])c2c(c1[2H])c1c([2H])c(-c3ccc4c5c6c7ccccc7n(-c7cccc(-c8ccccc8)c7)c6ccc5n(-c5ccccc5)c4c3)c([2H])c([2H])c1n2-c1ccccc1 ; [2H]c1cccc(-c2ccc3c4c5c6ccccc6n(-c6ccc(-c7cccc([2H])c7)c([2H])c6)c5ccc4n(-c4ccccc4)c3c2)c1 ; [2H]c1c([2H])c([2H])c(-c2ccc3c(c2)c2c4c5ccccc5n(-c5ccccc5)c4ccc2n3-c2ccc(-c3ccccc3)cc2)c([2H])c1[2H]]

The first compound and the second compound may be included in a weight ratio of, for example, 1:99 to 99:1. Within the above range, bipolar properties may be implemented by matching an appropriate weight ratio using electron transport capability of the first compound and the hole transport capability of the second compound, to improve efficiency and life-span. Within this range, for example, they may be included in a weight ratio of about 10:90 to 90:10, about 20:80 to 80:20, for example, about 20:80 to about 70:30, about 20:80 to about 60:40, and about 30:70 to about 60:40. As a specific example, they may be included in a weight ratio of 40:60, 50:50, or 60:40.

In addition to the first compound and the second compound described above, one or more additional compounds may be included.

The aforementioned compound for an organic optoelectronic device or composition for an organic optoelectronic device may be a composition further including a dopant.

The dopant may be, for example, a phosphorescent dopant, for example, a red, green, or blue phosphorescent dopant, and may be, for example, a red or green phosphorescent dopant.

The dopant is a material mixed with the compound or composition for an organic optoelectronic device in a small amount to cause light emission, and may be generally a material such as a metal complex that emits light by multiple excitation into a triplet or more. The dopant may be, for example an inorganic, organic, or organic/inorganic compound, and one or more types thereof may be used.

Examples of the dopant may be a phosphorescent dopant and examples of the phosphorescent dopant may be an organic metal compound including Ir, Pt, Os, Ti, Zr, Hf, Eu, Tb, Tm, Fe, Co, Ni, Ru, Rh, Pd, or a combination thereof. The phosphorescent dopant may be, for example a compound represented by Chemical Formula Z, but is not limited thereto.

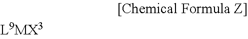

In Chemical Formula Z, M is a metal, and L⁹ and X³ are the same or different and are a ligand to form a complex compound with M.

The M may be for example Ir, Pt, Os, Ti, Zr, Hf, Eu, Tb, Tm, Fe, Co, Ni, Ru, Rh, Pd, or a combination thereof and L⁹ and X³ may be for example a bidentate ligand.

Examples of ligands represented by L⁹ and X³ may be selected from the Chemical Formulas listed in Group A, but are not limited thereto.

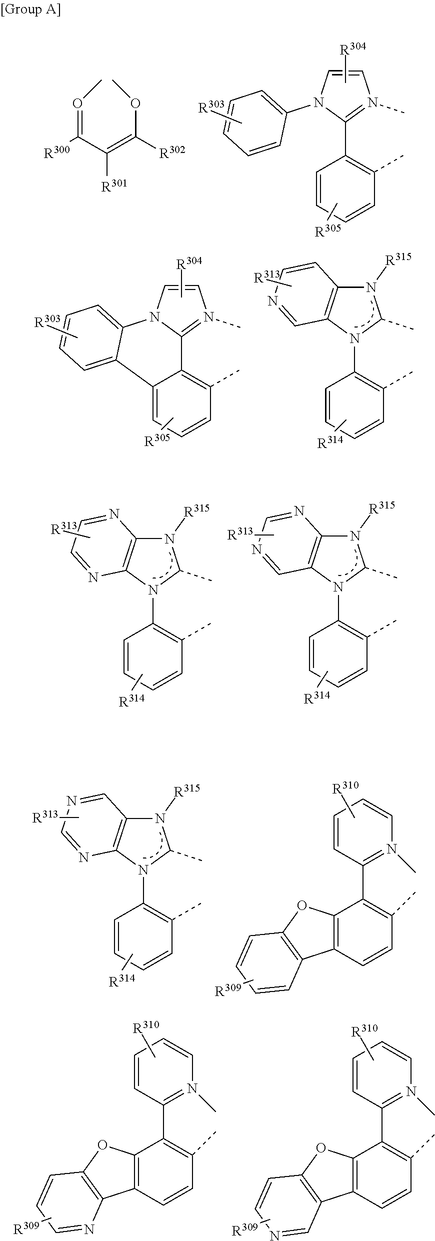

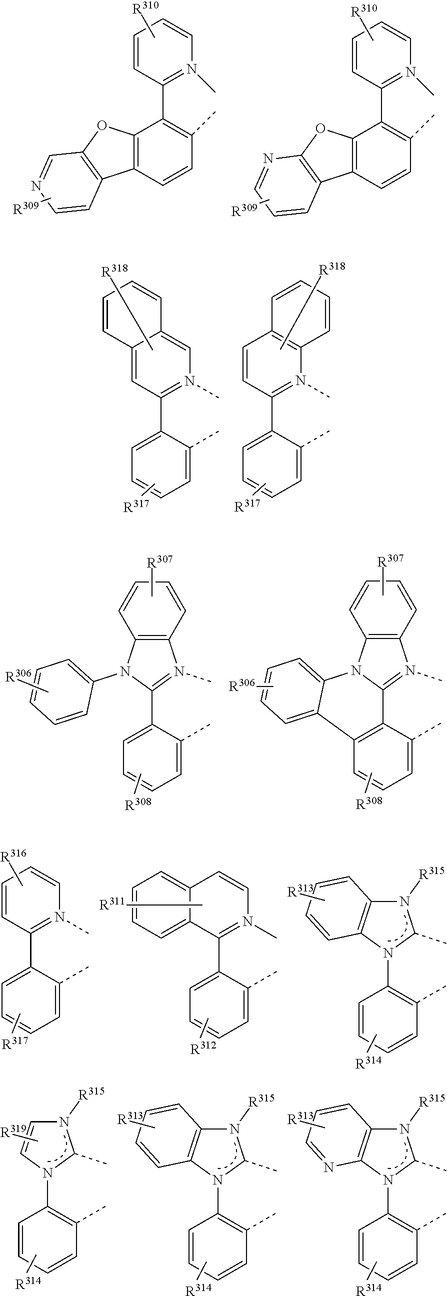

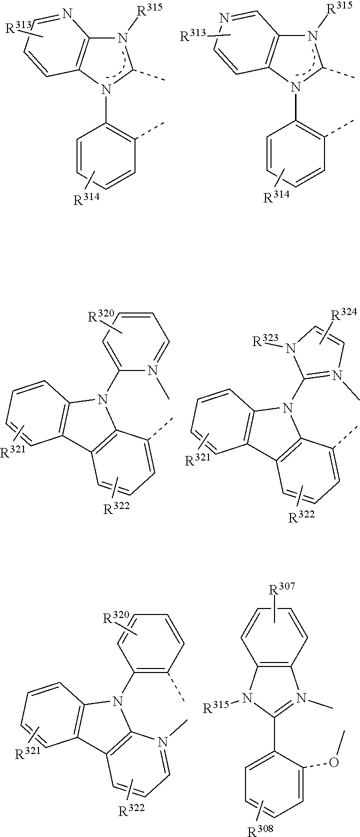

In Group A,
R³⁰⁰ to R³⁰² are each independently hydrogen, deuterium, a C1 to C30 alkyl group that is substituted or unsubstituted with a halogen, a C6 to C30 aryl group that is substituted or unsubstituted with a C1 to C30 alkyl, or a halogen, and
R³⁰³ to R³²⁴ are each independently hydrogen, deuterium, halogen, a substituted or unsubstituted C1 to C30 alkyl group, a substituted or unsubstituted C1 to C30 alkoxy group, a substituted or unsubstituted C3 to C30 cycloalkyl group, a substituted or unsubstituted C2 to C30 alkenyl group, a substituted or unsubstituted C6 to C30 aryl group, a substituted or unsubstituted C1 to C30 heteroaryl group, a substituted or unsubstituted C1 to C30 amino group, a substituted or unsubstituted C6 to C30 arylamino group, SF₅, a trialkylsilyl group having a substituted or unsubstituted C1 to C30 alkyl group, a dialkylarylsilyl group having a substituted or unsubstituted C1 to C30 alkyl group and a C6 to C30 aryl group, or a triarylsilyl group having a substituted or unsubstituted C6 to C30 aryl group.

As an example, it may include a dopant represented by Chemical Formula V.

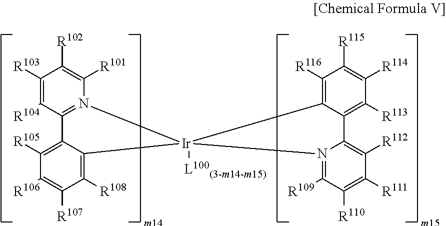

In Chemical Formula V,
R¹⁰¹ to R¹¹⁶ are each independently hydrogen, deuterium, a substituted or unsubstituted C1 to C10 alkyl group, a substituted or unsubstituted C6 to C20 aryl group, or —SiR¹³²R¹³³R¹³⁴,
R¹³² to R¹³⁴ are each independently a C1 to C6 alkyl group,
at least one of R¹⁰ to R¹¹⁶ is a functional group represented by Chemical Formula V-1,
L¹⁰⁰ is a bidentate ligand of a monovalent anion, and is a ligand that coordinates to iridium through a lone pair of carbons or heteroatoms, and
m14 and m15 are each independently any one of integers of 0 to 3, and m14+m15 is any one of integers of 1 to 3,

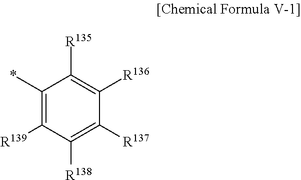

wherein, in Chemical Formula V-1,
R¹³⁵ to R¹³⁹ are each independently hydrogen, deuterium, a substituted or unsubstituted C1 to C10 alkyl group, a substituted or unsubstituted C6 to C20 aryl group, or —SiR¹³²R¹³³R¹³⁴, and
* means a portion linked to a carbon atom.

As an example, a dopant represented by Chemical Formula Z-1 may be included.

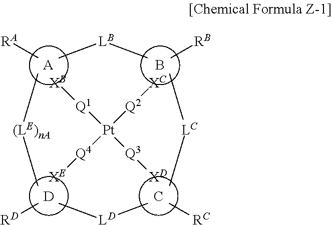

In Chemical Formula Z-1, rings A, B, C, and D independently represent a 5-membered or 6-membered carbocyclic or heterocyclic ring;
R^{A}, R^{B}, R^{c}, and R^{D} independently represent mono-, di-, tri-, or tetra-substitution, or unsubstitution;
L^{B}, L^{C}, and L^{D} are each independent selected from a direct bond, BR, NR, PR, O, S, Se, C═O, S═O, SO₂, CRR′, SiRR′, GeRR′, and a combination thereof,
when nA is 1, L^{E} is selected from a direct bond, BR, NR, PR, O, S, Se, C═O, S═O, SO₂, CRR′, SiRR′, GeRR′, and a combination thereof, when nA is 0, L^{E} does not exist; and
R^{A}, R^{B}, R^{c}, R^{D}, R, and R′ are each independently selected from hydrogen, deuterium, a halogen, alkyl group, a cycloalkyl group, a heteroalkyl group, an arylalkyl group, an alkoxy group, an aryloxy group, an amino group, a silyl group, an alkenyl group, a cycloalkenyl group, a heteroalkenyl group, an alkynyl group, an aryl group, a heteroaryl group, an acyl group, a carbonyl group, a carboxylic acid group, an ester group, a nitrile group, an isonitrile group, a sulfanyl group, a sulfinyl group, a sulfonyl group, a phosphino group, and a combination thereof, any adjacent R^{A}, R^{B}, R^{c}, R^{D}, R, and R′ are optionally linked to each other to provide a ring; X^{B}, X^{C}, X^{D}, and X^{E} are each independently selected from carbon and nitrogen; and Q¹, Q², Q³, and Q⁴ each represent oxygen or a direct bond.

The dopant according to an embodiment may be a platinum complex, and may be, for example, represented by Chemical Formula VI.

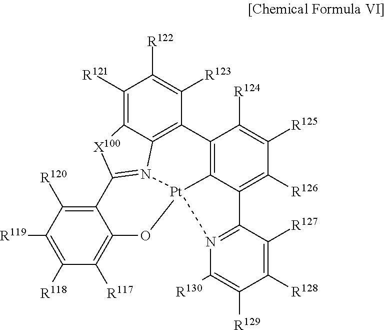

In Chemical Formula VI,
X¹⁰⁰ is selected from O, S, and NR¹³¹,
R¹¹⁷ to R¹³¹ are each independently hydrogen, deuterium, a substituted or unsubstituted C1 to C10 alkyl group, a substituted or unsubstituted C6 to C20 aryl group, or —SiR¹³²R¹³³R¹³⁴,
R¹³² to R¹³⁴ are each independently a C1 to C6 alkyl group,
at least one of R¹¹⁷ to R¹³¹ is —SiR¹³²R¹³³R¹³⁴ or a tert-butyl group, and
R¹³² to R¹³⁴ are each independently a C1 to C6 alkyl group.

Hereinafter, an organic optoelectronic device using the aforementioned compound for an organic optoelectronic device or composition for an organic optoelectronic device is described.

The organic optoelectronic device may be a suitable device to convert electrical energy into photoenergy and vice versa, e.g., an organic photoelectric device, an organic light emitting diode, an organic solar cell, or an organic photoconductor drum.

Herein, an organic light emitting diode as one example of an organic optoelectronic device is described referring to drawings.

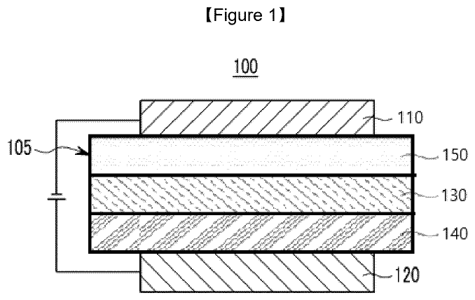

FIG. 1 is a cross-sectional view showing an organic light emitting diode according to an embodiment.

Referring to FIG. 1, an organic light emitting diode 100 according to an embodiment includes an anode 120 and a cathode 110 facing each other and an organic layer 105 disposed between the anode 120 and cathode 110.

The anode 120 may be made of a conductor having a large work function to help hole injection, and may be for example a metal, a metal oxide and/or a conductive polymer. The anode 120 may be, for example a metal such as nickel, platinum, vanadium, chromium, copper, zinc, gold, and the like or an alloy thereof; a metal oxide such as zinc oxide, indium oxide, indium tin oxide (ITO), indium zinc oxide (IZO), and the like; a combination of a metal and an oxide such as ZnO and Al or SnO₂ and Sb; a conductive polymer such as poly(3-methylthiophene), poly(3,4-(ethylene-1,2-dioxy)thiophene) (PEDOT), polypyrrole, and polyaniline, but is not limited thereto.

The cathode 110 may be made of a conductor having a small work function to help electron injection, and may be for example a metal, a metal oxide, and/or a conductive polymer. The cathode 110 may include a metal such as magnesium, calcium, sodium, potassium, titanium, indium, yttrium, lithium, gadolinium, aluminum, silver, tin, lead, cesium, barium, or an alloy thereof; a multilayer structure material such as LiF/Al, LiO₂/Al, LiF/Ca, LiF/Al, and BaF₂/Ca, but is not limited thereto.

The organic layer 105 may include the aforementioned compound for an organic optoelectronic device or composition for an organic optoelectronic device.

The organic layer 105 may include a light emitting layer 130 and the light emitting layer 130 may include the aforementioned compound for an organic optoelectronic device or composition for an organic optoelectronic device.

The composition for an organic optoelectronic device further including a dopant may be, for example, a green light emitting composition.

The light emitting layer 130 may include, for example, the aforementioned compound for an organic optoelectronic device, as a phosphorescent host.

The organic layer may further include a charge transport region in addition to the light emitting layer.

The charge transport region may be, for example, a hole transport region 140.

The hole transport region 140 may further increase hole injection and/or hole mobility between the anode 120 and the light emitting layer 130 and block electrons.

Specifically, the hole transport region 140 may include a hole transport layer between the anode 120 and the light emitting layer 130, and a hole transport auxiliary layer between the light emitting layer 130 and the hole transport layer, and at least one of the compounds of Group B may be included in at least one of the hole transport layer and the hole transport auxiliary layer.

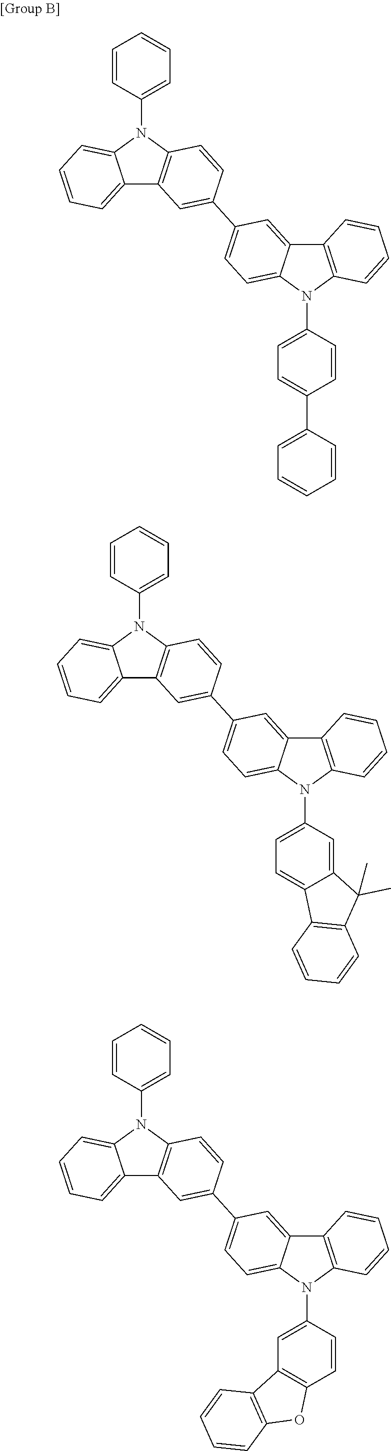 [SMILES: c1ccc(-c2ccc(-n3c4ccccc4c4cc(-c5ccc6c(c5)c5ccccc5n6-c5ccccc5)ccc43)cc2)cc1 ; CC1(C)c2ccccc2-c2ccc(-n3c4ccccc4c4cc(-c5ccc6c(c5)c5ccccc5n6-c5ccccc5)ccc43)cc21 ; c1ccc(-n2c3ccccc3c3cc(-c4ccc5c(c4)c4ccccc4n5-c4ccc5oc6ccccc6c5c4)ccc32)cc1]

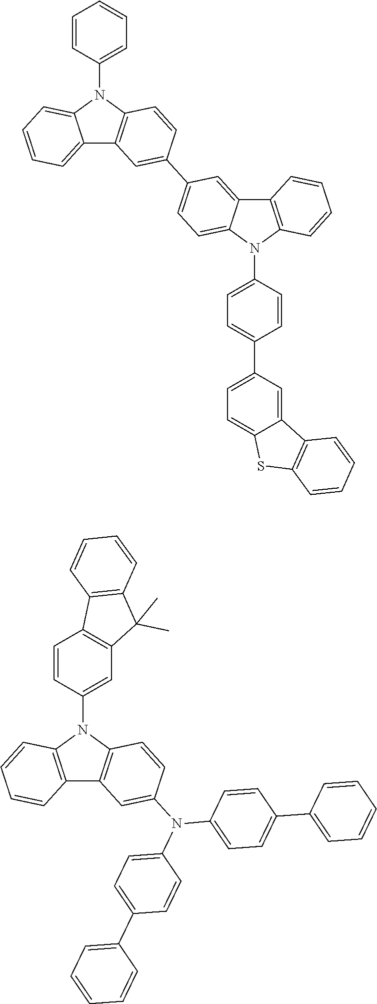 [SMILES: c1ccc(-n2c3ccccc3c3cc(-c4ccc5c(c4)c4ccccc4n5-c4ccc(-c5ccc6sc7ccccc7c6c5)cc4)ccc32)cc1 ; CC1(C)c2ccccc2-c2ccc(-n3c4ccccc4c4cc(N(c5ccc(-c6ccccc6)cc5)c5ccc(-c6ccccc6)cc5)ccc43)cc21]

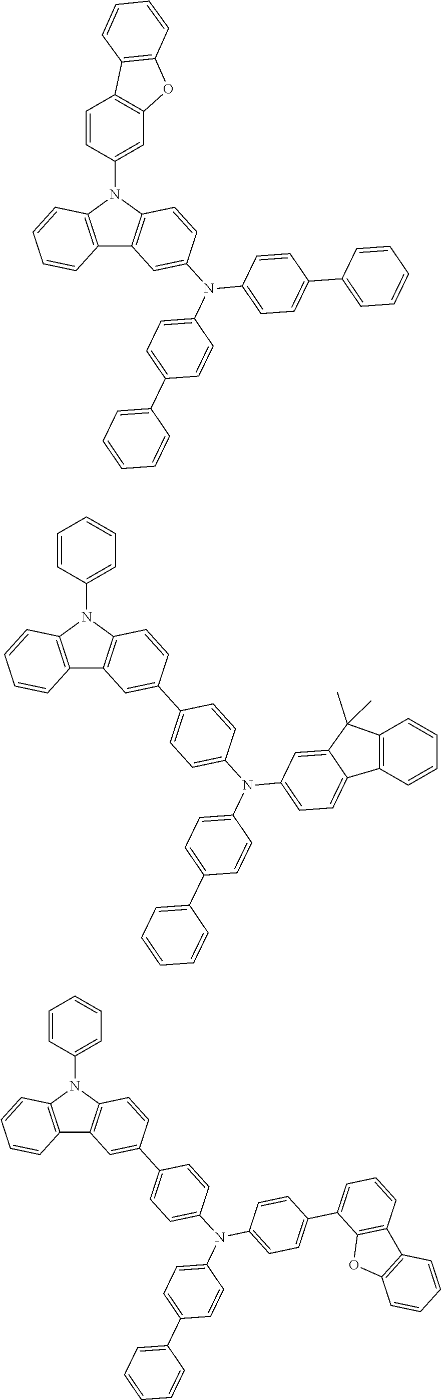 [SMILES: c1ccc(-c2ccc(N(c3ccc(-c4ccccc4)cc3)c3ccc4c(c3)c3ccccc3n4-c3ccc4c(c3)oc3ccccc34)cc2)cc1 ; CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(-c2ccc(N(c3ccc(-c4ccc5c(c4)c4ccccc4n5-c4ccccc4)cc3)c3ccc(-c4cccc5c4oc4ccccc45)cc3)cc2)cc1]

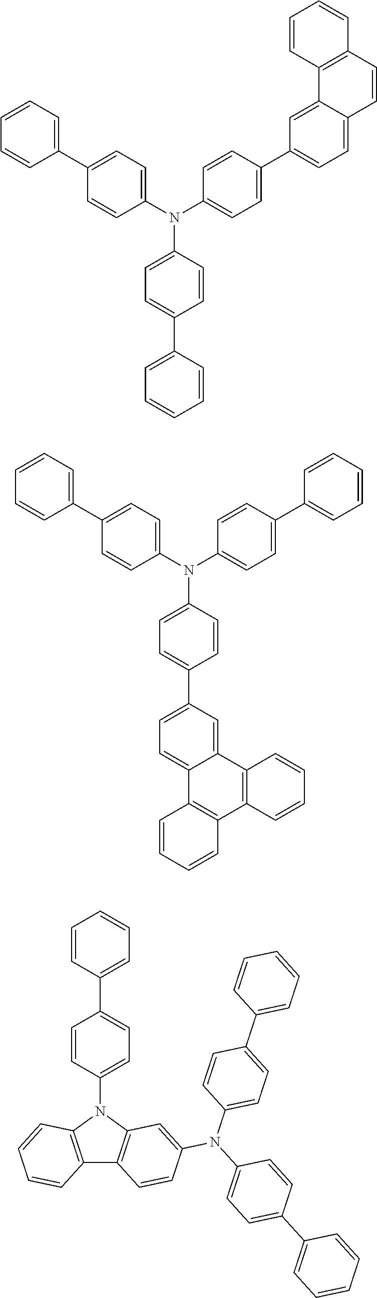 [SMILES: c1ccc(-c2ccc(N(c3ccc(-c4ccccc4)cc3)c3ccc(-c4ccc5ccc6ccccc6c5c4)cc3)cc2)cc1 ; c1ccc(-c2ccc(N(c3ccc(-c4ccccc4)cc3)c3ccc(-c4ccc5c6ccccc6c6ccccc6c5c4)cc3)cc2)cc1 ; c1ccc(-c2ccc(N(c3ccc(-c4ccccc4)cc3)c3ccc4c5ccccc5n(-c5ccc(-c6ccccc6)cc5)c4c3)cc2)cc1]

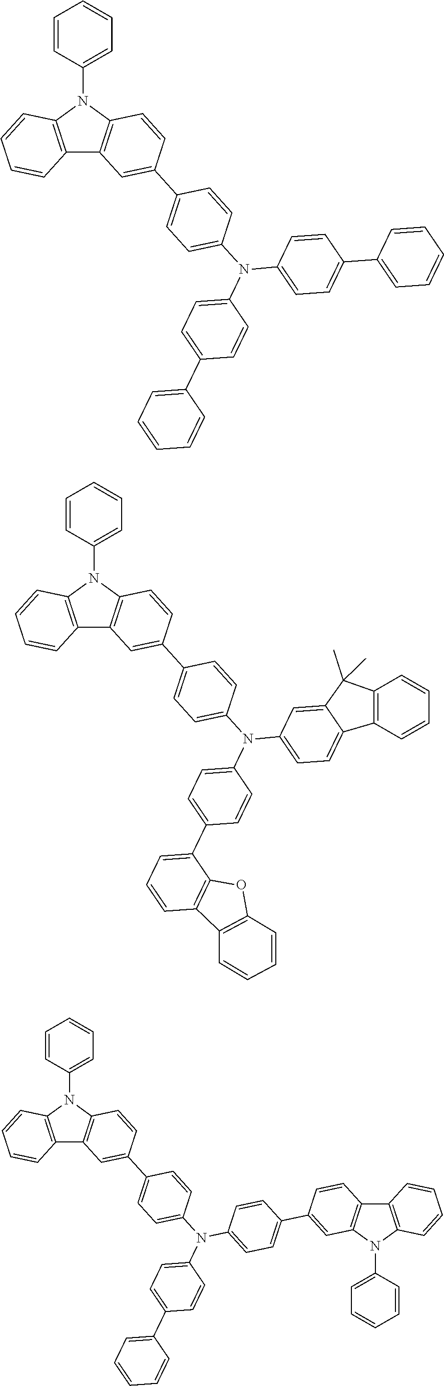 [SMILES: c1ccc(-c2ccc(N(c3ccc(-c4ccccc4)cc3)c3ccc(-c4ccc5c(c4)c4ccccc4n5-c4ccccc4)cc3)cc2)cc1 ; CC1(C)c2ccccc2-c2ccc(N(c3ccc(-c4ccc5c(c4)c4ccccc4n5-c4ccccc4)cc3)c3ccc(-c4cccc5c4oc4ccccc45)cc3)cc21 ; c1ccc(-c2ccc(N(c3ccc(-c4ccc5c(c4)c4ccccc4n5-c4ccccc4)cc3)c3ccc(-c4ccc5c6ccccc6n(-c6ccccc6)c5c4)cc3)cc2)cc1]

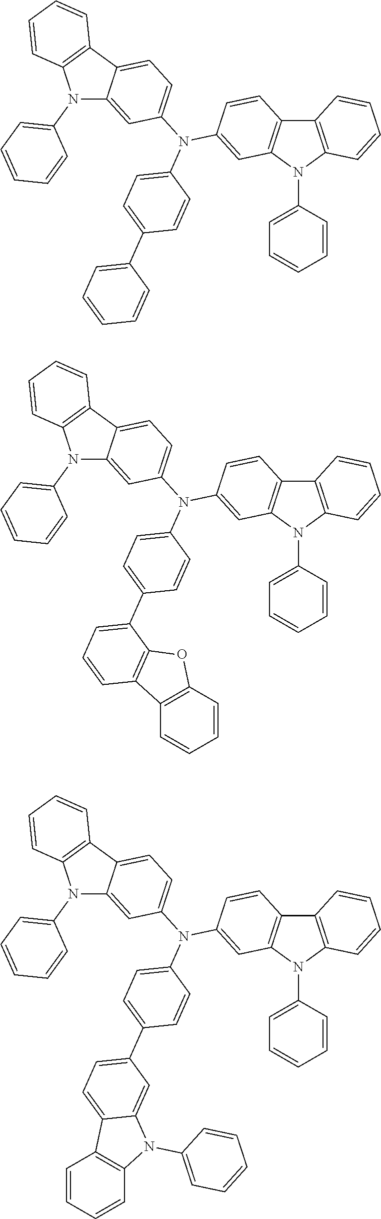 [SMILES: c1ccc(-c2ccc(N(c3ccc4c5ccccc5n(-c5ccccc5)c4c3)c3ccc4c5ccccc5n(-c5ccccc5)c4c3)cc2)cc1 ; c1ccc(-n2c3ccccc3c3ccc(N(c4ccc(-c5cccc6c5oc5ccccc56)cc4)c4ccc5c6ccccc6n(-c6ccccc6)c5c4)cc32)cc1 ; c1ccc(-n2c3ccccc3c3ccc(-c4ccc(N(c5ccc6c7ccccc7n(-c7ccccc7)c6c5)c5ccc6c7ccccc7n(-c7ccccc7)c6c5)cc4)cc32)cc1]

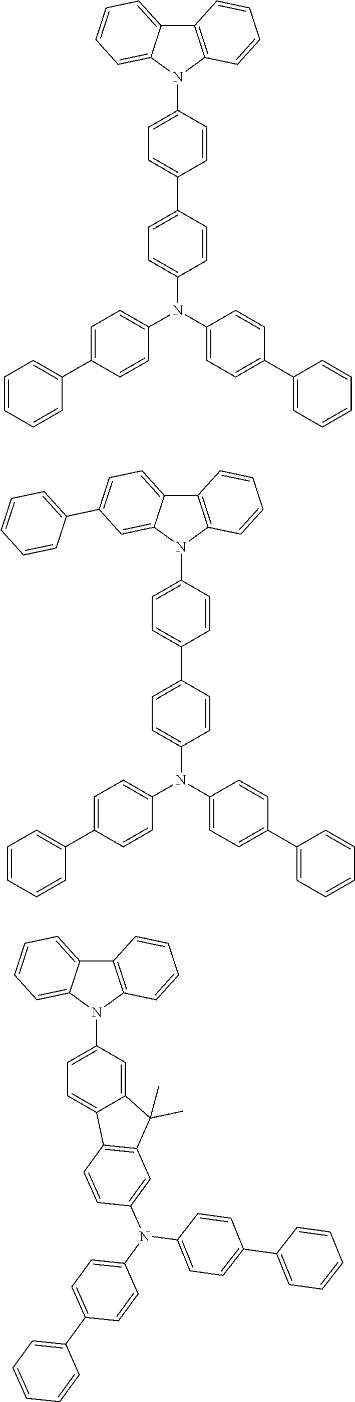

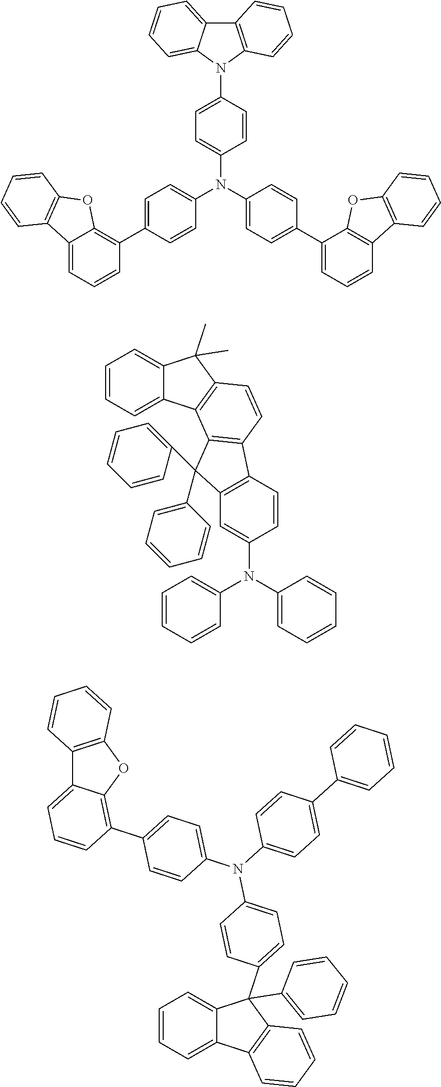 [SMILES: c1ccc2c(c1)oc1c(-c3ccc(N(c4ccc(-c5cccc6c5oc5ccccc56)cc4)c4ccc(-n5c6ccccc6c6ccccc65)cc4)cc3)cccc12 ; CC1(C)c2ccccc2-c2c1ccc1c2C(c2ccccc2)(c2ccccc2)c2cc(N(c3ccccc3)c3ccccc3)ccc2-1 ; c1ccc(-c2ccc(N(c3ccc(-c4cccc5c4oc4ccccc45)cc3)c3ccc(C4(c5ccccc5)c5ccccc5-c5ccccc54)cc3)cc2)cc1]

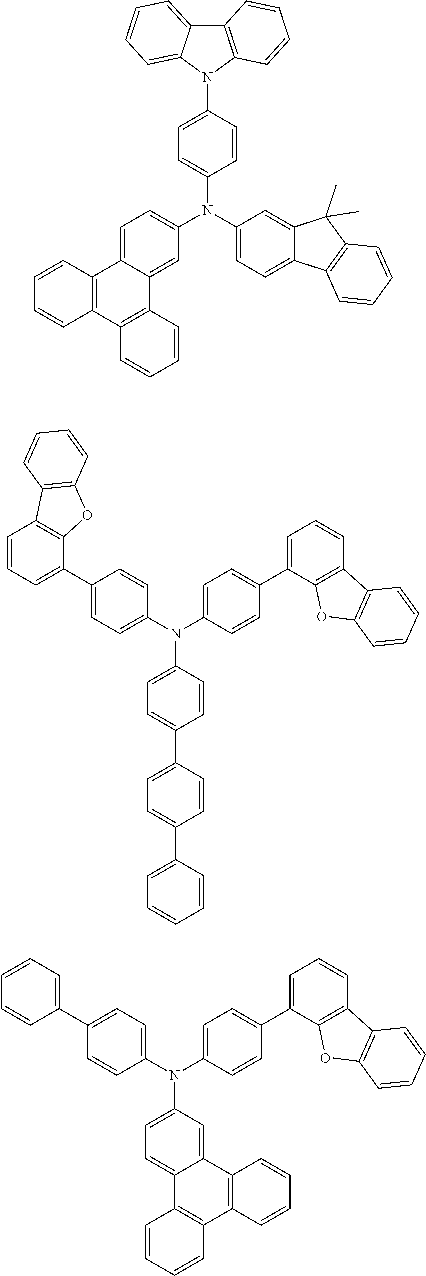 [SMILES: CC1(C)c2ccccc2-c2ccc(N(c3ccc(-n4c5ccccc5c5ccccc54)cc3)c3ccc4c5ccccc5c5ccccc5c4c3)cc21 ; c1ccc(-c2ccc(-c3ccc(N(c4ccc(-c5cccc6c5oc5ccccc56)cc4)c4ccc(-c5cccc6c5oc5ccccc56)cc4)cc3)cc2)cc1 ; c1ccc(-c2ccc(N(c3ccc(-c4cccc5c4oc4ccccc45)cc3)c3ccc4c5ccccc5c5ccccc5c4c3)cc2)cc1]

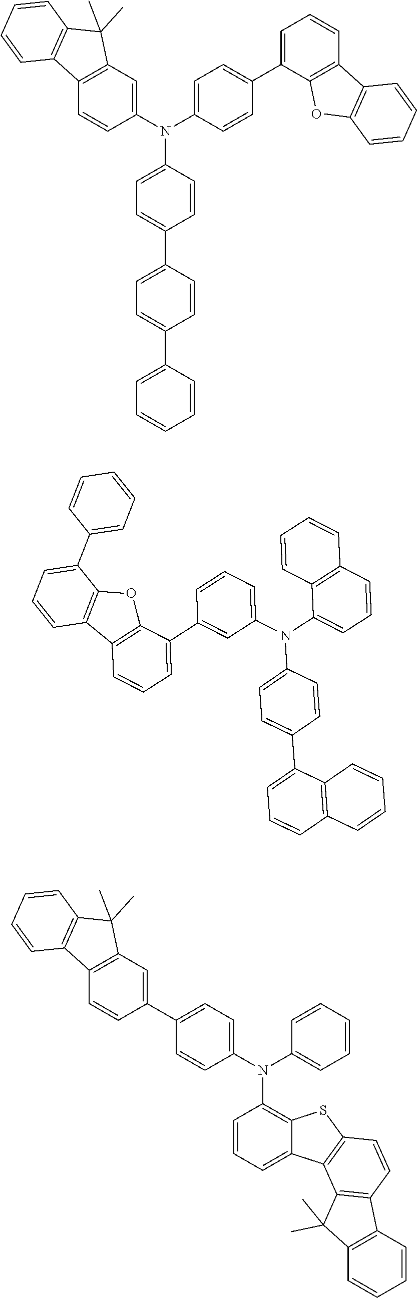 [SMILES: CC1(C)c2ccccc2-c2ccc(N(c3ccc(-c4ccc(-c5ccccc5)cc4)cc3)c3ccc(-c4cccc5c4oc4ccccc45)cc3)cc21 ; c1ccc(-c2cccc3c2oc2c(-c4cccc(N(c5ccc(-c6cccc7ccccc67)cc5)c5cccc6ccccc56)c4)cccc23)cc1 ; CC1(C)c2ccccc2-c2ccc(-c3ccc(N(c4ccccc4)c4cccc5c4sc4ccc6c(c45)C(C)(C)c4ccccc4-6)cc3)cc21]

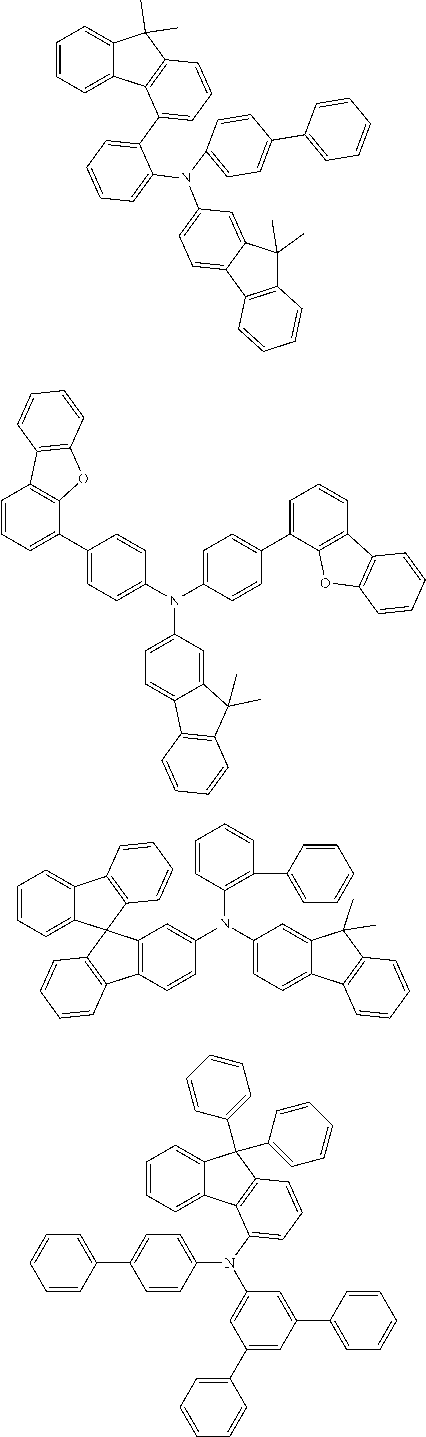

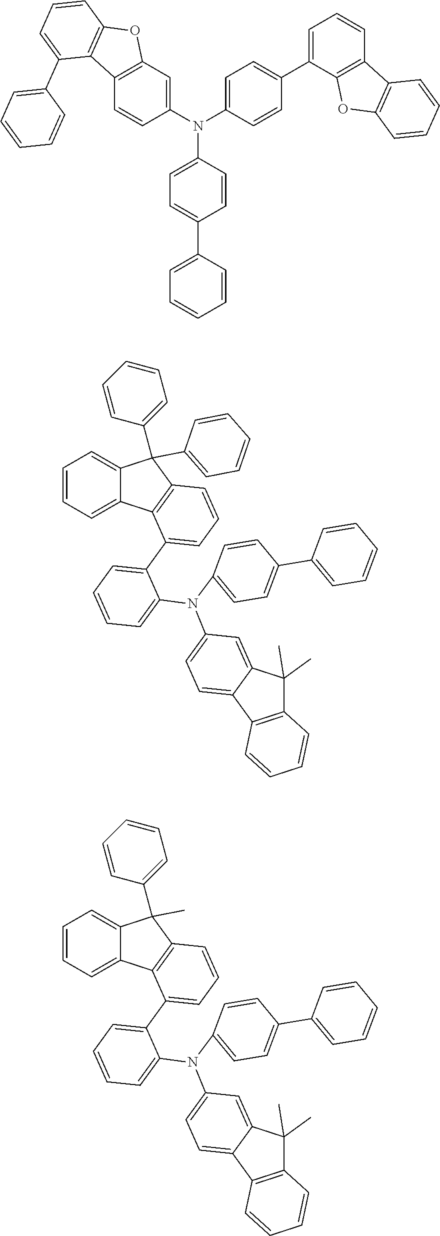 [SMILES: c1ccc(-c2ccc(N(c3ccc(-c4cccc5c4oc4ccccc45)cc3)c3ccc4c(c3)oc3cccc(-c5ccccc5)c34)cc2)cc1 ; CC1(C)c2ccccc2-c2ccc(N(c3ccc(-c4ccccc4)cc3)c3ccccc3-c3cccc4c3-c3ccccc3C4(c3ccccc3)c3ccccc3)cc21 ; CC1(C)c2ccccc2-c2ccc(N(c3ccc(-c4ccccc4)cc3)c3ccccc3-c3cccc4c3-c3ccccc3C4(C)c3ccccc3)cc21]

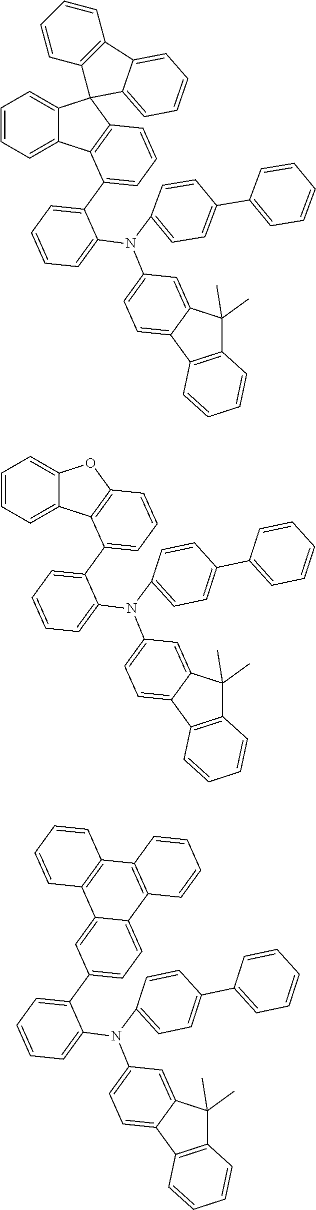 [SMILES: CC1(C)c2ccccc2-c2ccc(N(c3ccc(-c4ccccc4)cc3)c3ccccc3-c3cccc4c3-c3ccccc3C43c4ccccc4-c4ccccc43)cc21 ; CC1(C)c2ccccc2-c2ccc(N(c3ccc(-c4ccccc4)cc3)c3ccccc3-c3cccc4oc5ccccc5c34)cc21 ; CC1(C)c2ccccc2-c2ccc(N(c3ccc(-c4ccccc4)cc3)c3ccccc3-c3ccc4c5ccccc5c5ccccc5c4c3)cc21]

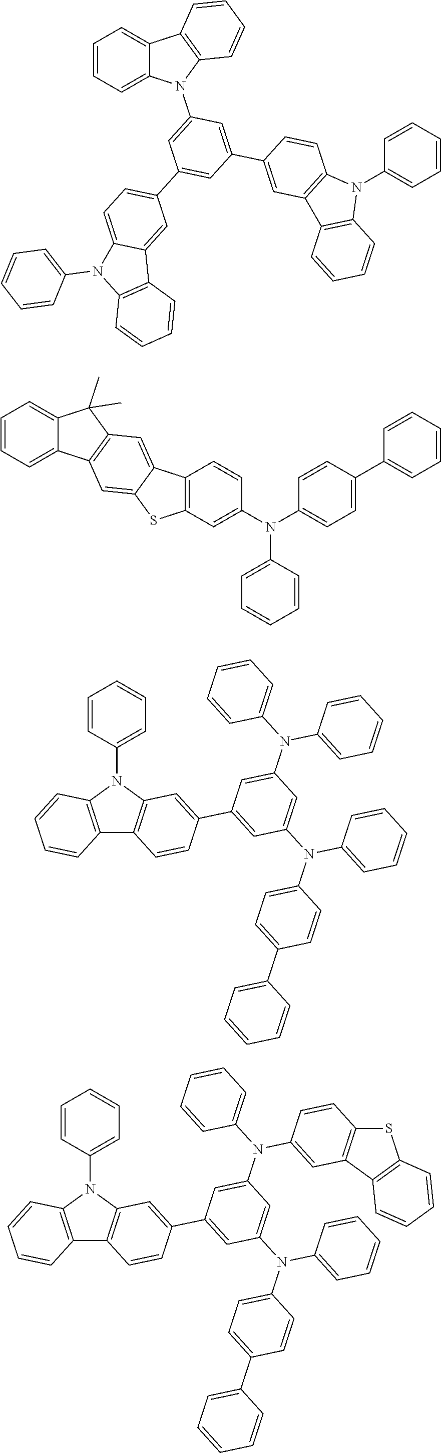

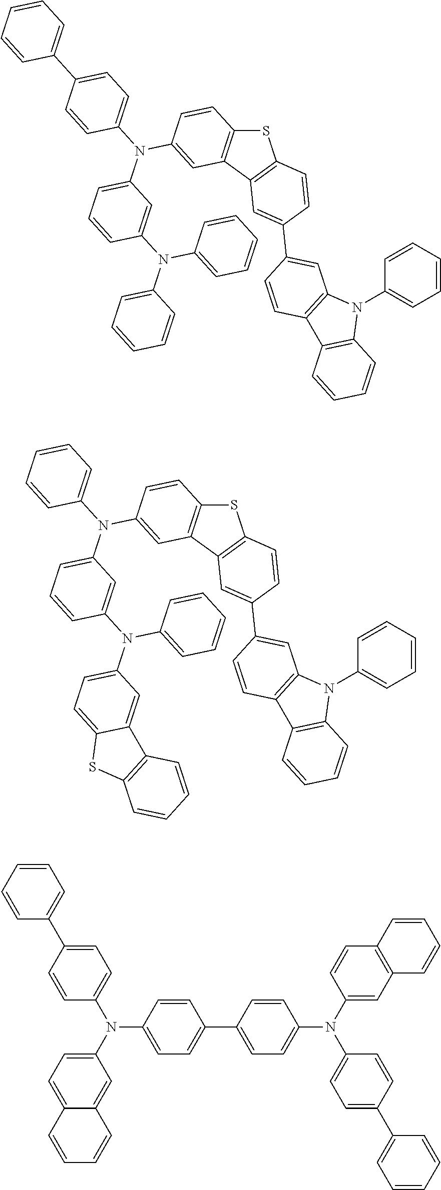 [SMILES: c1ccc(-c2ccc(N(c3cccc(N(c4ccccc4)c4ccccc4)c3)c3ccc4sc5ccc(-c6ccc7c8ccccc8n(-c8ccccc8)c7c6)cc5c4c3)cc2)cc1 ; c1ccc(N(c2cccc(N(c3ccccc3)c3ccc4sc5ccc(-c6ccc7c8ccccc8n(-c8ccccc8)c7c6)cc5c4c3)c2)c2ccc3sc4ccccc4c3c2)cc1 ; c1ccc(-c2ccc(N(c3ccc(-c4ccc(N(c5ccc(-c6ccccc6)cc5)c5ccc6ccccc6c5)cc4)cc3)c3ccc4ccccc4c3)cc2)cc1]

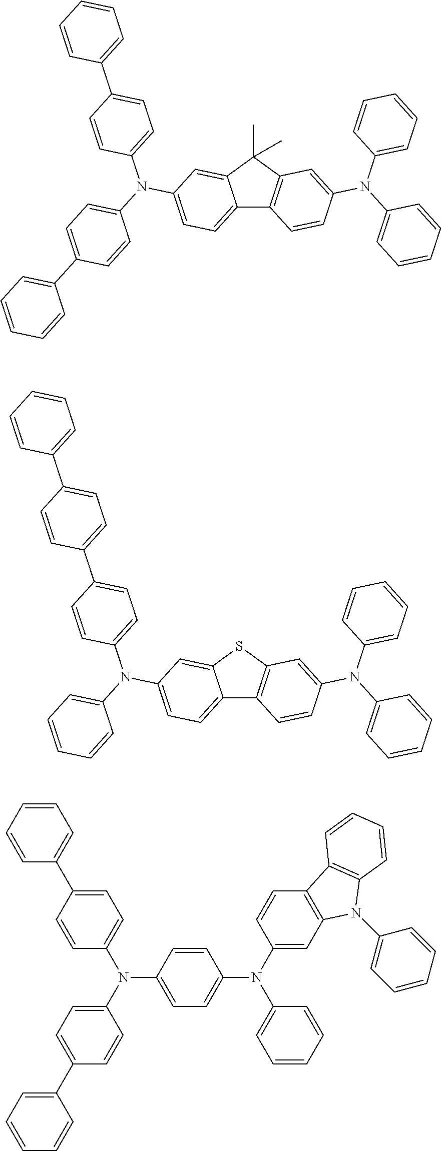 [SMILES: CC1(C)c2cc(N(c3ccccc3)c3ccccc3)ccc2-c2ccc(N(c3ccc(-c4ccccc4)cc3)c3ccc(-c4ccccc4)cc3)cc21 ; c1ccc(-c2ccc(-c3ccc(N(c4ccccc4)c4ccc5c(c4)sc4cc(N(c6ccccc6)c6ccccc6)ccc45)cc3)cc2)cc1 ; c1ccc(-c2ccc(N(c3ccc(-c4ccccc4)cc3)c3ccc(N(c4ccccc4)c4ccc5c6ccccc6n(-c6ccccc6)c5c4)cc3)cc2)cc1]

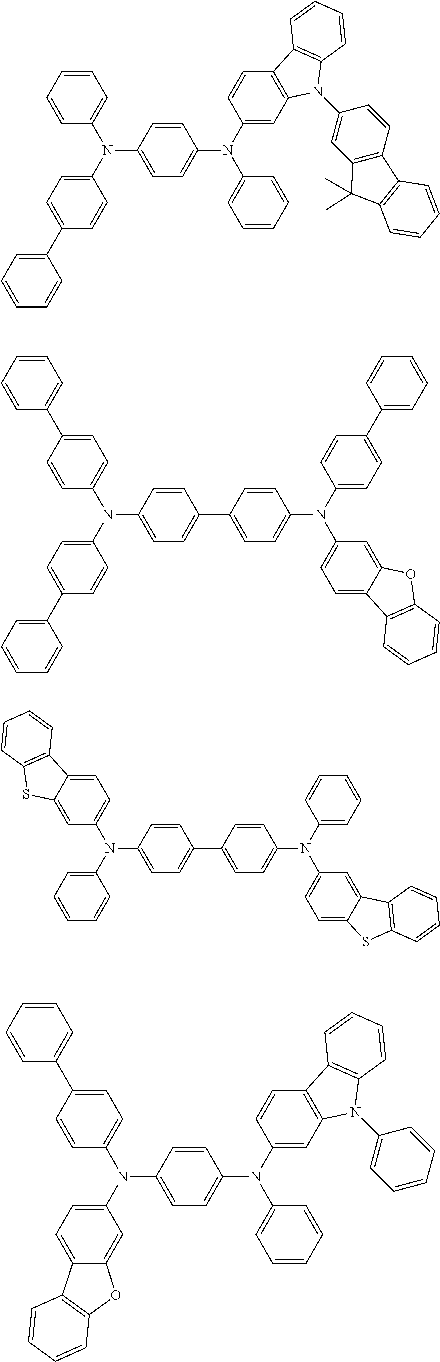

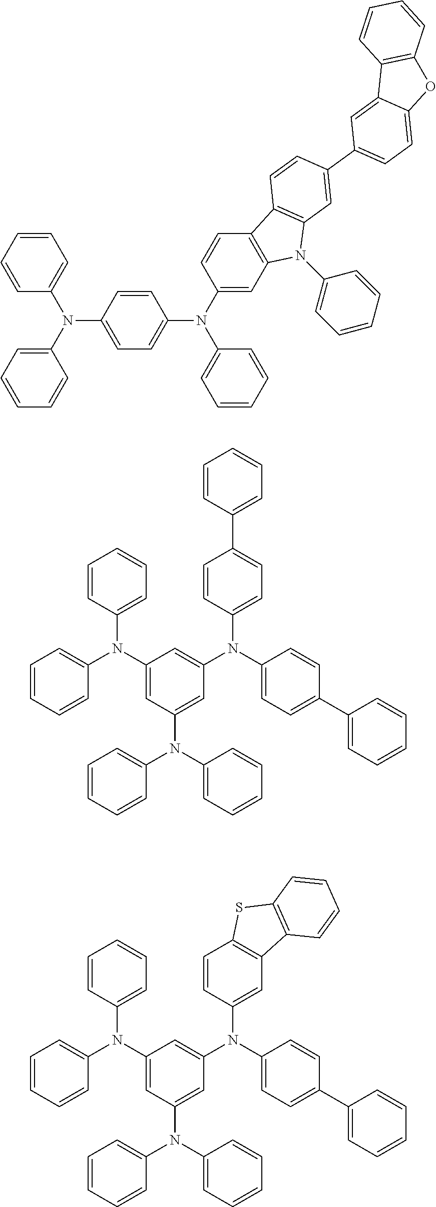

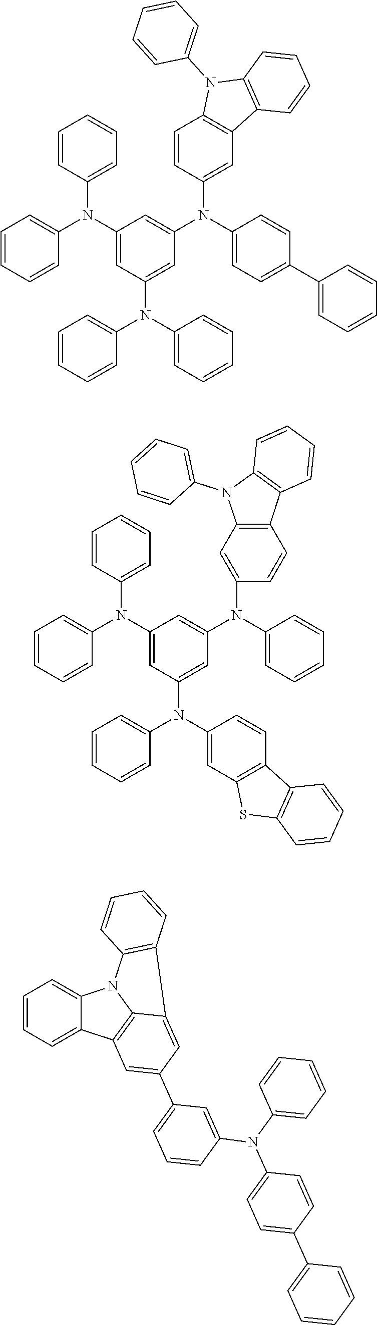 [SMILES: c1ccc(-c2ccc(N(c3cc(N(c4ccccc4)c4ccccc4)cc(N(c4ccccc4)c4ccccc4)c3)c3ccc4c(c3)c3ccccc3n4-c3ccccc3)cc2)cc1 ; c1ccc(N(c2ccccc2)c2cc(N(c3ccccc3)c3ccc4c(c3)sc3ccccc34)cc(N(c3ccccc3)c3ccc4c5ccccc5n(-c5ccccc5)c4c3)c2)cc1 ; c1ccc(-c2ccc(N(c3ccccc3)c3cccc(-c4cc5c6ccccc6n6c7ccccc7c(c4)c56)c3)cc2)cc1]

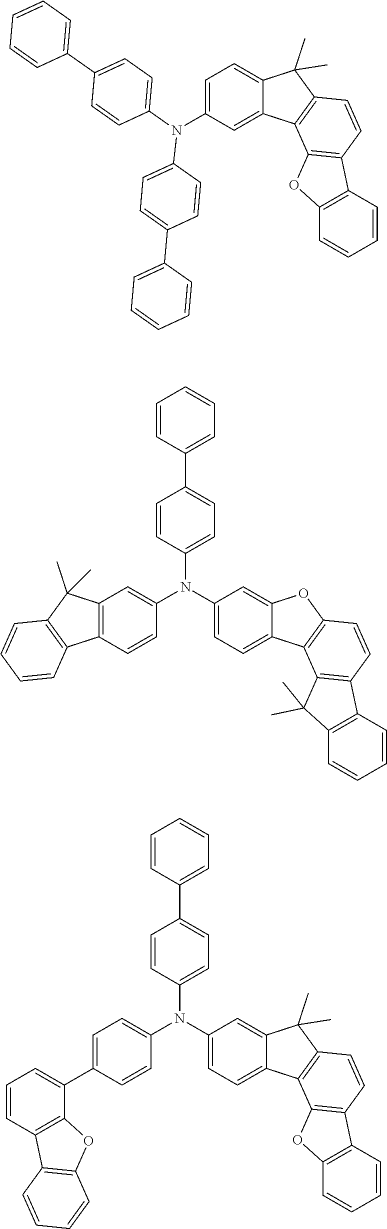 [SMILES: CC1(C)c2ccc(N(c3ccc(-c4ccccc4)cc3)c3ccc(-c4ccccc4)cc3)cc2-c2c1ccc1c2oc2ccccc21 ; CC1(C)c2ccccc2-c2ccc(N(c3ccc(-c4ccccc4)cc3)c3ccc4c(c3)oc3ccc5c(c34)C(C)(C)c3ccccc3-5)cc21 ; CC1(C)c2cc(N(c3ccc(-c4ccccc4)cc3)c3ccc(-c4cccc5c4oc4ccccc45)cc3)ccc2-c2c1ccc1c2oc2ccccc21]

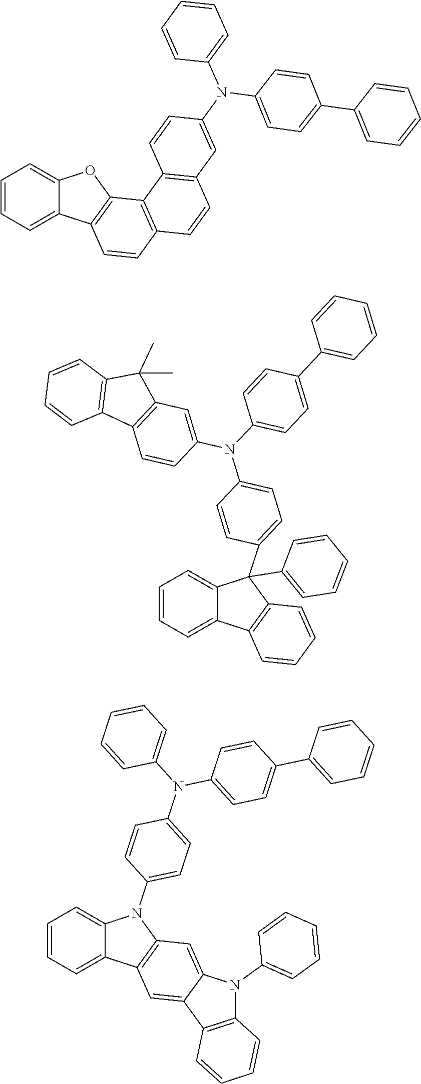 [SMILES: c1ccc(-c2ccc(N(c3ccccc3)c3ccc4c(ccc5ccc6c7ccccc7oc6c54)c3)cc2)cc1 ; CC1(C)c2ccccc2-c2ccc(N(c3ccc(-c4ccccc4)cc3)c3ccc(C4(c5ccccc5)c5ccccc5-c5ccccc54)cc3)cc21 ; c1ccc(-c2ccc(N(c3ccccc3)c3ccc(-n4c5ccccc5c5cc6c7ccccc7n(-c7ccccc7)c6cc54)cc3)cc2)cc1]

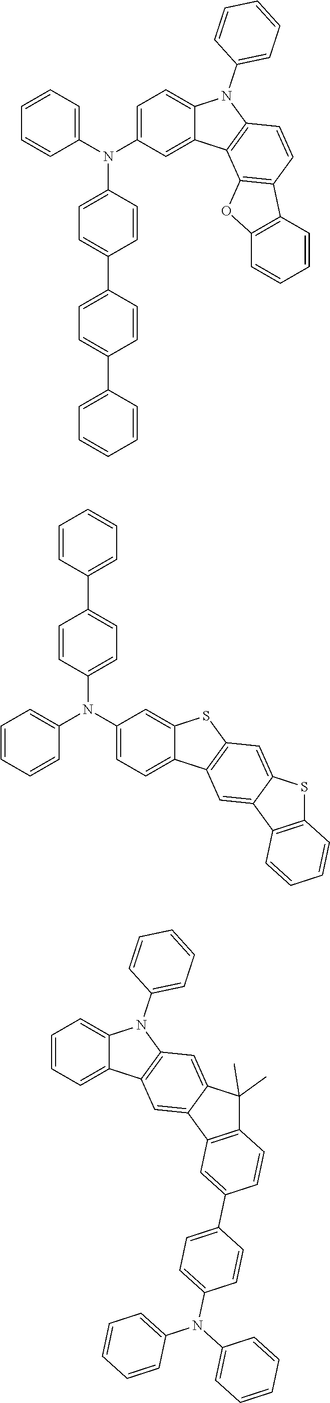 [SMILES: c1ccc(-c2ccc(-c3ccc(N(c4ccccc4)c4ccc5c(c4)c4c6oc7ccccc7c6ccc4n5-c4ccccc4)cc3)cc2)cc1 ; c1ccc(-c2ccc(N(c3ccccc3)c3ccc4c(c3)sc3cc5sc6ccccc6c5cc34)cc2)cc1 ; CC1(C)c2ccc(-c3ccc(N(c4ccccc4)c4ccccc4)cc3)cc2-c2cc3c4ccccc4n(-c4ccccc4)c3cc21]

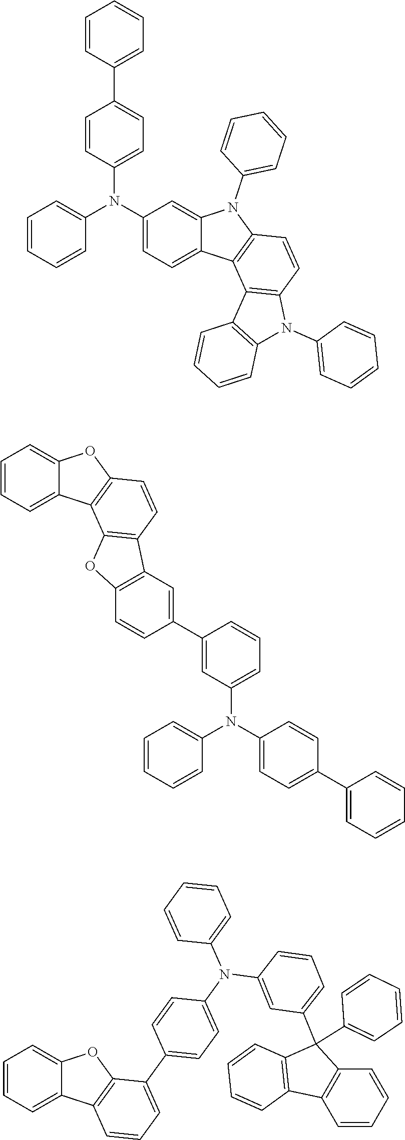 [SMILES: c1ccc(-c2ccc(N(c3ccccc3)c3ccc4c5c6c7ccccc7n(-c7ccccc7)c6ccc5n(-c5ccccc5)c4c3)cc2)cc1 ; c1ccc(-c2ccc(N(c3ccccc3)c3cccc(-c4ccc5oc6c(ccc7oc8ccccc8c76)c5c4)c3)cc2)cc1 ; c1ccc(N(c2ccc(-c3cccc4c3oc3ccccc34)cc2)c2cccc(C3(c4ccccc4)c4ccccc4-c4ccccc43)c2)cc1]

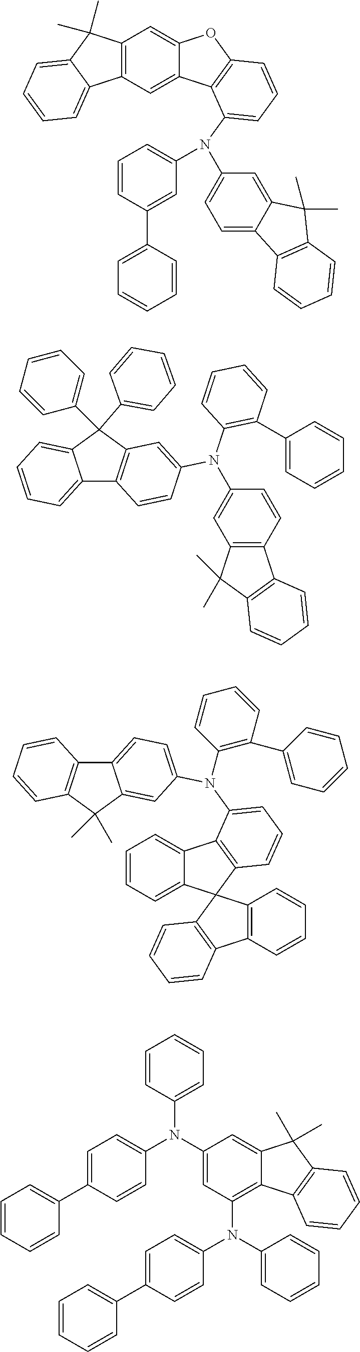

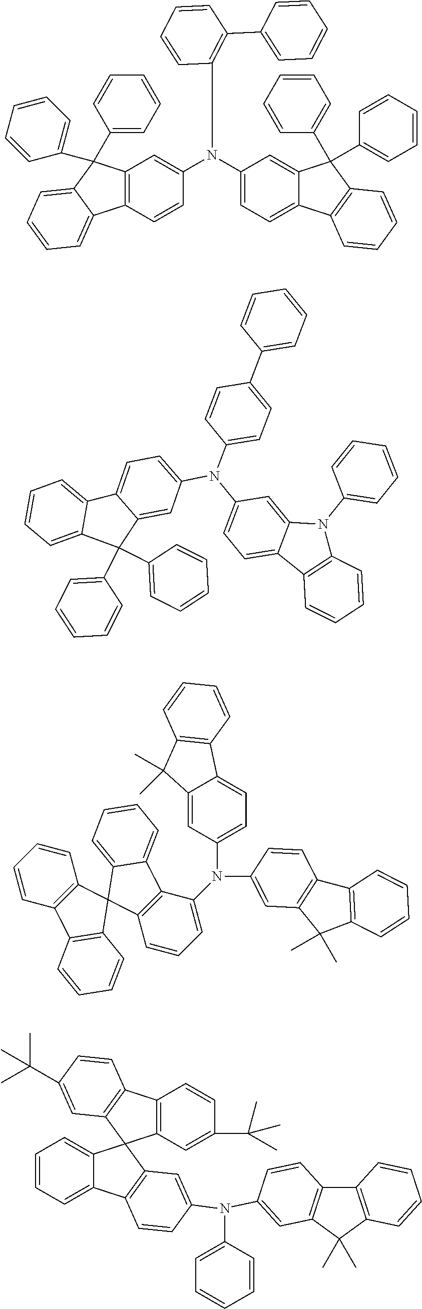 [SMILES: c1ccc(-c2ccccc2N(c2ccc3c(c2)C(c2ccccc2)(c2ccccc2)c2ccccc2-3)c2ccc3c(c2)C(c2ccccc2)(c2ccccc2)c2ccccc2-3)cc1 ; c1ccc(-c2ccc(N(c3ccc4c(c3)C(c3ccccc3)(c3ccccc3)c3ccccc3-4)c3ccc4c5ccccc5n(-c5ccccc5)c4c3)cc2)cc1 ; CC1(C)c2ccccc2-c2ccc(N(c3ccc4c(c3)C(C)(C)c3ccccc3-4)c3cccc4c3-c3ccccc3C43c4ccccc4-c4ccccc43)cc21 ; CC(C)(C)c1ccc2c(c1)C1(c3ccccc3-c3ccc(N(c4ccccc4)c4ccc5c(c4)C(C)(C)c4ccccc4-5)cc31)c1cc(C(C)(C)C)ccc1-2]

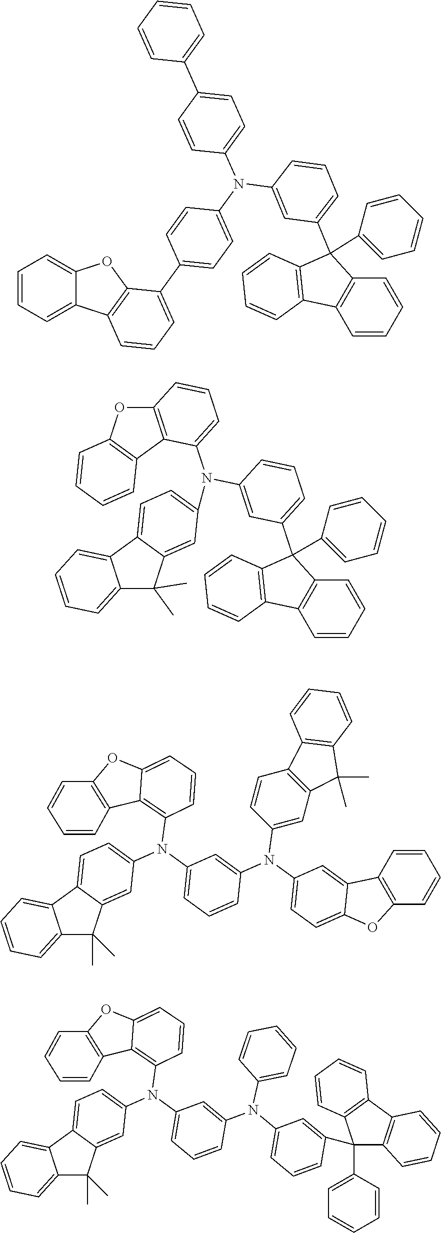

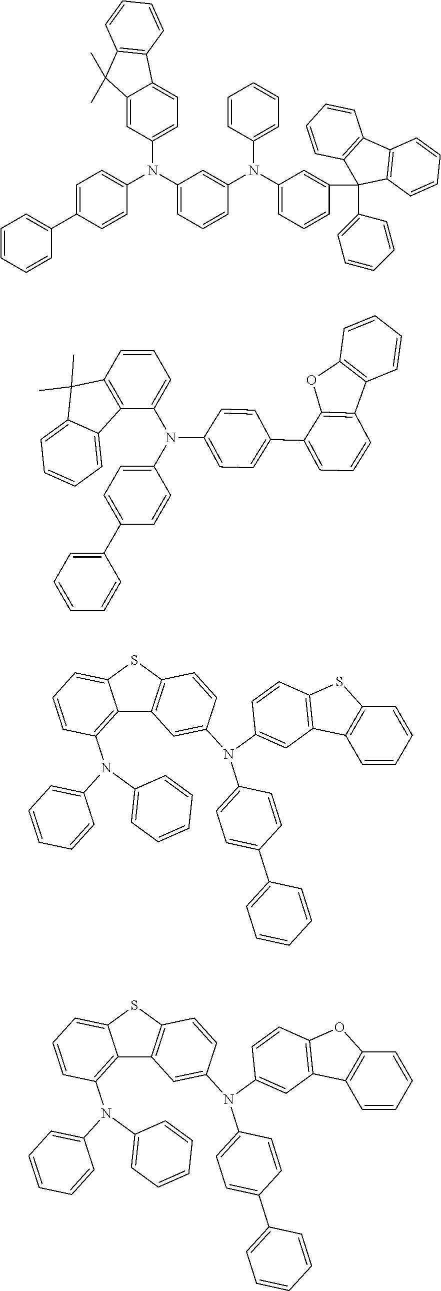

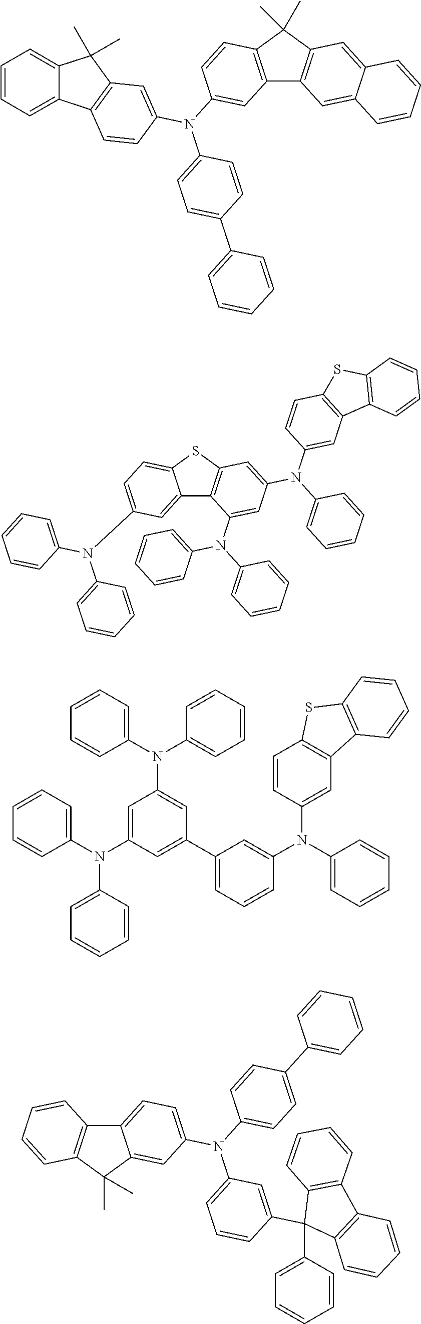 [SMILES: CC1(C)c2ccccc2-c2ccc(N(c3ccc(-c4ccccc4)cc3)c3ccc4c(c3)-c3cc5ccccc5cc3C4(C)C)cc21 ; c1ccc(N(c2cc(N(c3ccccc3)c3ccccc3)c3c(c2)sc2ccc(N(c4ccccc4)c4ccccc4)cc23)c2ccc3sc4ccccc4c3c2)cc1 ; c1ccc(N(c2ccccc2)c2cc(-c3cccc(N(c4ccccc4)c4ccc5sc6ccccc6c5c4)c3)cc(N(c3ccccc3)c3ccccc3)c2)cc1 ; CC1(C)c2ccccc2-c2ccc(N(c3ccc(-c4ccccc4)cc3)c3cccc(C4(c5ccccc5)c5ccccc5-c5ccccc54)c3)cc21]

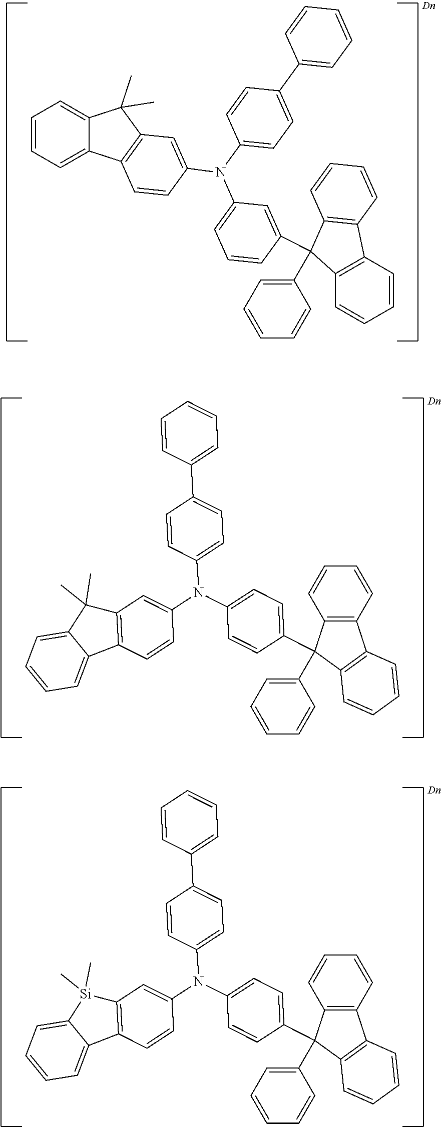

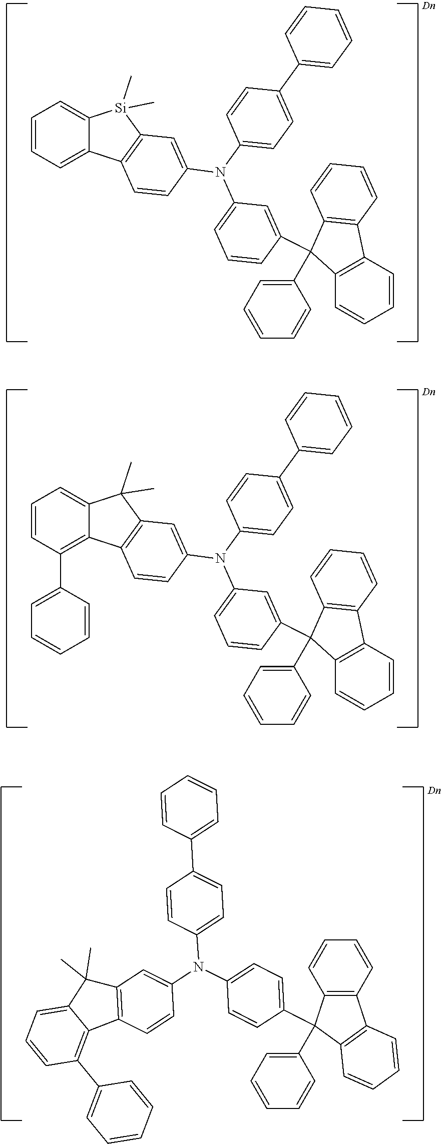

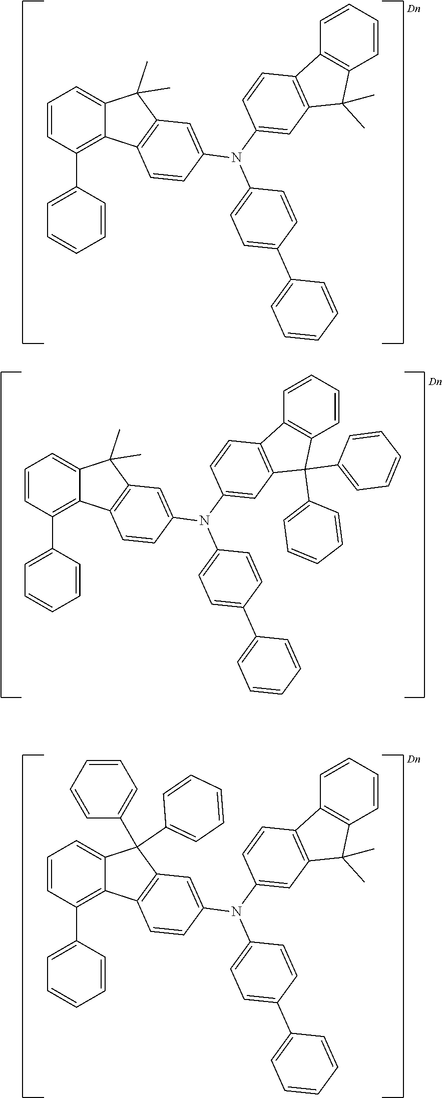

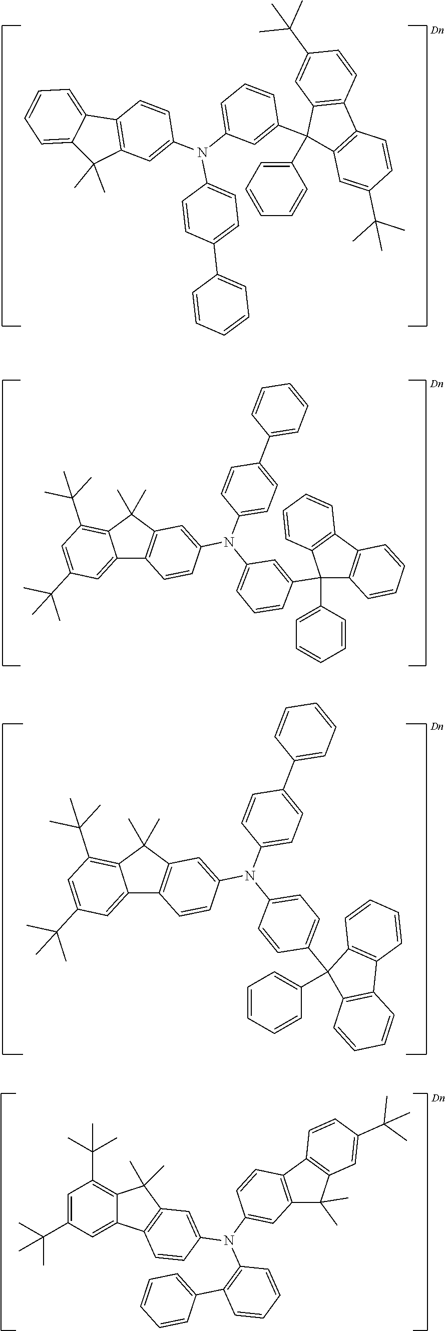

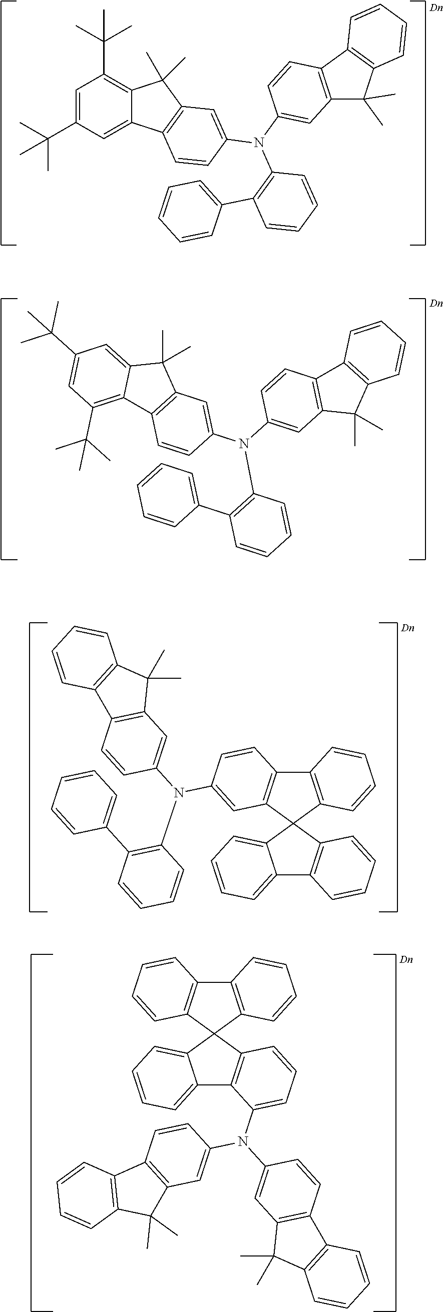

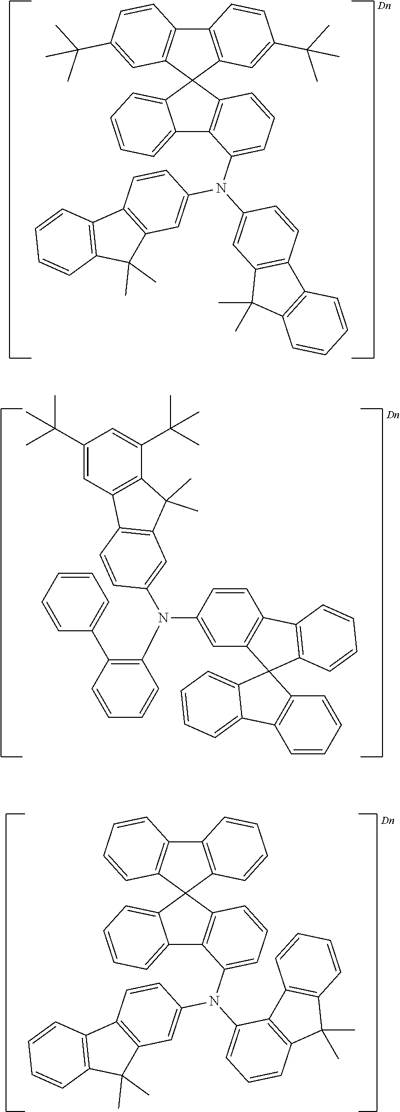

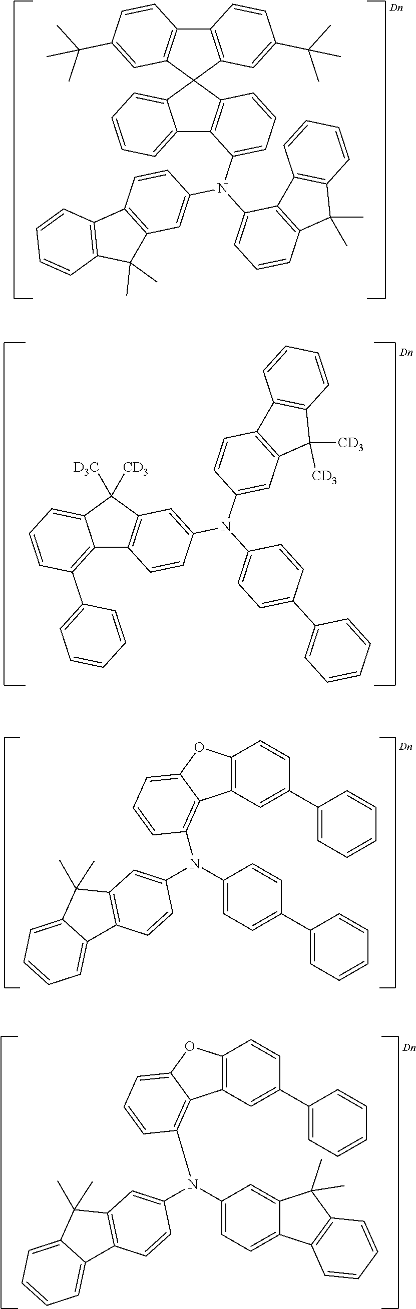

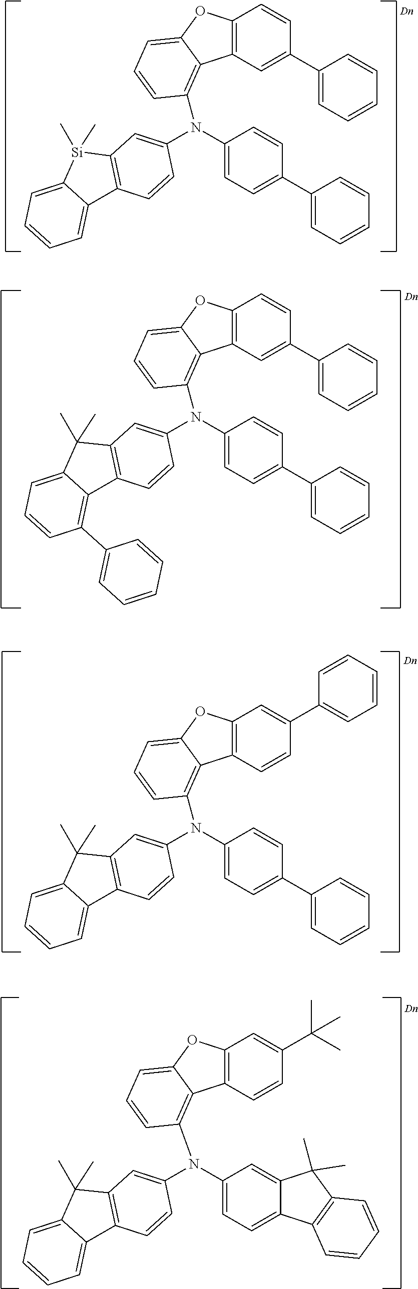

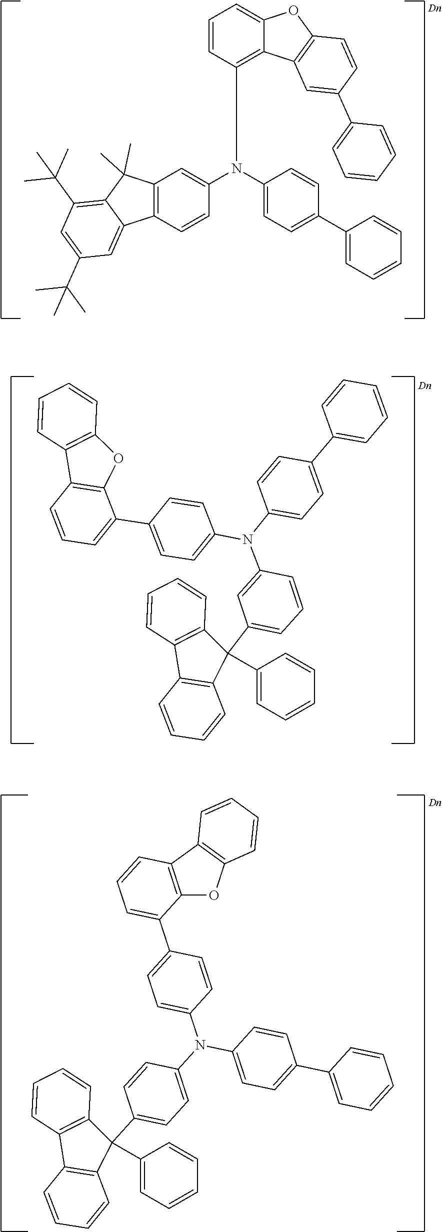

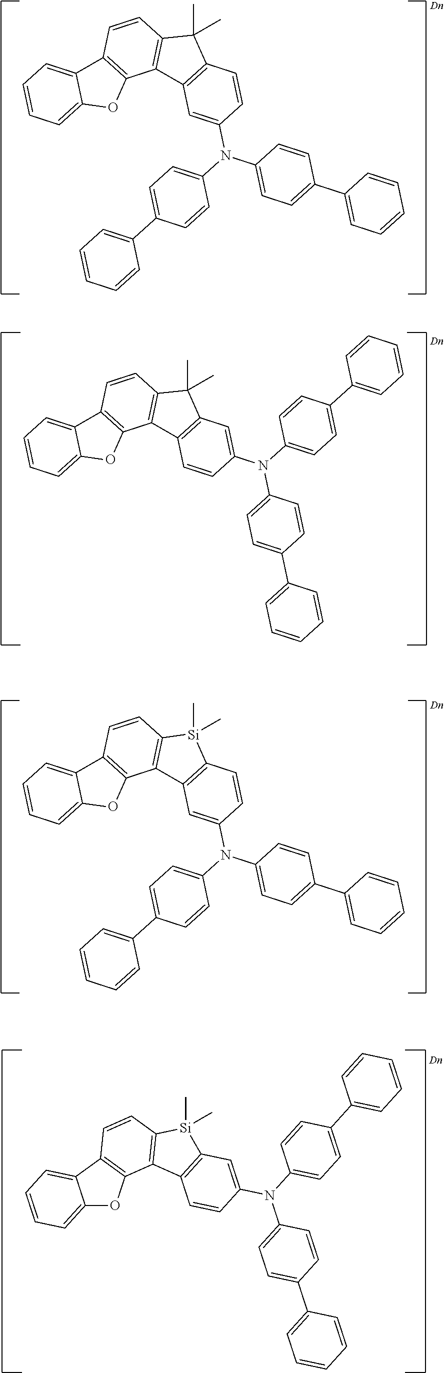

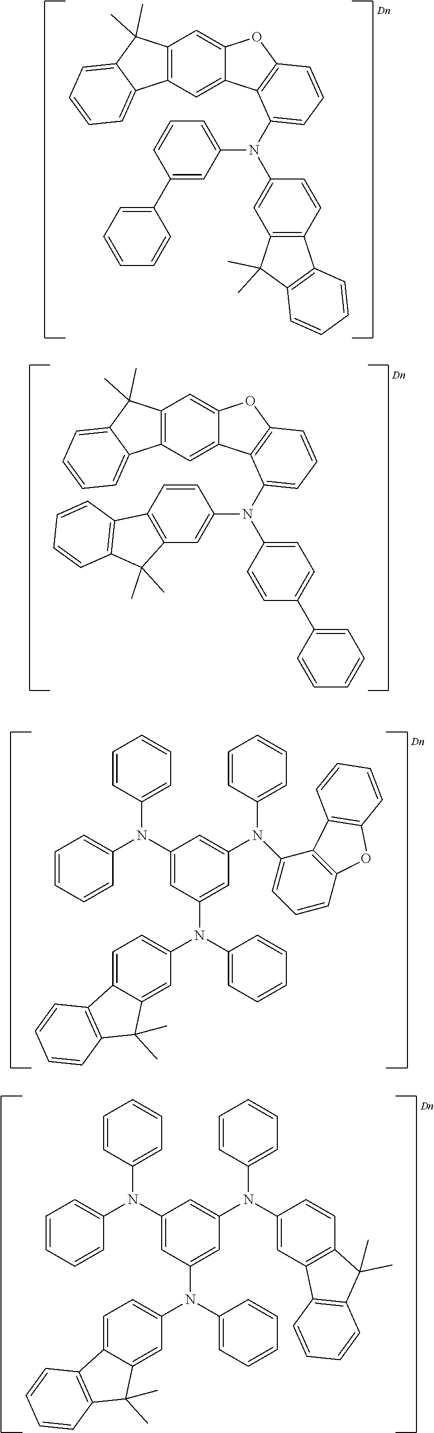

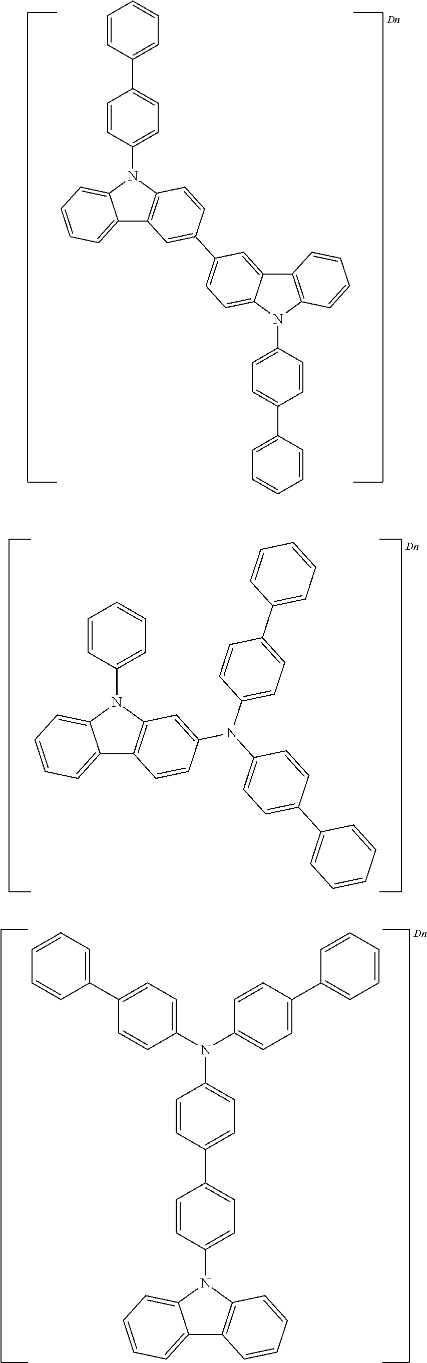

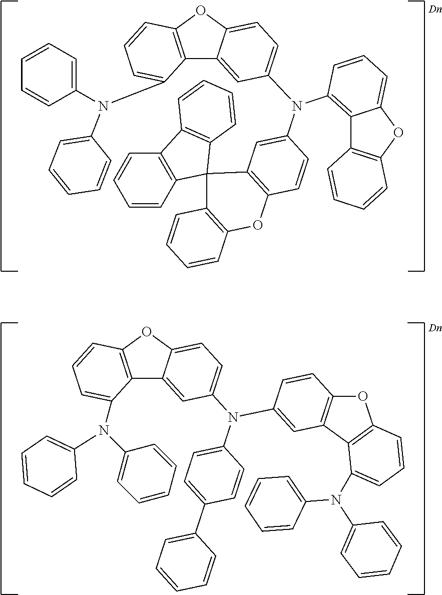

(Dn refers to the number of deuterium substitutions and indicates a structure substituted with one or more deuteriums)

In the hole transport region, in addition to the compounds described above, known compounds disclosed in U.S. Pat. No. 5,061,569A, JP1993-009471A, WO1995-009147A1, JP1995-126615A, JP1998-095973A, etc. and compounds having a similar structure may also be used.

Also, the charge transport region may be, for example, the electron transport region 150.

The electron transport region 150 may further increase electron injection and/or electron mobility and block holes between the cathode 110 and the light emitting layer 130.

Specifically, the electron transport region 150 may include an electron transport layer between the cathode 110 and the light emitting layer 130, and an electron transport auxiliary layer between the light emitting layer 130 and the electron transport layer, and at least one of the compounds of Group C may be included in at least one of the electron transport layer and the electron transport auxiliary layer.

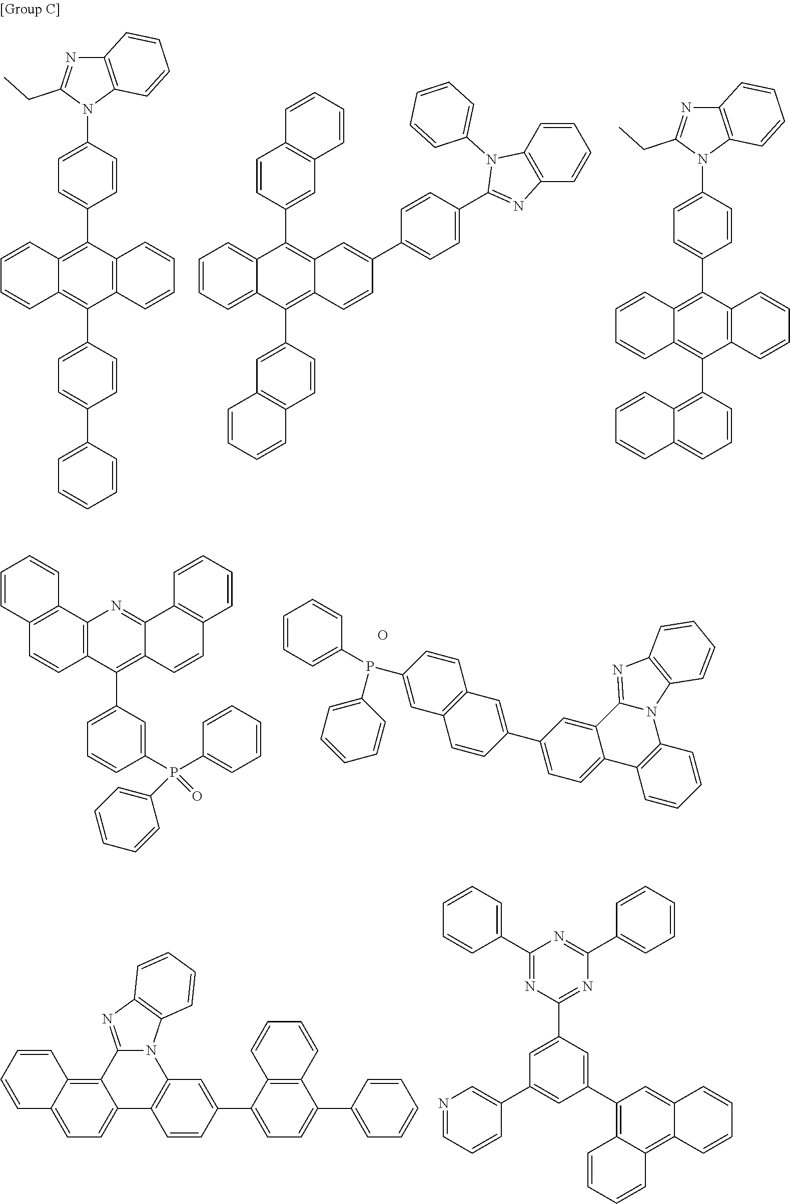

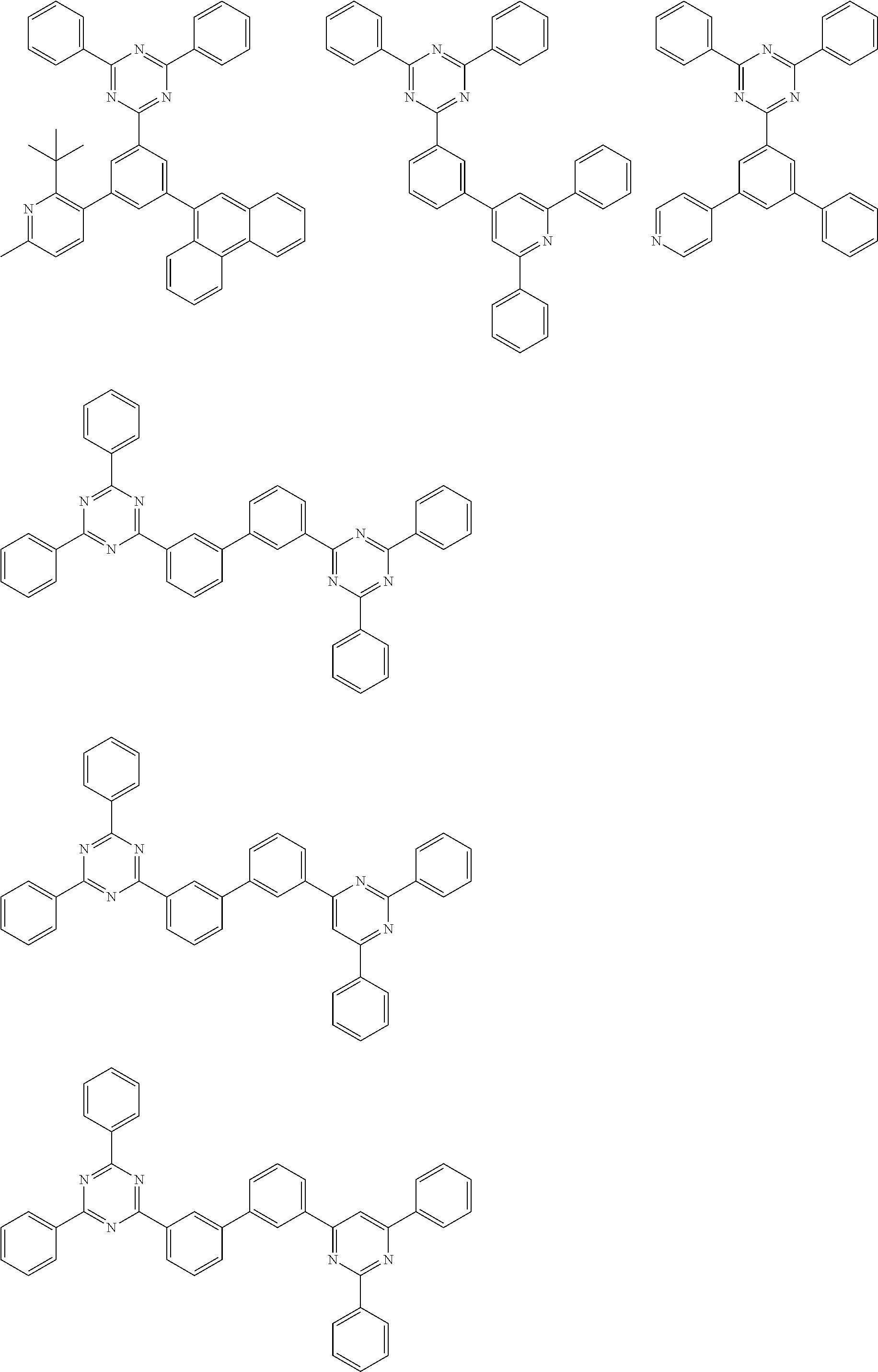

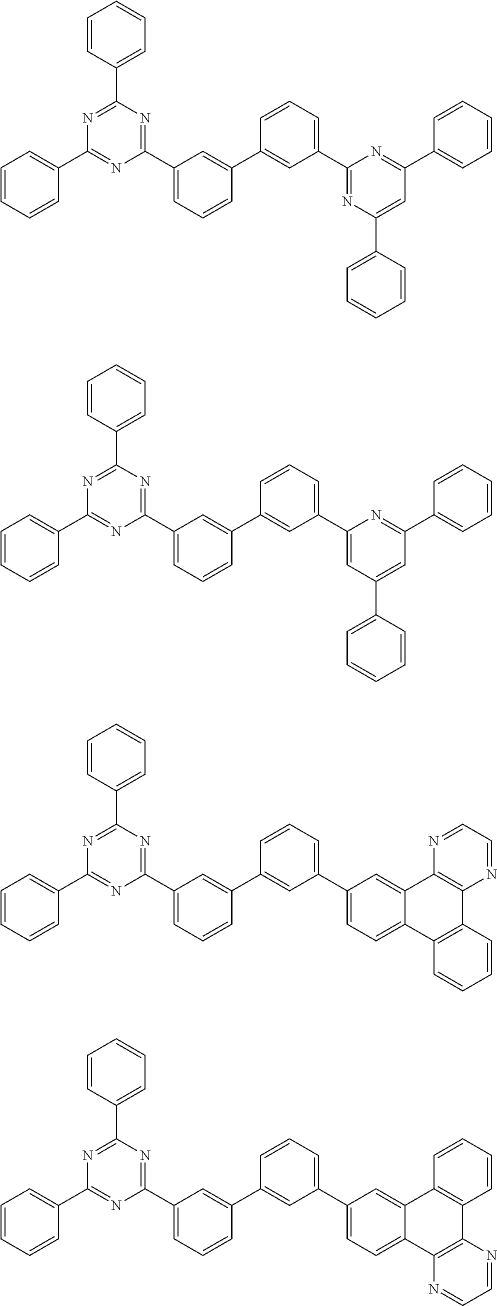 [SMILES: c1ccc(-c2cc(-c3ccccc3)nc(-c3cccc(-c4cccc(-c5nc(-c6ccccc6)nc(-c6ccccc6)n5)c4)c3)n2)cc1 ; c1ccc(-c2cc(-c3ccccc3)nc(-c3cccc(-c4cccc(-c5nc(-c6ccccc6)nc(-c6ccccc6)n5)c4)c3)c2)cc1 ; c1ccc(-c2nc(-c3ccccc3)nc(-c3cccc(-c4cccc(-c5ccc6c7ccccc7c7nccnc7c6c5)c4)c3)n2)cc1 ; c1ccc(-c2nc(-c3ccccc3)nc(-c3cccc(-c4cccc(-c5ccc6c(c5)c5ccccc5c5nccnc65)c4)c3)n2)cc1]

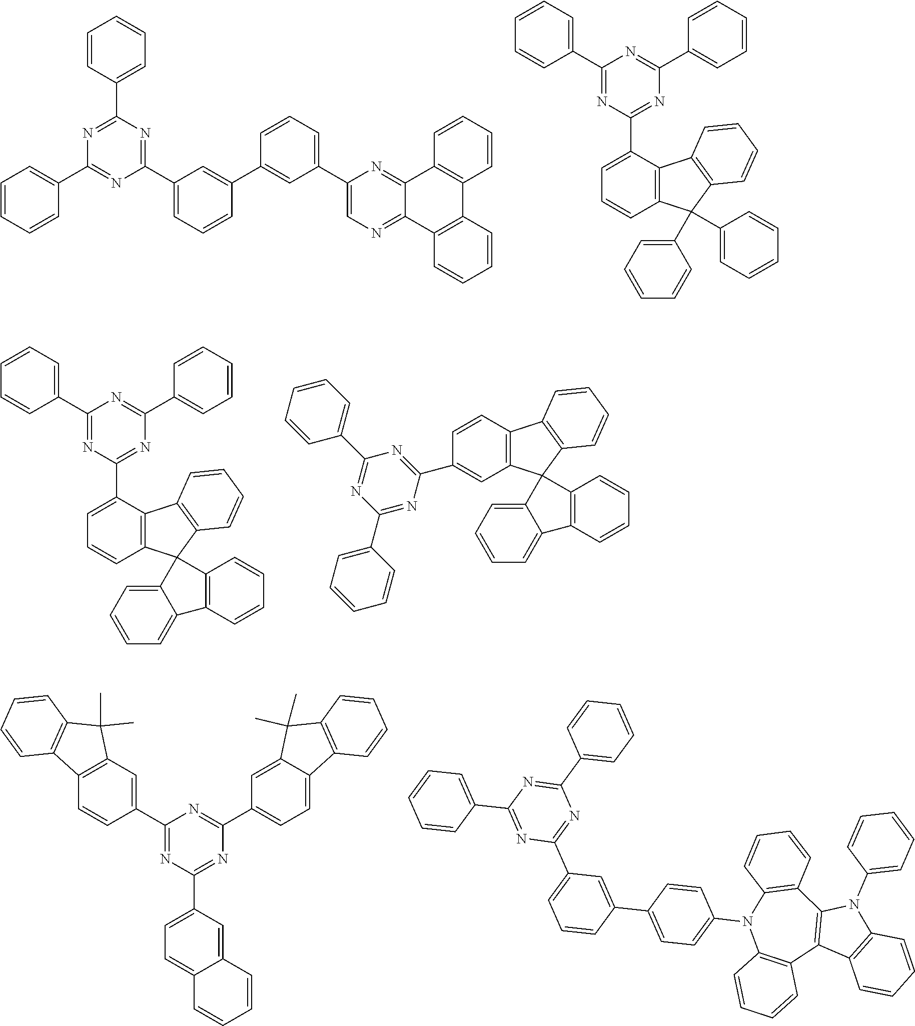 [SMILES: c1ccc(-c2nc(-c3ccccc3)nc(-c3cccc(-c4cccc(-c5cnc6c7ccccc7c7ccccc7c6n5)c4)c3)n2)cc1 ; c1ccc(-c2nc(-c3ccccc3)nc(-c3cccc4c3-c3ccccc3C4(c3ccccc3)c3ccccc3)n2)cc1 ; c1ccc(-c2nc(-c3ccccc3)nc(-c3cccc4c3-c3ccccc3C43c4ccccc4-c4ccccc43)n2)cc1 ; c1ccc(-c2nc(-c3ccccc3)nc(-c3ccc4c(c3)C3(c5ccccc5-c5ccccc53)c3ccccc3-4)n2)cc1 ; CC1(C)c2ccccc2-c2ccc(-c3nc(-c4ccc5c(c4)C(C)(C)c4ccccc4-5)nc(-c4ccc5ccccc5c4)n3)cc21 ; c1ccc(-c2nc(-c3ccccc3)nc(-c3cccc(-c4ccc(N5c6ccccc6-c6c(n(-c7ccccc7)c7ccccc67)-c6ccccc65)cc4)c3)n2)cc1]

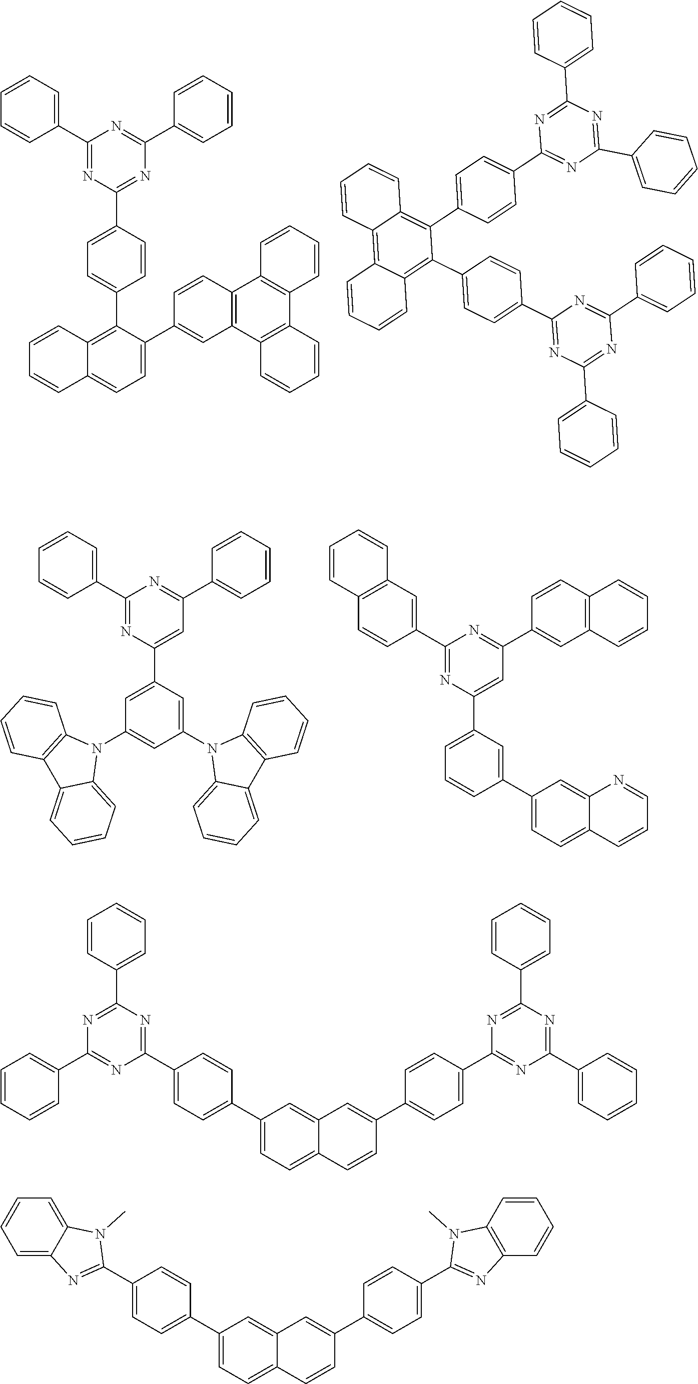

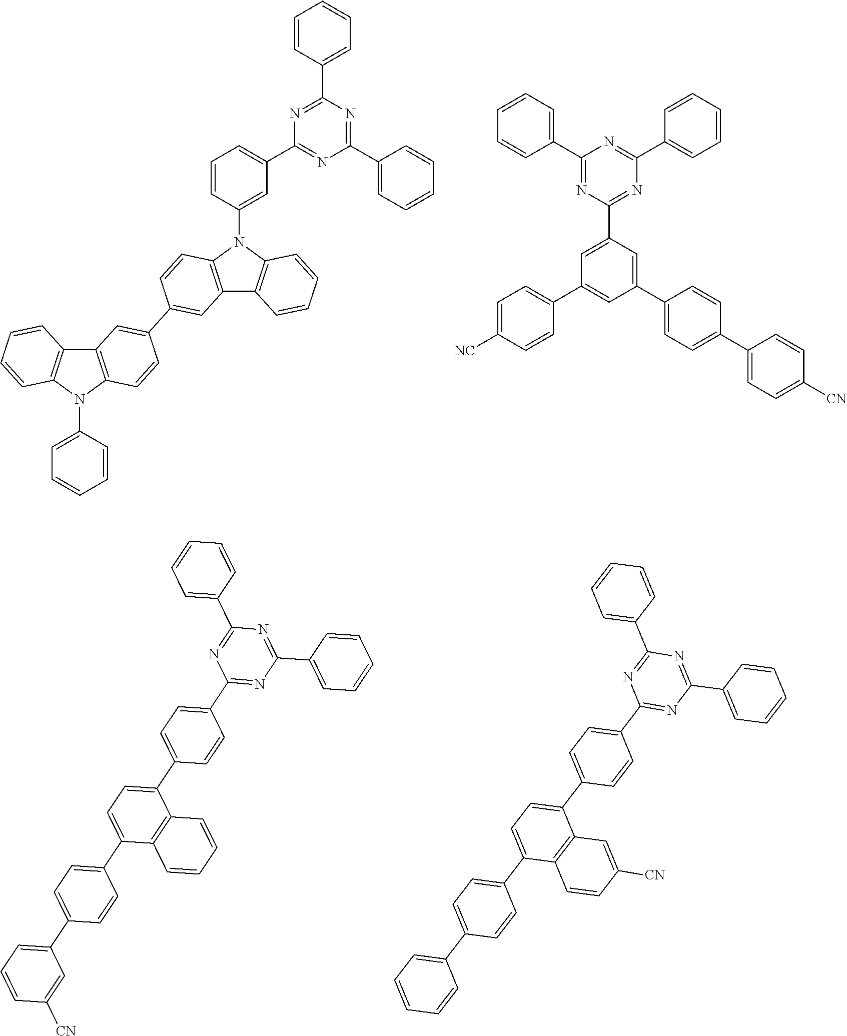 [SMILES: c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1 ; N#Cc1ccc(-c2ccc(-c3cc(-c4ccc(C#N)cc4)cc(-c4nc(-c5ccccc5)nc(-c5ccccc5)n4)c3)cc2)cc1 ; N#Cc1cccc(-c2ccc(-c3ccc(-c4ccc(-c5nc(-c6ccccc6)nc(-c6ccccc6)n5)cc4)c4ccccc34)cc2)c1 ; N#Cc1ccc2c(-c3ccc(-c4ccccc4)cc3)ccc(-c3ccc(-c4nc(-c5ccccc5)nc(-c5ccccc5)n4)cc3)c2c1]

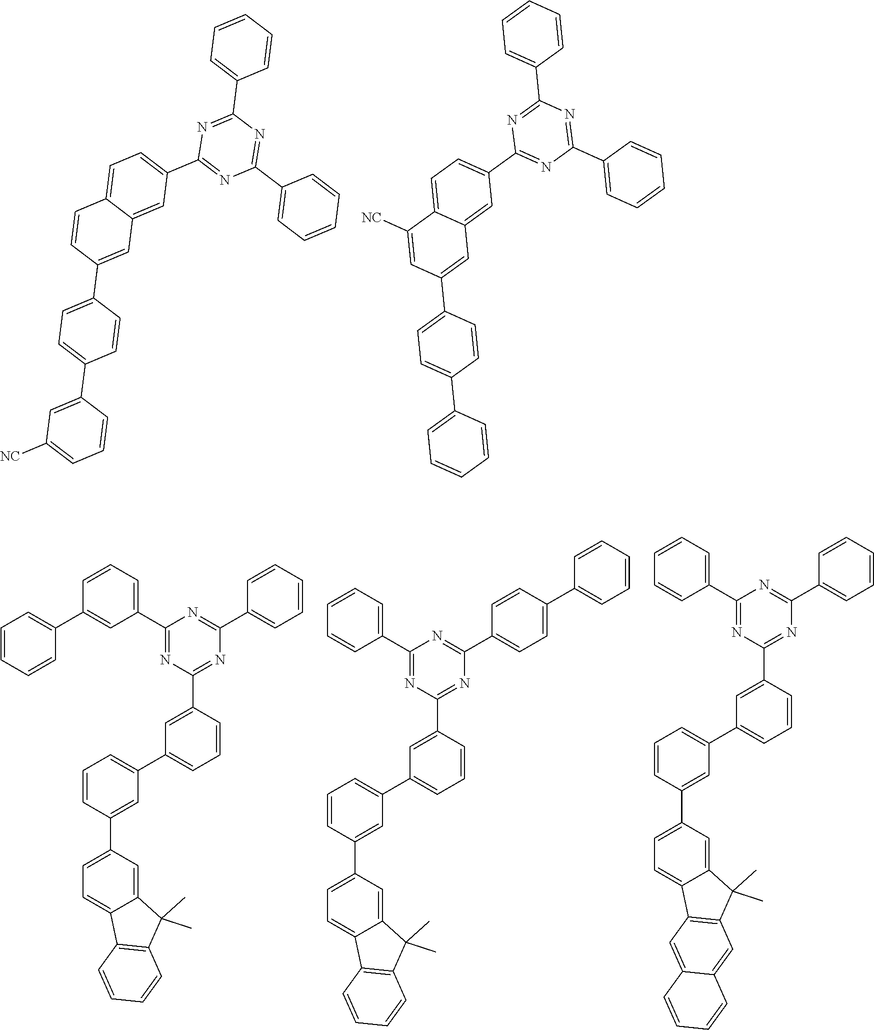

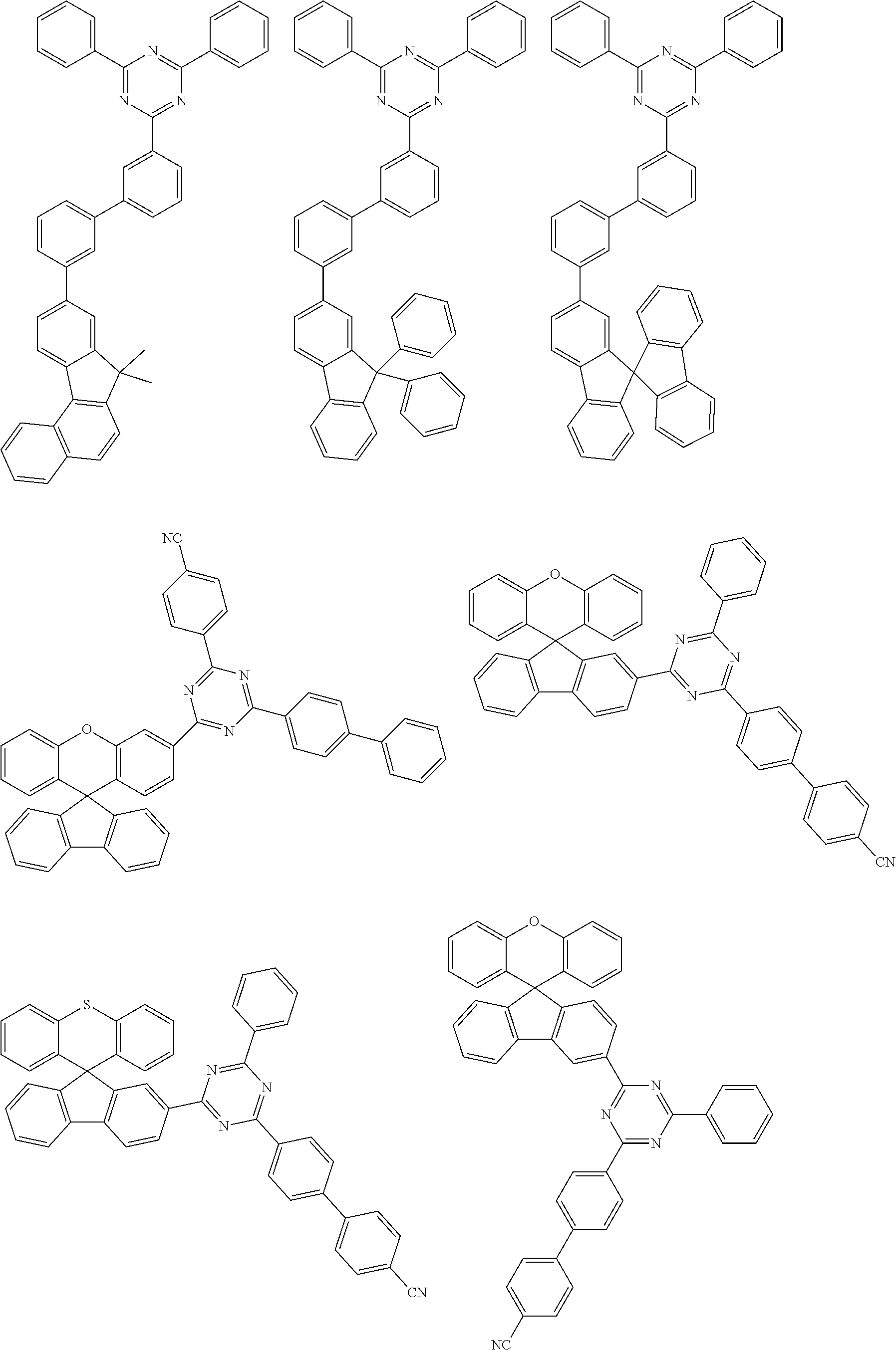

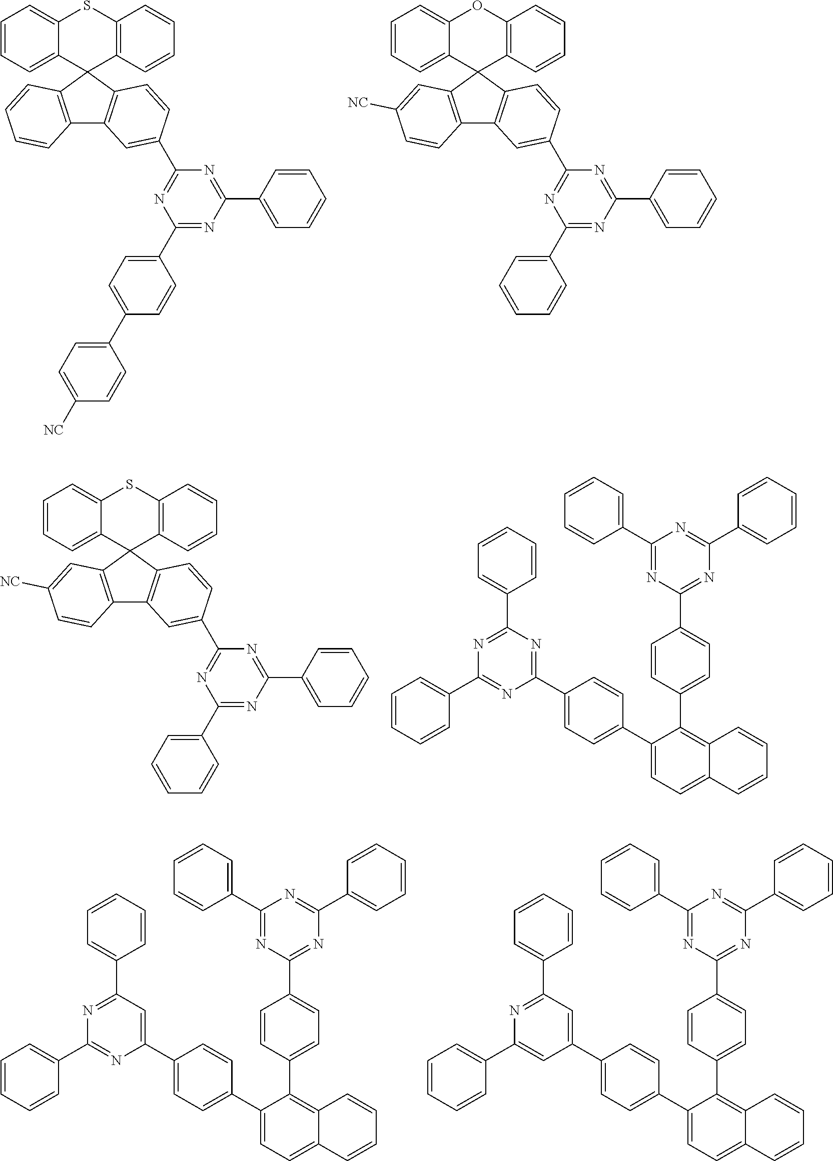

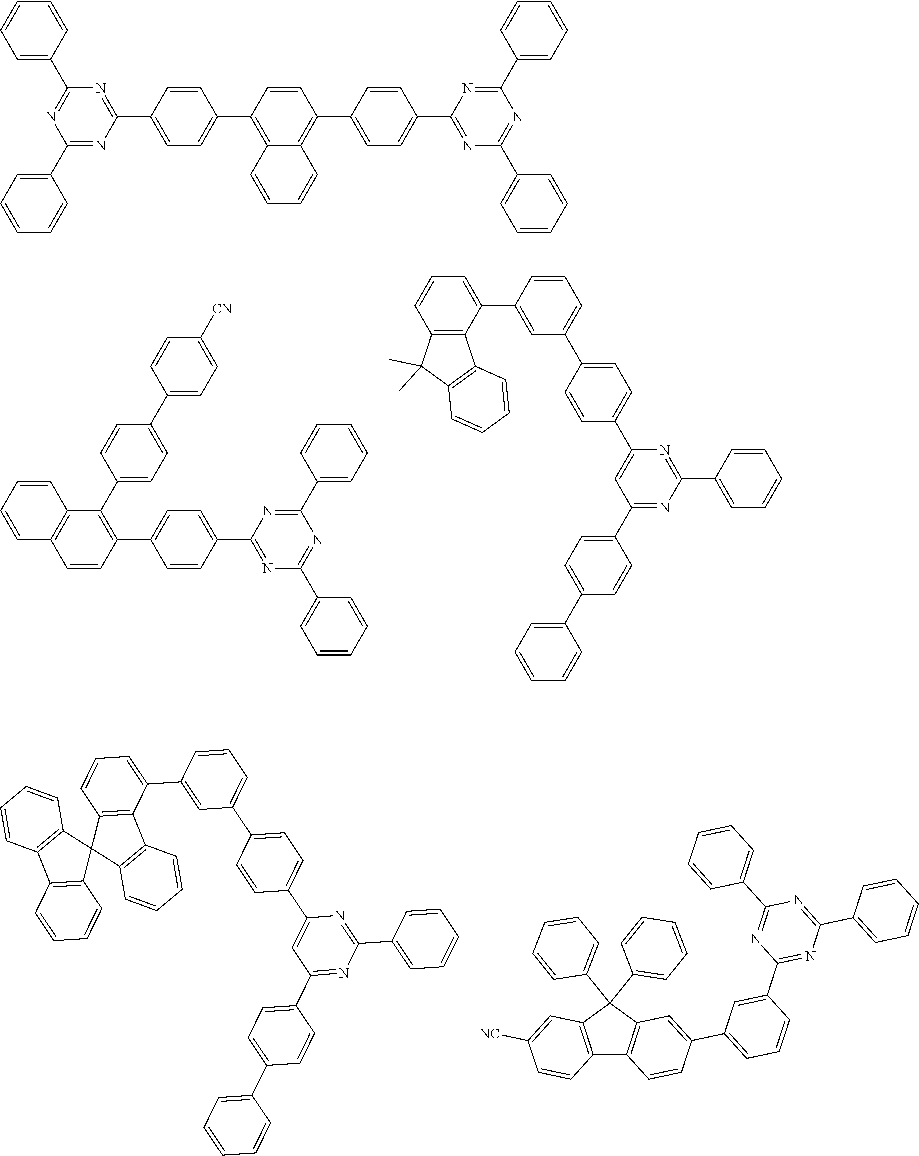 [SMILES: c1ccc(-c2nc(-c3ccccc3)nc(-c3ccc(-c4ccc(-c5ccc(-c6nc(-c7ccccc7)nc(-c7ccccc7)n6)cc5)c5ccccc45)cc3)n2)cc1 ; N#Cc1ccc(-c2ccc(-c3c(-c4ccc(-c5nc(-c6ccccc6)nc(-c6ccccc6)n5)cc4)ccc4ccccc34)cc2)cc1 ; CC1(C)c2ccccc2-c2c(-c3cccc(-c4ccc(-c5cc(-c6ccc(-c7ccccc7)cc6)nc(-c6ccccc6)n5)cc4)c3)cccc21 ; c1ccc(-c2ccc(-c3cc(-c4ccc(-c5cccc(-c6cccc7c6-c6ccccc6C76c7ccccc7-c7ccccc76)c5)cc4)nc(-c4ccccc4)n3)cc2)cc1 ; N#Cc1ccc2c(c1)C(c1ccccc1)(c1ccccc1)c1cc(-c3cccc(-c4nc(-c5ccccc5)nc(-c5ccccc5)n4)c3)ccc1-2]

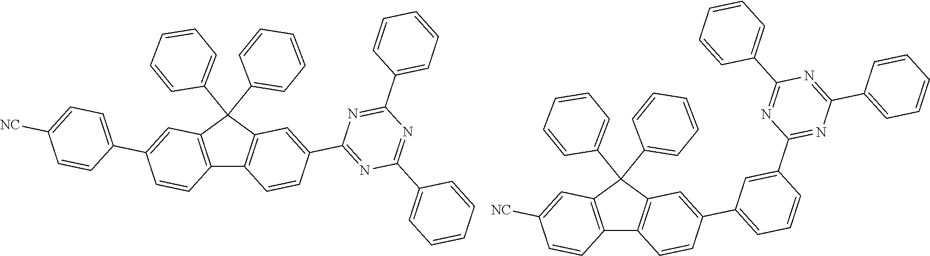

An embodiment may be an organic light emitting diode including the light emitting layer as the organic layer.

Another embodiment may be an organic light emitting diode including a light emitting layer and a hole transport region as the organic layer.

Another embodiment may be an organic light emitting diode including a light emitting layer and an electron transport region as the organic layer.

Another embodiment of the present invention may provide an organic light emitting diode including a hole transport region 140 and an electron transport region 150 in addition to the light emitting layer 130 as the organic layer 105, as shown in FIG. 1.

On the other hand, an organic light emitting diode may further include an electron injection layer (not shown), a hole injection layer (not shown), etc. in addition to the light emitting layer as the organic layer.

The organic light emitting diodes 100 may be manufactured by forming an anode or a cathode on a substrate, and then forming an organic layer by a dry film method such as vacuum deposition, sputtering, plasma plating and ion plating, and forming a cathode or an anode thereon.

The aforementioned organic light emitting diode may be applied to an organic light emitting display device.

### MODE FOR INVENTION

Hereinafter, the embodiments are illustrated in more detail with reference to examples. However, these examples are exemplary, and the scope of claims is not limited thereto.

Hereinafter, starting materials and reactants used in Examples and Synthesis Examples were purchased from Sigma-Aldrich Co. Ltd., TCI Inc., Tokyo chemical industry, or P&H tech as far as there in no particular comment or were synthesized by known methods.

### Preparation of Compound for Organic Optoelectronic Device

### Synthesis Example 1: Synthesis of Intermediate I-1

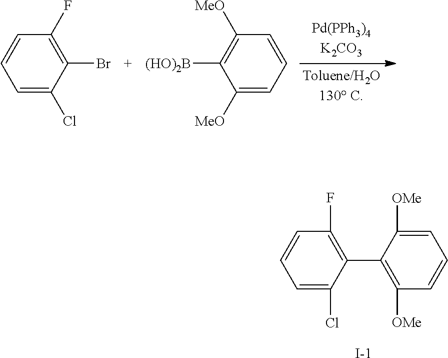 [SMILES: Fc1cccc(Cl)c1Br ; COc1cccc(OC)c1B(O)O ; COc1cccc(OC)c1-c1c(F)cccc1Cl ; [I-]]

In a nitrogen environment, 2-bromo-1-chloro-3-fluorobenzene (1000 g, 4,775 mmol) purchased from Henan Tianfu Chemical Co., Ltd. (www.tianfuchem.net) was dissolved in 10 L of toluene, and 2,6-dimethoxyphenylboronic acid (1043 g, 5,730 mmol) and tetrakis(triphenylphosphine)palladium (110 g, 95.5 mmol) purchased from Bide Pharmatech Ltd. (https://jlchem.co.kr/) were added thereto and then, stirred. Subsequently, potassium carbonate (1,650 g, 11,938 mmol) saturated in water was added thereto and then, heated under reflux at 130° C. for 3 days. When a reaction was completed, after adding water to the reaction solution, the mixture was extracted with dichloromethane (DCM) and treated with magnesium sulfate anhydrous to remove moisture and then, filtered and concentrated under a reduced pressure. The obtained residue was separated and purified through flash column chromatography to obtain Intermediate I-1 (535 g, 42%).

HRMS (70 eV, EI+): m/z calcd for C14H12ClFO2: 266.0510, found: 266.

Elemental Analysis: C, 63%; H, 5%

### Synthesis Example 2: Synthesis of Intermediate I-2

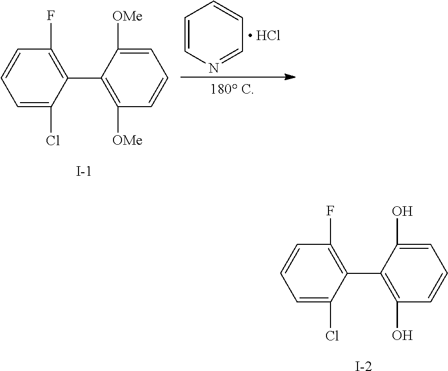

In a nitrogen environment, Intermediate I-1 (500 g, 1,875 mmol) and pyridine hydrochloride (1,483 g, 18,748 mmol) were added and heated under reflux at 180° C. for 12 hours. When a reaction was completed, after adding water to the reaction solution, the mixture was extracted with ethylacetate (EA) and treated with magnesium sulfate anhydrous to remove moisture and then, filtered and concentrated under a reduced pressure. The obtained residue was separated and purified through flash column chromatography to obtain Intermediate I-2 (361 g, 81%).

HRMS (70 eV, EI+): m/z calcd for C12H8ClFO2: 238.0197, found: 238.

Elemental Analysis: C, 60%; H, 3%

### Synthesis Example 3: Synthesis of Intermediate T-3

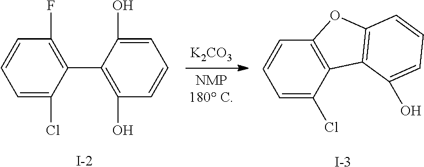

In a nitrogen environment, Intermediate I-2 (350 g, 1,467 mmol) was dissolved in 0.3 L of N-methyl-2-pyrrolidone (NMP), and potassium carbonate (406 g, 2,934 mmol) was added thereto and then, heated under reflux for 3 hours. When a reaction was completed, after adding water to the reaction solution, the mixture was extracted with dichloromethane (DCM) and treated with magnesium sulfate anhydrous to remove moisture and then, filtered and concentrated under a reduced pressure. The obtained residue was separated and purified through flash column chromatography to obtain Intermediate I-3 (103 g, 32%).

HRMS (70 eV, EI+): m/z calcd for C12H7ClO2: 218.0135, found: 218.

Elemental Analysis: C, 66%; H, 3%

### Synthesis Example 4: Synthesis of Intermediate I-4

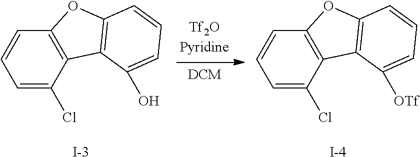

In a nitrogen environment, Intermediate I-3 (100 g, 457 mmol) was dissolved in 1.0 L of dichloromethane (DCM) and then, cooled to 0° C. After adding pyridine (43.4 g, 549 mmol) thereto and stirring the mixture for 30 minutes, tifluoromethanesulfonic anhydride (155 g, 549 mmol) was slowly added thereto and then, stirred. After 3 hours, the reaction solution was cooled to 0° C., and after slowly adding water thereto for 30 minutes, the mixture was extracted with dichloromethane (DCM) and treated with magnesium sulfate anhydrous to remove moisture and then, filtered and concentrated under a reduced pressure. The obtained residue was separated and purified through flash column chromatography to obtain Intermediate I-4 (157 g, 98%).

HRMS (70 eV, EI+): m/z calcd for C13H6ClF3O4S: 349.9627, found: 350.

Elemental Analysis: C, 45%; H, 2%

### Synthesis Example 5: Synthesis of Intermediate T-5

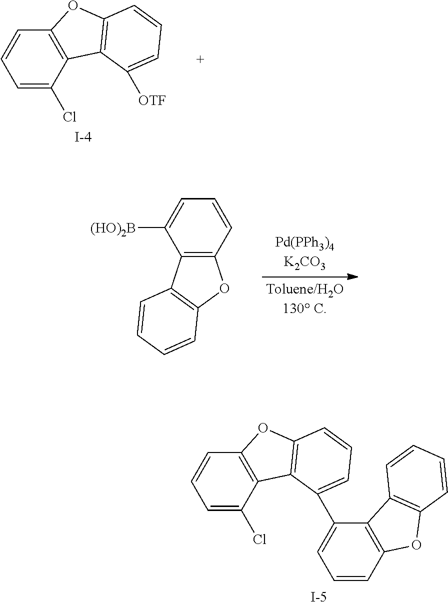

Intermediate I-5 (103 g, 65%) was obtained in the same manner as in Synthesis Example 1 except that Intermediate I-4 (150 g, 428 mmol) and dibenzofuran-1-boronic acid (99.8 g, 471 mmol) purchased from Tokyo Chemical Industry Co., Ltd. were used.

HRMS (70 eV, EI+): m/z calcd for C24H13ClO2: 368.0604, found: 368.

Elemental Analysis: C, 78%; H, 4%

### Synthesis Example 6: Synthesis of Intermediate I-6

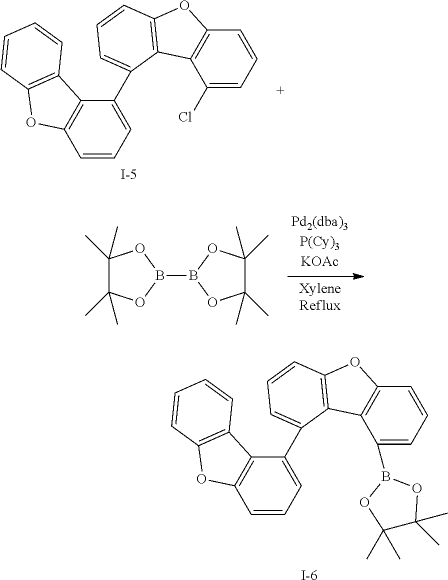

In a nitrogen environment, Intermediate I-5 (100 g, 271 mmol) was dissolved in 0.1 L of xylene, and then, bis(pinacolato)diboron (82.6 g, 325 mmol), tris(dibenzylideneacetone)dipalladium (0) (2.48 g, 2.71 mmol), tricyclohexylphosphine (3.04 g, 10.8 mmol), and potassium acetate (79.8 g, 813 mmol) were added thereto and then, heated under reflux for 12 hours. When a reaction was completed, after adding water to the reaction solution, the mixture was extracted with ethylacetate (EA) and treated with magnesium sulfate anhydrous to remove moisture and then, filtered and concentrated under a reduced pressure. The obtained residue was separated and purified through flash column chromatography to obtain Intermediate I-6 (62.4 g, 50%).

HRMS (70 eV, EI+): m/z calcd for C30H25BO4: 460.1846, found: 460.

Elemental Analysis: C, 78%; H, 5%

### Synthesis Example 7: Synthesis of Compound 1

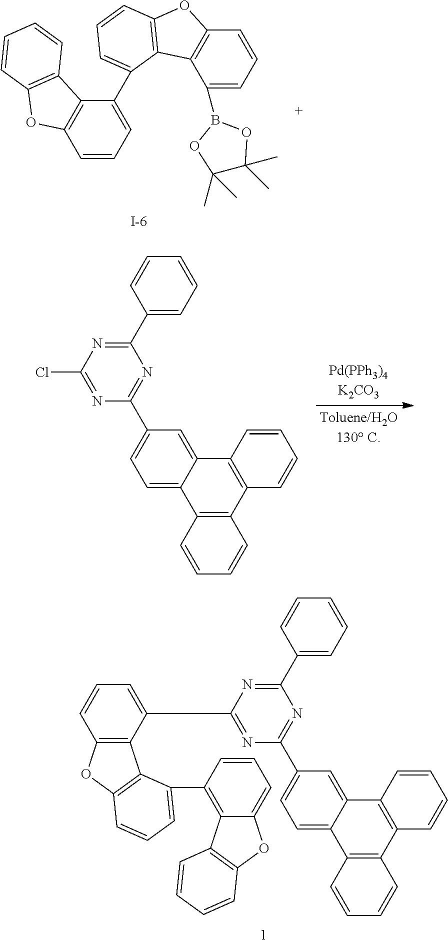

Compound 1 (28.0 g, 90%) was obtained in the same manner as in Synthesis Example 1 except that Intermediate I-6 (20 g, 43.4 mmol) and 2-chloro-4-phenyl-6-(triphenylen-2-yl)-1,3,5-triazine (18.2 g, 43.4 mmol) purchased from P&H Tech (http://www.phtech.co.kr/) were used.

HRMS (70 eV, EI+): m/z calcd for C51H29N3O2: 715.2260, found: 715.

Elemental Analysis: C, 86%; H, 4%

### Synthesis Example 8: Synthesis of Intermediate I-7

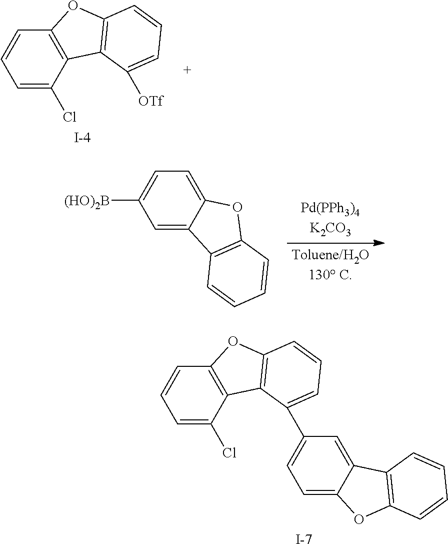

Intermediate I-7 (42.2 g, 80%) was obtained in the same manner as in Synthesis Example 1 except that Intermediate I-4 (50 g, 143 mmol) and dibenzofuran-2-boronic acid (33.2 g, 157 mmol) purchased from Tokyo Chemical Industry Co., Ltd. were used.

HRMS (70 eV, EI+): m/z calcd for C24H13ClO2: 368.0604, found: 368.

Elemental Analysis: C, 78%; H, 4%

### Synthesis Example 9: Synthesis of Intermediate I-8

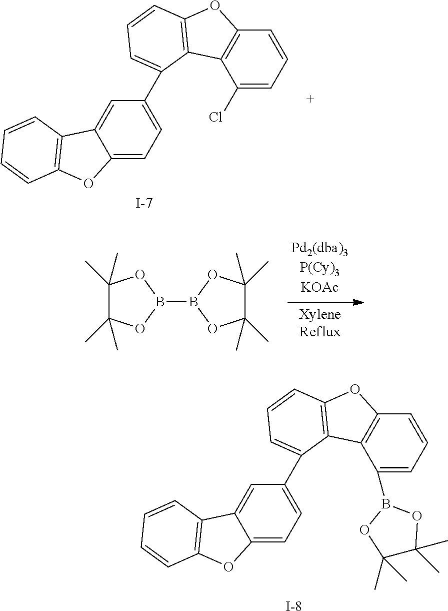

Intermediate I-8 (37.3 g, 75%) was obtained in the same manner as in Synthesis Example 6 except that Intermediate I-7 (40 g, 108 mmol) was used.

HRMS (70 eV, EI+): m/z calcd for C30H25BO4: 460.1846, found: 460.

Elemental Analysis: C, 78%; H, 5%

### Synthesis Example 10: Synthesis of Compound 2

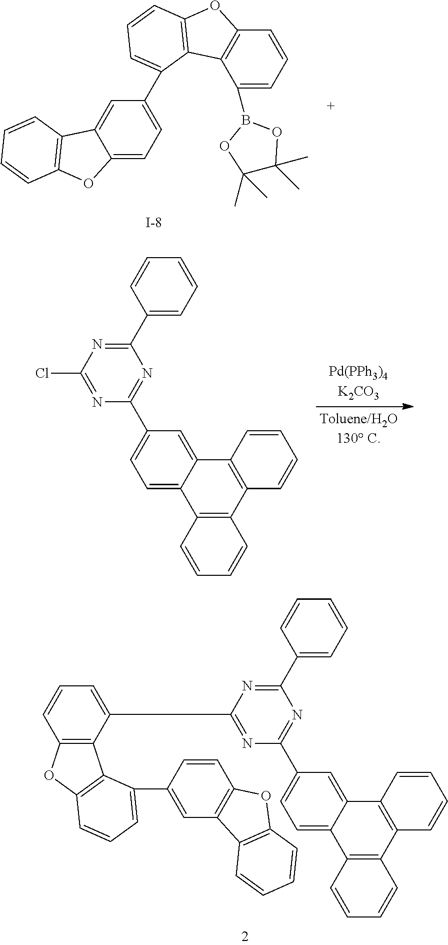

Compound 1 (28.9 g, 93%) was obtained in the same manner as in Synthesis Example 1 except that Intermediate I-8 (20 g, 43.4 mmol) and 2-chloro-4-phenyl-6-(triphenylen-2-yl)-1,3,5-triazine purchased from P&H Tech (18.2 g, 43.4 mmol) were used.

HRMS (70 eV, EI+): m/z calcd for C51H29N3O2: 715.2260, found: 715.

Elemental Analysis: C, 86%; H, 4%

### Synthesis Example 11: Synthesis of Intermediate I-9

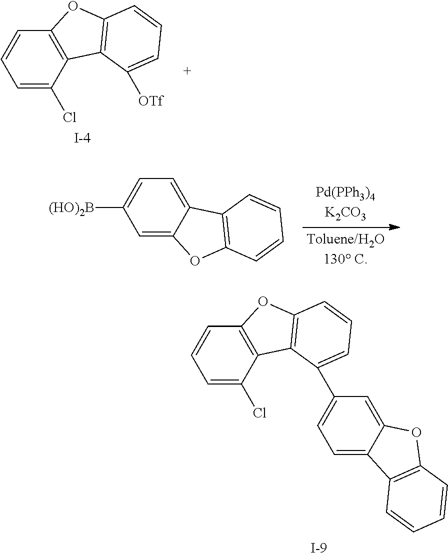

Intermediate I-9 (40.6 g, 77%) was obtained in the same manner as in Synthesis Example 1 except that Intermediate I-4 (50 g, 143 mmol) and dibenzofuran-3-boronic acid purchased from Tokyo Chemical Industry Co., Ltd. (33.2 g, 157 mmol) were used.

HRMS (70 eV, EI+): m/z calcd for C24H13ClO2: 368.0604, found: 368.

Elemental Analysis: C, 78%; H, 4%

### Synthesis Example 12: Synthesis of Intermediate T-10

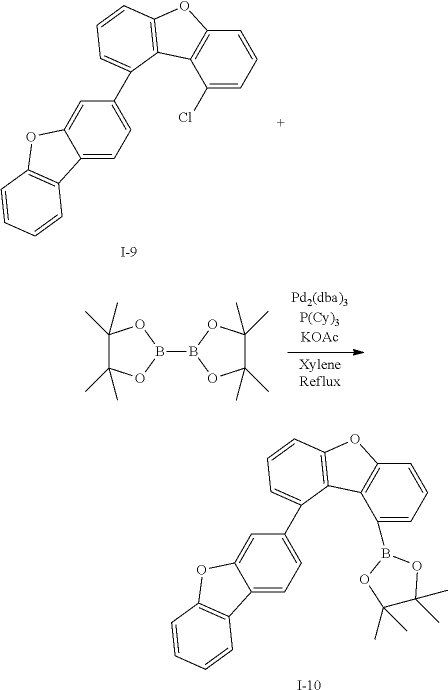

Intermediate I-10 (40.3 g, 81%) was obtained in the same manner as in Synthesis Example 6 except that Intermediate I-9 (40 g, 108 mmol) was used.

HRMS (70 eV, EI+): m/z calcd for C30H25BO4: 460.1846, found: 460.

Elemental Analysis: C, 78%; H, 5%

### Synthesis Example 13: Synthesis of Compound 3

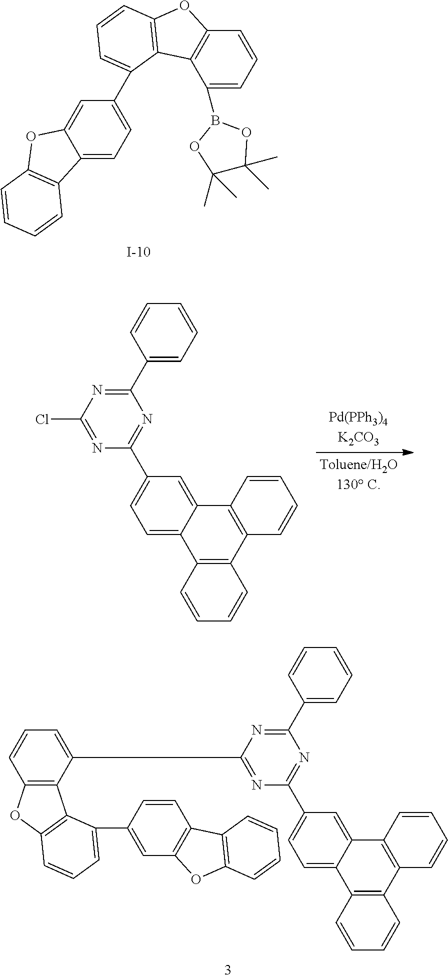

Compound 3 (29.5 g, 95%) was obtained in the same manner as in Synthesis Example 1 except that Intermediate I-10 (20 g, 43.4 mmol) and 2-chloro-4-phenyl-6-(triphenylen-2-yl)-1,3,5-triazine purchased from P&H Tech (18.2 g, 43.4 mmol) were used.

HRMS (70 eV, EI+): m/z calcd for C51H29N3O2: 715.2260, found: 715.

Elemental Analysis: C, 86%; H, 4%

### Synthesis Example 14: Synthesis of Intermediate I-11

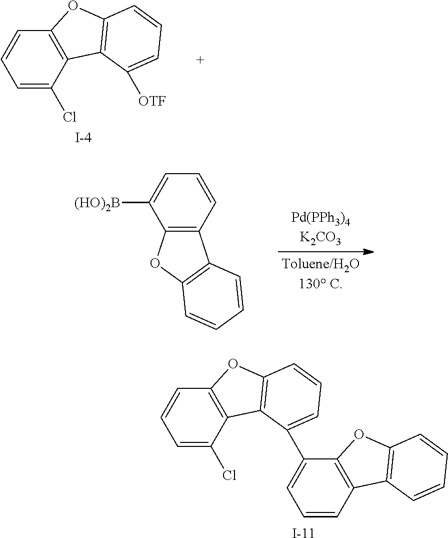

Intermediate I-11 (32.7 g, 62%) was obtained in the same manner as in Synthesis Example 1 except that Intermediate I-4 (50 g, 143 mmol) and dibenzofuran-4-boronic acid purchased from Tokyo Chemical Industry Co., Ltd. (33.2 g, 157 mmol) were used.

HRMS (70 eV, EI+): m/z calcd for C24H13ClO2: 368.0604, found: 368.

### Elemental Analysis: C, 78%; H, 4%

### Synthesis Example 15: Synthesis of Intermediate I-12

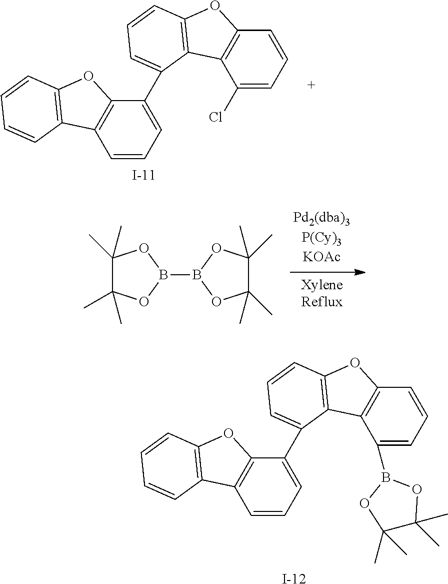

Intermediate I-12 (21.4 g, 43%) was obtained in the same manner as in Synthesis Example 6 except that Intermediate I-11 (40 g, 108 mmol) was used.

HRMS (70 eV, EI+): m/z calcd for C30H25BO4: 460.1846, found: 460.

Elemental Analysis: C, 78%; H, 5%

### Synthesis Example 16: Synthesis of Compound 4

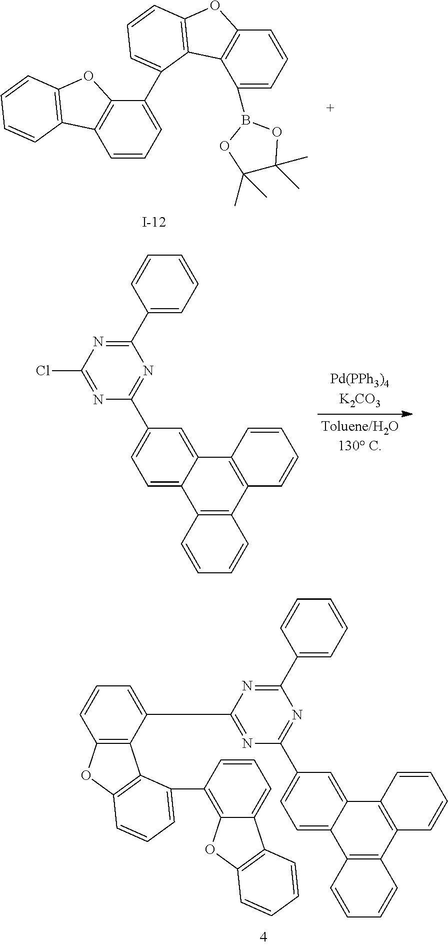

Compound 4 (26.4 g, 85%) was obtained in the same manner as in Synthesis Example 1 except that Intermediate I-12 (20 g, 43.4 mmol) and 2-chloro-4-phenyl-6-(triphenylen-2-yl)-1,3,5-triazine purchased from P&H Tech (18.2 g, 43.4 mmol) were used.

HRMS (70 eV, EI+): m/z calcd for C51H29N3O2: 715.2260, found: 715.

Elemental Analysis: C, 86%; H, 4%

### Synthesis Example 17: Synthesis of Intermediate I-13

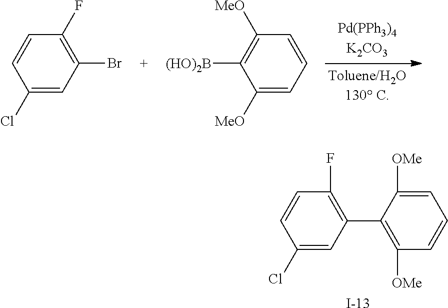

Intermediate I-13 (102 g, 80%) was obtained in the same manner as in Synthesis Example 1 except that 2-bromo-4-chloro-1-fluorobenzene purchased from Tokyo Chemical Industry Co., Ltd. (100 g, 477 mmol) and 2,6-dimethoxyphenylboronic acid purchased from P&H Tech Bide Pharmatech Ltd. (101 g, 525 mmol) were used.

HRMS (70 eV, EI+): m/z calcd for C14H12ClFO2: 266.0510, found: 266.

Elemental Analysis: C, 63%; H, 5%

### Synthesis Example 18: Synthesis of Intermediate I-14

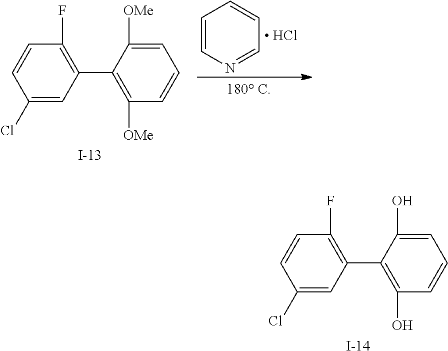

Intermediate I-14 (85.0 g, 95%) was obtained in the same manner as in Synthesis Example 2 except that Intermediate I-13 (100 g, 375 mmol) was used.

HRMS (70 eV, EI+): m/z calcd for C12H8ClFO2: 238.0197, found: 238.

Elemental Analysis: C, 60%; H, 3%

### Synthesis Example 19: Synthesis of Intermediate I-15

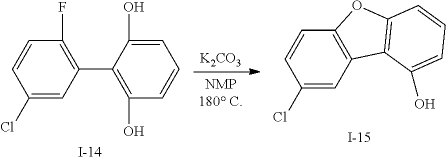

Intermediate I-15 (67.8 g, 65%) was obtained in the same manner as in Synthesis Example 3 except that Intermediate I-14 (100 g, 477 mmol) was used.

HRMS (70 eV, EI+): m/z calcd for C12H7ClO2: 218.0135, found: 218.

Elemental Analysis: C, 66%; H, 3%

### Synthesis Example 20: Synthesis of Intermediate I-16

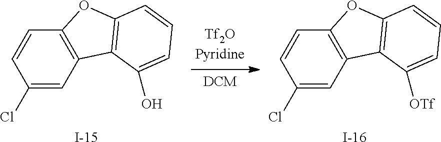

Intermediate I-16 (63.7 g, 98%) was obtained in the same manner as in Synthesis Example 4 except that Intermediate I-15 (65 g, 185 mmol) was used.

HRMS (70 eV, EI+): m/z calcd for C13H6ClF3O4S: 349.9627, found: 350.

Elemental Analysis: C, 45%; H, 2%

### Synthesis Example 21: Synthesis of Intermediate I-17

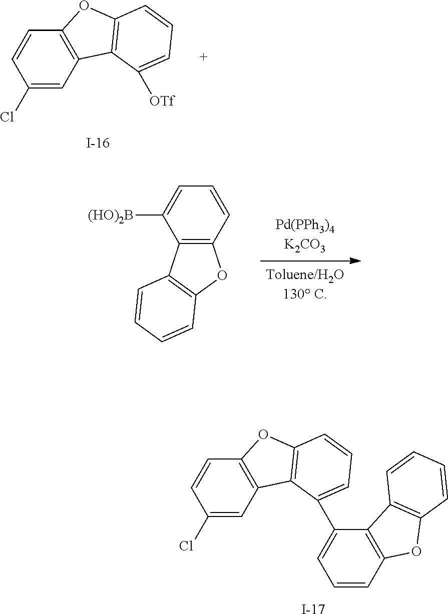

Intermediate I-17 (38.5 g, 61%) was obtained in the same manner as in Synthesis Example 1 except that Intermediate I-16 (60 g, 171 mmol) and dibenzofuran-1-boronic acid (39.9 g, 188 mmol) purchased from Tokyo Chemical Industry Co., Ltd. were used.

HRMS (70 eV, EI+): m/z calcd for C24H13ClO2: 368.0604, found: 368.

Elemental Analysis: C, 78%; H, 4%

### Synthesis Example 22: Synthesis of Intermediate I-18

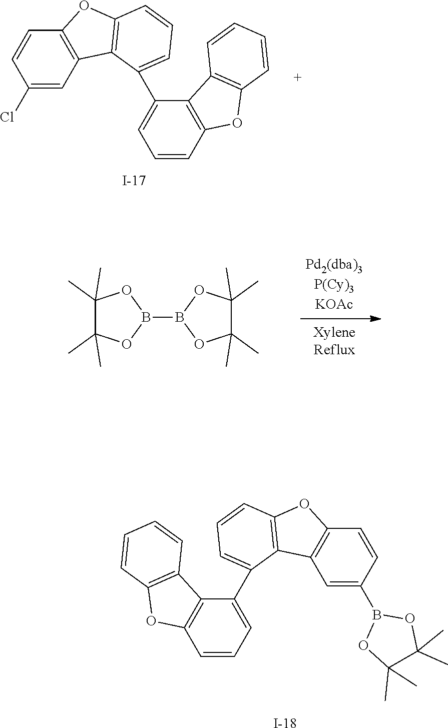

Intermediate I-18 (12.6 g, 78%) was obtained in the same manner as in Synthesis Example 6 except that Intermediate I-17 (35 g, 94.9 mmol) was used.

HRMS (70 eV, EI+): m/z calcd for C30H25BO4: 460.1846, found: 460.

Elemental Analysis: C, 78%; H, 5%

### Synthesis Example 23: Synthesis of Compound 5

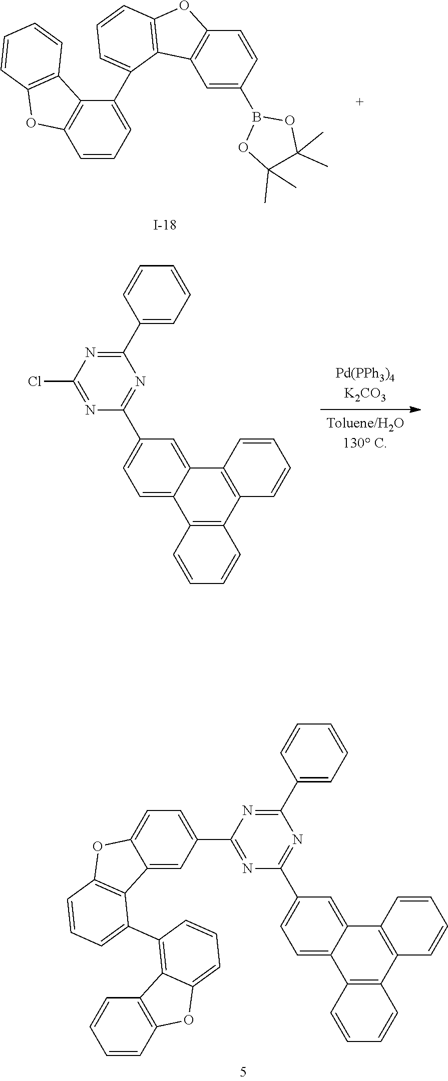

Compound 5 (14.3 g, 92%) was obtained in the same manner as in Synthesis Example 1 except that Intermediate I-18 (10 g, 21.7 mmol) and 2-chloro-4-phenyl-6-(triphenylen-2-yl)-1,3,5-triazine (9.08 g, 21.7 mmol) purchased from P&H Tech were used.

HRMS (70 eV, EI+): m/z calcd for C51H29N3O2: 715.2260, found: 715.

Elemental Analysis: C, 86%; H, 4%

### Synthesis Example 24: Synthesis of Intermediate I-19

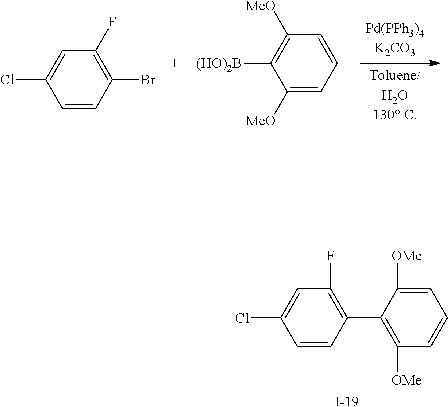

Intermediate I-19 (122 g, 96%) was obtained in the same manner as in Synthesis Example 1 except that 1-bromo-4-chloro-2-fluorobenzene (100 g, 477 mmol) purchased from Tokyo Chemical Industry Co., Ltd. and 2,6-dimethoxyphenylboronic acid (101 g, 525 mmol) purchased from P&H Tech or Bide Pharmatech Ltd P&H Tech Bide Pharmatech Ltd were used.

HRMS (70 eV, EI+): m/z calcd for C14H12ClFO2: 266.0510, found: 266.

Elemental Analysis: C, 63%; H, 5%

### Synthesis Example 25: Synthesis of Intermediate I-20

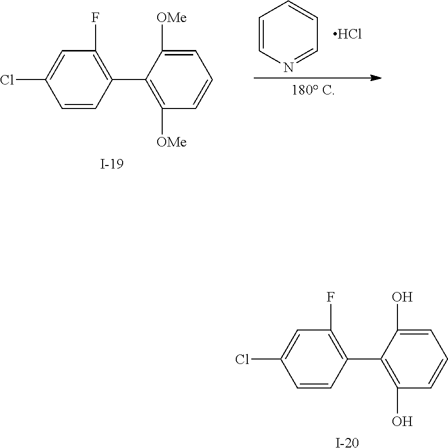

Intermediate I-20 (97.7 g, 91%) was obtained in the same manner as in Synthesis Example 2 except that Intermediate I-19 (120 g, 450 mmol) was used.

HRMS (70 eV, EI+): m/z calcd for C12H8ClFO2: 238.0197, found: 238.

Elemental Analysis: C, 60%; H, 3%

### Synthesis Example 26: Synthesis of Intermediate I-21

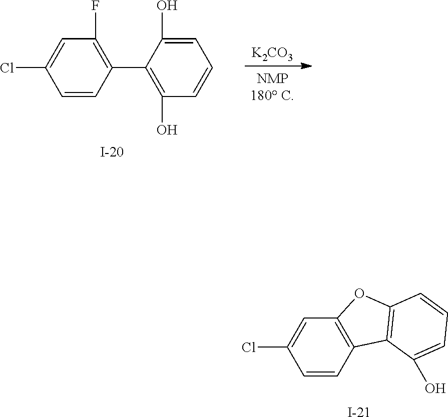

Intermediate I-21 (60.9 g, 70%) was obtained in the same manner as in Synthesis Example 3 except that Intermediate I-20 (95 g, 398 mmol) was used.

HRMS (70 eV, EI+): m/z calcd for C12H7ClO2: 218.0135, found: 218.

Elemental Analysis: C, 66%; H, 3%

### Synthesis Example 27: Synthesis of Intermediate I-22

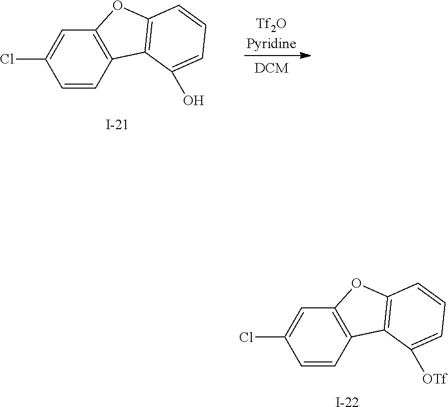

Intermediate I-22 (83.8 g, 95%) was obtained in the same manner as in Synthesis Example 4 except that Intermediate I-21 (55 g, 252 mmol) was used.

HRMS (70 eV, EI+): m/z calcd for C13H6ClF3O4S: 349.9627, found: 350.

Elemental Analysis: C, 45%; H, 2%

### Synthesis Example 28: Synthesis of Intermediate I-23

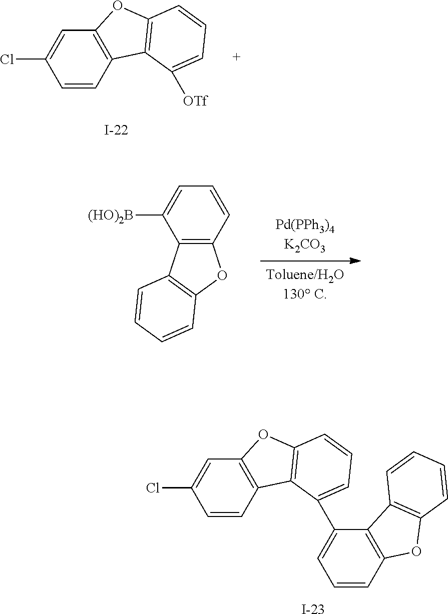

Intermediate I-23 (63.1 g, 75%) was obtained in the same manner as in Synthesis Example 1 except that Intermediate I-22 (80 g, 228 mmol) and dibenzofuran-1-boronic acid (53.2 g, 251 mmol) purchased from Tokyo Chemical Industry Co., Ltd. were used.

HRMS (70 eV, EI+): m/z calcd for C24H13ClO2: 368.0604, found: 368.

Elemental Analysis: C, 78%; H, 4%

### Synthesis Example 29: Synthesis of Intermediate I-24

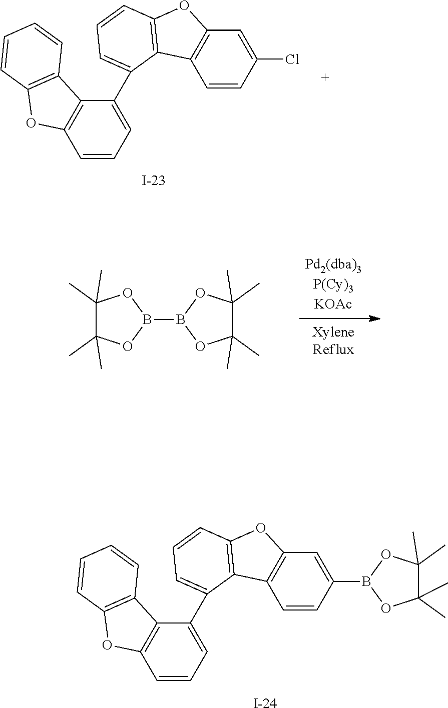

Intermediate I-24 (54.1 g, 70%) was obtained in the same manner as in Synthesis Example 6 except that Intermediate I-23 (62 g, 168 mmol) was used.

HRMS (70 eV, EI+): m/z calcd for C30H25BO4: 460.1846, found: 460.

Elemental Analysis: C, 78%; H, 5%

### Synthesis Example 30: Synthesis of Compound 6

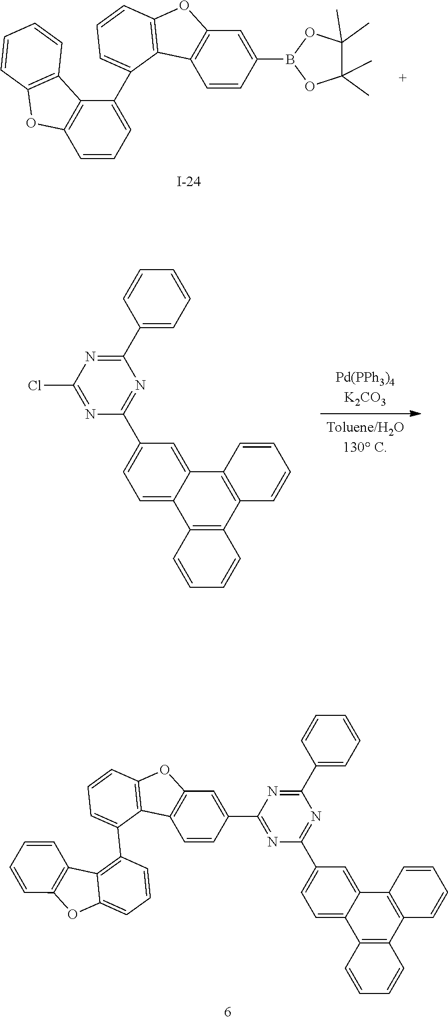

Compound 5 (31.1 g, 85%) was obtained in the same manner as in Synthesis Example 1 except that Intermediate I-24 (20 g, 43.4 mmol) and 2-chloro-4-phenyl-6-(triphenylen-2-yl)-1,3,5-triazine (18.2 g, 43.4 mmol) purchased from P&H Tech were used.

HRMS (70 eV, EI+): m/z calcd for C51H29N3O2: 715.2260, found: 715.

Elemental Analysis: C, 86%; H, 4%

### Synthesis Example 31: Synthesis of Intermediate I-25

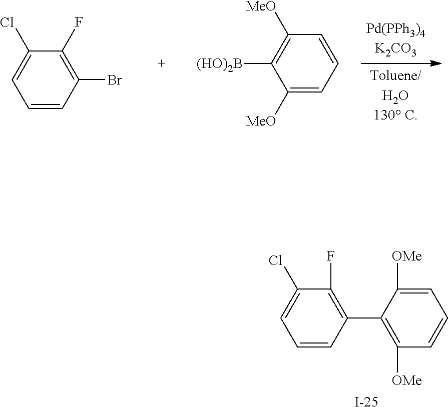

Intermediate I-25 (114 g, 90%) was obtained in the same manner as in Synthesis Example 1 except that 1-bromo-3-chloro-2-fluorobenzene (100 g, 477 mmol) purchased from Tokyo Chemical Industry Co., Ltd. and 2,6-dimethoxyphenylboronic acid (101 g, 525 mmol) purchased from P&H Tech Bide PharmaTech Ltd. were used.

HRMS (70 eV, EI+): m/z calcd for C14H12ClFO2: 266.0510, found: 266.

Elemental Analysis: C, 63%; H, 5%

### Synthesis Example 32: Synthesis of Intermediate I-26

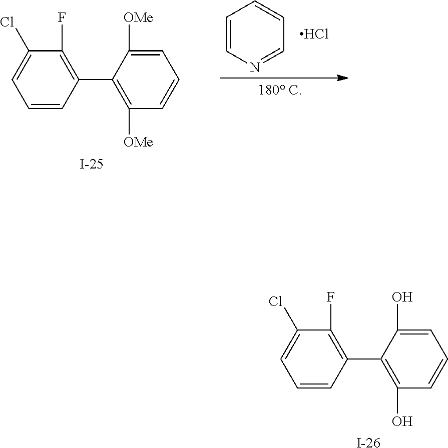

Intermediate I-26 (100 g, 99%) was obtained in the same manner as in Synthesis Example 2 except that Intermediate I-25 (113 g, 424 mmol) was used.

HRMS (70 eV, EI+): m/z calcd for C12H8ClFO2: 238.0197, found: 238.

Elemental Analysis: C, 60%; H, 3%

### Synthesis Example 33: Synthesis of Intermediate I-27

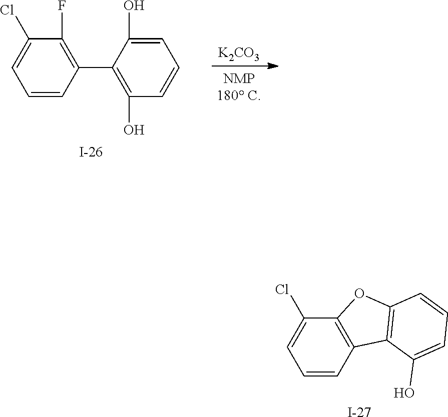

Intermediate I-27 (46.7 g, 52%) was obtained in the same manner as in Synthesis Example 3 except that Intermediate I-26 (98 g, 411 mmol) was used.

HRMS (70 eV, EI+): m/z calcd for C12H7ClO2: 218.0135, found: 218.

Elemental Analysis: C, 66%; H, 3%

### Synthesis Example 34: Synthesis of Intermediate I-28

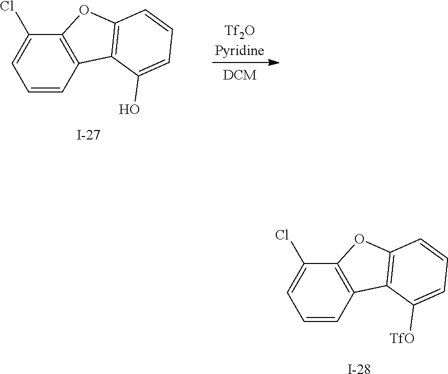

Intermediate I-28 (70.8 g, 98%) was obtained in the same manner as in Synthesis Example 4 except that Intermediate I-27 (45 g, 206 mmol) was used.

HRMS (70 eV, EI+): m/z calcd for C13H6ClF3O4S: 349.9627, found: 350.

Elemental Analysis: C, 45%; H, 2%

### Synthesis Example 35: Synthesis of Intermediate T-29

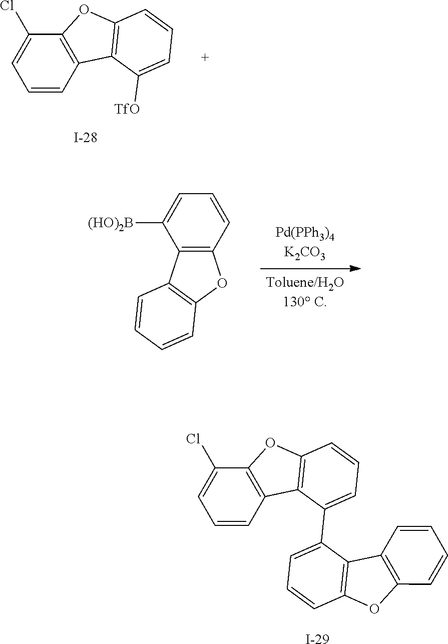

Intermediate I-29 (50.9 g, 70%) was obtained in the same manner as in Synthesis Example 1 except that Intermediate I-28 (69 g, 197 mmol) and dibenzofuran-1-boronic acid (45.8 g, 216 mmol) purchased from Tokyo Chemical Industry Co., Ltd. were used.

HRMS (70 eV, EI+): m/z calcd for C24H13ClO2: 368.0604, found: 368.

Elemental Analysis: C, 78%; H, 4%

### Synthesis Example 36: Synthesis of Intermediate I-30

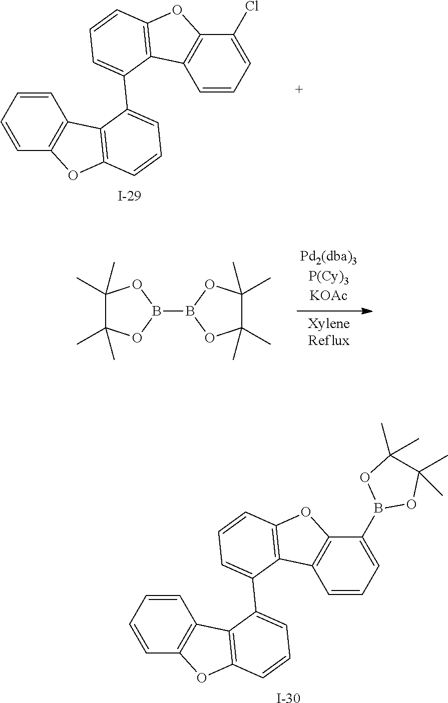

Intermediate I-30 (44.9 g, 72%) was obtained in the same manner as in Synthesis Example 6 except that Intermediate I-29 (50 g, 136 mmol) was used.

HRMS (70 eV, EI+): m/z calcd for C30H25BO4: 460.1846, found: 460.

Elemental Analysis: C, 78%; H, 5%

### Synthesis Example 37: Synthesis of Compound 7

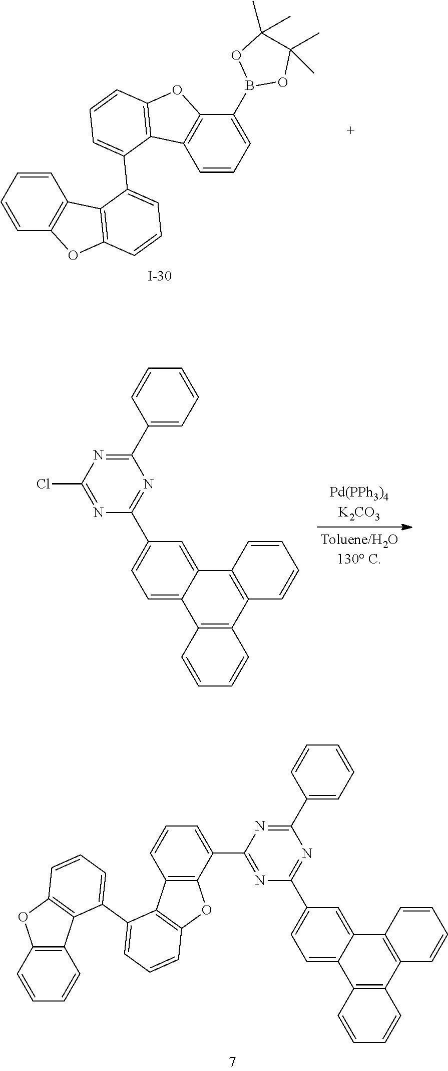

Compound 5 (28.0 g, 90%) was obtained in the same manner as in Synthesis Example 1 except that Intermediate I-30 (20 g, 43.4 mmol) and 2-chloro-4-phenyl-6-(triphenylen-2-yl)-1,3,5-triazine (18.2 g, 43.4 mmol) purchased from P&H Tech were used.

HRMS (70 eV, EI+): m/z calcd for C51H29N3O2: 715.2260, found: 715.

Elemental Analysis: C, 86%; H, 4%

### Synthesis Example 38: Synthesis of Intermediate I-31

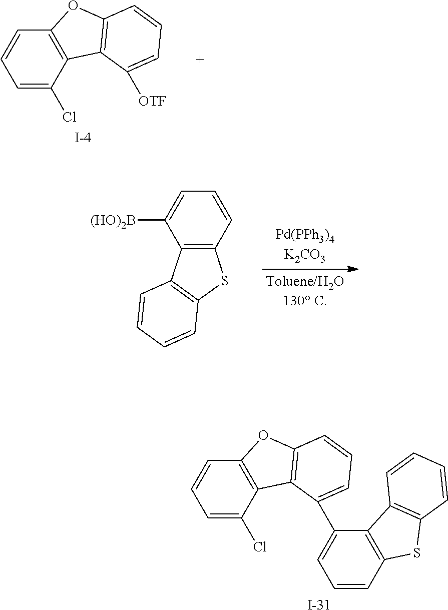

Intermediate I-31 (76.8 g, 70%) was obtained in the same manner as in Synthesis Example 1 except that Intermediate I-4 (100 g, 285 mmol) and dibenzothiophene-1-boronic acid (78.0 g, 342 mmol) were used.

HRMS (70 eV, EI+): m/z calcd for C24H13ClOS: 384.0376, found: 384.

Elemental Analysis: C, 75%; H, 3%

### Synthesis Example 39: Synthesis of Intermediate I-32

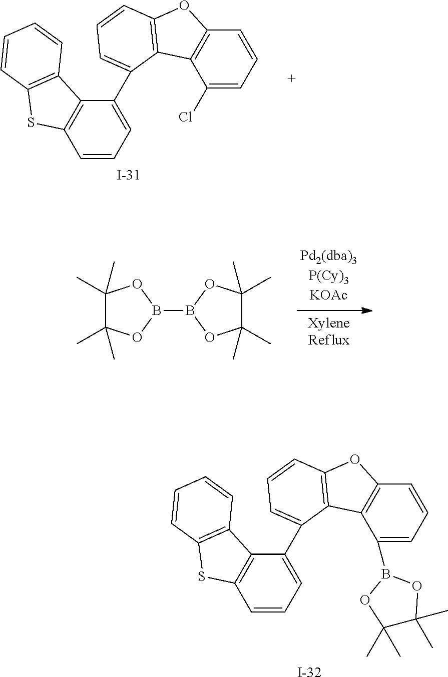

Intermediate I-32 (40.8 g, 44%) was obtained in the same manner as in Synthesis Example 6 except that Intermediate I-31 (75 g, 195 mmol) was used.

HRMS (70 eV, EI+): m/z calcd for C30H25BO3S: 476.1617, found: 476.

Elemental Analysis: C, 76%; H, 5%

### Synthesis Example 40: Synthesis of Compound 8

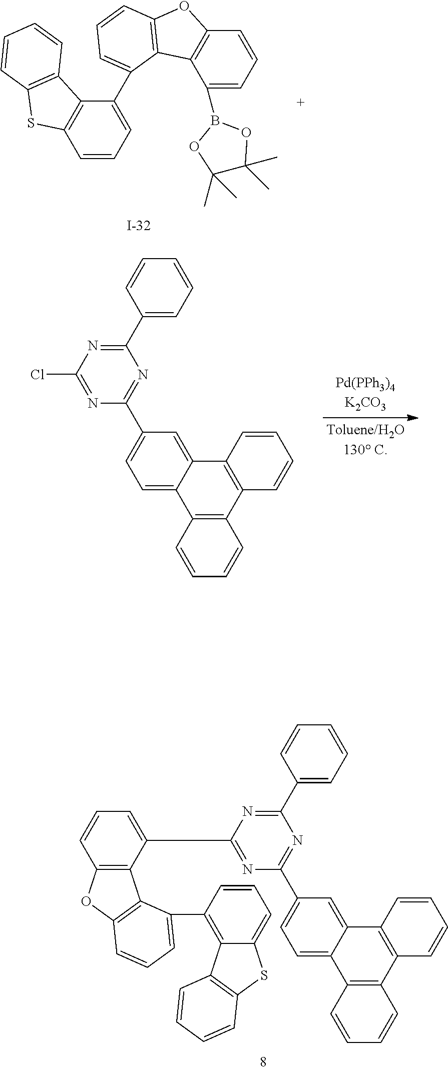

Compound 8 (26.7 g, 87%) was obtained in the same manner as in Synthesis Example 1 except that Intermediate I-32 (20 g, 42.0 mmol) and 2-chloro-4-phenyl-6-(triphenylen-2-yl)-1,3,5-triazine (17.5 g, 42.0 mmol) purchased from P&H Tech were used.

HRMS (70 eV, EI+): m/z calcd for C51H29N30S: 731.2031, found: 731.

Elemental Analysis: C, 84%; H, 4%

### Synthesis Example 41: Synthesis of Intermediate I-33

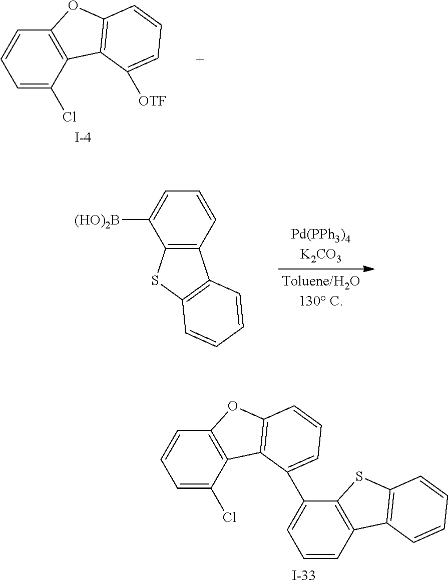

Intermediate I-33 (71.3 g, 65%) was obtained in the same manner as in Synthesis Example 1 except that Intermediate I-4 (100 g, 285 mmol) and dibenzothiophene-4-boronic acid (78.0 g, 342 mmol) were used.

HRMS (70 eV, EI+): m/z calcd for C24H13ClOS: 384.0376, found: 384.

Elemental Analysis: C, 75%; H, 3%

### Synthesis Example 42: Synthesis of Intermediate I-34

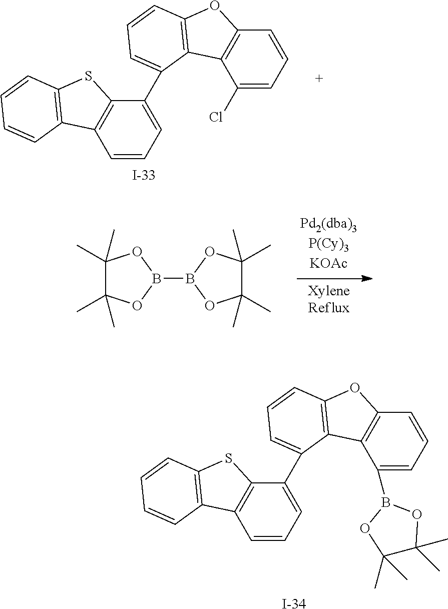

Intermediate I-34 (35.5 g, 41%) was obtained in the same manner as in Synthesis Example 6 except that Intermediate I-33 (70 g, 182 mmol) was used.

HRMS (70 eV, EI+): m/z calcd for C30H25BO3S: 476.1617, found: 476.

### Elemental Analysis: C, 76%; H, 5%

### Synthesis Example 43: Synthesis of Compound 12

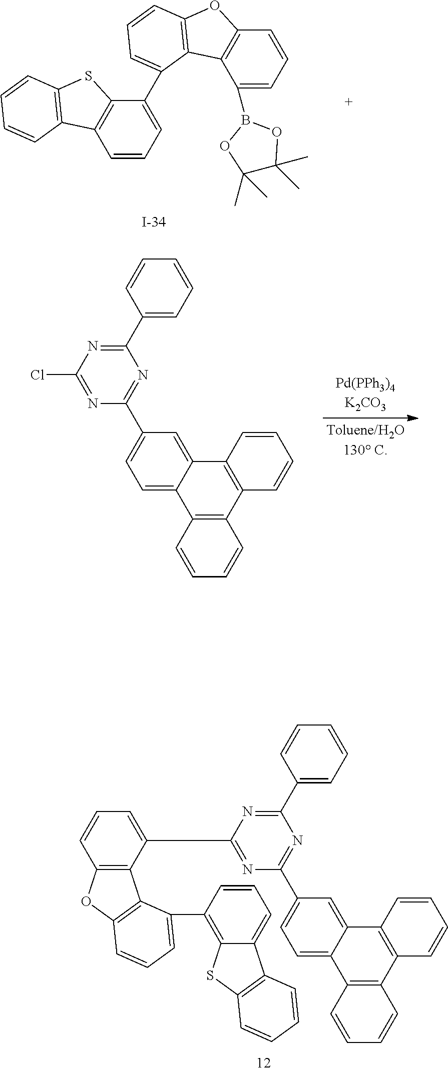

Compound 12 (27.0 g, 88%) was obtained in the same manner as in Synthesis Example 1 except that Intermediate I-34 (20 g, 42.0 mmol) and 2-chloro-4-phenyl-6-(triphenylen-2-yl)-1,3,5-triazine (17.5 g, 42.0 mmol) purchased from P&H Tech were used.

HRMS (70 eV, EI+): m/z calcd for C51H29N30S: 731.2031, found: 731.

Elemental Analysis: C, 84%; H, 4%

### Synthesis Example 44: Synthesis of Compound 13

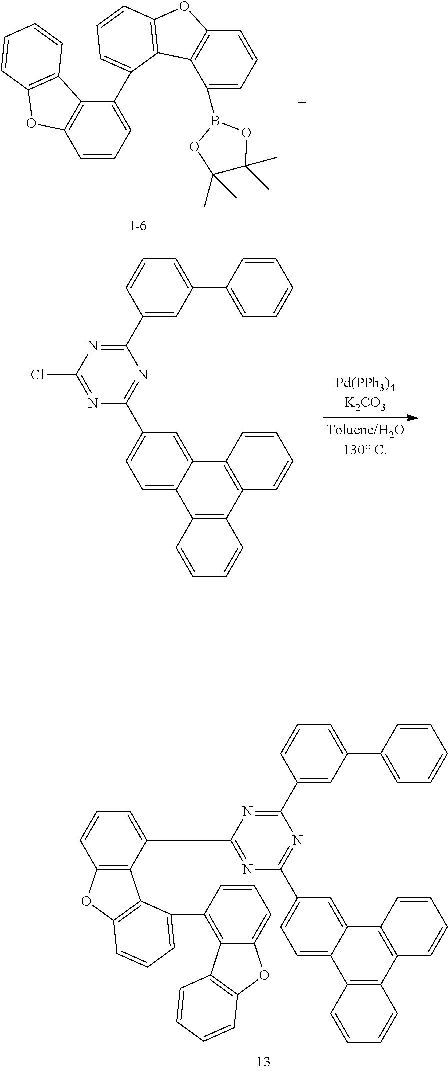

Compound 13 (29.2 g, 85%) was obtained in the same manner as in Synthesis Example 1 except that Intermediate I-6 (20 g, 43.4 mmol) and 2-(biphenyl-3-yl)-4-chloro-6-(triphenylen-2-yl)-1,3,5-triazine (19.1 g, 43.4 mmol) purchased from P&H Tech were used.

HRMS (70 eV, EI+): m/z calcd for C57H33N3O2: 791.2573, found: 791.

Elemental Analysis: C, 86%; H, 4%

### Synthesis Example 45: Synthesis of Intermediate T-35

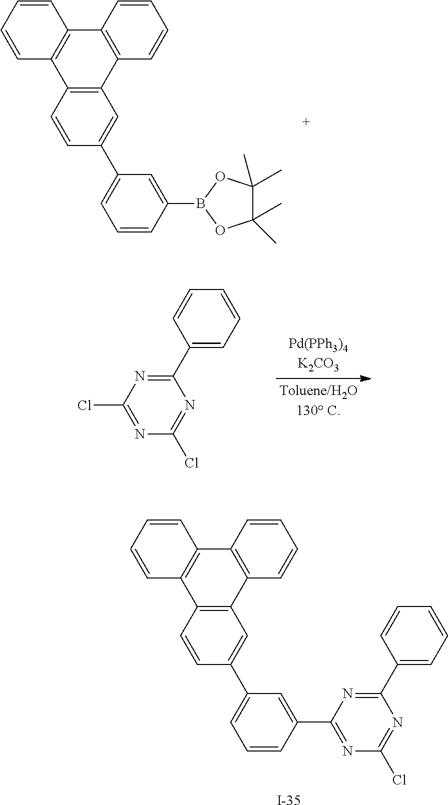

Intermediate I-35 (20.0 g, 35%) was obtained in the same manner as in Synthesis Example 1 except that 4,4,5,5-tetramethyl-2-(3-(triphenylen-2-yl)phenyl)-1,3,2-dioxaborolane (50 g, 116 mmol) and 2,4-dichloro-6-phenyl-1,3,5-triazine (39.4 g, 174 mmol) were used.

HRMS (70 eV, EI+): m/z calcd for C33H20C1N3: 493.1346, found: 493.

Elemental Analysis: C, 80%; H, 4%

### Synthesis Example 46: Synthesis of Compound 25

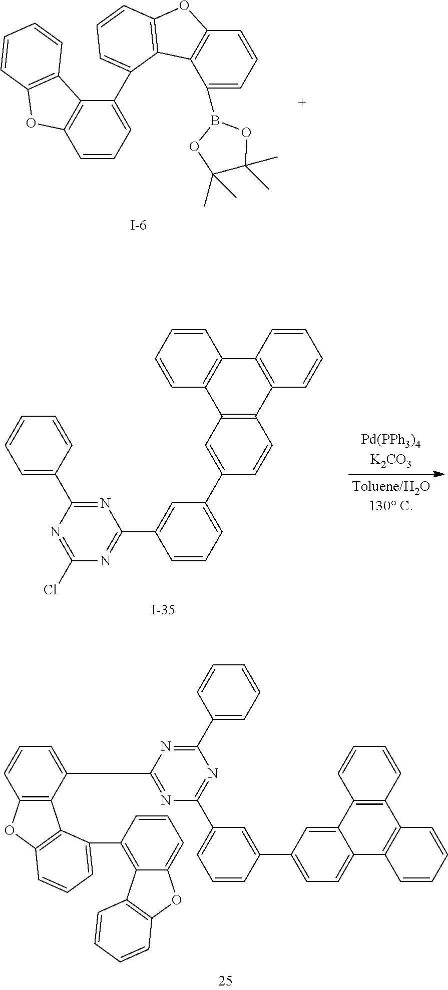

Compound 25 (15.3 g, 89%) was obtained in the same manner as in Synthesis Example 1 except that Intermediate I-6 (10 g, 21.7 mmol) and Intermediate I-35 (10.7 g, 21.7 mmol) were used.

HRMS (70 eV, EI+): m/z calcd for C57H33N3O2: 791.2573, found: 791.

Elemental Analysis: C, 86%; H, 4%

### Synthesis Example 47: Synthesis of Intermediate I-36

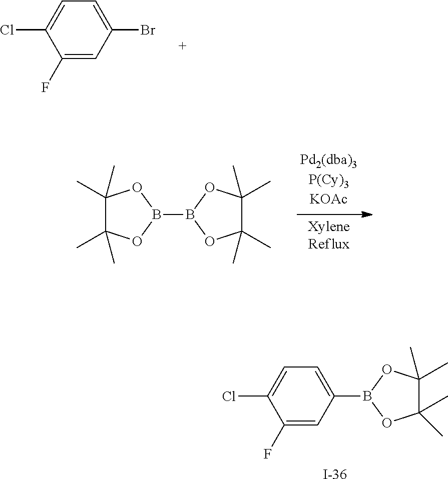

Intermediate I-36 (113 g, 92%) was obtained in the same manner as in Synthesis Example 6 except that 4-bromo-1-chloro-2-fluorobenzene (100 g, 477 mmol) was used.

HRMS (70 eV, EI+): m/z calcd for C30H25BO3S: 256.0838, found: 256.

Elemental Analysis: C, 56%; H, 6%

### Synthesis Example 48: Synthesis of Intermediate I-37

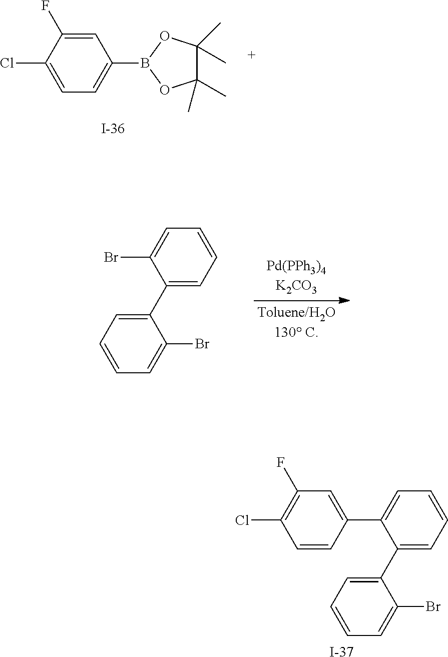

Intermediate I-37 (124 g, 80%) was obtained in the same manner as in Synthesis Example 1 except that Intermediate I-36 (110 g, 429 mmol) and 2,2′-dibromobiphenyl (201 g, 643 mmol) were used.

HRMS (70 eV, EI+): m/z calcd for C18H11BrClF: 359.9717, found: 359.

Elemental Analysis: C, 60%; H, 3%

### Synthesis Example 49: Synthesis of Intermediate I-38

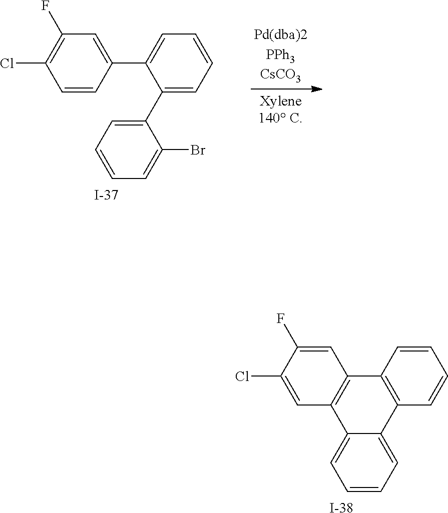

In a nitrogen environment, Intermediate I-37 (120 g, 332 mmol) was dissolved in 1.2 L of xylene, and bis(dibenzylideneacetone)palladium (0) (15.2 g, 16.6 mmol), triphenylphosphine (17.5 g, 66.6 mmol), and cesium carbonate (130 g, 400 mmol) were added thereto and then, heated under reflux at 140° C. for 24 hours. When a reaction was completed, after adding water to the reaction solution, the mixture was extracted with dichloromethane (DCM) and treated with magnesium sulfate anhydrous to remove moisture and then, filtered and concentrated under a reduced pressure. The obtained residue was separated and purified through flash column chromatography to obtain Intermediate I-38 (14.9 g, 16%).

HRMS (70 eV, EI+): m/z calcd for C18H10ClF: 280.0455, found: 280.

Elemental Analysis: C, 77%; H, 4%

### Synthesis Example 50: Synthesis of Intermediate T-39

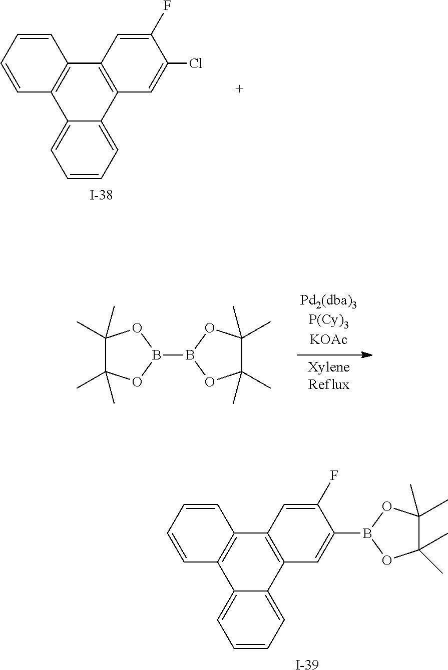

Intermediate I-39 (14.9 g, 80%) was obtained in the same manner as in Synthesis Example 6 except that Intermediate I-38 (14 g, 49.9 mmol) was used.

HRMS (70 eV, EI+): m/z calcd for C24H22BFO2: 372.1697, found: 372.

Elemental Analysis: C, 77%; H, 6%

### Synthesis Example 51: Synthesis of Intermediate I-40

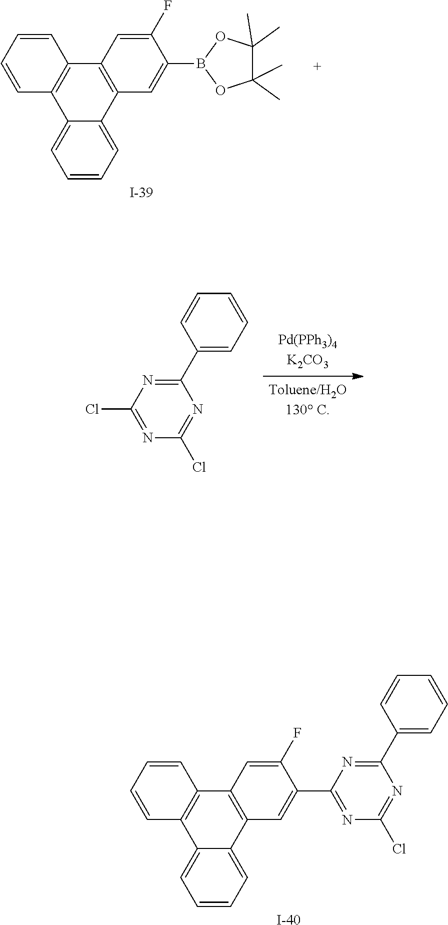

Intermediate I-40 (7.4 g, 45%) was obtained in the same manner as in Synthesis Example 1 except that Intermediate I-39 (14 g, 37.6 mmol) and 2,4-dichloro-6-phenyl-1,3,5-triazine (12.8 g, 56.4 mmol) were used.

HRMS (70 eV, EI+): m/z calcd for C27H15ClFN3: 435.0939, found: 435.

Elemental Analysis: C, 74%; H, 3%

### Synthesis Example 52: Synthesis of Intermediate I-41

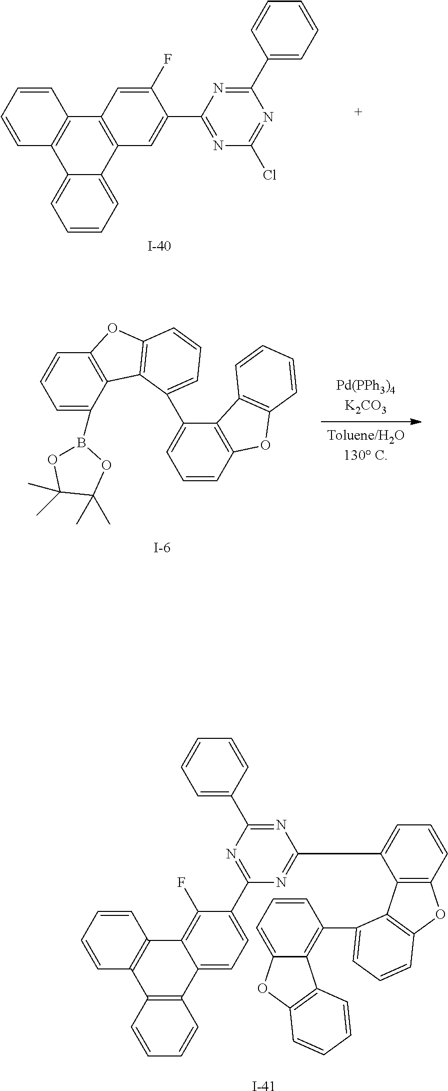

Intermediate I-41 (15.3 g, 89%) was obtained in the same manner as in Synthesis Example 1 except that Intermediate I-40 (6.5 g, 14.9 mmol) and Intermediate I-6 (8.24 g, 17.9 mmol) were used.

HRMS (70 eV, EI+): m/z calcd for C51H28FN3O2: 733.2166, found: 733.

Elemental Analysis: C, 83%; H, 4%

### Synthesis Example 53: Synthesis of Compound 45

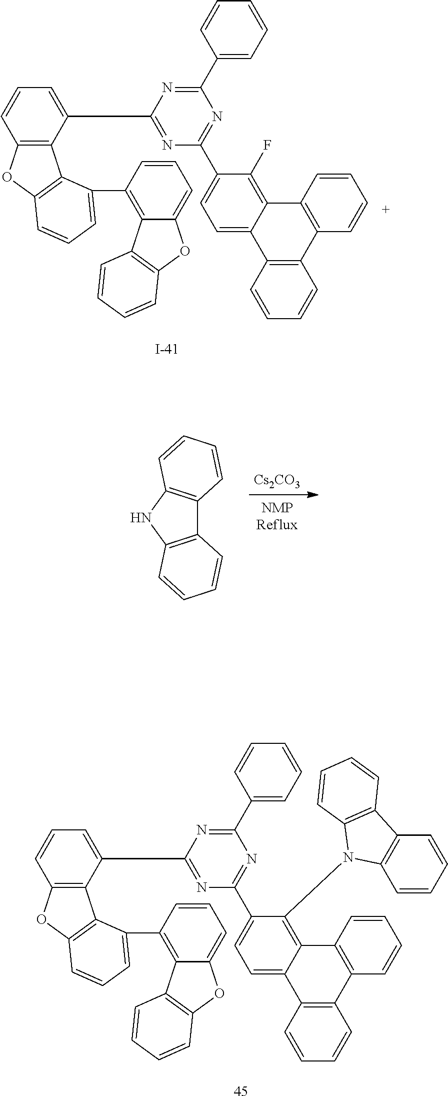

In a nitrogen environment, Intermediate I-41 (14 g, 19.1 mmol) was dissolved in 0.2 L of N-methyl-2-pyrrolidone (NMP), and 9H-carbazole (3.51 g, 21.0 mmol) and cesium carbonate (12.4 g, 38.2 mmol) were added thereto and heated under reflux for 24 hours. When a reaction was completed, after distilling and removing the solvent and then, adding water to the reaction solution, the mixture was extracted with dichloromethane (DCM) and treated with magnesium sulfate anhydrous to remove moisture and then, filtered and concentrated under a reduced pressure. The obtained residue was separated and purified through flash column chromatography to obtain Compound 45 (11.8 g, 70%).

HRMS (70 eV, EI+): m/z calcd for C63H36N4O2: 880.2838, found: 880.

Elemental Analysis: C, 86%; H, 5%

### Synthesis Example 54: Synthesis of Intermediate I-42

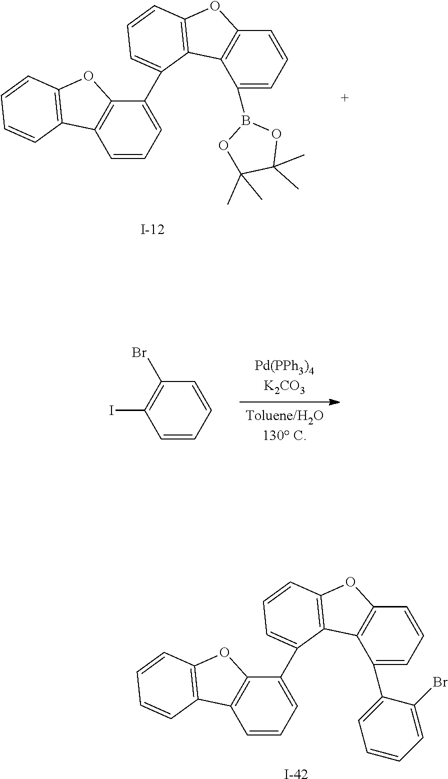

Intermediate I-42 (48.5 g, 91%) was obtained in the same manner as in Synthesis Example 1 except that Intermediate I-12 (50 g, 109 mmol) and 1-bromo-2-iodobenzene (46.1 g, 163 mmol) were used.

HRMS (70 eV, EI+): m/z calcd for C30H17BrO2: 488.0412, found: 488.

### Elemental Analysis: C, 74%; H, 4%

### Synthesis Example 55: Synthesis of Intermediate I-43

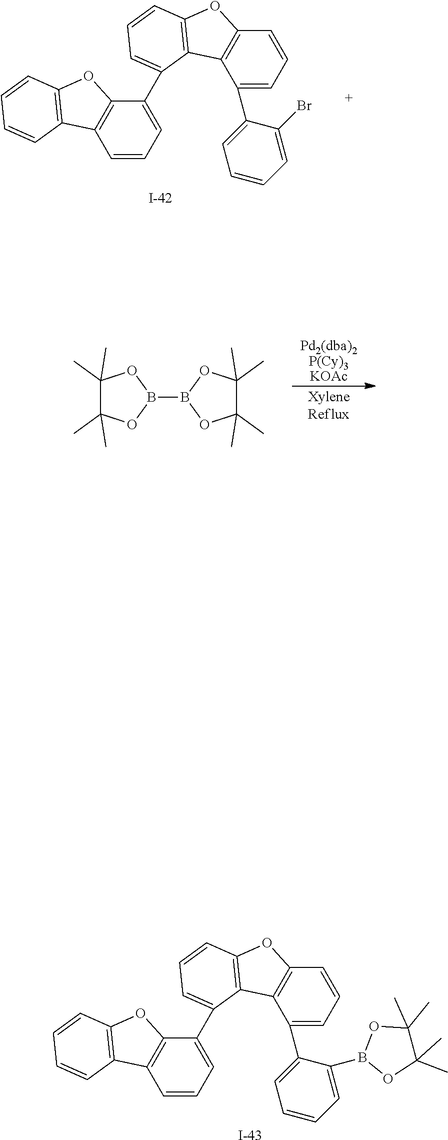

Intermediate I-43 (39.5 g, 75%) was obtained in the same manner as in Synthesis Example 6 except that Intermediate I-42 (14 g, 98.1 mmol) was used.

HRMS (70 eV, EI+): m/z calcd for C36H29BO4: 536.2159, found: 536.

Elemental Analysis: C, 81%; H, 5%

### Synthesis Example 56: Synthesis of Compound 69

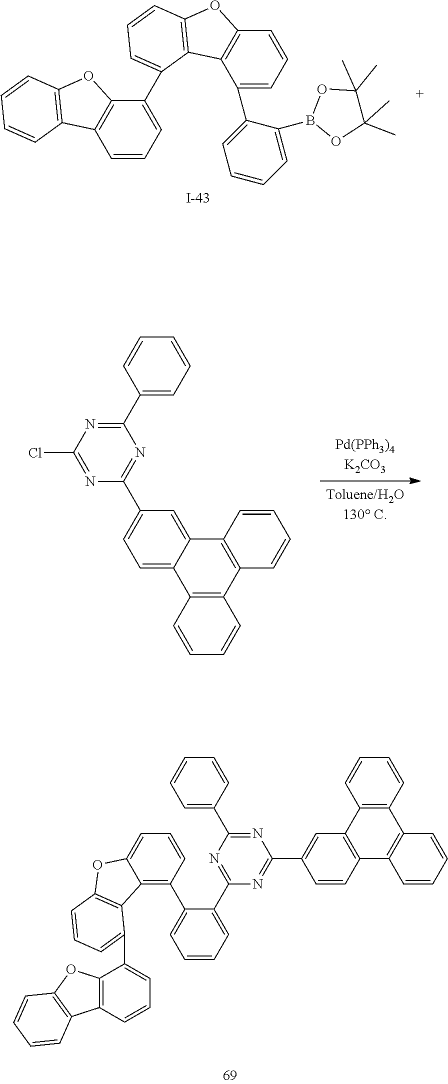

Compound 69 (20.7 g, 70%) was obtained in the same manner as in Synthesis Example 1 except that Intermediate I-43 (20 g, 37.3 mmol) and 2-chloro-4-phenyl-6-(triphenylen-2-yl)-1,3,5-triazine (15.6 g, 37.3 mmol) were used.

HRMS (70 eV, EI+): m/z calcd for C57H33N3O2: 791.2573, found: 791.

Elemental Analysis: C, 86%; H, 4%

### Synthesis Example 57: Synthesis of Intermediate I-44

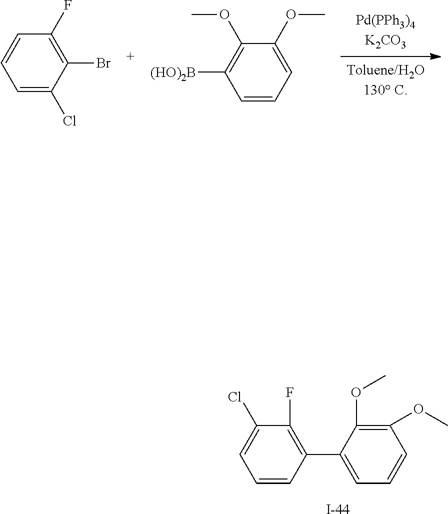

Intermediate I-44 (121 g, 95%) was obtained in the same manner as in Synthesis Example 1 except that 2-bromo-1-chloro-3-fluorobenzene (100 g, 477 mmol) and 2,3-dimethoxyphenylboronic acid (95.6 g, 525 mmol) were used.

HRMS (70 eV, EI+): m/z calcd for C14H12ClFO2: 266.0510, found: 266.

Elemental Analysis: C, 63%; H, 5%

### Synthesis Example 58: Synthesis of Intermediate I-45

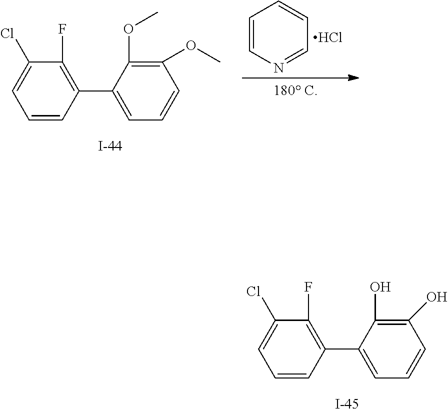

Intermediate I-45 (106 g, 99%) was obtained in the same manner as in Synthesis Example 2 except that Intermediate I-44 (120 g, 450 mmol) was used.

HRMS (70 eV, EI+): m/z calcd for C12H8ClFO2: 238.0197, found: 238.

Elemental Analysis: C, 60%; H, 3%

### Synthesis Example 59: Synthesis of Intermediate I-46

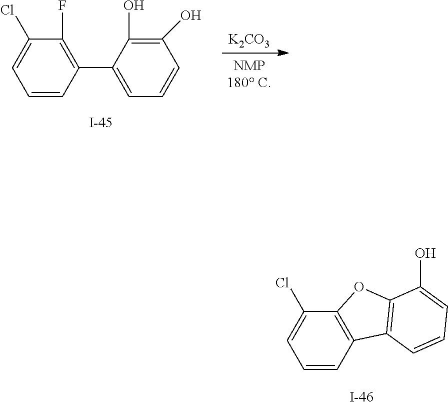

Intermediate I-46 (67.3 g, 70%) was obtained in the same manner as in Synthesis Example 3 except that Intermediate I-45 (105 g, 440 mmol) was used.

HRMS (70 eV, EI+): m/z calcd for C12H7ClO2: 218.0135, found: 218.

Elemental Analysis: C, 66%; H, 3%

### Synthesis Example 60: Synthesis of Intermediate I-47

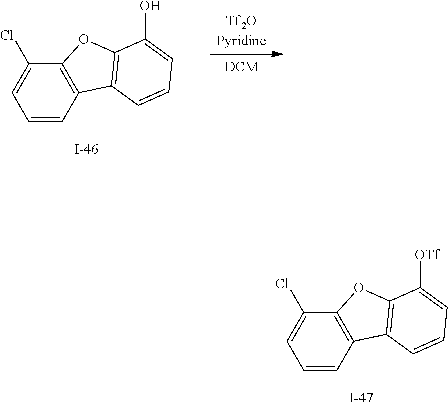

Intermediate I-47 (103 g, 99%) was obtained in the same manner as in Synthesis Example 4 except that Intermediate I-46 (65 g, 297 mmol) was used.

HRMS (70 eV, EI+): m/z calcd for C13H6ClF3O4S: 349.9627, found: 349.

### Elemental Analysis: C, 45%; H, 2%

### Synthesis Example 61: Synthesis of Intermediate I-48

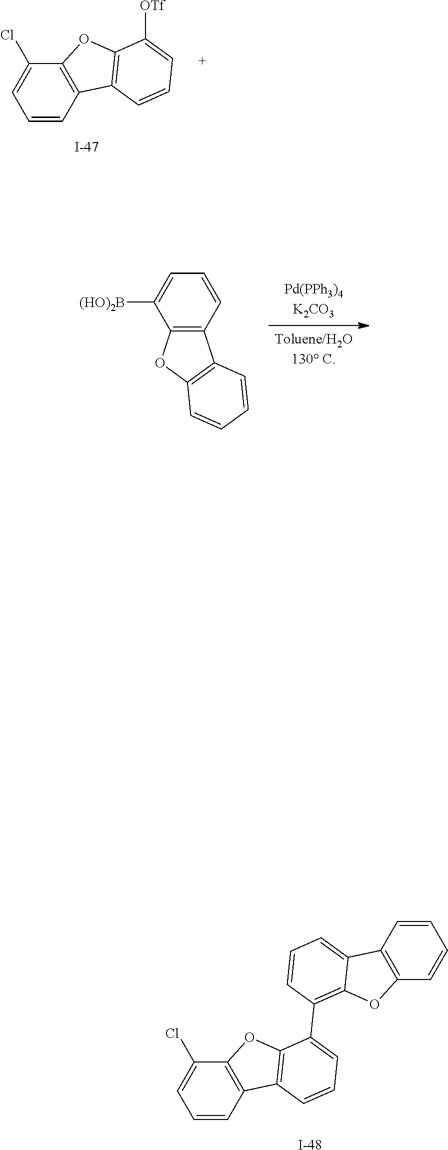

Intermediate I-48 (76.7 g, 73%) was obtained in the same manner as in Synthesis Example 1 except that Intermediate I-47 (100 g, 285 mmol) and dibenzofuran-4-boronic acid (66.5 g, 314 mmol) were used.

HRMS (70 eV, EI+): m/z calcd for C24H13ClO2: 368.0604, found: 368.

Elemental Analysis: C, 78%; H, 4%

### Synthesis Example 62: Synthesis of Intermediate I-49

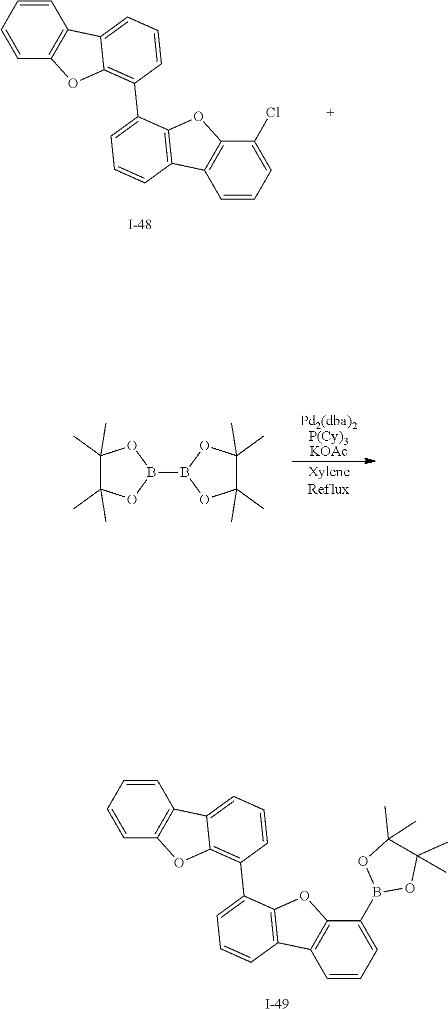

Intermediate I-49 (47.7 g, 51%) was obtained in the same manner as in Synthesis Example 6 except that Intermediate I-48 (75 g, 203 mmol) was used.

HRMS (70 eV, EI+): m/z calcd for C30H25BO4: 460.1846, found: 460.

Elemental Analysis: C, 78%; H, 5%

### Synthesis Example 63: Synthesis of Compound 225

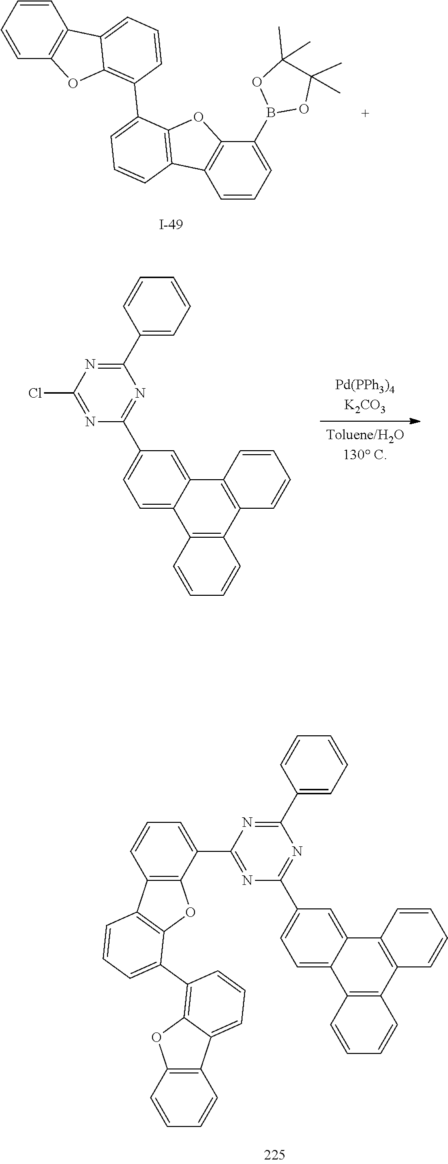

Compound 225 (27.3 g, 88%) was obtained in the same manner as in Synthesis Example 1 except that Intermediate I-49 (20 g, 43.4 mmol) and 2-chloro-4-phenyl-6-(triphenylen-2-yl)-1,3,5-triazine (18.2 g, 43.4 mmol) purchased from P&H Tech were used.

HRMS (70 eV, EI+): m/z calcd for C51H29N3O2: 715.2260, found: 715.

Elemental Analysis: C, 86%; H, 4%

### Synthesis Example 64: Synthesis of Intermediate I-50

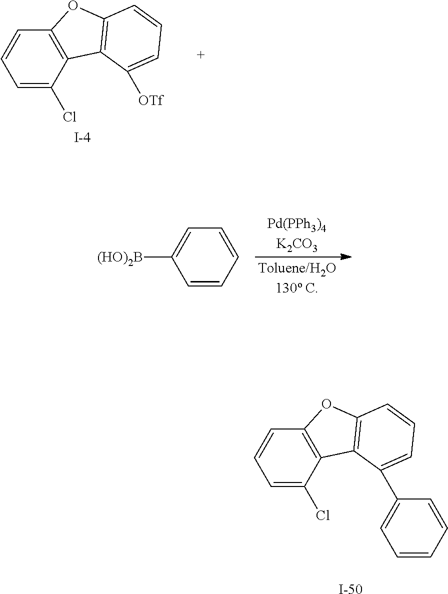

Intermediate I-50 (67.5 g, 85%) was obtained in the same manner as in Synthesis Example 1 except that Intermediate I-4 (100 g, 285 mmol) and phenylboronic acid (41.7 g, 342 mmol) were used.

HRMS (70 eV, EI+): m/z calcd for C18H11C10: 278.0498, found: 278.

Elemental Analysis: C, 78%; H, 4%

### Synthesis Example 65: Synthesis of Intermediate I-51

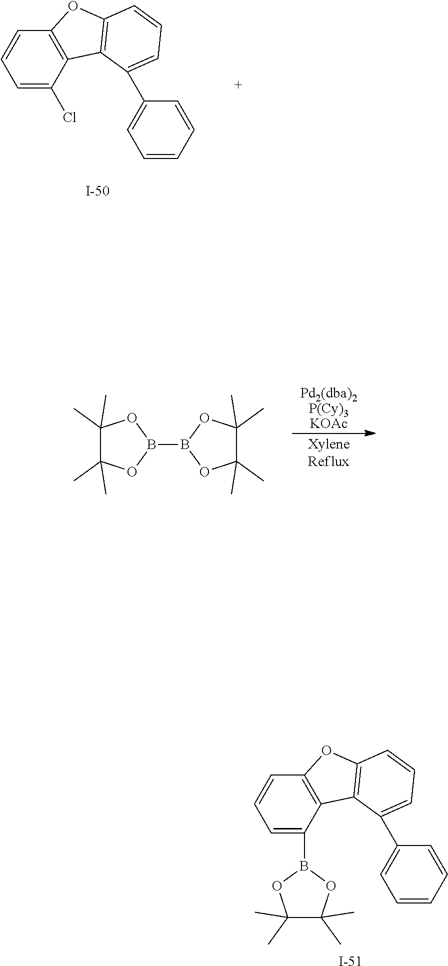

Intermediate I-51 (46.6 g, 54%) was obtained in the same manner as in Synthesis Example 6 except that Intermediate I-50 (65 g, 233 mmol) was used.

HRMS (70 eV, EI+): m/z calcd for C24H23BO3: 370.1740, found: 370.

Elemental Analysis: C, 78%; H, 6%

### Synthesis Example 66: Synthesis of Intermediate I-52

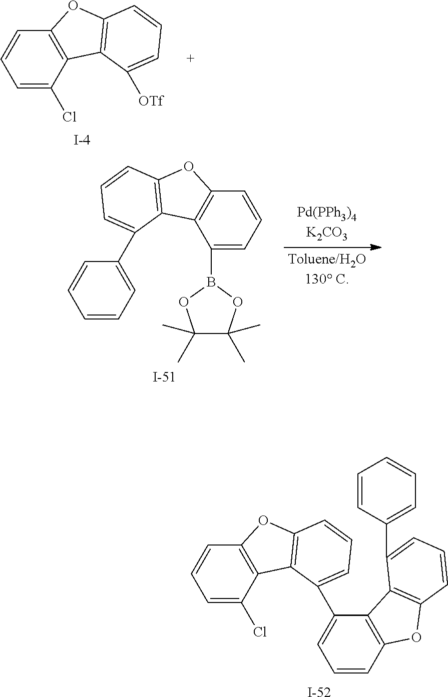

Intermediate I-52 (64.7 g, 51%) was obtained in the same manner as in Synthesis Example 1 except that Intermediate I-4 (100 g, 285 mmol) and Intermediate I-51 (116 g, 314 mmol) were used.

HRMS (70 eV, EI+): m/z calcd for C30H17ClO2: 444.0917, found: 444.

Elemental Analysis: C, 81%; H, 4%

### Synthesis Example 67: Synthesis of Intermediate I-53

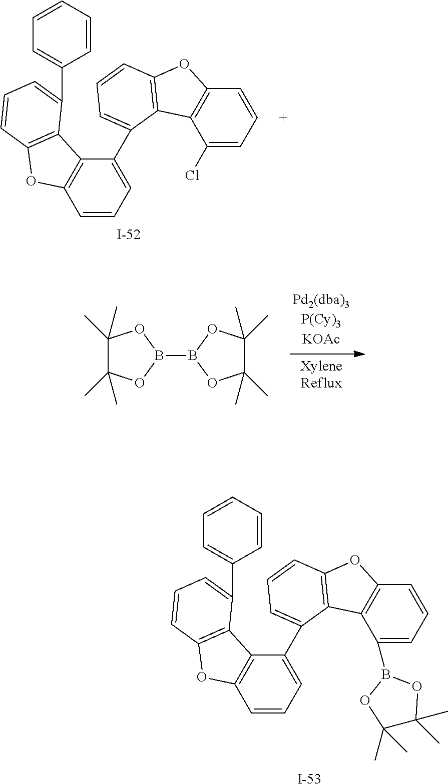

Intermediate I-53 (36.5 g, 48%) was obtained in the same manner as in Synthesis Example 6 except that Intermediate I-52 (63 g, 142 mmol) was used.

HRMS (70 eV, EI+): m/z calcd for C36H29BO4: 536.2159, found: 536.

Elemental Analysis: C, 81%; H, 5%

### Synthesis Example 68: Synthesis of Compound 57

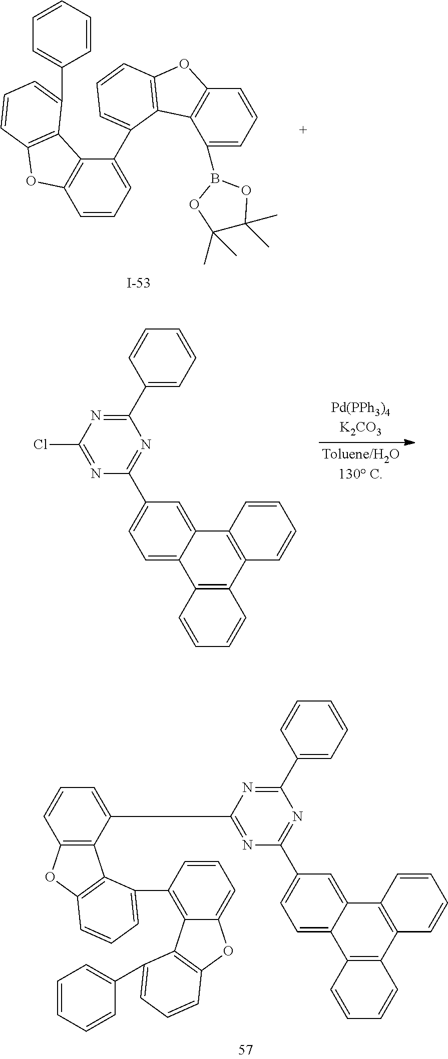

Compound 57 (25.1 g, 85%) was obtained in the same manner as in Synthesis Example 1 except that Intermediate I-53 (20 g, 37.3 mmol) and 2-chloro-4-phenyl-6-(triphenylen-2-yl)-1,3,5-triazine (15.6 g, 37.3 mmol) purchased from P&H Tech were used.

HRMS (70 eV, EI+): m/z calcd for C57H33N3O2: 791.2573, found: 791.

Elemental Analysis: C, 86%; H, 4%

### Synthesis Example 69: Synthesis of Intermediate I-54

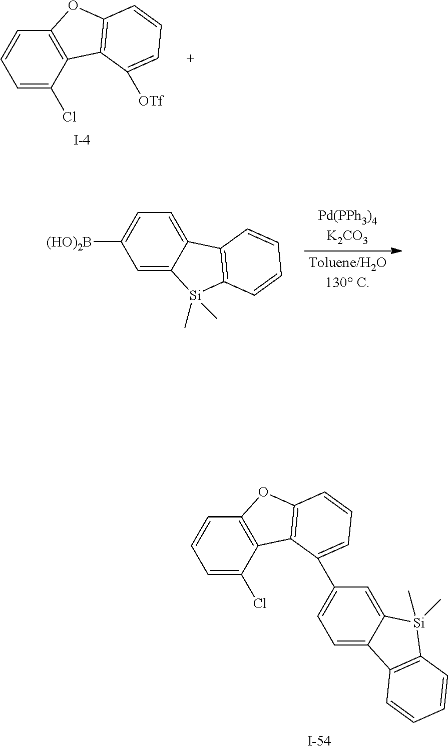

Intermediate I-54 (44.1 g, 75%) was obtained in the same manner as in Synthesis Example 1 except that Intermediate I-4 (50 g, 143 mmol) and 5,5-dimethyl-5H-dibenzosilol-3-ylboronic acid (39.9 g, 157 mmol) were used.

HRMS (70 eV, EI+): m/z calcd for C26H19ClOSi: 410.0894, found: 410.

Elemental Analysis: C, 76%; H, 5%

### Synthesis Example 70: Synthesis of Intermediate I-55

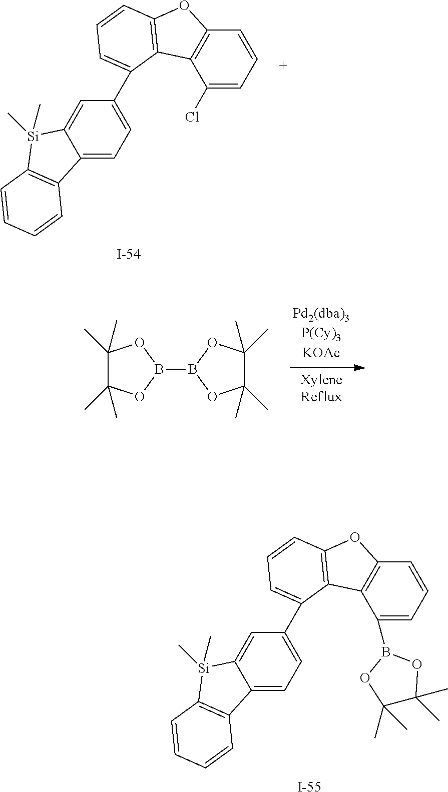

Intermediate I-55 (26.3 g, 50%) was obtained in the same manner as in Synthesis Example 6 except that Intermediate I-54 (43 g, 105 mmol) was used.

HRMS (70 eV, EI+): m/z calcd for C32H31BO3Si: 502.2136, found: 502.

Elemental Analysis: C, 76%; H, 6%

### Synthesis Example 71: Synthesis of Compound 119

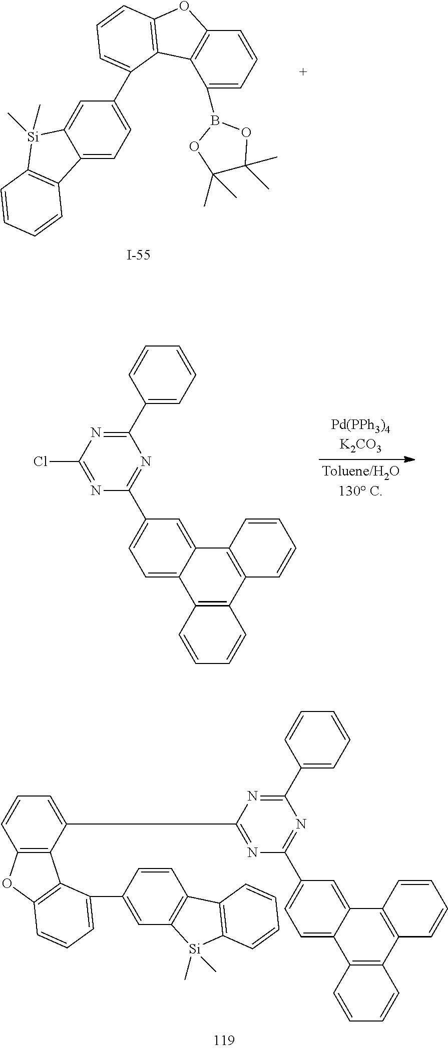

Compound 119 (2.4 g, 81%) was obtained in the same manner as in Synthesis Example 1 except that Intermediate I-55 (20 g, 39.8 mmol) and 2-chloro-4-phenyl-6-(triphenylen-2-yl)-1,3,5-triazine (16.6 g, 39.8 mmol) purchased from P&H Tech were used.

HRMS (70 eV, EI+): m/z calcd for C53H35N3OSi: 757.2549, found: 757.

Elemental Analysis: C, 84%; H, 5%

### Synthesis Example 72: Synthesis of Intermediate I-56

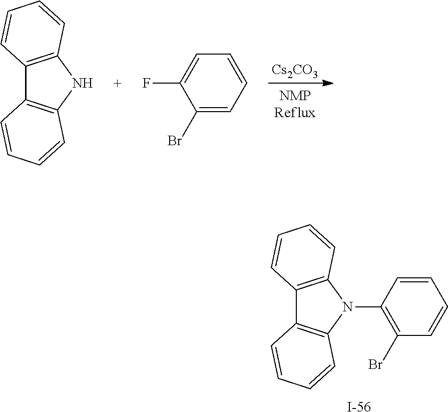

Intermediate I-56 (150 g, 78%) was obtained in the same manner as in Synthesis Example 53 except that 9H-carbazole (100 g, 598 mmol) and 1-bromo-2-fluorobenzene (115 g, 658 mmol) were used.

HRMS (70 eV, EI+): m/z calcd for C18H12BrN: 321.0153, found: 321.

Elemental Analysis: C, 67%; H, 4%

### Synthesis Example 73: Synthesis of Intermediate T-57

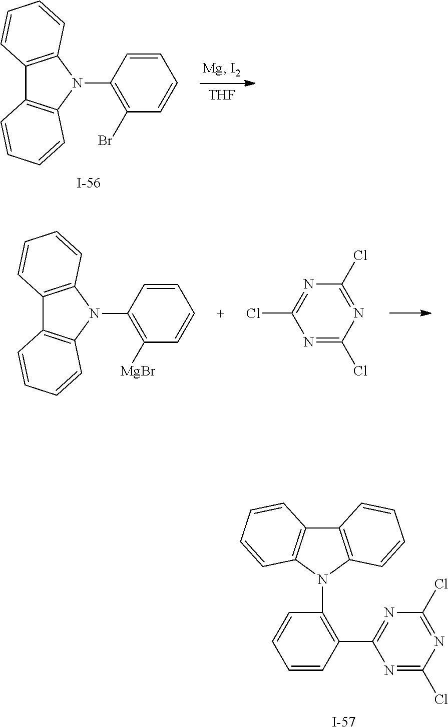

In a nitrogen environment, magnesium (10.5 g, 435 mmol) and iodine (2.21 g, 8.7 mmol) were dissolved in 0.1 L of tetrahydrofuran (THF) and then, stirred for 30 minutes. Subsequently, Intermediate I-56 (140 g, 435 mmol) dissolved in 0.1 L of THF was slowly added thereto for 30 minutes. A grignard reagent prepared in this way was slowly added to a solution prepared by dissolving cyanuric chloride (96.3 g, 522 mmol) purchased in Tokyo Chemical Industry Co., Ltd. in 1 L of THF for 30 minutes and then, stirred for 3 hours. When a reaction was completed, after adding water to the reaction solution, the mixture was extracted with dichloromethane (DCM) and treated with magnesium sulfate anhydrous to remove moisture and then, filtered and concentrated under a reduced pressure. The obtained residue was separated and purified through flash column chromatography to obtain Intermediate I-57 (73.2 g, 43%).

HRMS (7 eV, EI+): m/z calcd for C21H12C12N4: 390.0439, found: 390.

Elemental Analysis: C, 64%; H, 3%

### Synthesis Example 74: Synthesis of Intermediate I-58

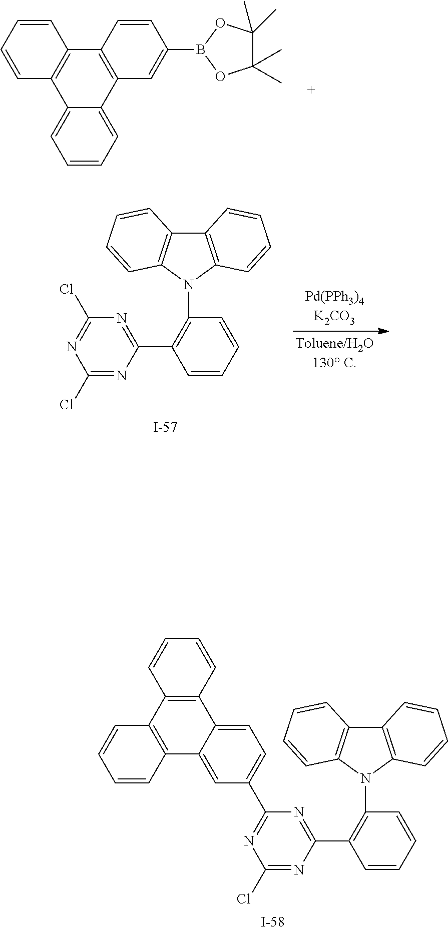

Intermediate I-58 (30.9 g, 50%) was obtained in the same manner as in Synthesis Example 1 except that 4,4,5,5-tetramethyl-2-(triphenylen-2-yl)-1,3,2-dioxaborolane (37.7 g, 106 mmol) and Intermediate I-57 (50 g, 128 mmol) were used.

HRMS (70 eV, EI+): m/z calcd for C39H23C1N4: 582.1611, found: 582.

Elemental Analysis: C, 80%; H, 4%

### Synthesis Example 75: Synthesis of Compound 181

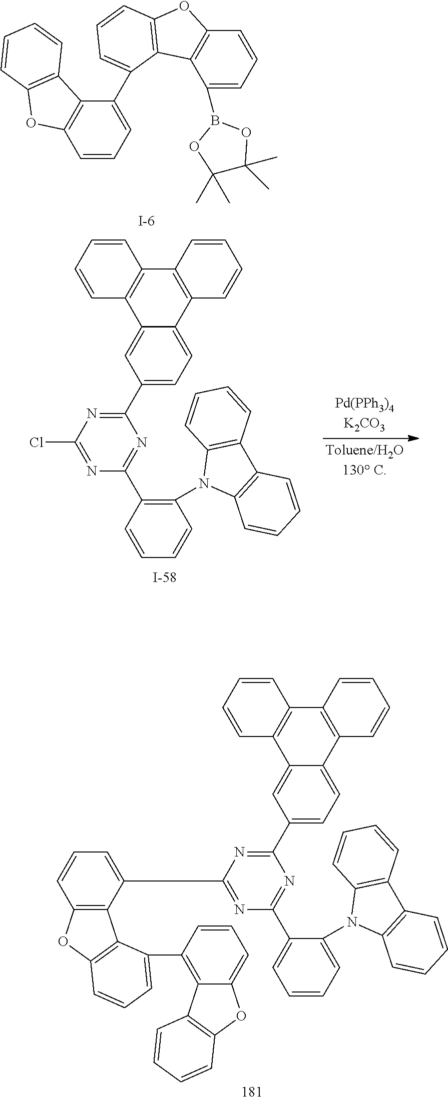

Compound 181 (30.6 g, 80%) was obtained in the same manner as in Synthesis Example 1 except that Intermediate I-6 (20 g, 43.4 mmol) and Intermediate I-58 (25.3 g, 43.4 mmol) were used.

HRMS (70 eV, EI+): m/z calcd for C63H36N4O2: 880.2838, found: 880.

Elemental Analysis: C, 86%; H, 4%

### Synthesis Example 76: Synthesis of Intermediate I-59

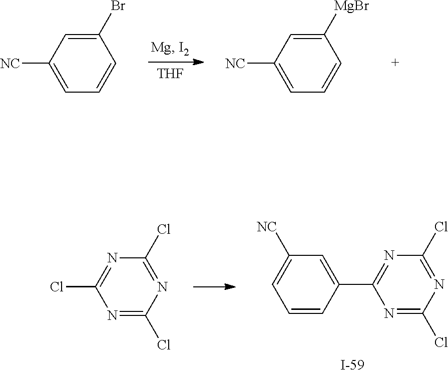

Intermediate I-59 (31.1 g, 45%) was obtained in the same manner as in Synthesis Example 73 except that 3-bromobenzonitrile (50 g, 275 mmol) was used.

HRMS (70 eV, EI+): m/z calcd for C10H4C12N4: 249.9813, found: 249.

Elemental Analysis: C, 48%; H, 1%

### Synthesis Example 77: Synthesis of Intermediate I-60

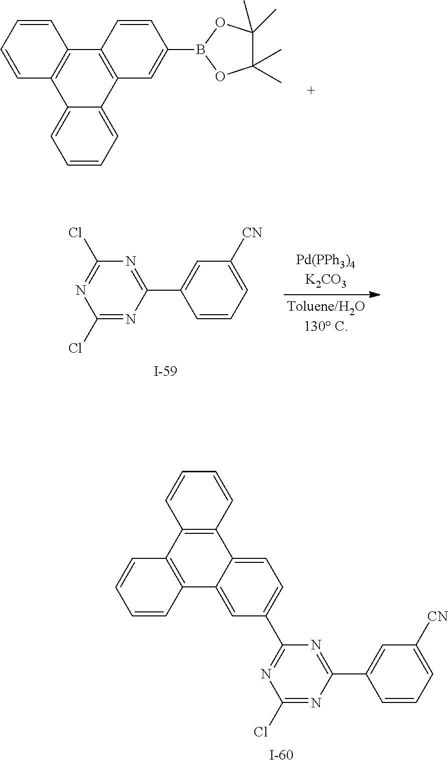

Intermediate I-60 (17.2 g, 39%) was obtained in the same manner as in Synthesis Example 1 except that 4,4,5,5-tetramethyl-2-(triphenylen-2-yl)-1,3,2-dioxaborolane (35.3 g, 99.6 mmol) and Intermediate I-59 (30 g, 119 mmol) were used.

HRMS (70 eV, EI+): m/z calcd for C28H15C1N4: 442.0985, found: 442.

Elemental Analysis: C, 76%; H, 3%

### Synthesis Example 78: Synthesis of Compound 221

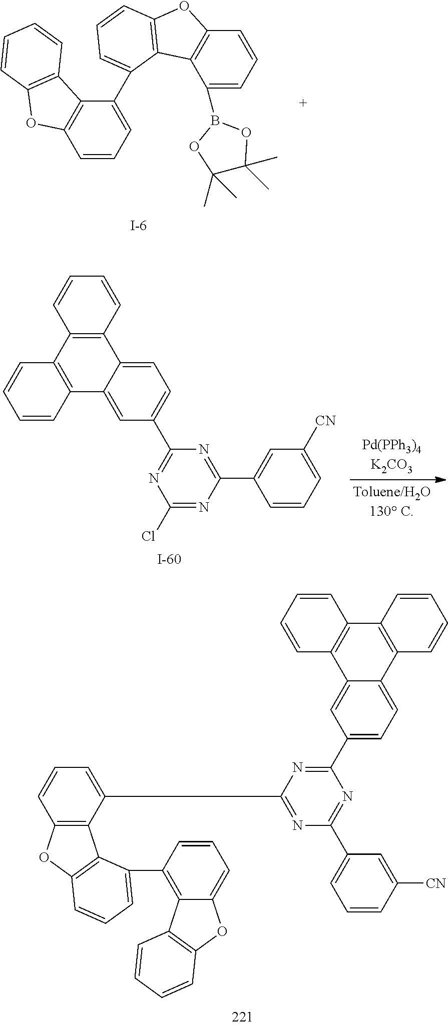

Compound 221 (26.7 g, 83%) was obtained in the same manner as in Synthesis Example 1 except that Intermediate I-6 (20 g, 43.4 mmol) and Intermediate I-60 (19.2 g, 43.4 mmol) were used.

HRMS (70 eV, EI+): m/z calcd for C52H28N4O2: 740.2212, found: 740.

Elemental Analysis: C, 84%; H, 4%

### Synthesis Example 79: Synthesis of Compound Host 1

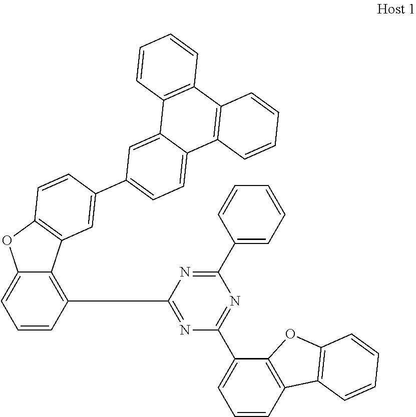 [SMILES: c1ccc(-c2nc(CCc3cccc4oc5ccc(-c6ccc7c8ccccc8c8ccccc8c7c6)cc5c34)nc(-c3cccc4c3oc3ccccc34)n2)cc1]

Compound Host 1 was synthesized by referring to the synthetic method of patent KR1959821.

HRMS (70 eV, EI+): m/z calcd for C51H29N3O2: 715.2260, found: 715.

Elemental Analysis: C. 86%: H, 4%

### Synthesis Example 80: Synthesis of Compound Host 2

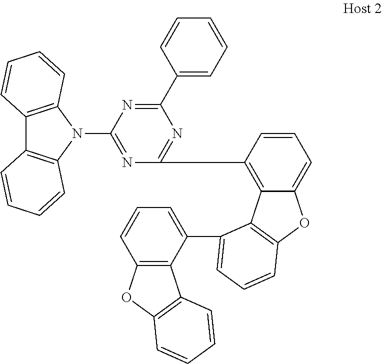 [SMILES: c1ccc(-c2nc(-c3cccc4oc5cccc(-c6cccc7oc8ccccc8c67)c5c34)nc(-n3c4ccccc4c4ccccc43)n2)cc1]

Compound Host 2 was synthesized by referring to the synthetic method of patent US20200377489.

HRMS (70 eV, EI+): m/z calcd for C45H26N4O2: 654.2056, found: 654.

Elemental Analysis: C, 83%; H, 4%

### Synthesis Example 81: Synthesis of Compound Host 3

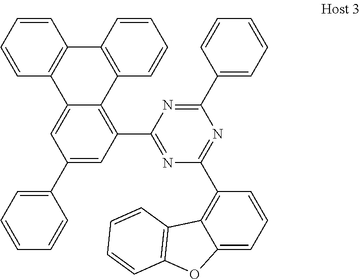 [SMILES: c1ccc(-c2cc(-c3nc(-c4ccccc4)nc(-c4cccc5oc6ccccc6c45)n3)c3c4ccccc4c4ccccc4c3c2)cc1]

Compound Host 3 was synthesized by referring to the synthetic method of patent KR2019135398.

HRMS (70 eV, EI+): m/z calcd for C45H27N3O: 625.2154, found: 625.

Elemental Analysis: C, 86%; H, 4%

### Synthesis Example 82: Synthesis of Compound Host 4

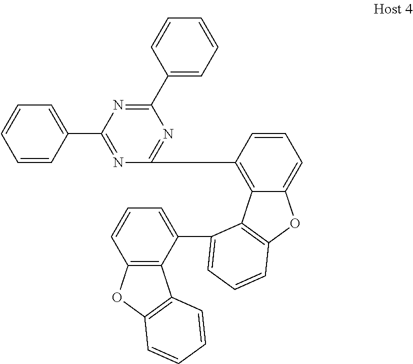 [SMILES: c1ccc(-c2nc(-c3ccccc3)nc(-c3cccc4oc5cccc(-c6cccc7oc8ccccc8c67)c5c34)n2)cc1]

Compound Host 4 was synthesized by referring to the synthetic method of patent WO2021182893.

HRMS (70 eV, EI+): m/z calcd for C39H23N3O2: 565.1790, found: 565.

Elemental Analysis: C, 83%; H, 4%

### Synthesis Example 83: Synthesis of Compound Host 5

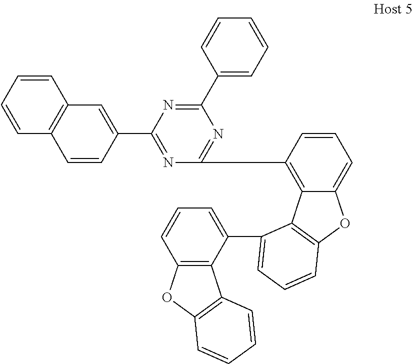 [SMILES: c1ccc(-c2nc(-c3ccc4ccccc4c3)nc(-c3cccc4oc5cccc(-c6cccc7oc8ccccc8c67)c5c34)n2)cc1]

Compound Host 5 was synthesized by referring to the synthetic method of patent WO2021182893.

HRMS (70 eV, EI+): m/z calcd for C43H25N3O2: 615.6775, found: 615.

Elemental Analysis: C, 84%; H, 4%

### Synthesis Example 84: Synthesis of Compound Host 6

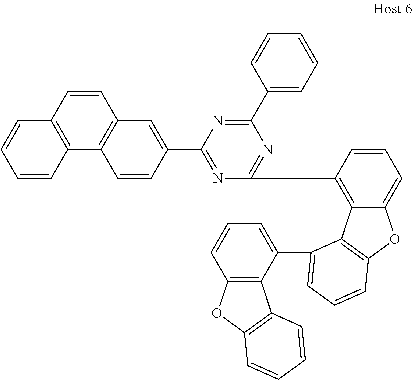 [SMILES: c1ccc(-c2nc(-c3ccc4c(ccc5ccccc54)c3)nc(-c3cccc4oc5cccc(-c6cccc7oc8ccccc8c67)c5c34)n2)cc1]

Compound Host 6 was synthesized by referring to the synthetic method of patent WO2021182893.

HRMS (70 eV, EI+): m/z calcd for C47H27N3O2: 665.2103, found: 665.

Elemental Analysis: C, 85%; H, 4%

### Synthesis Example 85: Synthesis of Compound Host 7

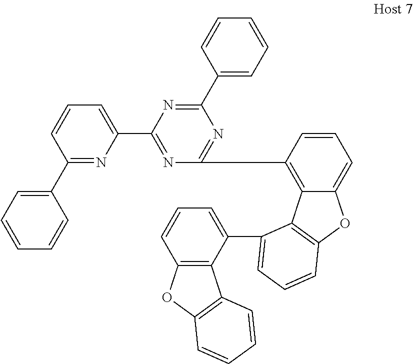 [SMILES: c1ccc(-c2cccc(-c3nc(-c4ccccc4)nc(-c4cccc5oc6cccc(-c7cccc8oc9ccccc9c78)c6c45)n3)n2)cc1]

Compound Host 7 was synthesized by referring to the synthetic method of patent WO2021182893.

HRMS (70 eV, EI+): m/z calcd for C45H27N3O2: 641.2103, found: 641.

Elemental Analysis: C, 84%; H, 4%

### Synthesis Example 86: Synthesis of Compound B-136

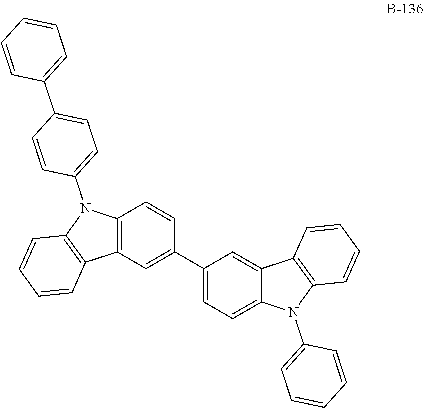 [SMILES: c1ccc(-c2ccc(-n3c4ccccc4c4cc(-c5ccc6c(c5)c5ccccc5n6-c5ccccc5)ccc43)cc2)cc1]

Compound B-136 was synthesized by referring to the synthetic method of patent EP3034581.

HRMS (70 eV, EI+): m/z calcd for C42H28N2: 560.2252, found: 560.

Elemental Analysis: C, 90%; H, 5%

### Synthesis Example 87: Synthesis of Compound B-99

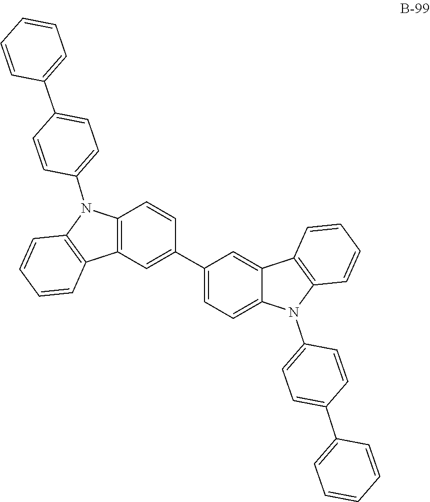 [SMILES: c1ccc(-c2ccc(-n3c4ccccc4c4cc(-c5ccc6c(c5)c5ccccc5n6-c5ccc(-c6ccccc6)cc5)ccc43)cc2)cc1]

Compound B-99 was synthesized by referring to the synthetic method of patent KR10-2019-0000597.

HRMS (70 eV, EI+): m/z calcd for C48H32N2: 636.2565, found: 636.

Elemental Analysis: C, 91%; H, 5%

### Synthesis Example 88: Synthesis of Compound B-31

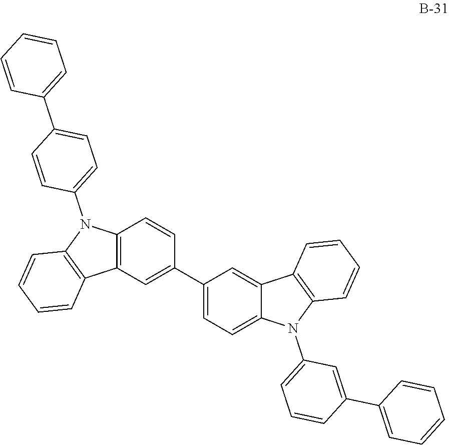 [SMILES: c1ccc(-c2ccc(-n3c4ccccc4c4cc(-c5ccc6c(c5)c5ccccc5n6-c5cccc(-c6ccccc6)c5)ccc43)cc2)cc1]

Compound B-31 was synthesized by referring to the synthetic method of patent EP2947071.

HRMS (70 eV, EI+): m/z calcd for C48H32N2: 636.2565, found: 636.

Elemental Analysis: C, 91%; H, 5%

### Synthesis Example 89: Synthesis of Compound C-4

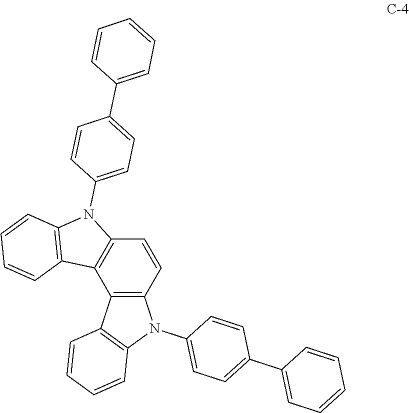 [SMILES: c1ccc(-c2ccc(-n3c4ccccc4c4c5c6ccccc6n(-c6ccc(-c7ccccc7)cc6)c5ccc43)cc2)cc1]

Compound C-4 was synthesized by referring to the synthetic method of patent KR2031300.

HRMS (70 eV, EI+): m/z calcd for C42H28N2: 560.2252, found: 560.

Elemental Analysis: C, 90%; H, 5%

### Synthesis Example 90: Synthesis of Compound C-57

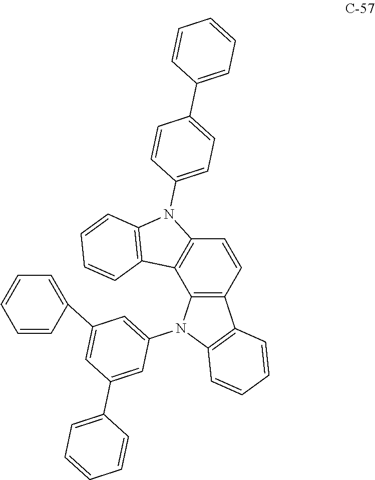 [SMILES: c1ccc(-c2ccc(-n3c4ccccc4c4c3ccc3c5ccccc5n(-c5cc(-c6ccccc6)cc(-c6ccccc6)c5)c34)cc2)cc1]

Compound C-57 was synthesized by referring to the synthetic method of patent WO2018-095391.

HRMS (70 eV, EI+): m/z calcd for C48H₃₂N₂: 636.2565, found: 636.

Elemental Analysis: C, 91%; H, 5%

### Example 1

A glass substrate coated with a thin film of ITO (indium tin oxide) was ultrasonically cleaned with distilled water. After washing with the distilled water, the glass substrate was washed with a solvent such as isopropyl alcohol, acetone, methanol, and the like ultrasonically and dried and then, moved to a plasma cleaner, cleaned by using oxygen plasma for 10 minutes, and moved to a vacuum depositor. This prepared ITO transparent electrode was used as an anode, Compound A doped with 3% NDP-9 (Novaled GmbH) was vacuum-deposited on the ITO substrate to form a 100 Å-thick hole injection layer, and Compound A is deposited on the hole injection layer to form a 1350 Å-thick hole transport layer. Compound B was deposited on the hole transport layer to form a 350 Å-thick hole transport auxiliary layer, and Compound 1 synthesized in Synthesis Example 7 was used as a host and PhGD was doped at 7 wt % as a dopant on the hole transport auxiliary layer to form a 400 Å-thick light emitting layer by vacuum deposition. The ratio is described separately for the following examples and comparative examples. Subsequently, on the light emitting layer, Compound C was deposited to form a 50 Å-thick electron transport auxiliary layer, and Compound D and Liq in a weight ratio of 1:1 were simultaneously vacuum-deposited to form a 300 Å-thick electron transport layer. On the electron transport layer, a cathode was formed by sequentially vacuum-depositing 15 Å of LiQ and 1,200 Å of Al, manufacturing an organic light emitting diode.

The organic light emitting diode was manufactured to have a structure of ITO/Compound A (3% NDP-9 doping, 100 Å)/Compound A (1350 Å)/Compound B (350 Å)/EML [Compound 1 (93 wt %): PhGD (7 wt %)](400 Å)/Compound C (50 Å)/Compound D: LiQ (300 Å)/LiQ (15 Å)/Al (1200A).
Compound A: N-(biphenyl-4-yl)-9,9-dimethyl-N-(4-(9-phenyl-9H-carbazol-3-yl)phenyl)-9H-fluoren-2-amine
Compound B: N-[4-(4-dibenzofuranyl)phenyl]-N-[4-(9-phenyl-9H-fluoren-9-yl)phenyl][1,1′-biphenyl]-4-amine
Compound C: 2,4-diphenyl-6-(4′, 5′, 6′-triphenyl[1,1′: 2′,1″: 3″,1′″:3′″,1″″-quinquephenyl]-3″″-yl)-1,3,5-triazine

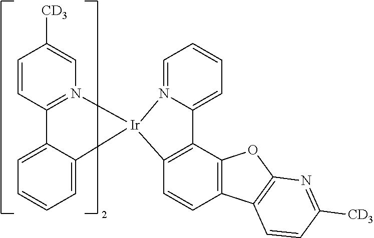

### Examples 2 to 18 and Comparative Examples 1 to 7

Each organic light emitting diode was manufactured in the same manner as in Example 1 except that the composition was changed as described in Table 1.

### Example 19

A glass substrate coated with a thin film of ITO (indium tin oxide) was ultrasonically cleaned with distilled water. After washing with the distilled water, the glass substrate was washed with a solvent such as isopropyl alcohol, acetone, methanol, and the like ultrasonically and dried and then, moved to a plasma cleaner, cleaned by using oxygen plasma for 10 minutes, and moved to a vacuum depositor. This prepared ITO transparent electrode was used as an anode, Compound A doped with 3% NDP-9 (Novaled GmbH) was vacuum-deposited on the ITO substrate to form a 100 Å-thick hole injection layer, and Compound A is deposited on the hole injection layer to a thickness of 1350 Å to form a hole transport layer. Compound E was deposited on the hole transport layer to a thickness of 350 Å to form a hole transport auxiliary layer. On the hole transport auxiliary layer, Compound 1 of Synthesis Example 7 and Compound B-136 of Synthesis Example 86, which were simultaneously used as a host and doped with 10 wt % of PhGD as a dopant, were vacuum-deposited to form a 400 Å-thick light emitting layer. Here, Compound 1 and Compound B-136 were used in a weight ratio of 3:7. Subsequently, on the light emitting layer, Compound F was deposited to form a 50 Å-thick electron transport auxiliary layer, and a 300 Å-thick electron transport layer was formed thereon by vacuum-depositing Compound G and Liq in a weight ratio of 1:1, simultaneously. On the electron transport layer, a cathode was formed by sequentially vacuum-depositing 15 Å of LiQ and 1200 Å of Al, manufacturing an organic light emitting diode.

The organic light emitting diode was manufactured to have a structure of ITO/Compound A (3% NDP-9 doping, 100 Å)/Compound A (1350 Å)/Compound E (350 Å)/EML [{host=Compound 1:Compound B-136: dopant=PhGD}=27:63:10 (wt %)](400 Å)/Compound F (50 Å)/Compound G:LiQ (300 Å)/LiQ (15 Å)/Al (1200 Å).
Compound A: N-(biphenyl-4-yl)-9,9-dimethyl-N-(4-(9-phenyl-9H-carbazol-3-yl)phenyl)-9H-fluoren-2-amine
Compound E: N,N-bis(9,9-dimethyl-9H-fluoren-4-yl)-9,9-spirobi(fluorene)-2-amine
Compound F: 2-[3′-(9,9-dimethyl-9H-fluoren-2-yl)[1,1′-biphenyl]-3-yl]-4,6-diphenyl-1,3,5-triazine
Compound G: 2-[4-[4-(4′-cyano-1,1′-biphenyl-4-yl)-1-naphthyl]phenyl]-4,6-diphenyl-1,3,5-triazine

### Examples 20 to 42 and Comparative Examples 8 to 14

Each organic light emitting diode was manufactured in the same manner as in Example 19 except that the composition was changed into each composition shown in Table 2.

### Evaluation

The organic light emitting diodes of Examples 1 to 42 and Comparative Examples 1 to 14 were evaluated with respect to a driving voltage, luminous efficiency, and life-span characteristics.

Specific measuring methods are as follows, and the results are shown in Tables 1 and 2.

(1) Measurement of Current Density Change according to Voltage Change

The manufactured organic light emitting diodes were measured with respect to a current flowing through a unit device by using a current-voltage meter (Keithley 2400), while increasing a voltage from 0 V to 10 V, and the measured current value was divided by an area to provide the results.

### (2) Measurement of Luminance Change Depending on Voltage Change

Luminance was measured by using a luminance meter (Minolta Cs-1000A), while increasing the voltage of the organic light emitting diodes from 0 V to 10 V.

### (3) Measurement of Luminous Efficiency

The luminance, current density, and voltage measured in (1) and (2) were used to calculate current efficiency (cd/A) at the same current density (10 mA/cm²).

The luminous efficiency values of Examples 1 to 18 and Comparative Examples 1 to 7 were calculated as relative values based on Comparative Example 1 and are shown in Table 1.

The luminous efficiency values of Examples 19 to 42 and Comparative Examples 8 to 14 were calculated as relative values based on Comparative Example 8 and are shown in Table 2.

### (4) Measurement of Life-Span

The results were obtained by measuring a time when current efficiency (cd/A) was decreased down to 97%, while luminance (cd/m²) was maintained to be 24000 cd/m².

The life-span measurement values of Examples 1 to 18 and Comparative Examples 1 to 7 were calculated as relative values based on Comparative Example 1 and are shown in Table 1.

The life-span measurement values of Examples 19 to 42 and Comparative Examples 8 to 14 were calculated as relative values based on Comparative Example 8 and are shown in Table 2.

### (5) Measurement of Driving Voltage

The results were obtained by measuring the driving voltage of each device at 15 mA/cm² using a current-voltage meter (Keithley 2400).

The driving voltages of Examples 1 to 18 and Comparative Examples 1 to 7 were calculated as relative values based on Comparative Example 1 and are shown in Table 1.

The driving voltages of Examples 19 to 42 and Comparative Examples 8 to 14 were calculated as relative values based on Comparative Example 8 and are shown in Table 2.

TABLE 1

| | | | Driving | Color | | Life- |
|---|---|---|---|---|---|---|
| | Compound | | voltage | (EL | Efficiency | span |
| No. | (wt %) | | (%) | color) | (%) | (%) |
| Example 1 | 1 | (10) | 90 | Green | 166 | 125 |
| Example 2 | 2 | (10) | 88 | Green | 169 | 136 |
| Example 3 | 3 | (10) | 89 | Green | 171 | 143 |
| Example 4 | 4 | (10) | 92 | Green | 164 | 179 |
| Example 5 | 5 | (10) | 88 | Green | 157 | 146 |
| Example 6 | 6 | (10) | 86 | Green | 166 | (10)7 |
| Example 7 | 7 | (10) | 91 | Green | 143 | 111 |
| Example 8 | 8 | (10) | 91 | Green | 171 | 125 |
| Example 9 | 12 | (10) | 93 | Green | 170 | 196 |
| Example 10 | 13 | (10) | 88 | Green | 173 | 143 |
| Example 11 | 25 | (10) | 94 | Green | 171 | 196 |
| Example 12 | 45 | (10) | 92 | Green | 170 | 232 |
| Example 13 | 57 | (10) | 90 | Green | 169 | 161 |
| Example 14 | 69 | (10) | 91 | Green | 157 | 114 |
| Example 15 | 119 | (10) | 88 | Green | 167 | 146 |
| Example 16 | 181 | (10) | 92 | Green | 186 | 179 |
| Example 17 | 221 | (10) | 94 | Green | 137 | 250 |
| Example 18 | 225 | (10) | 94 | Green | 140 | 161 |
| Comparative Example 1 | Host1 | (10) | 100 | Green | 100 | 100 |
| Comparative Example 2 | Host2 | (10) | 95 | Green | 114 | 71 |
| Comparative Example 3 | Host3 | (10) | 96 | Green | 86 | 89 |
| Comparative Example 4 | Host4 | (10) | 94 | Green | 123 | 79 |
| Comparative Example 5 | Host5 | (10) | 95 | Green | 57 | 0 |
| Comparative Example 6 | Host6 | (10) | 96 | Green | 86 | 18 |
| Comparative Example 7 | Host7 | (10) | 101 | Green | 129 | 36 |

TABLE 2

| | | | Driving | Color | | Life- |
|---|---|---|---|---|---|---|
| | | | voltage | (EL | Efficiency | span |
| No. | Compound (wt %) | | (%) | color) | (%) | (%) |
| Example 19 | 1/B-136 | (3:7) | 90 | Green | 151 | 133 |
| Example 20 | 1/B-136 | (4:6) | 86 | Green | 158 | 111 |
| Example 21 | 1/B-136 | (2:8) | 92 | Green | 144 | 144 |
| Example 22 | 1/B-99 | (3:7) | 89 | Green | 153 | 156 |
| Example 23 | 1/B-31 | (3:7) | 92 | Green | 156 | 178 |
| Example 24 | 1/C-4 | (3:7) | 83 | Green | 158 | 105 |
| Example 25 | 1/C-57 | (3:7) | 89 | Green | 144 | 110 |
| Example 26 | 2/B-136 | (3:7) | 89 | Green | 153 | 144 |
| Example 27 | 3/B-136 | (3:7) | 90 | Green | 156 | 156 |
| Example 28 | 4/B-136 | (3:7) | 93 | Green | 150 | 178 |
| Example 29 | 5/B-136 | (3:7) | 89 | Green | 144 | 156 |
| Example 30 | 6/B-136 | (3:7) | 87 | Green | 151 | 122 |
| Example 31 | 7/B-136 | (3:7) | 92 | Green | 133 | 113 |
| Example 32 | 8/B-136 | (3:7) | 92 | Green | 156 | 122 |
| Example 33 | 12/B-136 | (3:7) | 93 | Green | 154 | 189 |
| Example 34 | 13/B-136 | (3:7) | 89 | Green | 157 | 156 |
| Example 35 | 25/B-136 | (3:7) | 94 | Green | 156 | 189 |
| Example 36 | 45/B-136 | (3:7) | 92 | Green | 154 | 211 |
| Example 37 | 57/B-136 | (3:7) | 91 | Green | 153 | 167 |
| Example 38 | 69/B-136 | (3:7) | 92 | Green | 144 | 122 |
| Example 39 | 119/B-136 | (3:7) | 89 | Green | 152 | 158 |
| Example 40 | 181/B-136 | (3:7) | 93 | Green | 167 | 178 |
| Example 41 | 221/B-136 | (3:7) | 94 | Green | 122 | 222 |
| Example 42 | 225/B-136 | (3:7) | 94 | Green | 131 | 167 |
| Comparative Example 8 | Host1/B-136 | (3:7) | 100 | Green | 100 | 100 |
| Comparative Example 9 | Host2/B-136 | (3:7) | 95 | Green | 111 | 67 |
| Comparative Example 10 | Host3/B-136 | (3:7) | 96 | Green | 89 | 78 |
| Comparative Example 11 | Host4/B-136 | (3:7) | 95 | Green | 120 | 67 |
| Comparative Example 12 | Host5/B-136 | (3:7) | 95 | Green | 67 | 11 |
| Comparative Example 13 | Host6/B-136 | (3:7) | 96 | Green | 89 | 4 |
| Comparative Example 14 | Host7/B-136 | (3:7) | 101 | Green | 119 | 22 |

Referring to Table 1 and Table 2, the organic light emitting diodes according to Examples 1 to 42 exhibited significantly improved driving voltage, luminous efficiency and life-span characteristics compared to the organic light emitting diodes according to Comparative Examples 1 to 14.

The life-span of “0%” means that the organic light emitting diode was dead suddenly, with the current efficiency (cd/A) rapidly decreasing to 97%.

The invention claimed is:

1. A compound for an organic optoelectronic device represented by Chemical Formula 1: 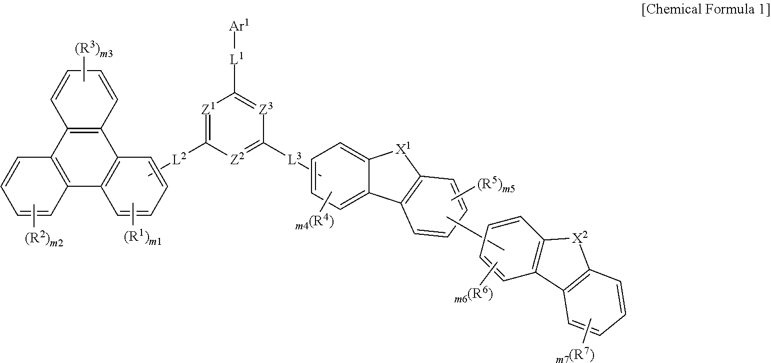
wherein, in Chemical Formula 1,
X¹ and X² are each independently O, S, or SiR^{a}R^{b}
Z¹ to Z³ are each independently N or CR^{c},
at least one of Z¹ to Z³ is N,
R^{a}, R^{b}, R^{c}, and R¹ to R⁷ are each independently hydrogen, deuterium, a cyano group, a substituted or unsubstituted C1 to C10 alkyl group, a substituted or unsubstituted C6 to C20 aryl group or a substituted or unsubstituted C2 to C20 heterocyclic group,
R⁴ to R⁷ are each independently present or adjacent groups are linked to form a substituted or unsubstituted aromatic monocyclic ring or a substituted or unsubstituted aromatic polycyclic ring,
Ar¹ is a substituted or unsubstituted C6 to C20 aryl group or a substituted or unsubstituted C2 to C20 heterocyclic group,
L¹ to L³ are each independently a single bond or a substituted or unsubstituted C6 to C20 arylene group,
m1, m4, m5, and m6 are each independently one of integers of 1 to 3, and
m2, m3, and m7 are each independently one of integers of 1 to 4.

2. The compound for an organic optoelectronic device as claimed in claim 1, wherein
Chemical Formula 1 is represented by any one of Chemical Formula 1-1 to Chemical Formula 1-4: 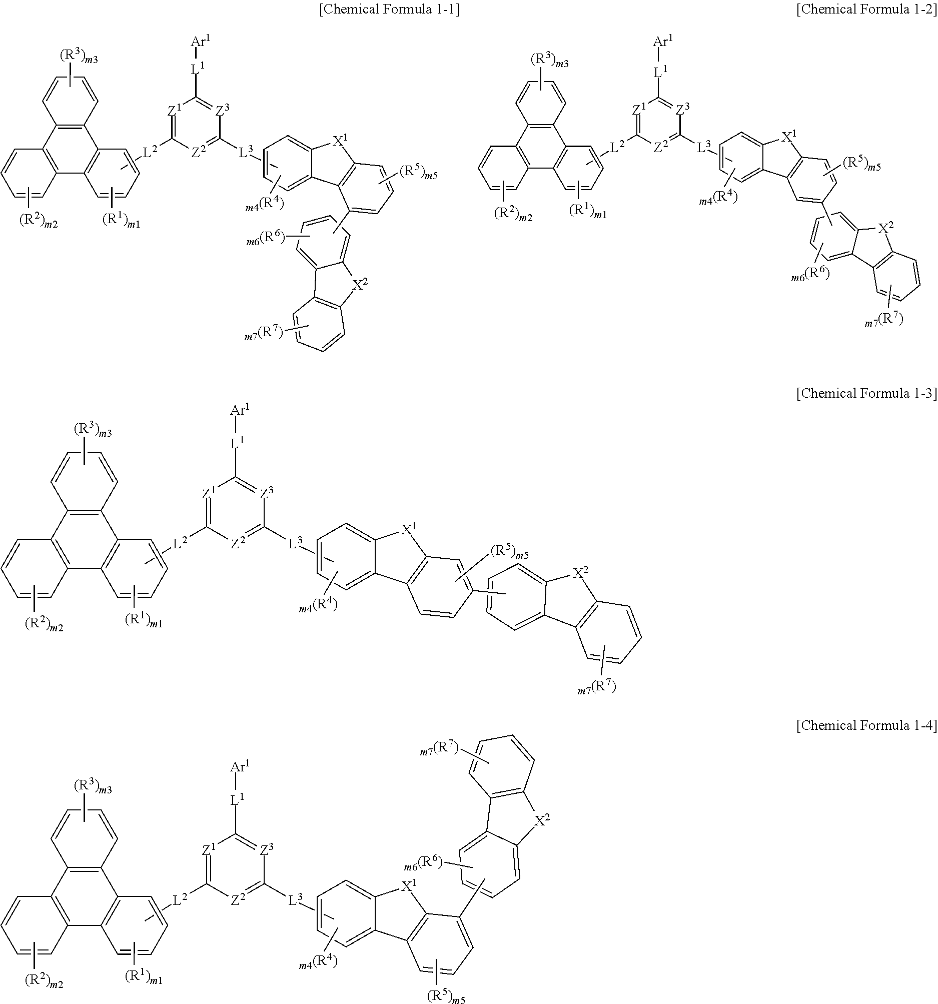
wherein, in Chemical Formula 1-1 to Chemical Formula 1-4,
X¹, X², Z¹ to Z³, R¹ to R⁷, Ar¹, L¹ to L³, and m1 to m7 are the same as defined in claim 1.

3. The compound for an organic optoelectronic device as claimed in claim 2, wherein
Chemical Formula 1-1 is represented by any one of Chemical Formula 1-1-(i), Chemical Formula 1-1-(ii), Chemical Formula 1-1-(iii), and Chemical Formula 1-1-(iv): 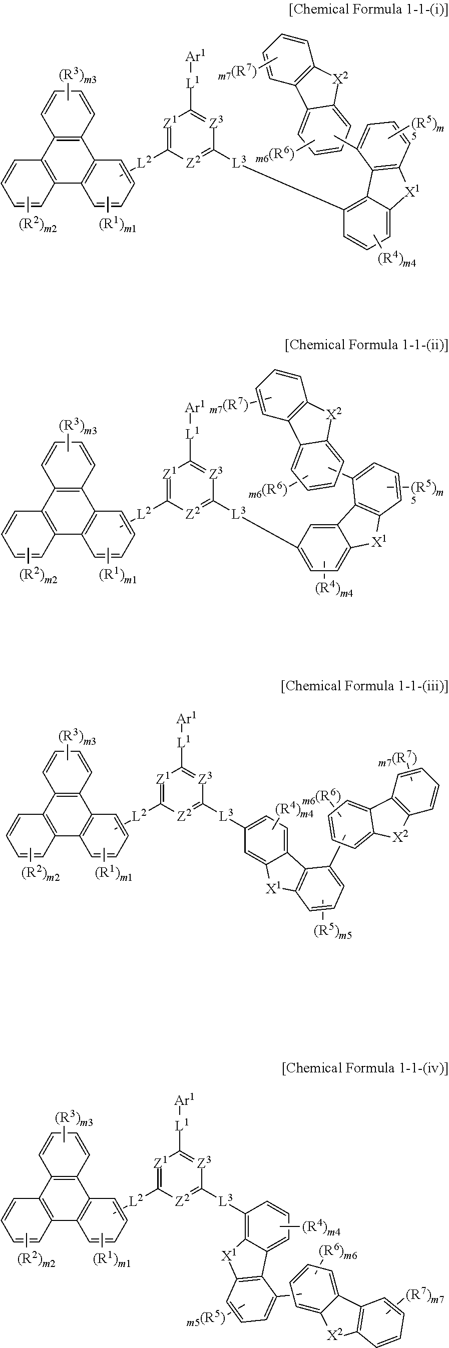
wherein, in Chemical Formula 1-1-(i), Chemical Formula 1-1-(ii), Chemical Formula 1-1-(iii), and Chemical Formula 1-1-(iv),
X¹, X², Z¹ to Z³, R¹ to R⁷, Ar¹, L¹ to L³, and m1 to m7 are the same as defined in claim 1.

4. The compound for an organic optoelectronic device as claimed in claim 2, wherein
Chemical Formula 1-4 is represented by any one of Chemical Formula 1-4-(i) to Chemical Formula 1-4-(iv): 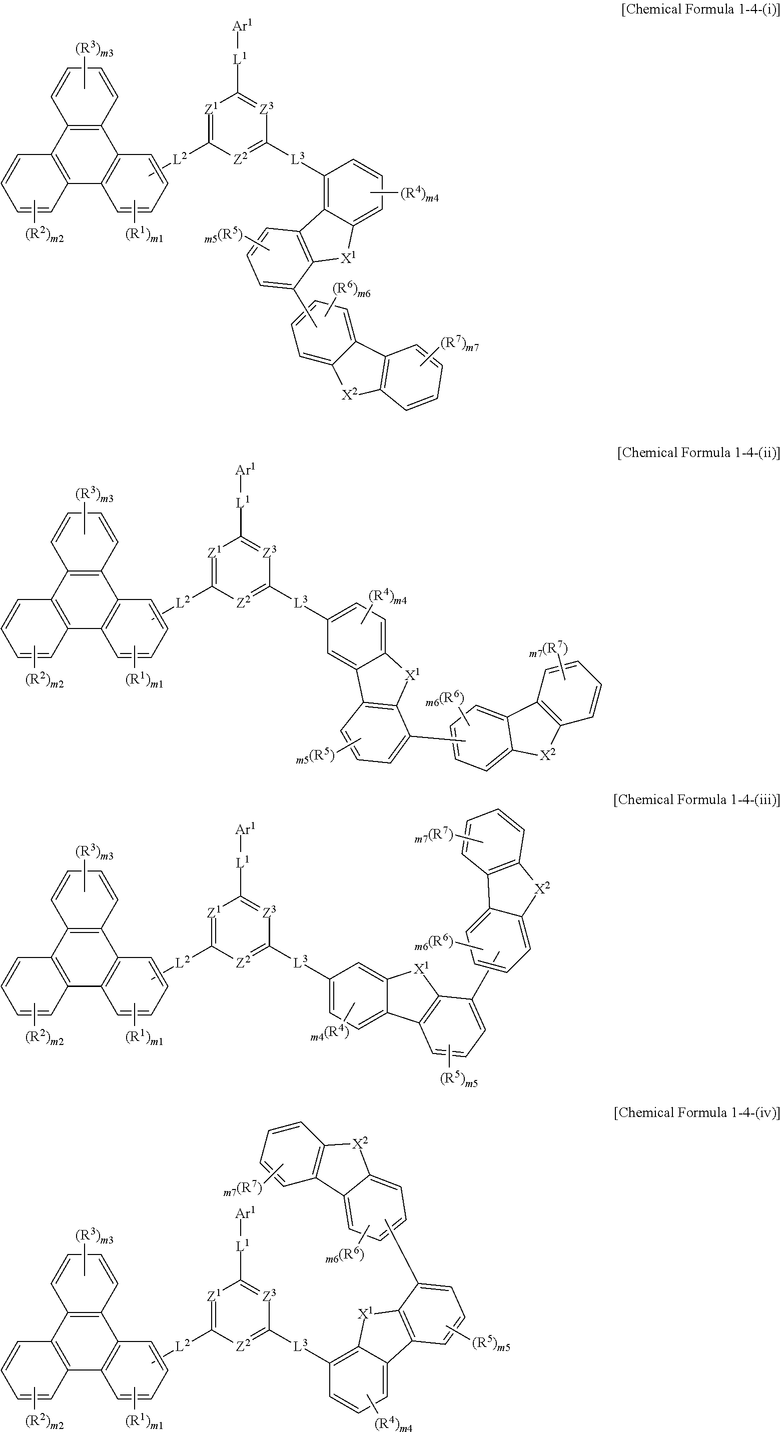
wherein, in Chemical Formula 1-4-(i), Chemical Formula 1-4-(ii), Chemical Formula 1-4-(iii), and Chemical Formula 1-4-(iv),
X¹, X², Z¹ to Z³, R¹ to R⁷, Ar¹, L¹ to L³, and m1 to m7 are the same as defined in claim 1.

5. The compound for an organic optoelectronic device as claimed in claim 1, wherein
in Chemical Formula 1, Ar¹ is a substituted or unsubstituted phenyl group, a substituted or unsubstituted biphenyl group, a substituted or unsubstituted fluorenyl group, a substituted or unsubstituted dibenzofuranyl group, a substituted or unsubstituted dibenzothiophenyl group, a substituted or unsubstituted dibenzosilolyl group, or a substituted or unsubstituted carbazolyl group.

6. The compound for an organic optoelectronic device as claimed in claim 1, wherein
the compound is selected from the compounds listed in Group 1:

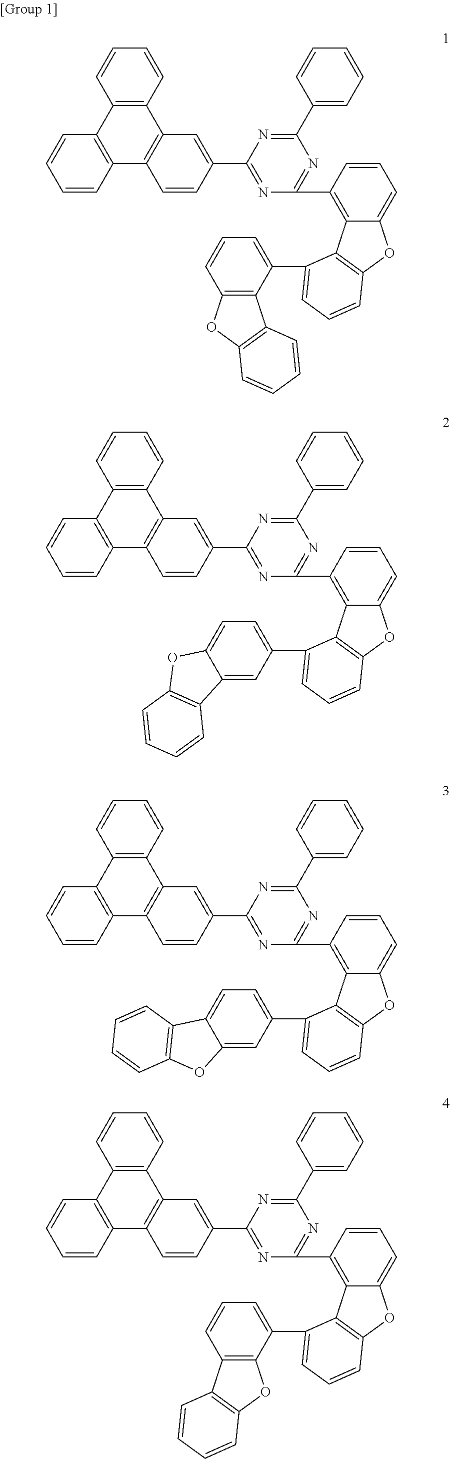

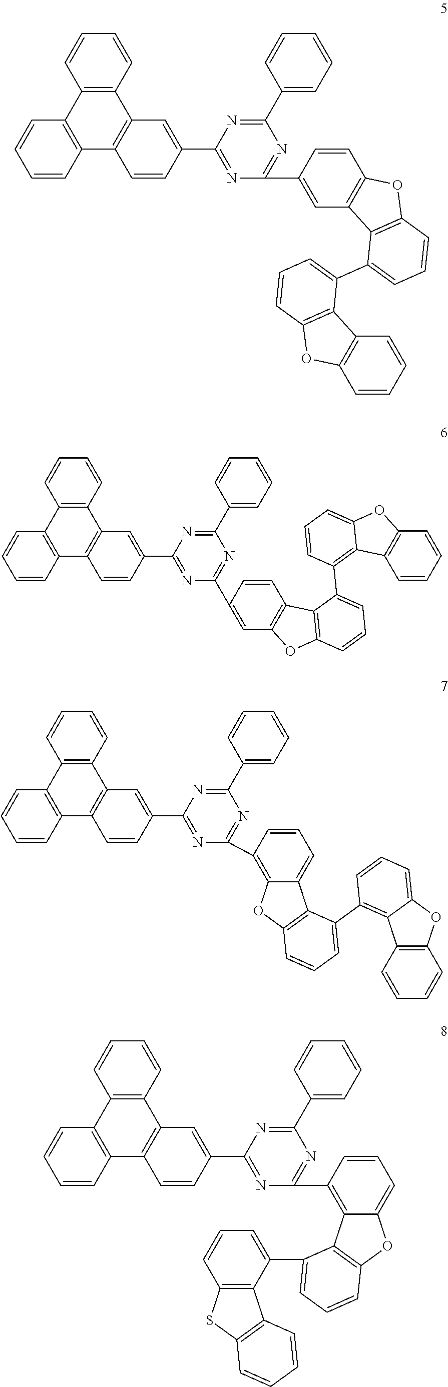 [SMILES: c1ccc(-c2nc(-c3ccc4c5ccccc5c5ccccc5c4c3)nc(-c3ccc4oc5cccc(-c6cccc7oc8ccccc8c67)c5c4c3)n2)cc1 ; c1ccc(-c2nc(-c3ccc4c(c3)oc3cccc(-c5cccc6oc7ccccc7c56)c34)nc(-c3ccc4c5ccccc5c5ccccc5c4c3)n2)cc1 ; c1ccc(-c2nc(-c3ccc4c5ccccc5c5ccccc5c4c3)nc(-c3cccc4c3oc3cccc(-c5cccc6oc7ccccc7c56)c34)n2)cc1 ; c1ccc(-c2nc(-c3ccc4c5ccccc5c5ccccc5c4c3)nc(-c3cccc4oc5cccc(-c6cccc7sc8ccccc8c67)c5c34)n2)cc1]

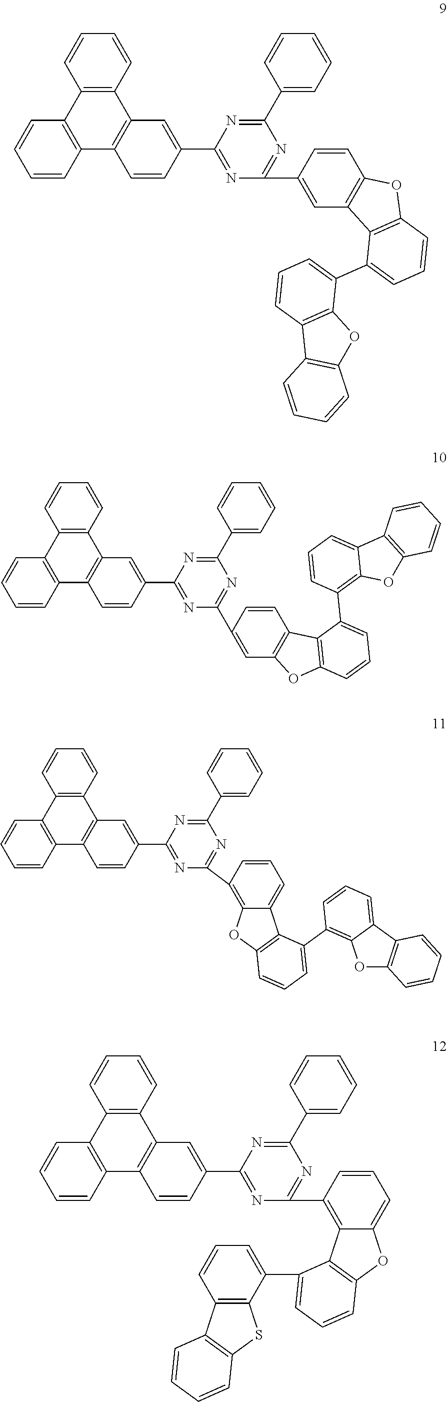 [SMILES: c1ccc(-c2nc(-c3ccc4c5ccccc5c5ccccc5c4c3)nc(-c3ccc4oc5cccc(-c6cccc7c6oc6ccccc67)c5c4c3)n2)cc1 ; c1ccc(-c2nc(-c3ccc4c(c3)oc3cccc(-c5cccc6c5oc5ccccc56)c34)nc(-c3ccc4c5ccccc5c5ccccc5c4c3)n2)cc1 ; c1ccc(-c2nc(-c3ccc4c5ccccc5c5ccccc5c4c3)nc(-c3cccc4c3oc3cccc(-c5cccc6c5oc5ccccc56)c34)n2)cc1 ; c1ccc(-c2nc(-c3ccc4c5ccccc5c5ccccc5c4c3)nc(-c3cccc4oc5cccc(-c6cccc7c6sc6ccccc67)c5c34)n2)cc1]

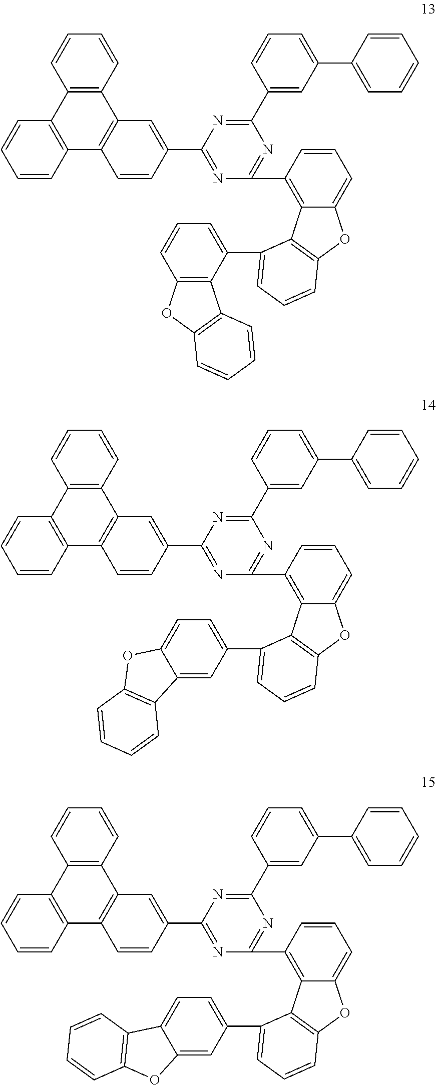 [SMILES: c1ccc(-c2cccc(-c3nc(-c4ccc5c6ccccc6c6ccccc6c5c4)nc(-c4cccc5oc6cccc(-c7cccc8oc9ccccc9c78)c6c45)n3)c2)cc1 ; c1ccc(-c2cccc(-c3nc(-c4ccc5c6ccccc6c6ccccc6c5c4)nc(-c4cccc5oc6cccc(-c7ccc8oc9ccccc9c8c7)c6c45)n3)c2)cc1 ; c1ccc(-c2cccc(-c3nc(-c4ccc5c6ccccc6c6ccccc6c5c4)nc(-c4cccc5oc6cccc(-c7ccc8c(c7)oc7ccccc78)c6c45)n3)c2)cc1]

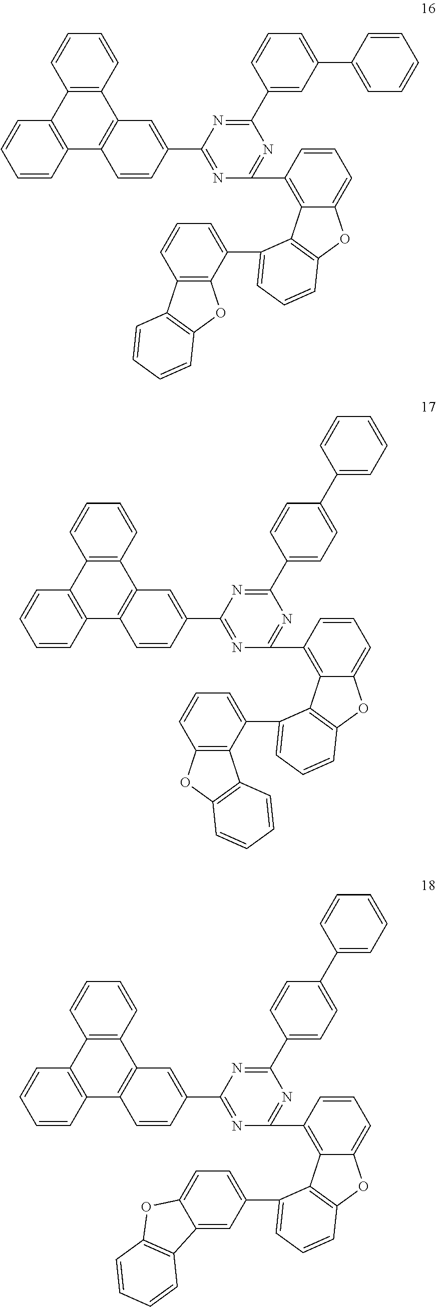 [SMILES: c1ccc(-c2cccc(-c3nc(-c4ccc5c6ccccc6c6ccccc6c5c4)nc(-c4cccc5oc6cccc(-c7cccc8c7oc7ccccc78)c6c45)n3)c2)cc1 ; c1ccc(-c2ccc(-c3nc(-c4ccc5c6ccccc6c6ccccc6c5c4)nc(-c4cccc5oc6cccc(-c7cccc8oc9ccccc9c78)c6c45)n3)cc2)cc1 ; c1ccc(-c2ccc(-c3nc(-c4ccc5c6ccccc6c6ccccc6c5c4)nc(-c4cccc5oc6cccc(-c7ccc8oc9ccccc9c8c7)c6c45)n3)cc2)cc1]

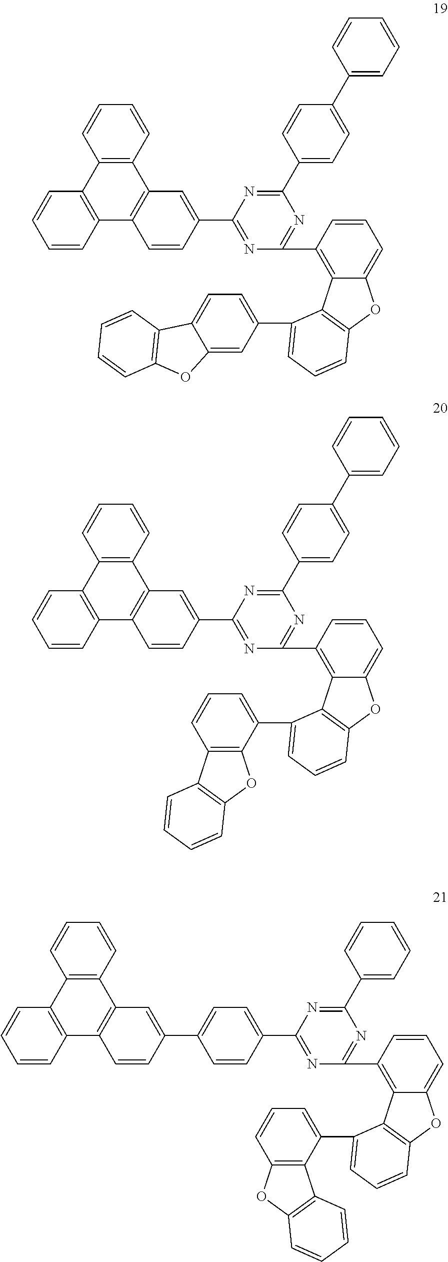 [SMILES: c1ccc(-c2ccc(-c3nc(-c4ccc5c6ccccc6c6ccccc6c5c4)nc(-c4cccc5oc6cccc(-c7ccc8c(c7)oc7ccccc78)c6c45)n3)cc2)cc1 ; c1ccc(-c2ccc(-c3nc(-c4ccc5c6ccccc6c6ccccc6c5c4)nc(-c4cccc5oc6cccc(-c7cccc8c7oc7ccccc78)c6c45)n3)cc2)cc1 ; c1ccc(-c2nc(-c3ccc(-c4ccc5c6ccccc6c6ccccc6c5c4)cc3)nc(-c3cccc4oc5cccc(-c6cccc7oc8ccccc8c67)c5c34)n2)cc1]

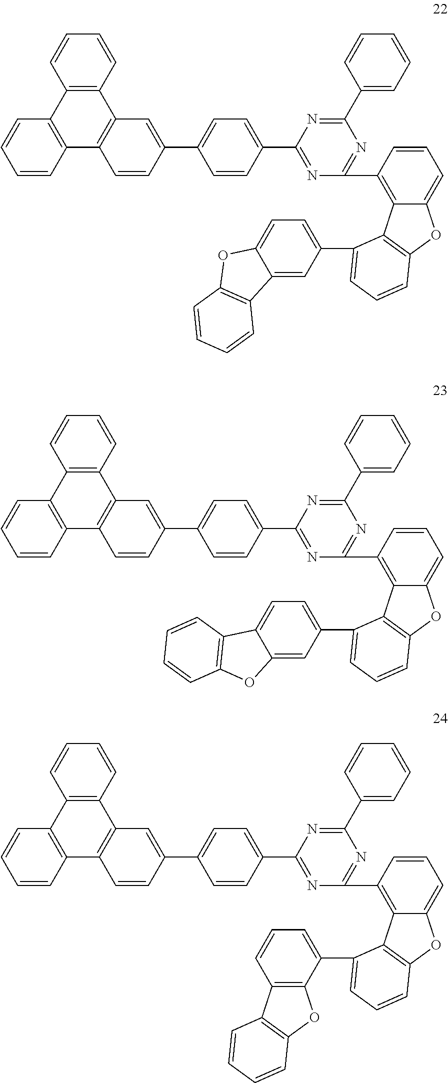 [SMILES: c1ccc(-c2nc(-c3ccc(-c4ccc5c6ccccc6c6ccccc6c5c4)cc3)nc(-c3cccc4oc5cccc(-c6ccc7oc8ccccc8c7c6)c5c34)n2)cc1 ; c1ccc(-c2nc(-c3ccc(-c4ccc5c6ccccc6c6ccccc6c5c4)cc3)nc(-c3cccc4oc5cccc(-c6ccc7c(c6)oc6ccccc67)c5c34)n2)cc1 ; c1ccc(-c2nc(-c3ccc(-c4ccc5c6ccccc6c6ccccc6c5c4)cc3)nc(-c3cccc4oc5cccc(-c6cccc7c6oc6ccccc67)c5c34)n2)cc1]

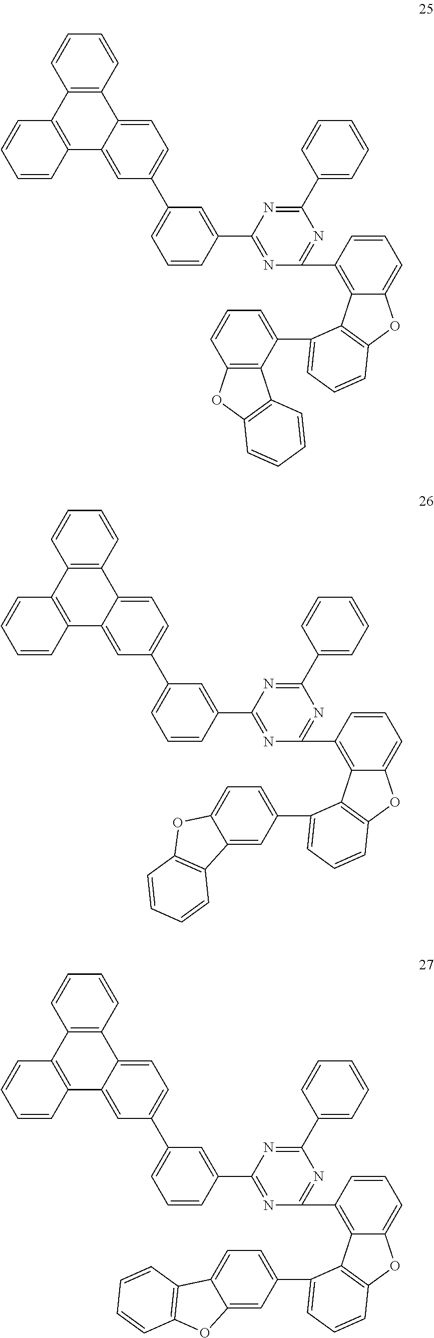 [SMILES: c1ccc(-c2nc(-c3cccc(-c4ccc5c6ccccc6c6ccccc6c5c4)c3)nc(-c3cccc4oc5cccc(-c6cccc7oc8ccccc8c67)c5c34)n2)cc1 ; c1ccc(-c2nc(-c3cccc(-c4ccc5c6ccccc6c6ccccc6c5c4)c3)nc(-c3cccc4oc5cccc(-c6ccc7oc8ccccc8c7c6)c5c34)n2)cc1 ; c1ccc(-c2nc(-c3cccc(-c4ccc5c6ccccc6c6ccccc6c5c4)c3)nc(-c3cccc4oc5cccc(-c6ccc7c(c6)oc6ccccc67)c5c34)n2)cc1]

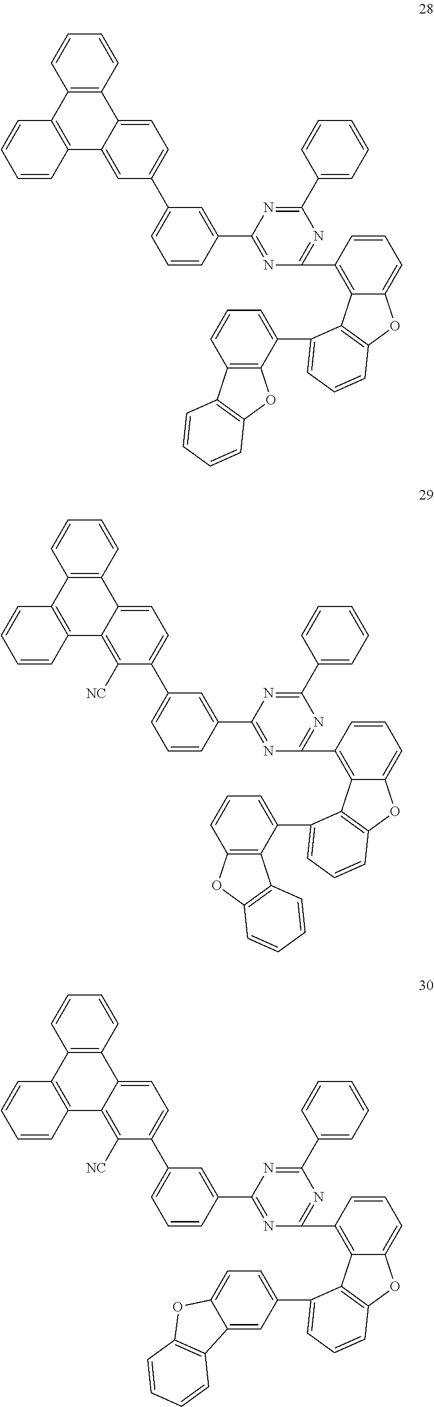 [SMILES: c1ccc(-c2nc(-c3cccc(-c4ccc5c6ccccc6c6ccccc6c5c4)c3)nc(-c3cccc4oc5cccc(-c6cccc7c6oc6ccccc67)c5c34)n2)cc1 ; N#Cc1c(-c2cccc(-c3nc(-c4ccccc4)nc(-c4cccc5oc6cccc(-c7cccc8oc9ccccc9c78)c6c45)n3)c2)ccc2c3ccccc3c3ccccc3c12 ; N#Cc1c(-c2cccc(-c3nc(-c4ccccc4)nc(-c4cccc5oc6cccc(-c7ccc8oc9ccccc9c8c7)c6c45)n3)c2)ccc2c3ccccc3c3ccccc3c12]

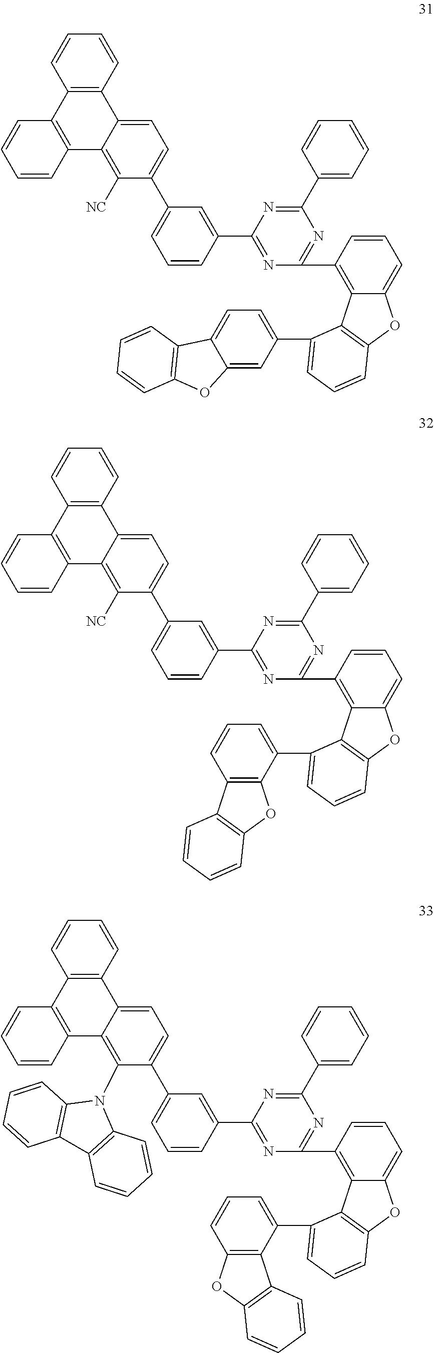 [SMILES: N#Cc1c(-c2cccc(-c3nc(-c4ccccc4)nc(-c4cccc5oc6cccc(-c7ccc8c(c7)oc7ccccc78)c6c45)n3)c2)ccc2c3ccccc3c3ccccc3c12 ; N#Cc1c(-c2cccc(-c3nc(-c4ccccc4)nc(-c4cccc5oc6cccc(-c7cccc8c7oc7ccccc78)c6c45)n3)c2)ccc2c3ccccc3c3ccccc3c12 ; c1ccc(-c2nc(-c3cccc(-c4ccc5c6ccccc6c6ccccc6c5c4-n4c5ccccc5c5ccccc54)c3)nc(-c3cccc4oc5cccc(-c6cccc7oc8ccccc8c67)c5c34)n2)cc1]

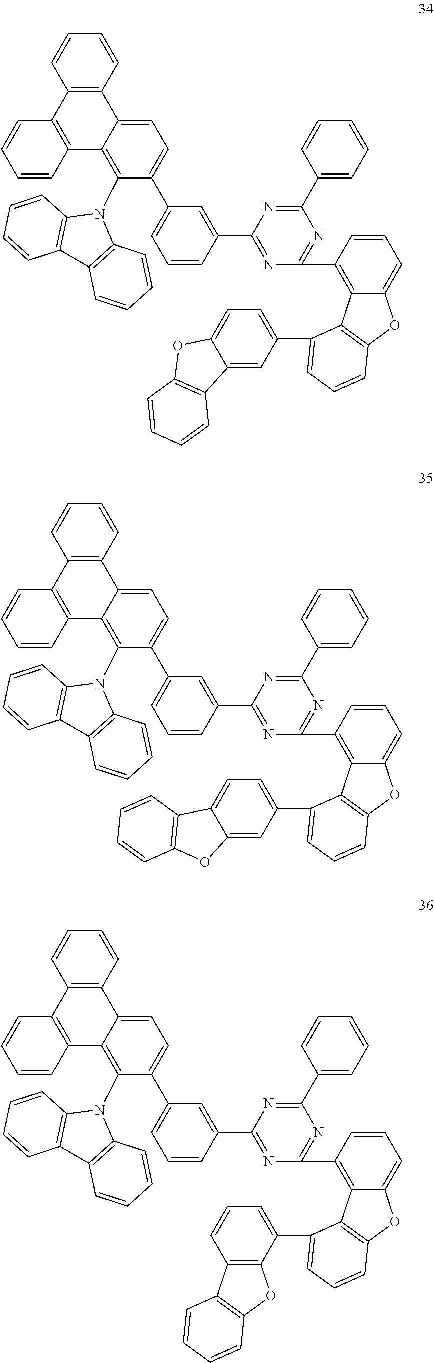 [SMILES: c1ccc(-c2nc(-c3cccc(-c4ccc5c6ccccc6c6ccccc6c5c4-n4c5ccccc5c5ccccc54)c3)nc(-c3cccc4oc5cccc(-c6ccc7oc8ccccc8c7c6)c5c34)n2)cc1 ; c1ccc(-c2nc(-c3cccc(-c4ccc5c6ccccc6c6ccccc6c5c4-n4c5ccccc5c5ccccc54)c3)nc(-c3cccc4oc5cccc(-c6ccc7c(c6)oc6ccccc67)c5c34)n2)cc1 ; c1ccc(-c2nc(-c3cccc(-c4ccc5c6ccccc6c6ccccc6c5c4-n4c5ccccc5c5ccccc54)c3)nc(-c3cccc4oc5cccc(-c6cccc7c6oc6ccccc67)c5c34)n2)cc1]

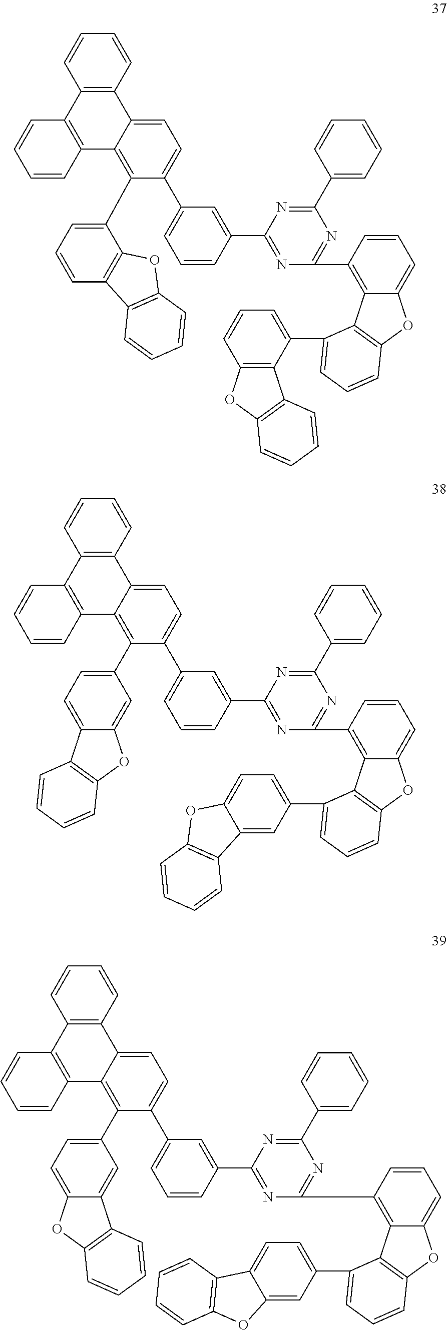 [SMILES: c1ccc(-c2nc(-c3cccc(-c4ccc5c6ccccc6c6ccccc6c5c4-c4cccc5c4oc4ccccc45)c3)nc(-c3cccc4oc5cccc(-c6cccc7oc8ccccc8c67)c5c34)n2)cc1 ; c1ccc(-c2nc(-c3cccc(-c4ccc5c6ccccc6c6ccccc6c5c4-c4ccc5c(c4)oc4ccccc45)c3)nc(-c3cccc4oc5cccc(-c6ccc7oc8ccccc8c7c6)c5c34)n2)cc1 ; c1ccc(-c2nc(-c3cccc(-c4ccc5c6ccccc6c6ccccc6c5c4-c4ccc5oc6ccccc6c5c4)c3)nc(-c3cccc4oc5cccc(-c6ccc7c(c6)oc6ccccc67)c5c34)n2)cc1]

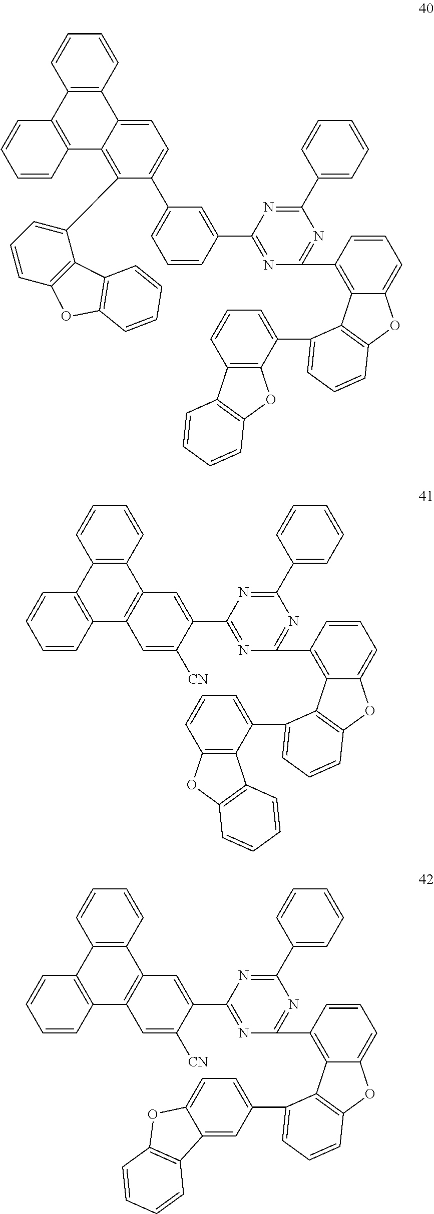 [SMILES: c1ccc(-c2nc(-c3cccc(-c4ccc5c6ccccc6c6ccccc6c5c4-c4cccc5oc6ccccc6c45)c3)nc(-c3cccc4oc5cccc(-c6cccc7c6oc6ccccc67)c5c34)n2)cc1 ; N#Cc1cc2c3ccccc3c3ccccc3c2cc1-c1nc(-c2ccccc2)nc(-c2cccc3oc4cccc(-c5cccc6oc7ccccc7c56)c4c23)n1 ; N#Cc1cc2c3ccccc3c3ccccc3c2cc1-c1nc(-c2ccccc2)nc(-c2cccc3oc4cccc(-c5ccc6oc7ccccc7c6c5)c4c23)n1]

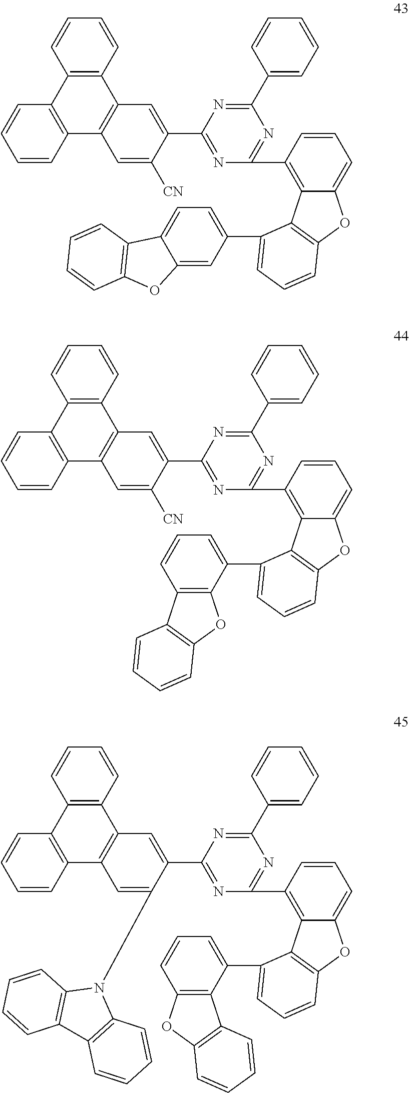 [SMILES: N#Cc1cc2c3ccccc3c3ccccc3c2cc1-c1nc(-c2ccccc2)nc(-c2cccc3oc4cccc(-c5ccc6c(c5)oc5ccccc56)c4c23)n1 ; N#Cc1cc2c3ccccc3c3ccccc3c2cc1-c1nc(-c2ccccc2)nc(-c2cccc3oc4cccc(-c5cccc6c5oc5ccccc56)c4c23)n1 ; c1ccc(-c2nc(-c3cc4c5ccccc5c5ccccc5c4cc3-n3c4ccccc4c4ccccc43)nc(-c3cccc4oc5cccc(-c6cccc7oc8ccccc8c67)c5c34)n2)cc1]

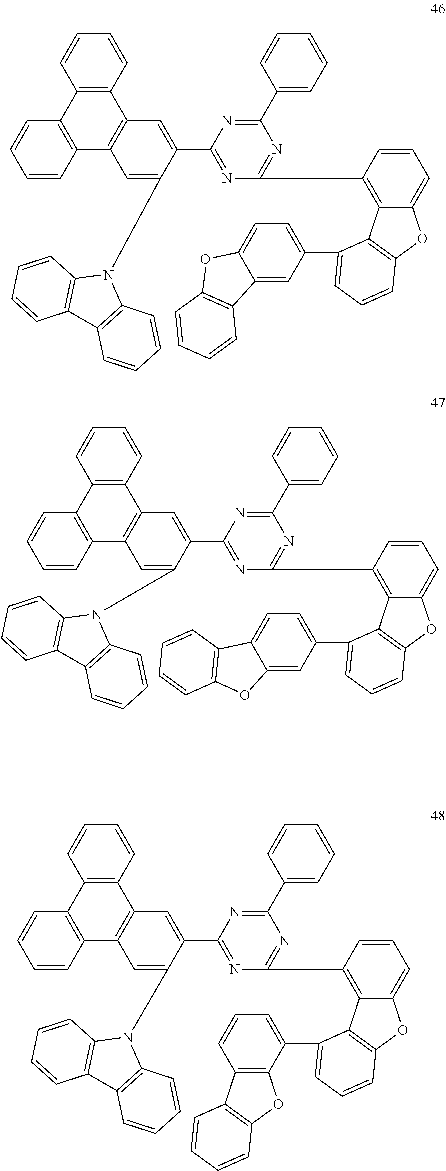 [SMILES: c1ccc(-c2nc(-c3cc4c5ccccc5c5ccccc5c4cc3-n3c4ccccc4c4ccccc43)nc(-c3cccc4oc5cccc(-c6ccc7oc8ccccc8c7c6)c5c34)n2)cc1 ; c1ccc(-c2nc(-c3cc4c5ccccc5c5ccccc5c4cc3-n3c4ccccc4c4ccccc43)nc(-c3cccc4oc5cccc(-c6ccc7c(c6)oc6ccccc67)c5c34)n2)cc1 ; c1ccc(-c2nc(-c3cc4c5ccccc5c5ccccc5c4cc3-n3c4ccccc4c4ccccc43)nc(-c3cccc4oc5cccc(-c6cccc7c6oc6ccccc67)c5c34)n2)cc1]

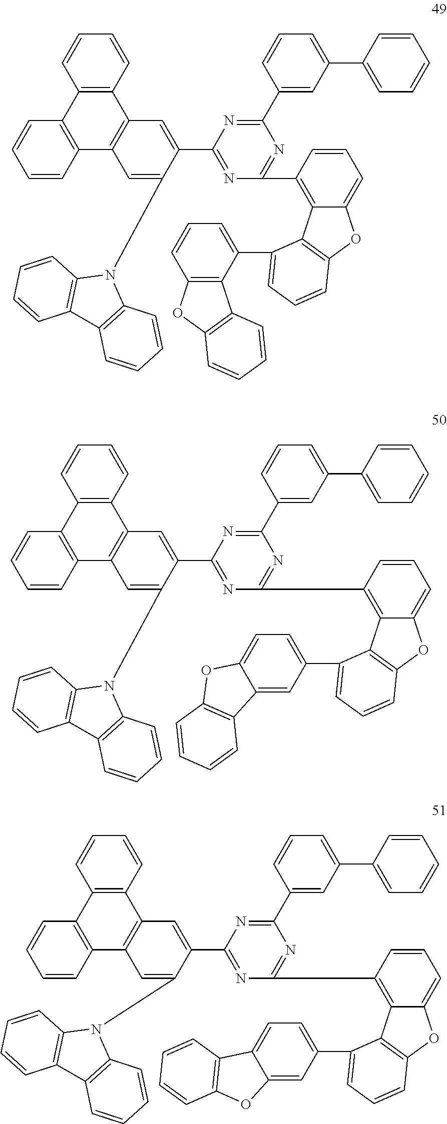 [SMILES: c1ccc(-c2cccc(-c3nc(-c4cc5c6ccccc6c6ccccc6c5cc4-n4c5ccccc5c5ccccc54)nc(-c4cccc5oc6cccc(-c7cccc8oc9ccccc9c78)c6c45)n3)c2)cc1 ; c1ccc(-c2cccc(-c3nc(-c4cc5c6ccccc6c6ccccc6c5cc4-n4c5ccccc5c5ccccc54)nc(-c4cccc5oc6cccc(-c7ccc8oc9ccccc9c8c7)c6c45)n3)c2)cc1 ; c1ccc(-c2cccc(-c3nc(-c4cc5c6ccccc6c6ccccc6c5cc4-n4c5ccccc5c5ccccc54)nc(-c4cccc5oc6cccc(-c7ccc8c(c7)oc7ccccc78)c6c45)n3)c2)cc1]

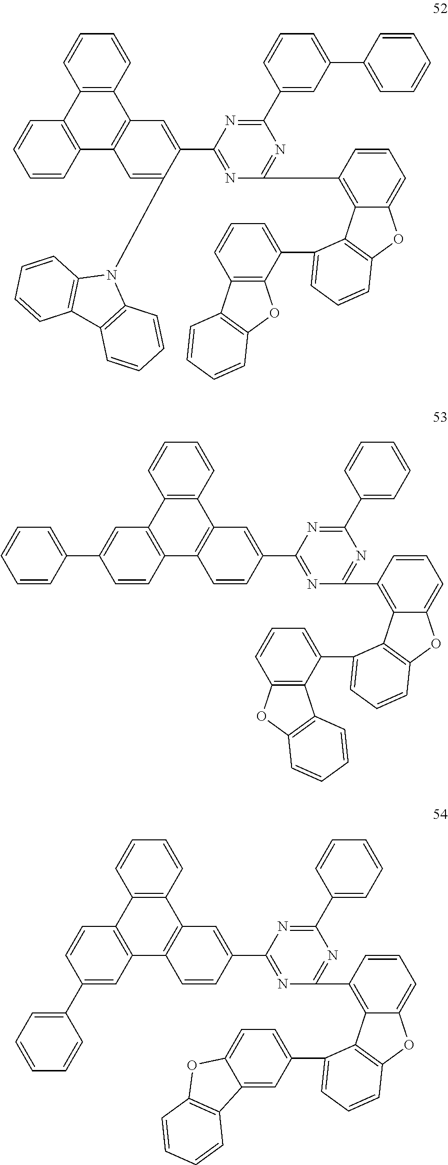 [SMILES: c1ccc(-c2cccc(-c3nc(-c4cc5c6ccccc6c6ccccc6c5cc4-n4c5ccccc5c5ccccc54)nc(-c4cccc5oc6cccc(-c7cccc8c7oc7ccccc78)c6c45)n3)c2)cc1 ; c1ccc(-c2ccc3c4ccc(-c5nc(-c6ccccc6)nc(-c6cccc7oc8cccc(-c9cccc%10oc%11ccccc%11c9%10)c8c67)n5)cc4c4ccccc4c3c2)cc1 ; c1ccc(-c2ccc3c4ccccc4c4cc(-c5nc(-c6ccccc6)nc(-c6cccc7oc8cccc(-c9ccc%10oc%11ccccc%11c%10c9)c8c67)n5)ccc4c3c2)cc1]

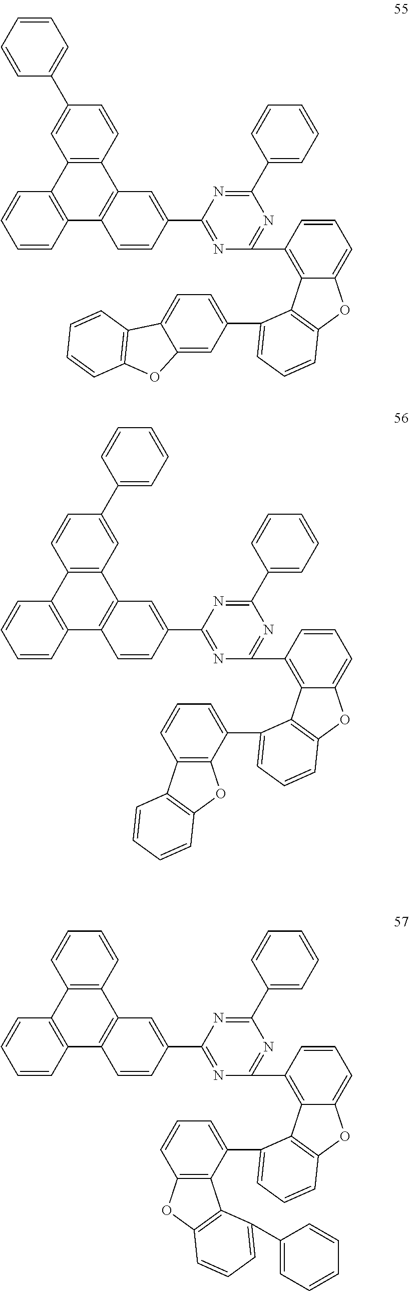 [SMILES: c1ccc(-c2ccc3c(c2)c2ccccc2c2ccc(-c4nc(-c5ccccc5)nc(-c5cccc6oc7cccc(-c8ccc9c(c8)oc8ccccc89)c7c56)n4)cc23)cc1 ; c1ccc(-c2ccc3c4ccccc4c4ccc(-c5nc(-c6ccccc6)nc(-c6cccc7oc8cccc(-c9cccc%10c9oc9ccccc9%10)c8c67)n5)cc4c3c2)cc1 ; c1ccc(-c2nc(-c3ccc4c5ccccc5c5ccccc5c4c3)nc(-c3cccc4oc5cccc(-c6cccc7oc8cccc(-c9ccccc9)c8c67)c5c34)n2)cc1]

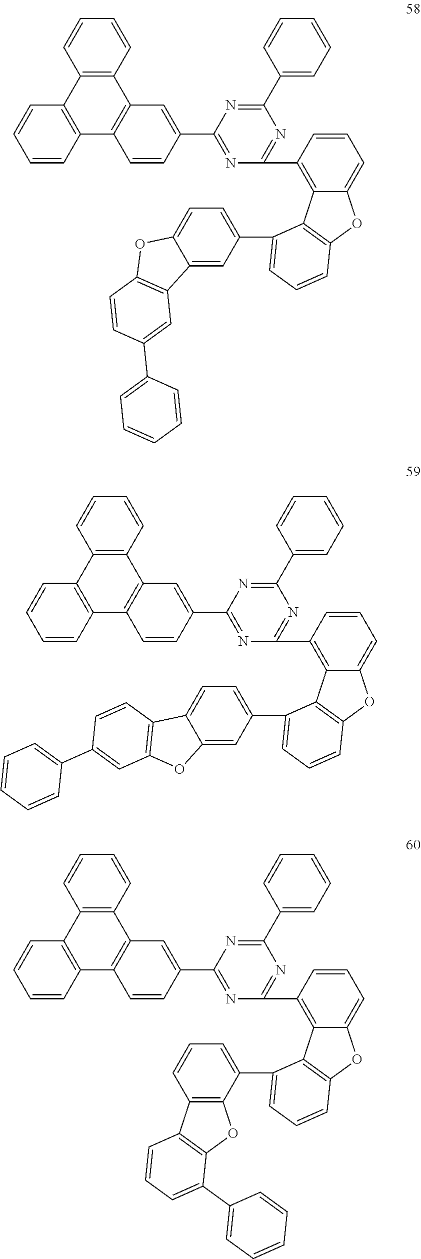 [SMILES: c1ccc(-c2ccc3oc4ccc(-c5cccc6oc7cccc(-c8nc(-c9ccccc9)nc(-c9ccc%10c%11ccccc%11c%11ccccc%11c%10c9)n8)c7c56)cc4c3c2)cc1 ; c1ccc(-c2ccc3c(c2)oc2cc(-c4cccc5oc6cccc(-c7nc(-c8ccccc8)nc(-c8ccc9c%10ccccc%10c%10ccccc%10c9c8)n7)c6c45)ccc23)cc1 ; c1ccc(-c2nc(-c3ccc4c5ccccc5c5ccccc5c4c3)nc(-c3cccc4oc5cccc(-c6cccc7c6oc6c(-c8ccccc8)cccc67)c5c34)n2)cc1]

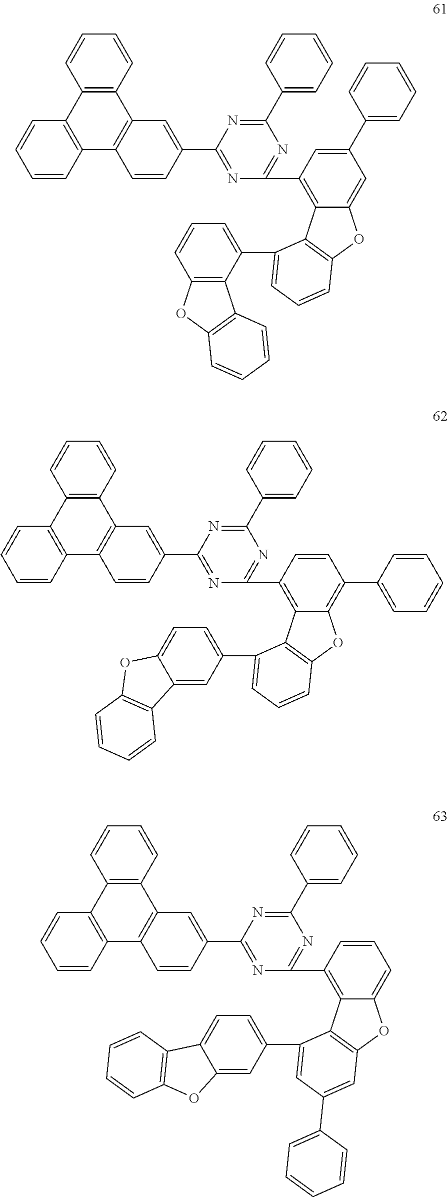 [SMILES: c1ccc(-c2cc(-c3nc(-c4ccccc4)nc(-c4ccc5c6ccccc6c6ccccc6c5c4)n3)c3c(c2)oc2cccc(-c4cccc5oc6ccccc6c45)c23)cc1 ; c1ccc(-c2nc(-c3ccc4c5ccccc5c5ccccc5c4c3)nc(-c3ccc(-c4ccccc4)c4oc5cccc(-c6ccc7oc8ccccc8c7c6)c5c34)n2)cc1 ; c1ccc(-c2cc(-c3ccc4c(c3)oc3ccccc34)c3c(c2)oc2cccc(-c4nc(-c5ccccc5)nc(-c5ccc6c7ccccc7c7ccccc7c6c5)n4)c23)cc1]

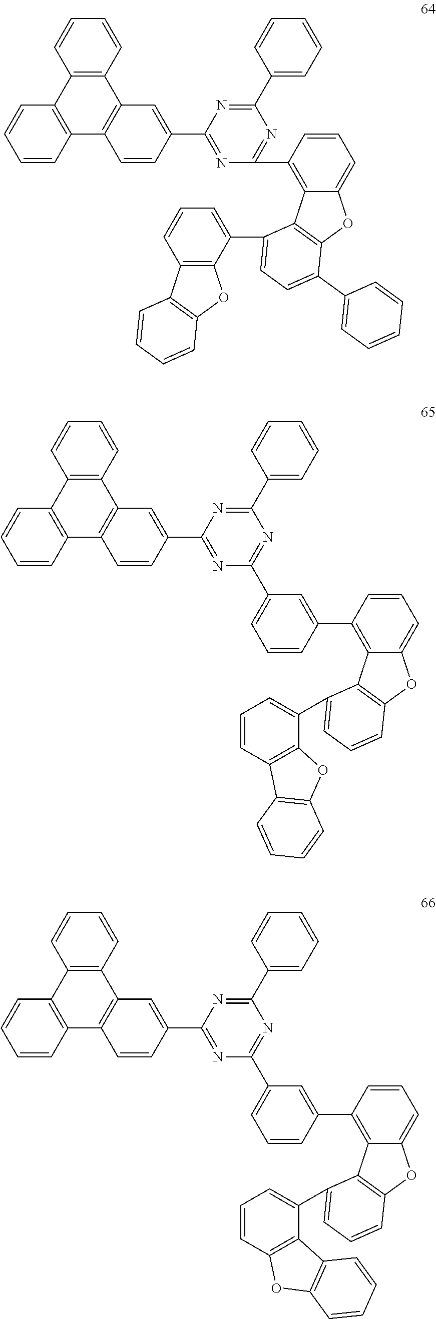 [SMILES: c1ccc(-c2nc(-c3ccc4c5ccccc5c5ccccc5c4c3)nc(-c3cccc4oc5c(-c6ccccc6)ccc(-c6cccc7c6oc6ccccc67)c5c34)n2)cc1 ; c1ccc(-c2nc(-c3cccc(-c4cccc5oc6cccc(-c7cccc8c7oc7ccccc78)c6c45)c3)nc(-c3ccc4c5ccccc5c5ccccc5c4c3)n2)cc1 ; c1ccc(-c2nc(-c3cccc(-c4cccc5oc6cccc(-c7cccc8oc9ccccc9c78)c6c45)c3)nc(-c3ccc4c5ccccc5c5ccccc5c4c3)n2)cc1]

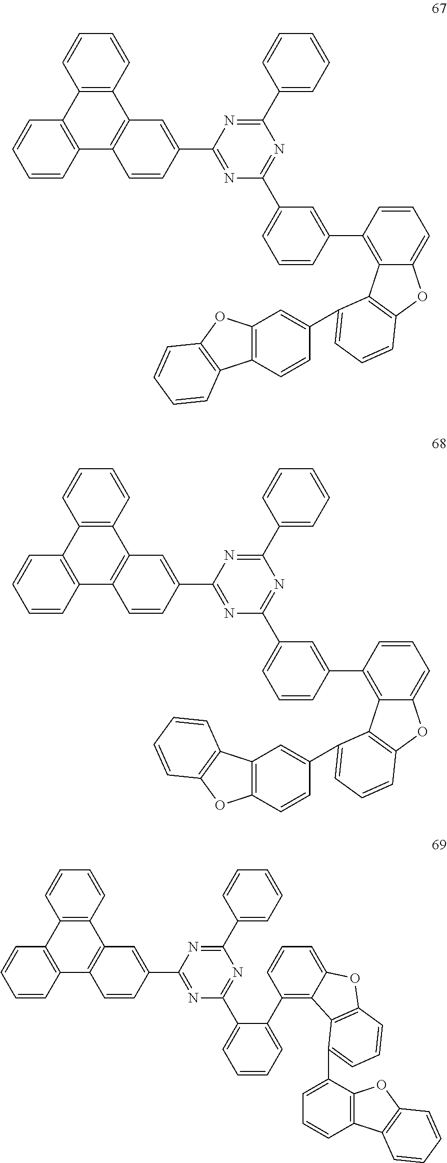 [SMILES: c1ccc(-c2nc(-c3cccc(-c4cccc5oc6cccc(-c7ccc8c(c7)oc7ccccc78)c6c45)c3)nc(-c3ccc4c5ccccc5c5ccccc5c4c3)n2)cc1 ; c1ccc(-c2nc(-c3cccc(-c4cccc5oc6cccc(-c7ccc8oc9ccccc9c8c7)c6c45)c3)nc(-c3ccc4c5ccccc5c5ccccc5c4c3)n2)cc1 ; c1ccc(-c2nc(-c3ccc4c5ccccc5c5ccccc5c4c3)nc(-c3ccccc3-c3cccc4oc5cccc(-c6cccc7c6oc6ccccc67)c5c34)n2)cc1]

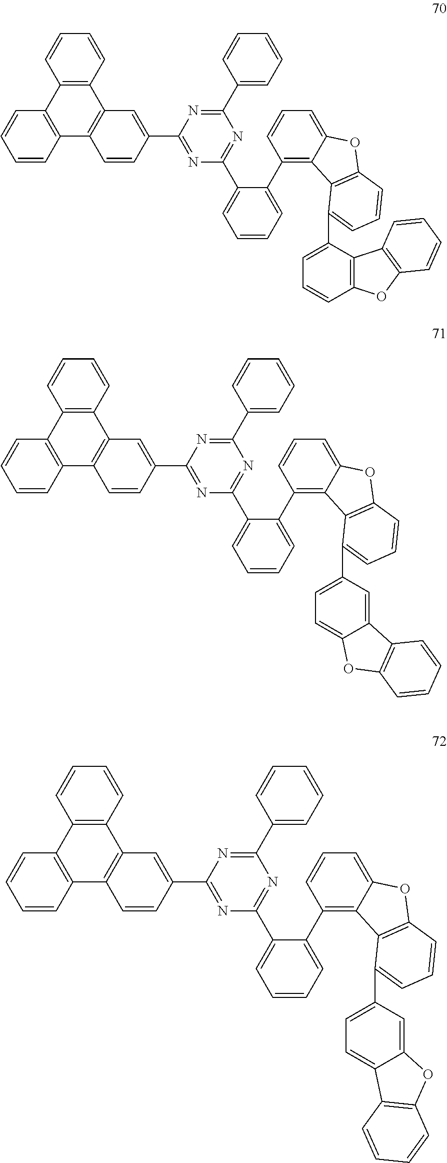 [SMILES: c1ccc(-c2nc(-c3ccc4c5ccccc5c5ccccc5c4c3)nc(-c3ccccc3-c3cccc4oc5cccc(-c6cccc7oc8ccccc8c67)c5c34)n2)cc1 ; c1ccc(-c2nc(-c3ccc4c5ccccc5c5ccccc5c4c3)nc(-c3ccccc3-c3cccc4oc5cccc(-c6ccc7oc8ccccc8c7c6)c5c34)n2)cc1 ; c1ccc(-c2nc(-c3ccc4c5ccccc5c5ccccc5c4c3)nc(-c3ccccc3-c3cccc4oc5cccc(-c6ccc7c(c6)oc6ccccc67)c5c34)n2)cc1]

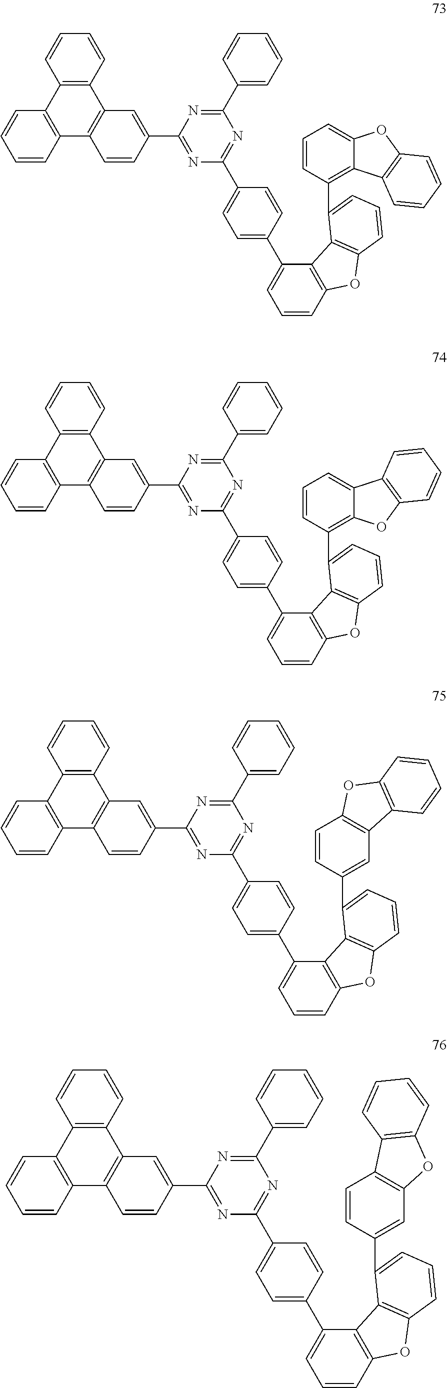 [SMILES: c1ccc(-c2nc(-c3ccc(-c4cccc5oc6cccc(-c7cccc8oc9ccccc9c78)c6c45)cc3)nc(-c3ccc4c5ccccc5c5ccccc5c4c3)n2)cc1 ; c1ccc(-c2nc(-c3ccc(-c4cccc5oc6cccc(-c7cccc8c7oc7ccccc78)c6c45)cc3)nc(-c3ccc4c5ccccc5c5ccccc5c4c3)n2)cc1 ; c1ccc(-c2nc(-c3ccc(-c4cccc5oc6cccc(-c7ccc8oc9ccccc9c8c7)c6c45)cc3)nc(-c3ccc4c5ccccc5c5ccccc5c4c3)n2)cc1 ; c1ccc(-c2nc(-c3ccc(-c4cccc5oc6cccc(-c7ccc8c(c7)oc7ccccc78)c6c45)cc3)nc(-c3ccc4c5ccccc5c5ccccc5c4c3)n2)cc1]

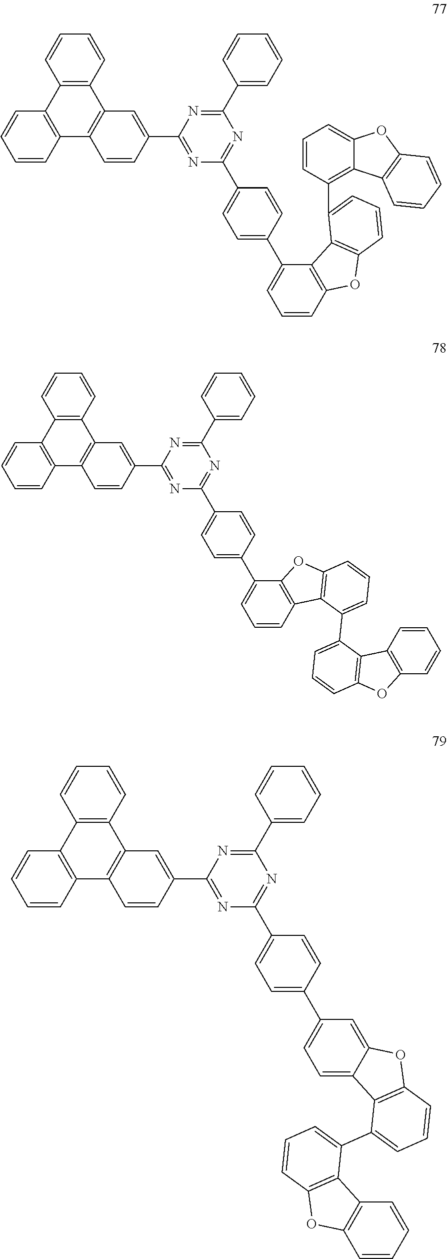 [SMILES: c1ccc(-c2nc(-c3ccc(-c4cccc5oc6cccc(-c7cccc8oc9ccccc9c78)c6c45)cc3)nc(-c3ccc4c5ccccc5c5ccccc5c4c3)n2)cc1 ; c1ccc(-c2nc(-c3ccc(-c4cccc5c4oc4cccc(-c6cccc7oc8ccccc8c67)c45)cc3)nc(-c3ccc4c5ccccc5c5ccccc5c4c3)n2)cc1 ; c1ccc(-c2nc(-c3ccc(-c4ccc5c(c4)oc4cccc(-c6cccc7oc8ccccc8c67)c45)cc3)nc(-c3ccc4c5ccccc5c5ccccc5c4c3)n2)cc1]

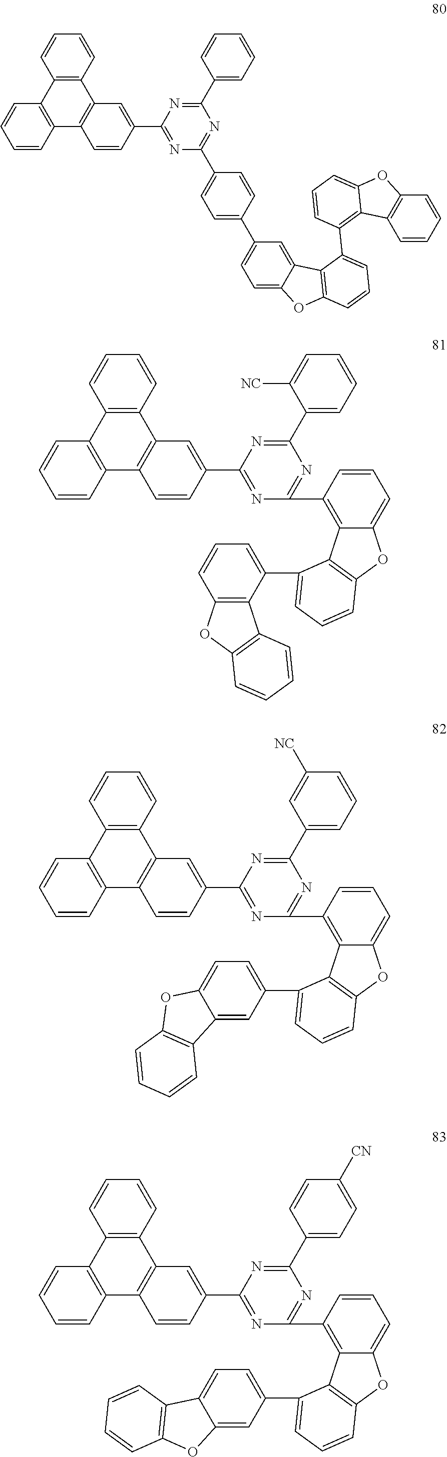 [SMILES: c1ccc(-c2nc(-c3ccc(-c4ccc5oc6cccc(-c7cccc8oc9ccccc9c78)c6c5c4)cc3)nc(-c3ccc4c5ccccc5c5ccccc5c4c3)n2)cc1 ; N#Cc1ccccc1-c1nc(-c2ccc3c4ccccc4c4ccccc4c3c2)nc(-c2cccc3oc4cccc(-c5cccc6oc7ccccc7c56)c4c23)n1 ; N#Cc1cccc(-c2nc(-c3ccc4c5ccccc5c5ccccc5c4c3)nc(-c3cccc4oc5cccc(-c6ccc7oc8ccccc8c7c6)c5c34)n2)c1 ; N#Cc1ccc(-c2nc(-c3ccc4c5ccccc5c5ccccc5c4c3)nc(-c3cccc4oc5cccc(-c6ccc7c(c6)oc6ccccc67)c5c34)n2)cc1]

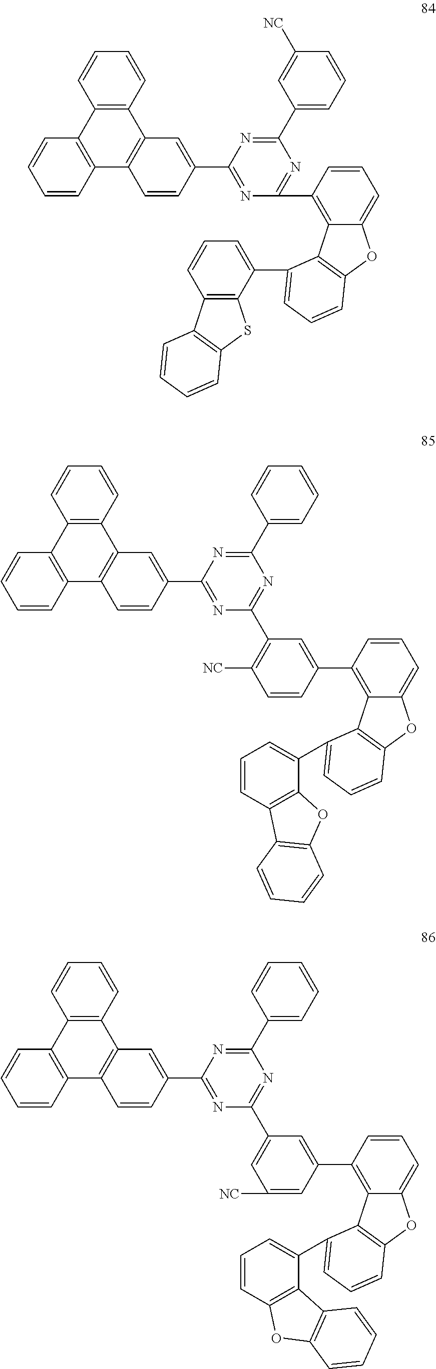 [SMILES: N#Cc1cccc(-c2nc(-c3ccc4c5ccccc5c5ccccc5c4c3)nc(-c3cccc4oc5cccc(-c6cccc7c6sc6ccccc67)c5c34)n2)c1 ; N#Cc1ccc(-c2cccc3oc4cccc(-c5cccc6c5oc5ccccc56)c4c23)cc1-c1nc(-c2ccccc2)nc(-c2ccc3c4ccccc4c4ccccc4c3c2)n1 ; N#Cc1cc(-c2nc(-c3ccccc3)nc(-c3ccc4c5ccccc5c5ccccc5c4c3)n2)cc(-c2cccc3oc4cccc(-c5cccc6oc7ccccc7c56)c4c23)c1]

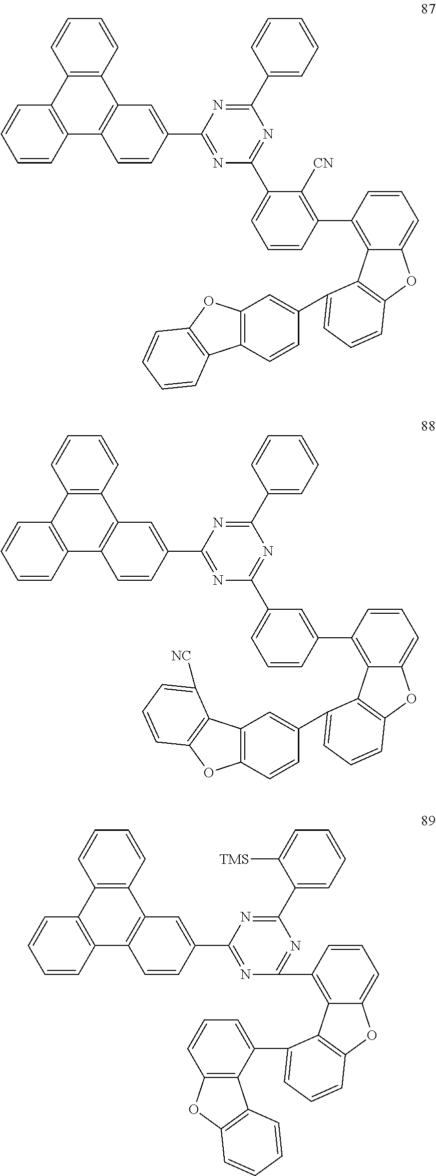 [SMILES: N#Cc1c(-c2nc(-c3ccccc3)nc(-c3ccc4c5ccccc5c5ccccc5c4c3)n2)cccc1-c1cccc2oc3cccc(-c4ccc5c(c4)oc4ccccc45)c3c12 ; N#Cc1cccc2oc3ccc(-c4cccc5oc6cccc(-c7cccc(-c8nc(-c9ccccc9)nc(-c9ccc%10c%11ccccc%11c%11ccccc%11c%10c9)n8)c7)c6c45)cc3c12 ; C[Si](C)(C)c1ccccc1-c1nc(-c2ccc3c4ccccc4c4ccccc4c3c2)nc(-c2cccc3oc4cccc(-c5cccc6oc7ccccc7c56)c4c23)n1]

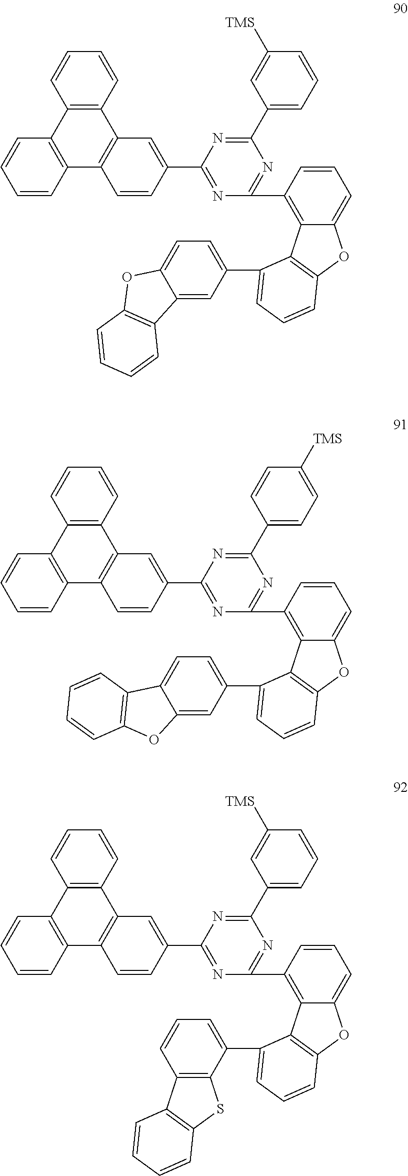 [SMILES: C[Si](C)(C)c1cccc(-c2nc(-c3ccc4c5ccccc5c5ccccc5c4c3)nc(-c3cccc4oc5cccc(-c6ccc7oc8ccccc8c7c6)c5c34)n2)c1 ; C[Si](C)(C)c1ccc(-c2nc(-c3ccc4c5ccccc5c5ccccc5c4c3)nc(-c3cccc4oc5cccc(-c6ccc7c(c6)oc6ccccc67)c5c34)n2)cc1 ; C[Si](C)(C)c1cccc(-c2nc(-c3ccc4c5ccccc5c5ccccc5c4c3)nc(-c3cccc4oc5cccc(-c6cccc7c6sc6ccccc67)c5c34)n2)c1]

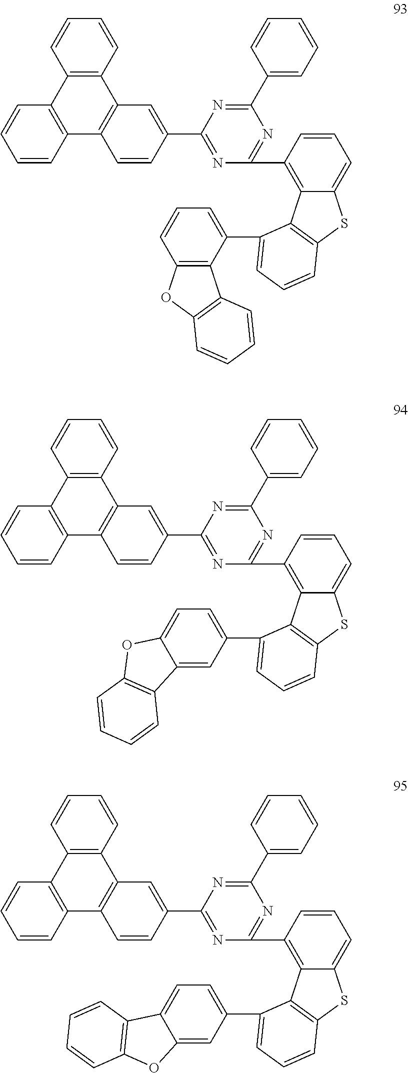 [SMILES: c1ccc(-c2nc(-c3ccc4c5ccccc5c5ccccc5c4c3)nc(-c3cccc4sc5cccc(-c6cccc7oc8ccccc8c67)c5c34)n2)cc1 ; c1ccc(-c2nc(-c3ccc4c5ccccc5c5ccccc5c4c3)nc(-c3cccc4sc5cccc(-c6ccc7oc8ccccc8c7c6)c5c34)n2)cc1 ; c1ccc(-c2nc(-c3ccc4c5ccccc5c5ccccc5c4c3)nc(-c3cccc4sc5cccc(-c6ccc7c(c6)oc6ccccc67)c5c34)n2)cc1]

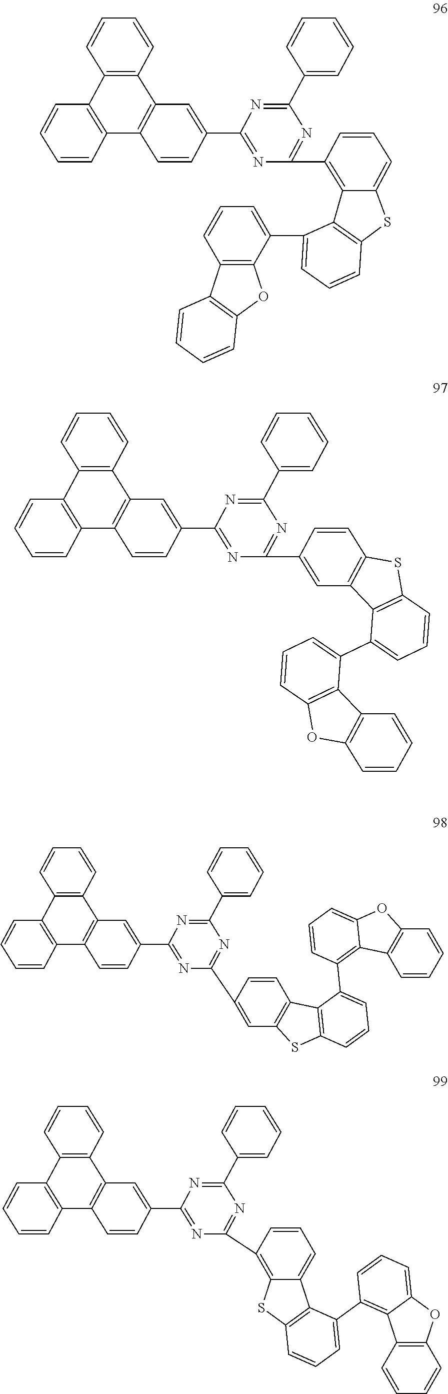 [SMILES: c1ccc(-c2nc(-c3ccc4c5ccccc5c5ccccc5c4c3)nc(-c3cccc4sc5cccc(-c6cccc7c6oc6ccccc67)c5c34)n2)cc1 ; c1ccc(-c2nc(-c3ccc4c5ccccc5c5ccccc5c4c3)nc(-c3ccc4sc5cccc(-c6cccc7oc8ccccc8c67)c5c4c3)n2)cc1 ; c1ccc(-c2nc(-c3ccc4c(c3)sc3cccc(-c5cccc6oc7ccccc7c56)c34)nc(-c3ccc4c5ccccc5c5ccccc5c4c3)n2)cc1 ; c1ccc(-c2nc(-c3ccc4c5ccccc5c5ccccc5c4c3)nc(-c3cccc4c3sc3cccc(-c5cccc6oc7ccccc7c56)c34)n2)cc1]

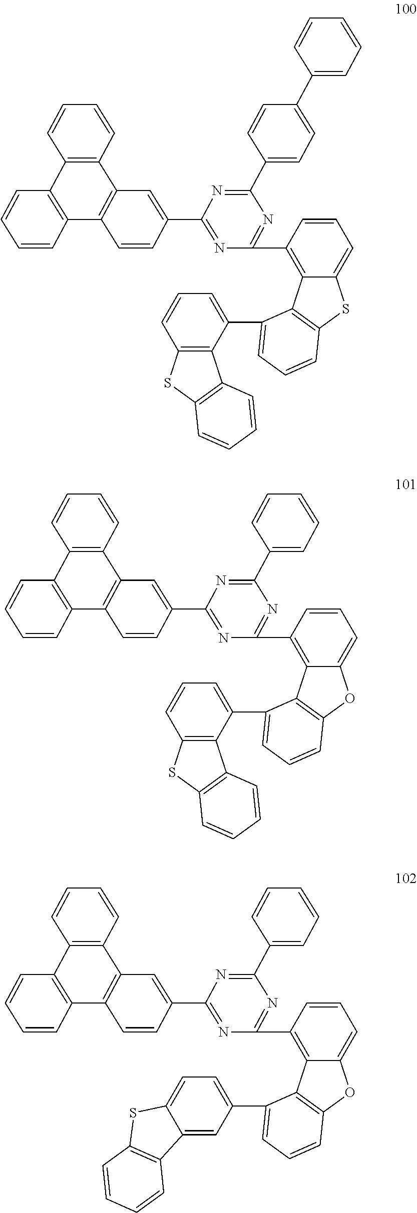 [SMILES: c1ccc(-c2ccc(-c3nc(-c4ccc5c6ccccc6c6ccccc6c5c4)nc(-c4cccc5sc6cccc(-c7cccc8sc9ccccc9c78)c6c45)n3)cc2)cc1 ; c1ccc(-c2nc(-c3ccc4c5ccccc5c5ccccc5c4c3)nc(-c3cccc4oc5cccc(-c6cccc7sc8ccccc8c67)c5c34)n2)cc1 ; c1ccc(-c2nc(-c3ccc4c5ccccc5c5ccccc5c4c3)nc(-c3cccc4oc5cccc(-c6ccc7sc8ccccc8c7c6)c5c34)n2)cc1]

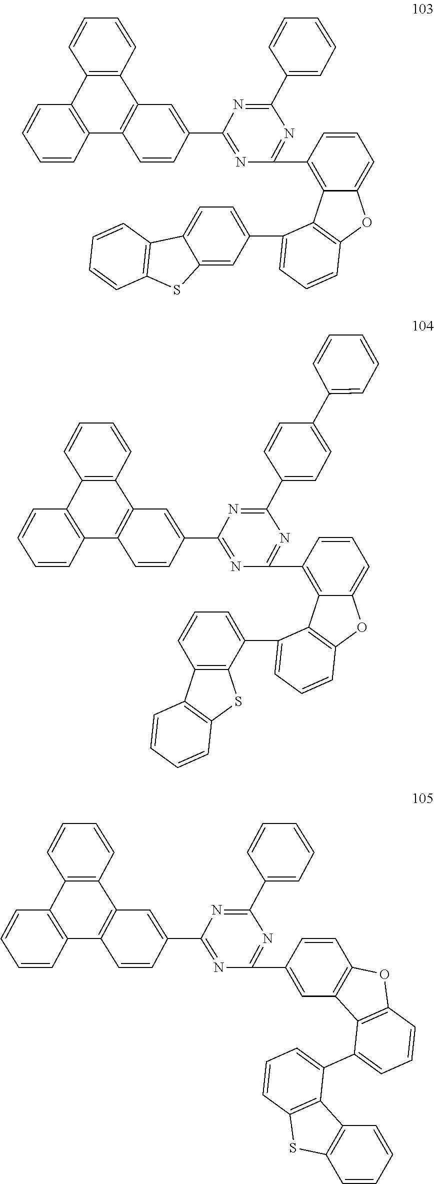 [SMILES: c1ccc(-c2nc(-c3ccc4c5ccccc5c5ccccc5c4c3)nc(-c3cccc4oc5cccc(-c6ccc7c(c6)sc6ccccc67)c5c34)n2)cc1 ; c1ccc(-c2ccc(-c3nc(-c4ccc5c6ccccc6c6ccccc6c5c4)nc(-c4cccc5oc6cccc(-c7cccc8c7sc7ccccc78)c6c45)n3)cc2)cc1 ; c1ccc(-c2nc(-c3ccc4c5ccccc5c5ccccc5c4c3)nc(-c3ccc4oc5cccc(-c6cccc7sc8ccccc8c67)c5c4c3)n2)cc1]

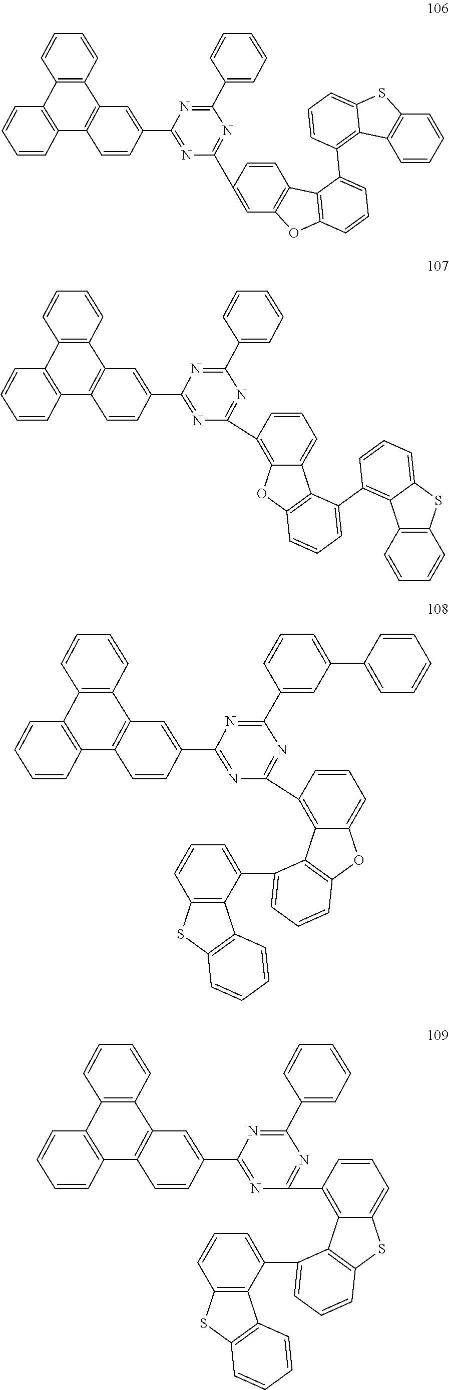 [SMILES: c1ccc(-c2nc(-c3ccc4c(c3)oc3cccc(-c5cccc6sc7ccccc7c56)c34)nc(-c3ccc4c5ccccc5c5ccccc5c4c3)n2)cc1 ; c1ccc(-c2nc(-c3ccc4c5ccccc5c5ccccc5c4c3)nc(-c3cccc4c3oc3cccc(-c5cccc6sc7ccccc7c56)c34)n2)cc1 ; c1ccc(-c2cccc(-c3nc(-c4ccc5c6ccccc6c6ccccc6c5c4)nc(-c4cccc5oc6cccc(-c7cccc8sc9ccccc9c78)c6c45)n3)c2)cc1 ; c1ccc(-c2nc(-c3ccc4c5ccccc5c5ccccc5c4c3)nc(-c3cccc4sc5cccc(-c6cccc7sc8ccccc8c67)c5c34)n2)cc1]

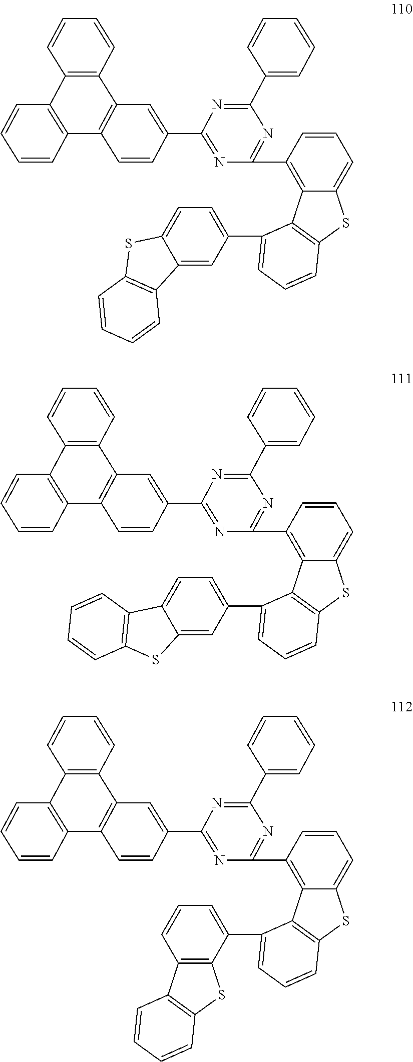 [SMILES: c1ccc(-c2nc(-c3ccc4c5ccccc5c5ccccc5c4c3)nc(-c3cccc4sc5cccc(-c6ccc7sc8ccccc8c7c6)c5c34)n2)cc1 ; c1ccc(-c2nc(-c3ccc4c5ccccc5c5ccccc5c4c3)nc(-c3cccc4sc5cccc(-c6ccc7c(c6)sc6ccccc67)c5c34)n2)cc1 ; c1ccc(-c2nc(-c3ccc4c5ccccc5c5ccccc5c4c3)nc(-c3cccc4sc5cccc(-c6cccc7c6sc6ccccc67)c5c34)n2)cc1]

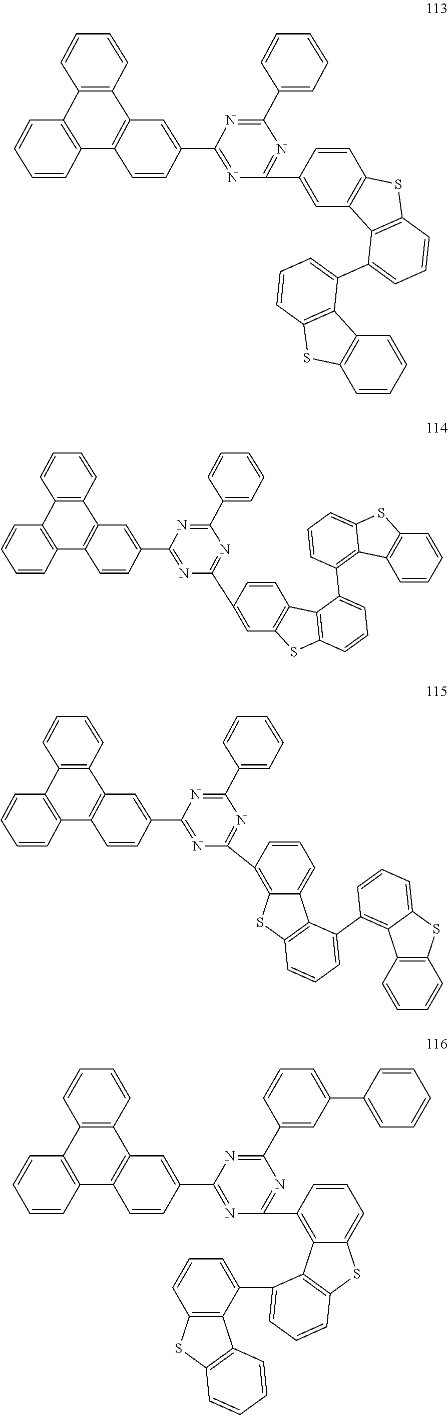 [SMILES: c1ccc(-c2nc(-c3ccc4c5ccccc5c5ccccc5c4c3)nc(-c3ccc4sc5cccc(-c6cccc7sc8ccccc8c67)c5c4c3)n2)cc1 ; c1ccc(-c2nc(-c3ccc4c(c3)sc3cccc(-c5cccc6sc7ccccc7c56)c34)nc(-c3ccc4c5ccccc5c5ccccc5c4c3)n2)cc1 ; c1ccc(-c2nc(-c3ccc4c5ccccc5c5ccccc5c4c3)nc(-c3cccc4c3sc3cccc(-c5cccc6sc7ccccc7c56)c34)n2)cc1 ; c1ccc(-c2cccc(-c3nc(-c4ccc5c6ccccc6c6ccccc6c5c4)nc(-c4cccc5sc6cccc(-c7cccc8sc9ccccc9c78)c6c45)n3)c2)cc1]

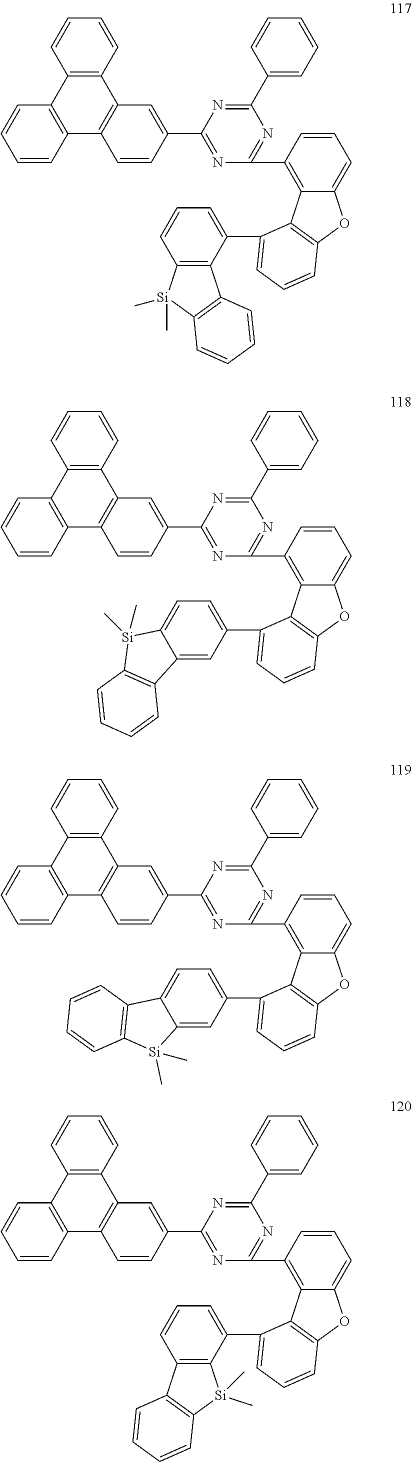 [SMILES: C[Si]1(C)c2ccccc2-c2c(-c3cccc4oc5cccc(-c6nc(-c7ccccc7)nc(-c7ccc8c9ccccc9c9ccccc9c8c7)n6)c5c34)cccc21 ; C[Si]1(C)c2ccccc2-c2cc(-c3cccc4oc5cccc(-c6nc(-c7ccccc7)nc(-c7ccc8c9ccccc9c9ccccc9c8c7)n6)c5c34)ccc21 ; C[Si]1(C)c2ccccc2-c2ccc(-c3cccc4oc5cccc(-c6nc(-c7ccccc7)nc(-c7ccc8c9ccccc9c9ccccc9c8c7)n6)c5c34)cc21 ; C[Si]1(C)c2ccccc2-c2cccc(-c3cccc4oc5cccc(-c6nc(-c7ccccc7)nc(-c7ccc8c9ccccc9c9ccccc9c8c7)n6)c5c34)c21]

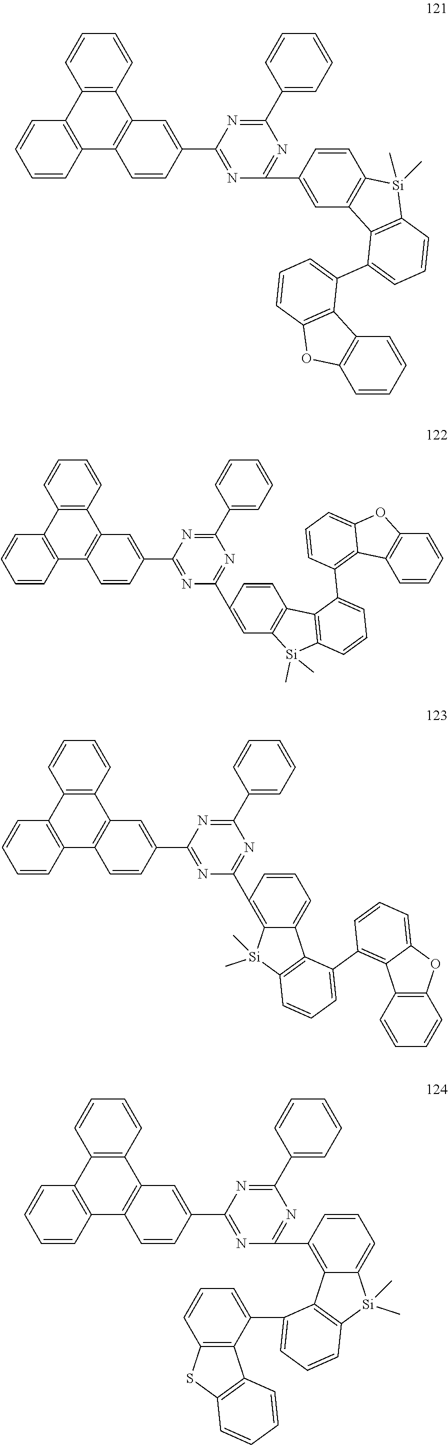 [SMILES: C[Si]1(C)c2ccc(-c3nc(-c4ccccc4)nc(-c4ccc5c6ccccc6c6ccccc6c5c4)n3)cc2-c2c(-c3cccc4oc5ccccc5c34)cccc21 ; C[Si]1(C)c2cc(-c3nc(-c4ccccc4)nc(-c4ccc5c6ccccc6c6ccccc6c5c4)n3)ccc2-c2c(-c3cccc4oc5ccccc5c34)cccc21 ; C[Si]1(C)c2cccc(-c3cccc4oc5ccccc5c34)c2-c2cccc(-c3nc(-c4ccccc4)nc(-c4ccc5c6ccccc6c6ccccc6c5c4)n3)c21 ; C[Si]1(C)c2cccc(-c3nc(-c4ccccc4)nc(-c4ccc5c6ccccc6c6ccccc6c5c4)n3)c2-c2c(-c3cccc4sc5ccccc5c34)cccc21]

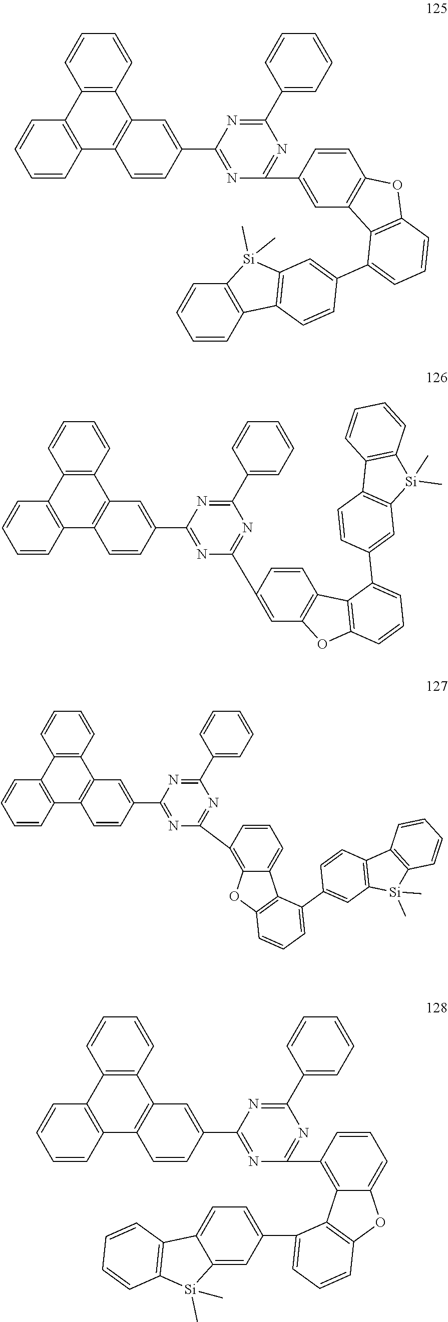 [SMILES: C[Si]1(C)c2ccccc2-c2ccc(-c3cccc4oc5ccc(-c6nc(-c7ccccc7)nc(-c7ccc8c9ccccc9c9ccccc9c8c7)n6)cc5c34)cc21 ; C[Si]1(C)c2ccccc2-c2ccc(-c3cccc4oc5cc(-c6nc(-c7ccccc7)nc(-c7ccc8c9ccccc9c9ccccc9c8c7)n6)ccc5c34)cc21 ; C[Si]1(C)c2ccccc2-c2ccc(-c3cccc4oc5c(-c6nc(-c7ccccc7)nc(-c7ccc8c9ccccc9c9ccccc9c8c7)n6)cccc5c34)cc21 ; C[Si]1(C)c2ccccc2-c2ccc(-c3cccc4oc5cccc(-c6nc(-c7ccccc7)nc(-c7ccc8c9ccccc9c9ccccc9c8c7)n6)c5c34)cc21]

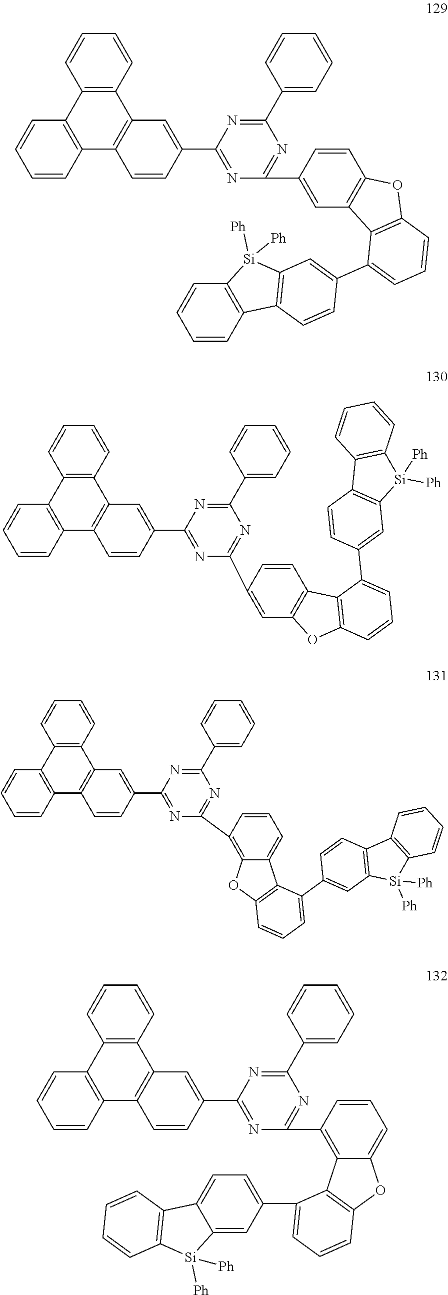 [SMILES: c1ccc(-c2nc(-c3ccc4c5ccccc5c5ccccc5c4c3)nc(-c3ccc4oc5cccc(-c6ccc7c(c6)[Si](c6ccccc6)(c6ccccc6)c6ccccc6-7)c5c4c3)n2)cc1 ; c1ccc(-c2nc(-c3ccc4c(c3)oc3cccc(-c5ccc6c(c5)[Si](c5ccccc5)(c5ccccc5)c5ccccc5-6)c34)nc(-c3ccc4c5ccccc5c5ccccc5c4c3)n2)cc1 ; c1ccc(-c2nc(-c3ccc4c5ccccc5c5ccccc5c4c3)nc(-c3cccc4c3oc3cccc(-c5ccc6c(c5)[Si](c5ccccc5)(c5ccccc5)c5ccccc5-6)c34)n2)cc1 ; c1ccc(-c2nc(-c3ccc4c5ccccc5c5ccccc5c4c3)nc(-c3cccc4oc5cccc(-c6ccc7c(c6)[Si](c6ccccc6)(c6ccccc6)c6ccccc6-7)c5c34)n2)cc1]

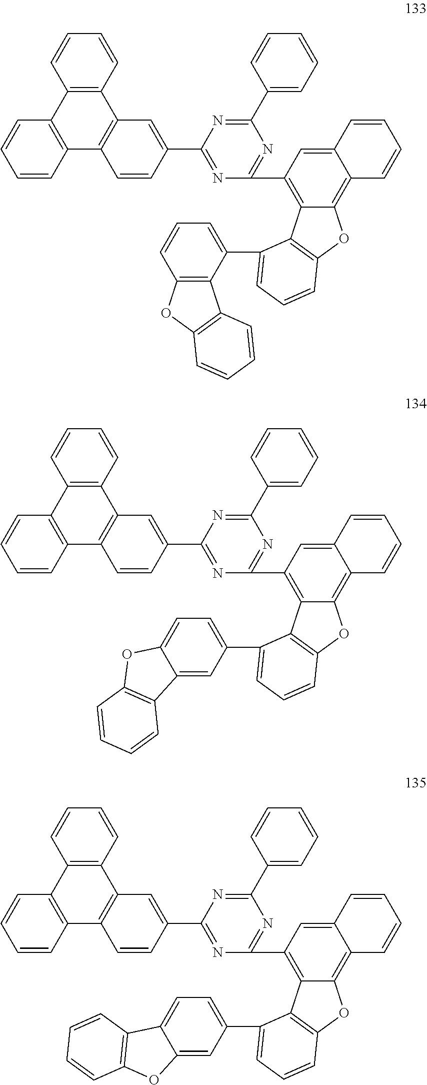 [SMILES: c1ccc(-c2nc(-c3ccc4c5ccccc5c5ccccc5c4c3)nc(-c3cc4ccccc4c4oc5cccc(-c6cccc7oc8ccccc8c67)c5c34)n2)cc1 ; c1ccc(-c2nc(-c3ccc4c5ccccc5c5ccccc5c4c3)nc(-c3cc4ccccc4c4oc5cccc(-c6ccc7oc8ccccc8c7c6)c5c34)n2)cc1 ; c1ccc(-c2nc(-c3ccc4c5ccccc5c5ccccc5c4c3)nc(-c3cc4ccccc4c4oc5cccc(-c6ccc7c(c6)oc6ccccc67)c5c34)n2)cc1]

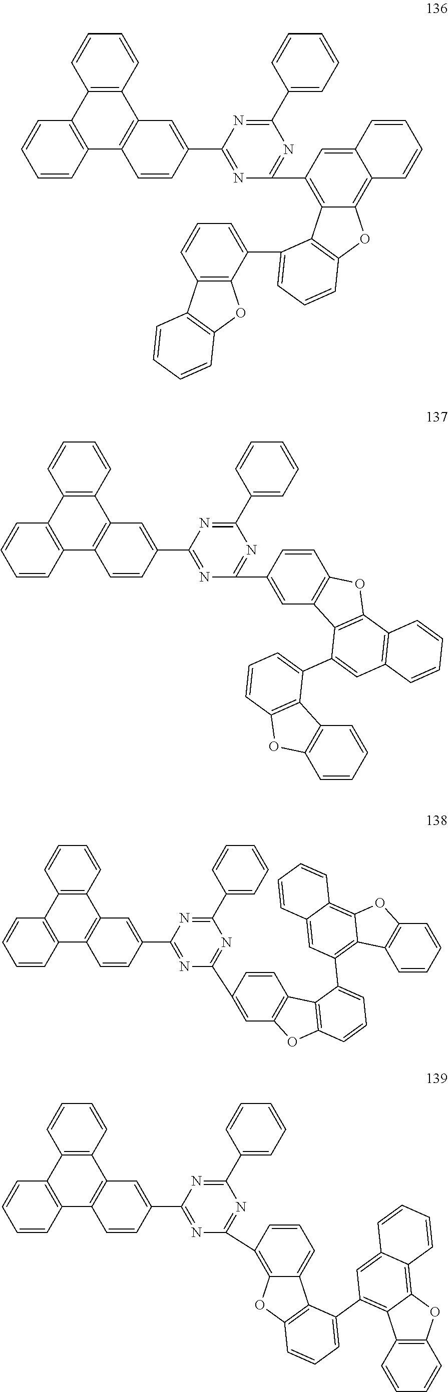 [SMILES: c1ccc(-c2nc(-c3ccc4c5ccccc5c5ccccc5c4c3)nc(-c3cc4ccccc4c4oc5cccc(-c6cccc7c6oc6ccccc67)c5c34)n2)cc1 ; c1ccc(-c2nc(-c3ccc4c5ccccc5c5ccccc5c4c3)nc(-c3ccc4oc5c6ccccc6cc(-c6cccc7oc8ccccc8c67)c5c4c3)n2)cc1 ; c1ccc(-c2nc(-c3ccc4c(c3)oc3cccc(-c5cc6ccccc6c6oc7ccccc7c56)c34)nc(-c3ccc4c5ccccc5c5ccccc5c4c3)n2)cc1 ; c1ccc(-c2nc(-c3ccc4c5ccccc5c5ccccc5c4c3)nc(-c3cccc4c3oc3cccc(-c5cc6ccccc6c6oc7ccccc7c56)c34)n2)cc1]

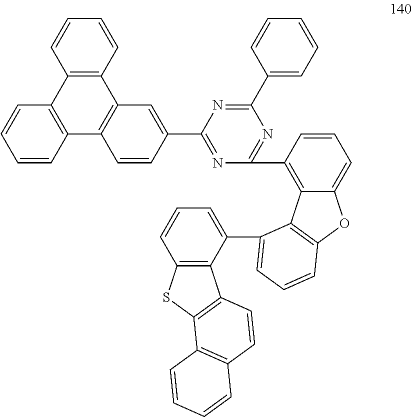 [SMILES: c1ccc(-c2nc(-c3ccc4c5ccccc5c5ccccc5c4c3)nc(-c3cccc4oc5cccc(-c6cccc7sc8c9ccccc9ccc8c67)c5c34)n2)cc1]

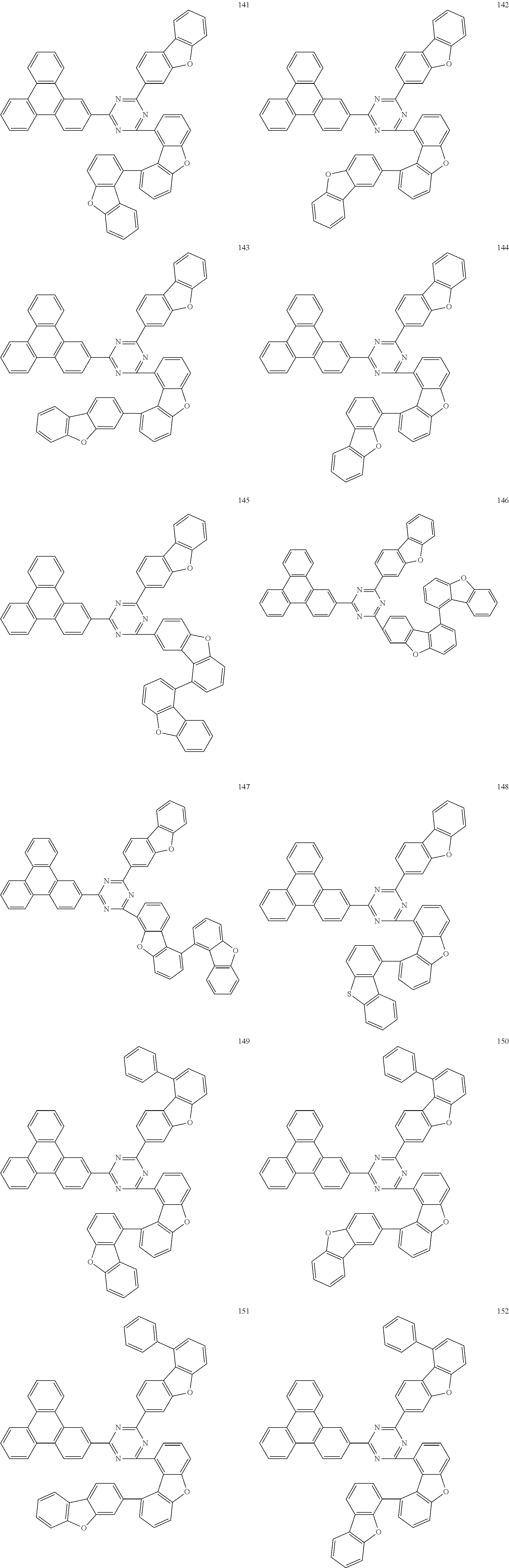

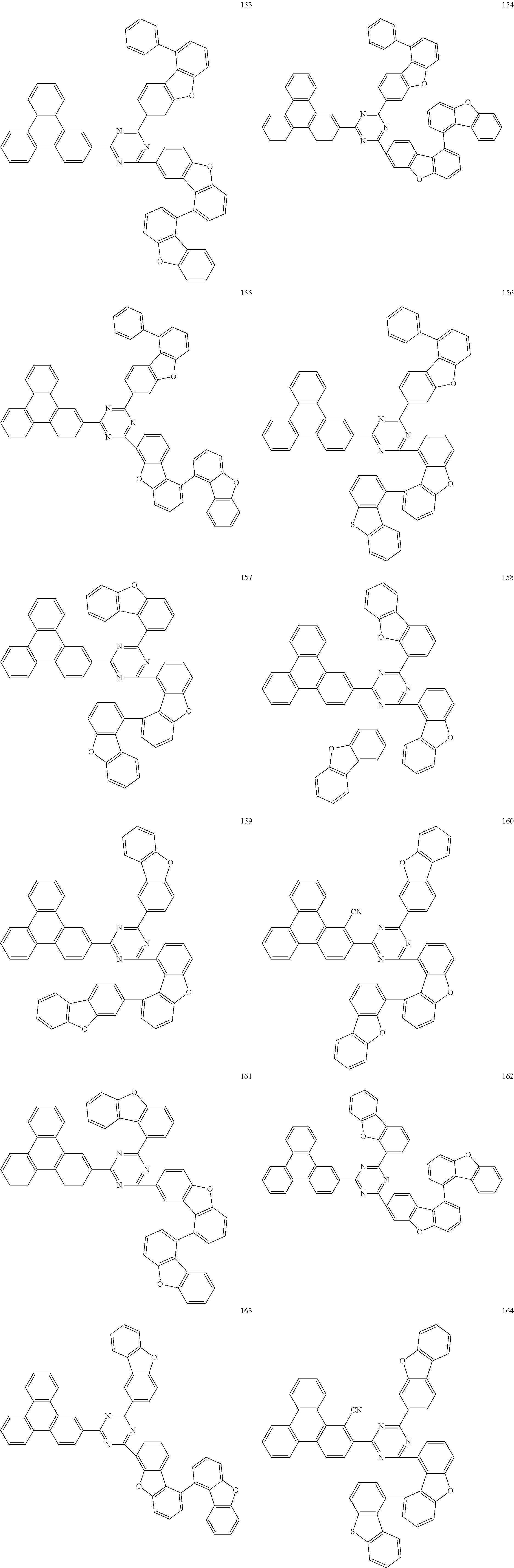

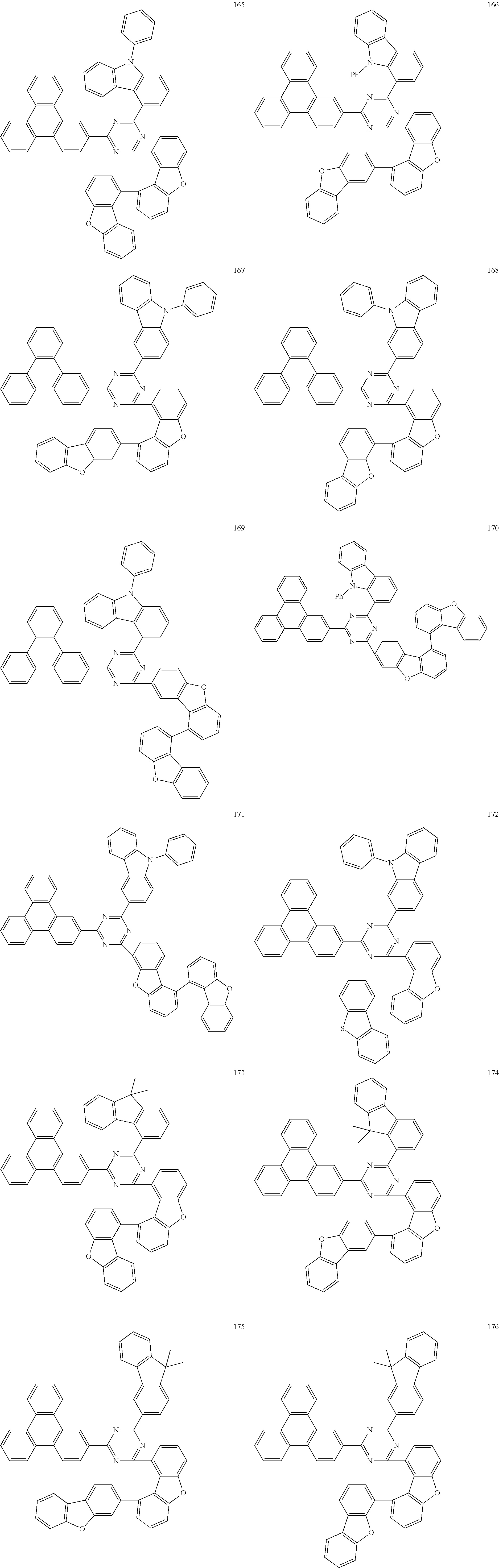

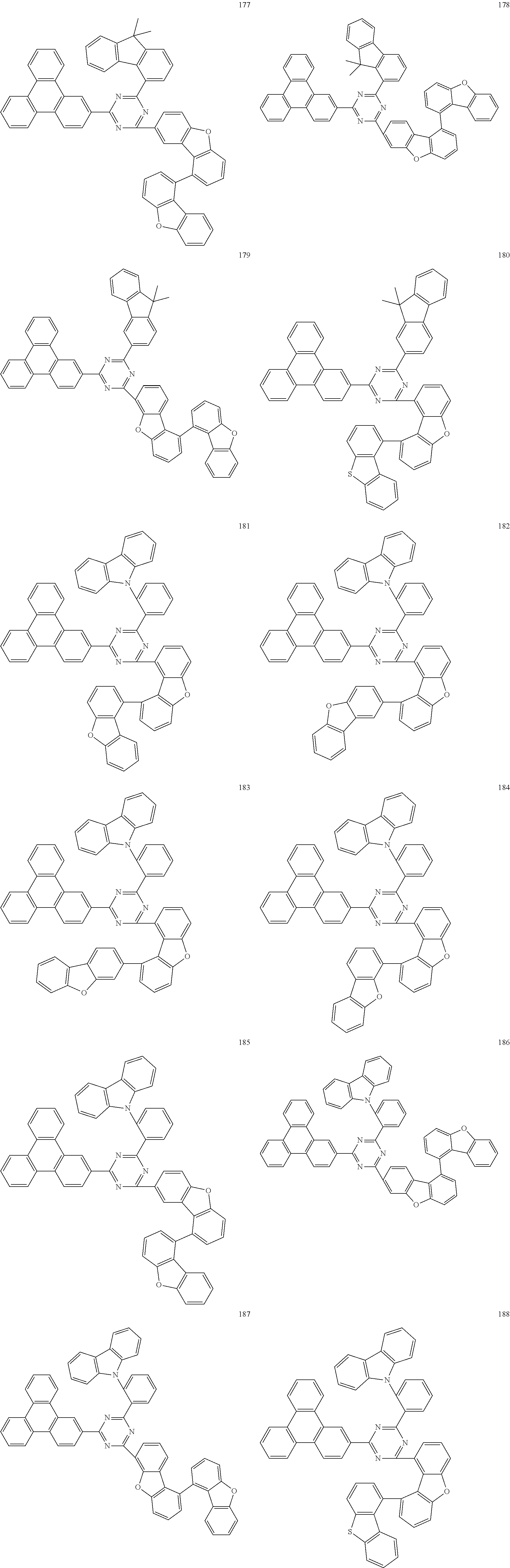

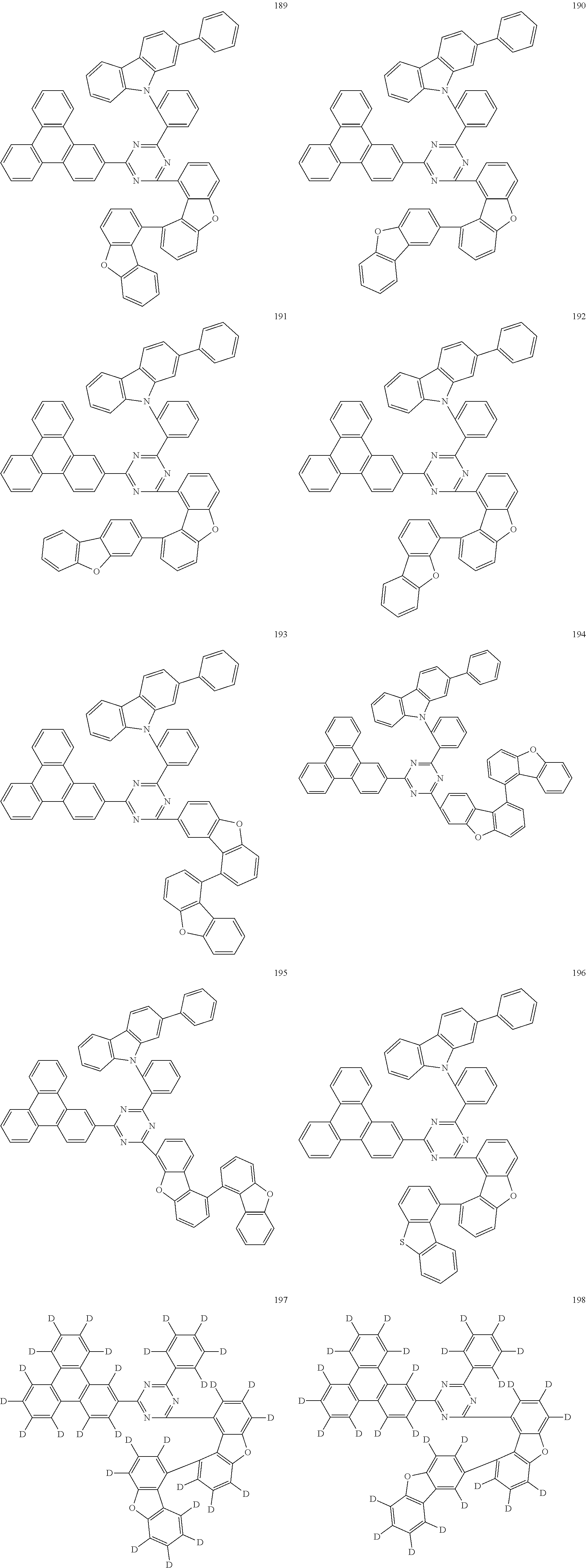

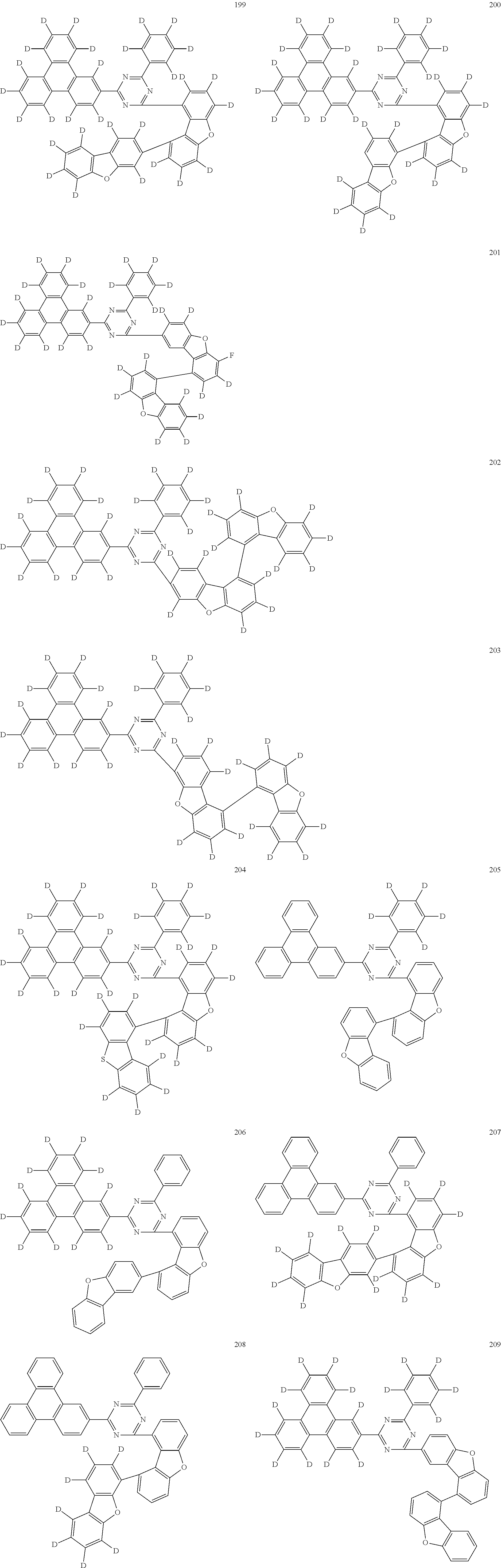

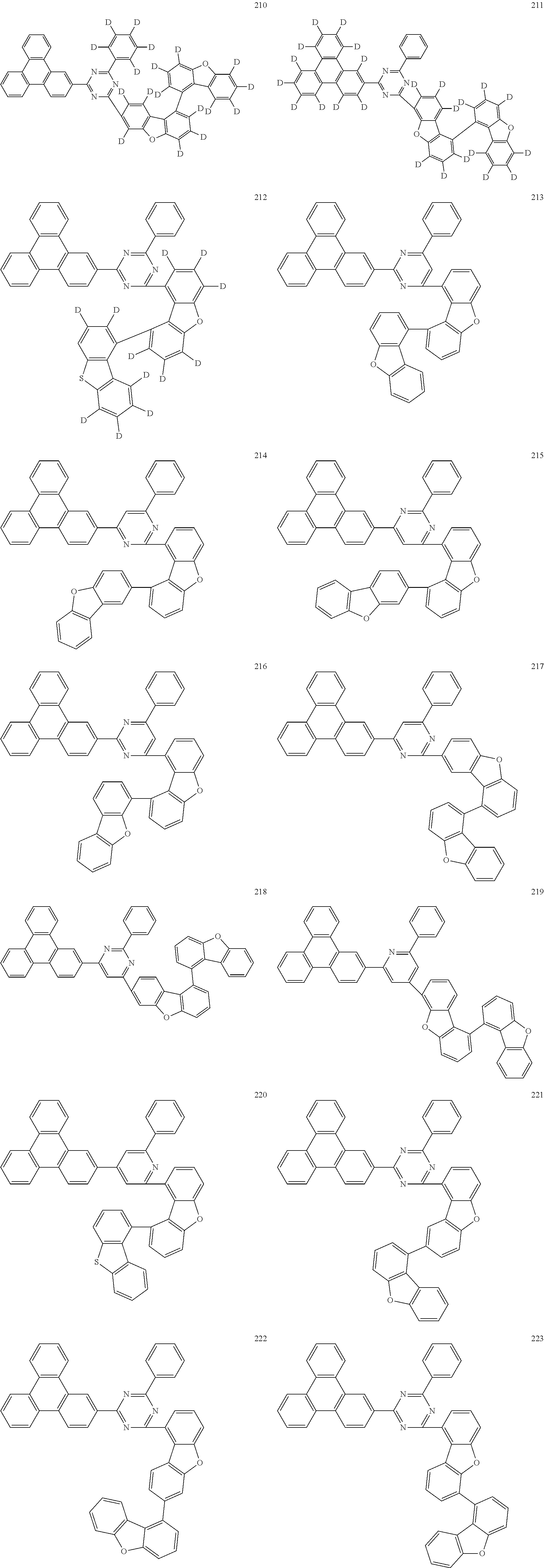

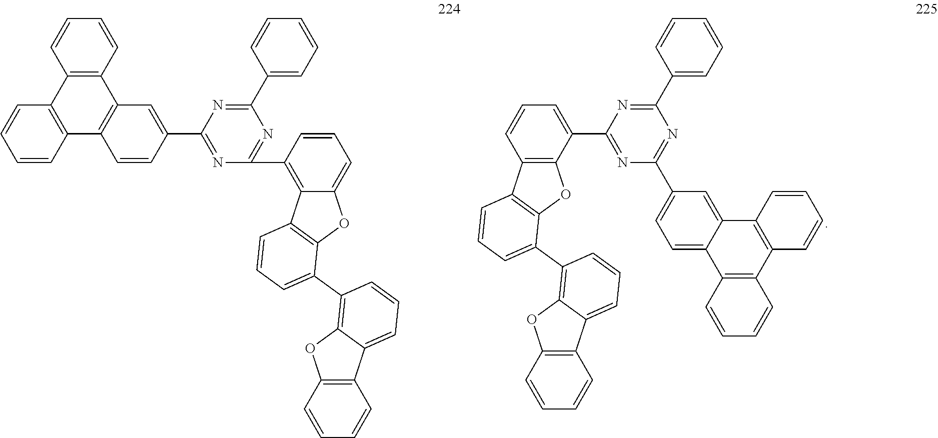 [SMILES: c1ccc(-c2nc(-c3ccc4c5ccccc5c5ccccc5c4c3)nc(-c3cccc4oc5c(-c6cccc7c6oc6ccccc67)cccc5c34)n2)cc1 ; c1ccc(-c2nc(-c3ccc4c5ccccc5c5ccccc5c4c3)nc(-c3cccc4c3oc3c(-c5cccc6c5oc5ccccc56)cccc34)n2)cc1]

7. A composition for an organic optoelectronic device, comprising
a first compound and a second compound,
wherein the first compound is the compound for an organic optoelectronic device of claim 1, and
the second compound is represented by Chemical Formula 2; or a combination of Chemical Formula 3 and Chemical Formula 4: 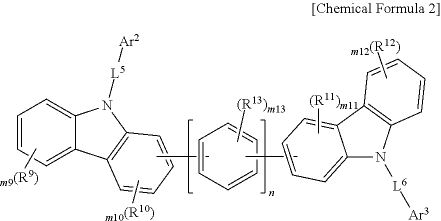
wherein, in Chemical Formula 2,
R⁹ to R¹³ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group,
Ar² and Ar³ are each independently a substituted or unsubstituted C6 to C20 aryl group or a substituted or unsubstituted C2 to C30 heterocyclic group,
L⁵ and L⁶ are each independently a single bond or a substituted or unsubstituted C6 to C20 arylene group,
m9, m12, and m13 are each independently one of integers of 1 to 4,
m10 and m11 are each independently one of integers of 1 to 3, and
n is one of integers of 0 to 2; 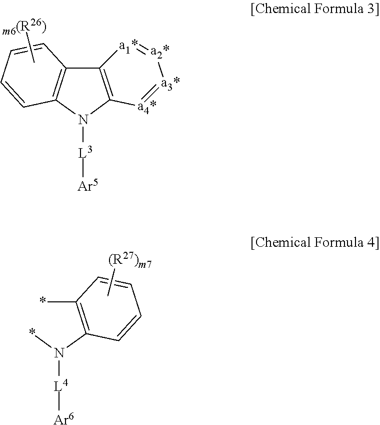
wherein, in Chemical Formula 3 and Chemical Formula 4,
a₁* to a₄* in Chemical Formula 3 are each independently a linking carbon (C) or C-L^{a}-R^{d},
among a₁* to a₄* in Chemical Formula 3, two adjacent ones are each linked to *s in Chemical Formula 4,
L^{a}, L⁷, and L⁸ are each independently a single bond or a substituted or unsubstituted C6 to C20 arylene group,
R^{d}, R¹⁴, and R¹⁵ are each independently hydrogen, deuterium, a cyano group, a halogen, a substituted or unsubstituted amine group, a substituted or unsubstituted C1 to C30 alkyl group, a substituted or unsubstituted C6 to C30 aryl group, or a substituted or unsubstituted C2 to C30 heterocyclic group,
Ar⁴ and Ar⁵ are each independently a substituted or unsubstituted C6 to C20 aryl group or a substituted or unsubstituted C2 to C30 heterocyclic group, and
m14 and m15 are each independently one of integers of 1 to 4.

8. The composition for an organic optoelectronic device as claimed in claim 7, wherein
Chemical Formula 2 is represented by Chemical Formula 2-8: 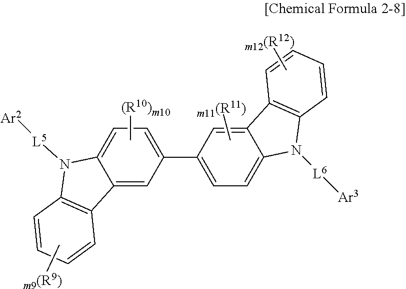
wherein, in Chemical Formula 2-8,
R⁹ to R¹² are each independently hydrogen, deuterium, or a substituted or unsubstituted C6 to C12 aryl group,
m9 and m12 are each independently one of integers of 1 to 4,
m10 and m11 are each independently one of integers of 1 to 3, and
*-L⁵-Ar² and *-L⁶-Ar³ are each independently one of the substituents listed in Group I, 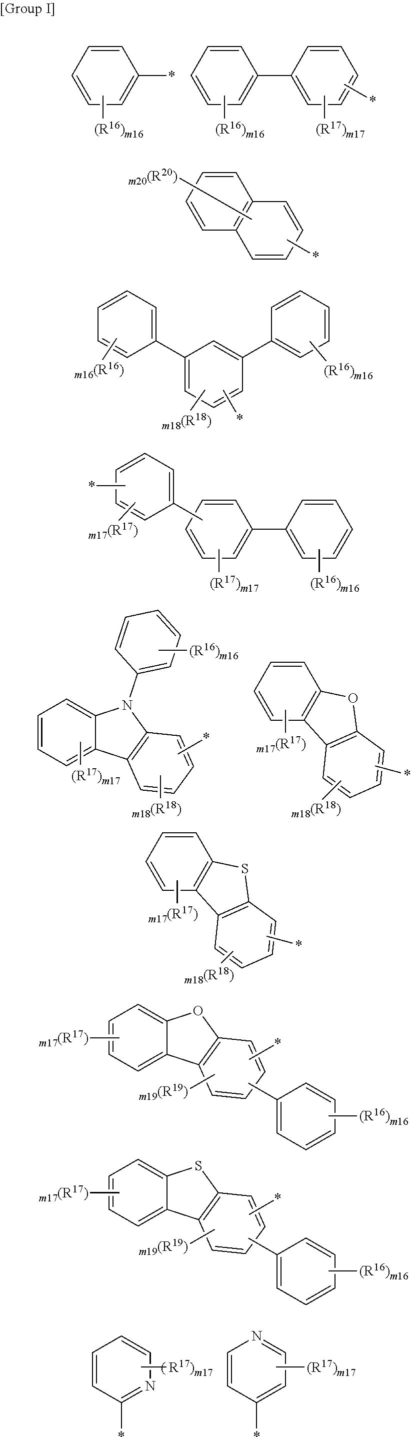 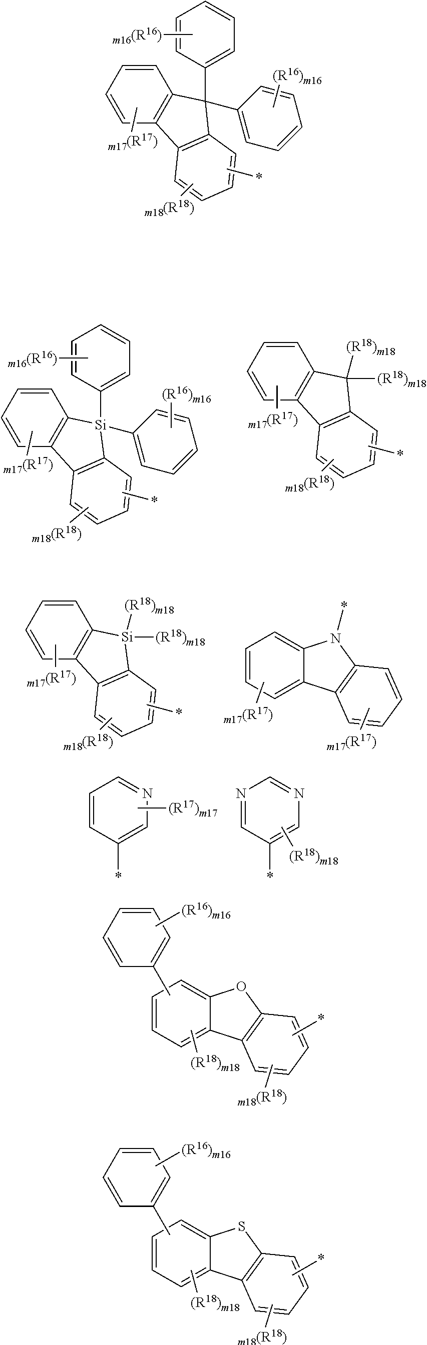 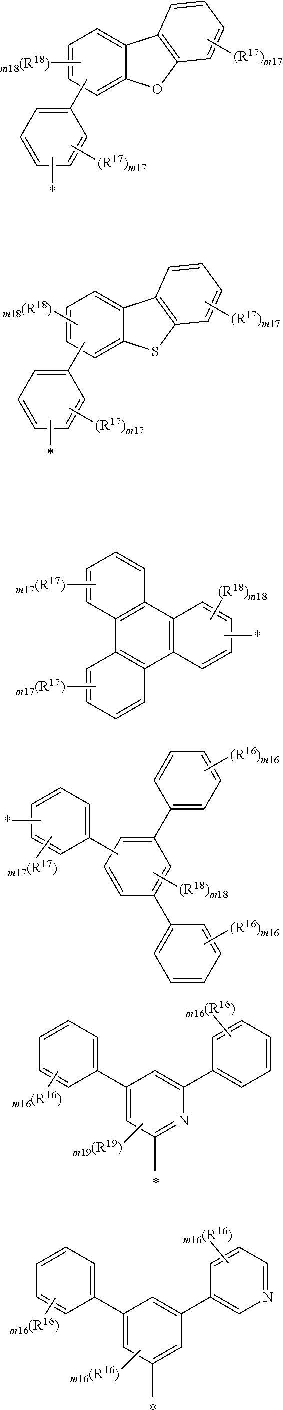 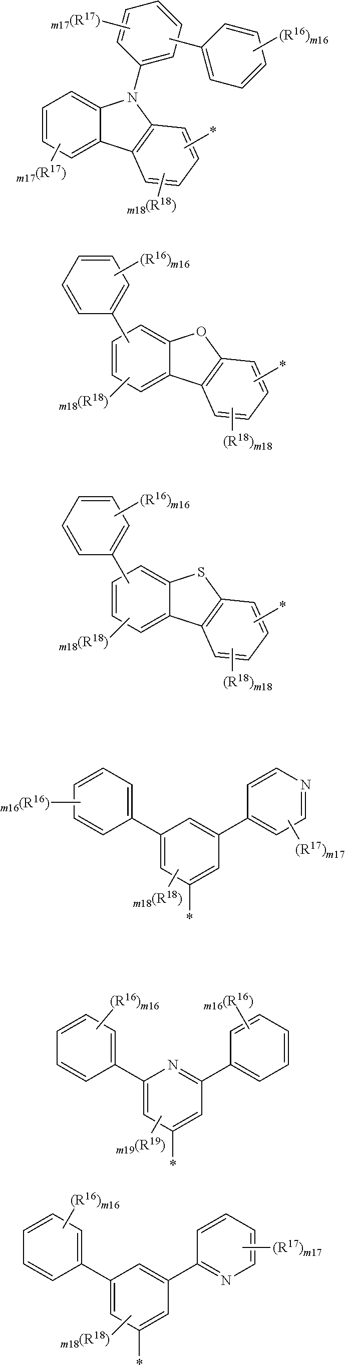
wherein, in Group I,
R¹⁶ to R²⁰ are each independently hydrogen, deuterium, a cyano group, a C1 to C10 alkyl group, or a C6 to C12 aryl group,
m16 is one of integers of 1 to 5,
m17 is one of integers of 1 to 4,
m18 is one of integers of 1 to 3,
m19 is an integer of 1 or 2,
m20 is one of integers of 1 to 7, and
* is a linking point.

9. The composition for an organic optoelectronic device as claimed in claim 7, wherein
the combination of Chemical Formula 3 and Chemical Formula 4 is represented by Chemical Formula 3C: 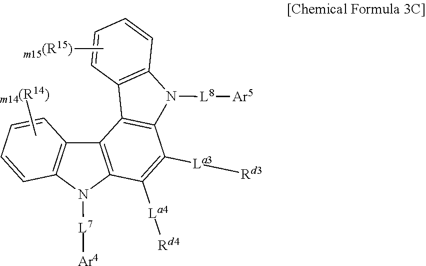
wherein, in Chemical Formula 3C,
L^{a3} and L^{a4} are a single bond,
R⁹, R¹⁰, R^{d3}, and R^{d4} are each independently hydrogen, deuterium or C6 to C12 aryl group,
m14 and m15 are each independently one of integers of 1 to 4,
*-L⁷-Ar⁴ and *-L⁸-Ar⁵ are each independently selected from the substituents listed in Group I, 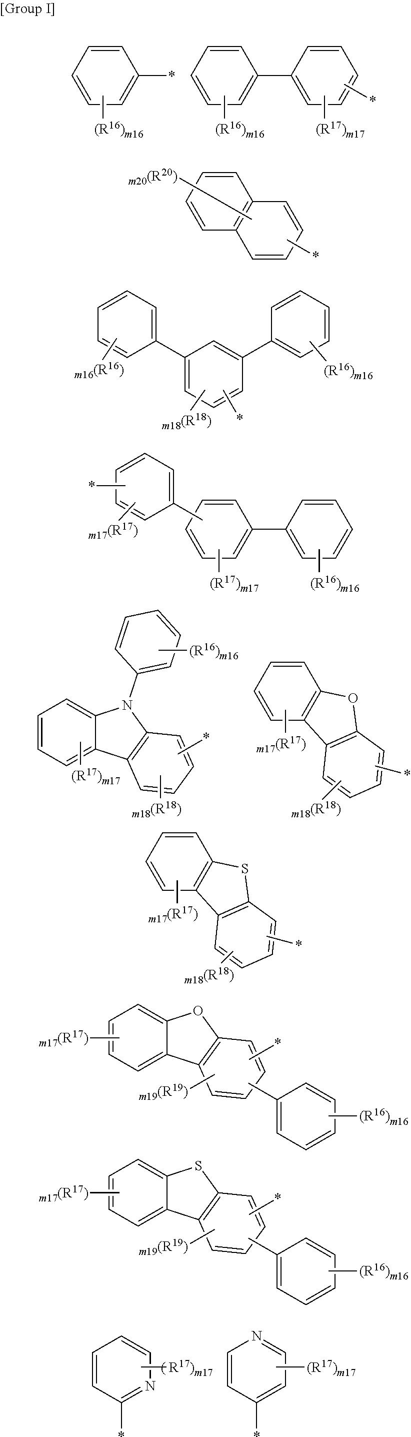 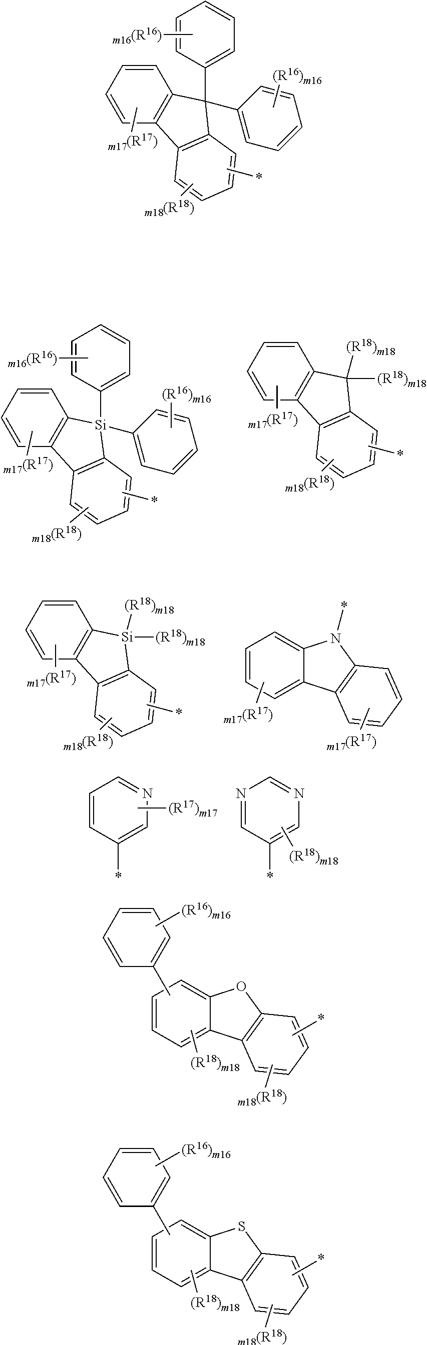 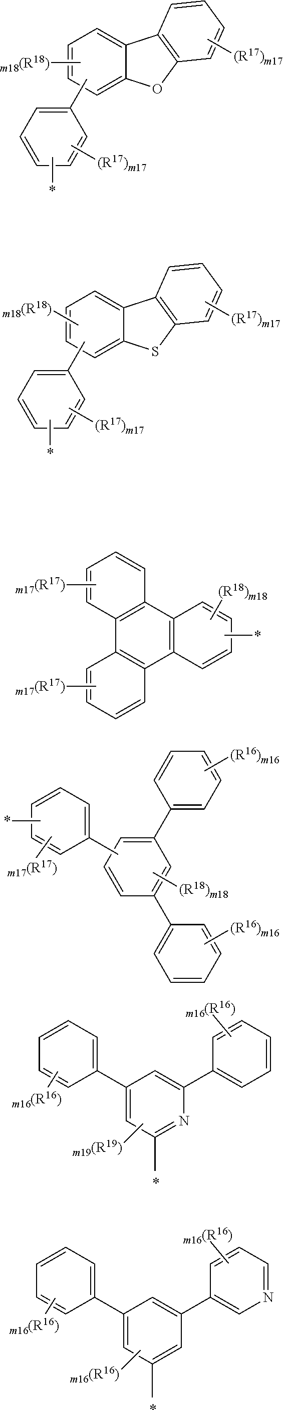 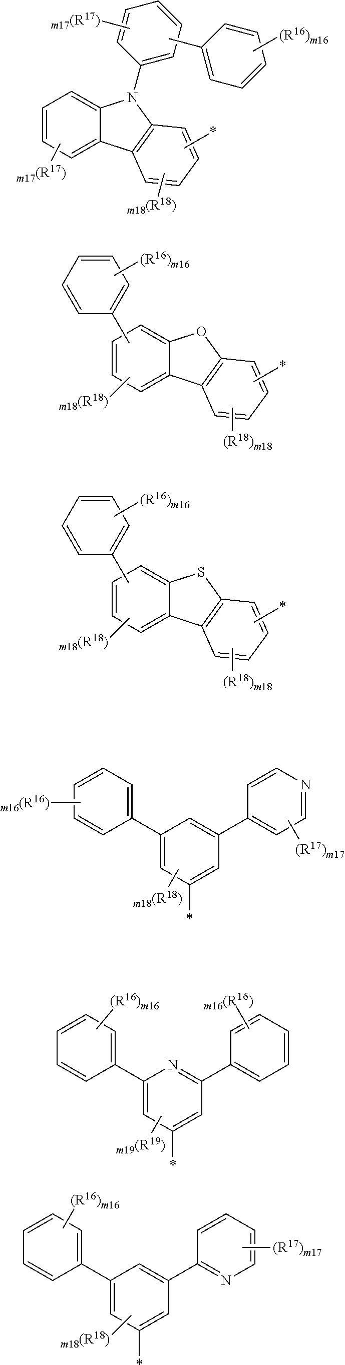
wherein, in Group I,
R¹³ to R⁷ are each independently hydrogen, deuterium, a cyano group, a C1 to C10 alkyl group, or a C6 to C12 aryl group,
m13 is one of integers of 1 to 5,
m14 is one of integers of 1 to 4,
m15 is one of integers of 1 to 3,
m16 is an integer of 1 or 2,
m17 is one of integers of 1 to 7, and
* is a linking point.

10. An organic optoelectronic device, comprising
an anode and a cathode facing each other, and
at least one organic layer between the anode and the cathode,
wherein the organic layer includes the compound for an organic optoelectronic device as claimed in any one of claim 1 to claim 6; or
the composition for an organic optoelectronic device as claimed in any one of claim 7 to claim 11.

11. The organic optoelectronic device as claimed in claim 10, wherein
the organic layer includes a light emitting layer, and
the light emitting layer includes the compound for an organic optoelectronic device or composition for an organic optoelectronic device.

12. A display device comprising the organic optoelectronic device as claimed in claim 10.
